(19) United States
(12) Patent Application Publication
(10) Pub. No.: US 2023/0135635 A1
(43) Pub. Date: May 4, 2023

(54) INHIBITORS OF ULK1/2 AND METHODS OF USING SAME

(71) Applicants: SALK INSTITUTE FOR BIOLOGICAL STUDIES, La Jolla, CA (US); SANFORD BURNHAM PREBYS MEDICAL DISCOVERY INSTITUTE, La Jolla, CA (US)

(72) Inventors: Nicholas D.P. COSFORD, La Jolla, CA (US); Nicole A. BAKAS, La Jolla, CA (US); Mitchell VAMOS, La Jolla, CA (US); Reuben J. SHAW, La Jolla, CA (US); Allison S. LIMPERT, La Jolla, CA (US); Sonja N. BRUN, La Jolla, CA (US)

(21) Appl. No.: 17/799,639

(22) PCT Filed: Feb. 12, 2021
(86) PCT No.: PCT/US2021/018040

§ 371 Date: Aug. 12, 2022

Related U.S. Application Data
(60) Provisional application No. 62/977,040, filed on Feb. 14, 2020.

(51) Int. Cl. C07D 405/14; C07D 401/12; C07D 401/14

(52) U.S. Cl. CPC C07D 405/14; C07D 401/12; C07D 401/14

(57) ABSTRACT

The present disclosure is directed to compounds, compositions, formulations and methods of use thereof in the treatment and prevention of ULK mediated diseases, including cancer.

### CROSS-REFERENCE TO RELATED APPLICATIONS

This application claims priority to U.S. Provisional Application No. 62/977,040 filed Feb. 14, 2020, entitled “Inhibitors of ULK1/2 and Methods of Using Same,” the disclosure of which is hereby incorporated by reference in its entirety.

### STATEMENT AS TO FEDERALLY SPONSORED RESEARCH

This invention was made with government support under T32 grant number 1T32CA211036 awarded by NIH/NCI. The government has certain rights in the invention

### BACKGROUND OF THE INVENTION

Autophagy is a central cellular mechanism for elimination of damaged proteins, protein complexes, and organelles. This conserved process plays crucial roles in the cellular response to nutrient deprivation and other stresses, in addition to being required for proper cellular and tissue homeostasis during embryonic development and in defense against pathogens. Defects in autophagy pathways are associated with certain human pathologies, including infectious diseases, neurodegenerative disorders, and cancer. In spite of these highly conserved fundamental cellular functions, the molecular and biochemical details of how autophagy is initiated for different cargoes, and the coordination of steps starting from autophagosome initiation to ultimate fusion with the lysosome remain poorly understood.

### SUMMARY OF THE INVENTION

Provided herein are inhibitors of unc-51 like autophagy activating kinase (ULK) proteins. In some embodiments, the inhibitors inhibit ULK1. In some embodiments, the inhibitors are specific for ULK1. In some embodiments, the inhibitors inhibit both ULK1 and ULK2. In some instances, the inhibitors provided herein are useful for the treatment of various diseases, including cancer.

In many instances, ULK1 and ULK2 are important proteins that regulate autophagy in mammalian cells. In certain instances, ULK1 and ULK2 are activated under conditions of nutrient deprivation by several upstream signals, which is followed by the initiation of autophagy. The requirement for ULK1 and ULK2 in autophagy initiation has been studied in the context of nutrient deprivation. While ULK1 appears to be the most essential for autophagy, in some instances, ULK1 and ULK2 show high functional redundancy. The kinase domains of ULK1 and ULK2 share 78% sequence homology, suggesting, in some instances, ULK2 may compensate for the loss of ULK1 in some instances. In some instances, nutrient dependent autophagy may only be eliminated if both ULK1 and ULK2 are inhibited. In some instances, inhibition of ULK1 alone is sufficient, e.g. for providing a therapeutic benefit, such as in any method provided herein, for normalizing autophagy in a cancer cell, or other beneficial result. In other instances, inhibition of ULK1 and ULK2 results in a therapeutic benefit, such as tumor shrinkage, tumor cell death, or slowed rate of tumor growth.

In some embodiments, the compounds provided herein are inhibitors of ULK. In some embodiments, the compounds inhibit ULK1. In some embodiments, the compounds are specific for ULK1. In some embodiments, the compounds inhibit both ULK1 and ULK2. In some embodiments, the diseases provided herein are treatable with an inhibitor specific for ULK1. In some instances, ULK2 may compensate for loss of ULK1 function. In some embodiments, the diseases provided herein require treatment with a compound that inhibits both ULK1 and ULK2.

Provided herein in certain embodiments are compounds useful as ULK inhibitors. In some embodiments, the compounds are useful for the treatment of various diseases, including cancer In one aspect, the present disclosure provides a compound having a structure of Formula (IA):

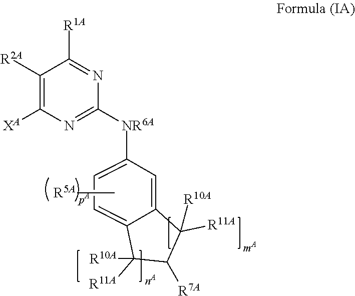

wherein;
R^{1A} is H, halogen, or substituted or unsubstituted alkyl;
R^{2A} is H, haloalkyl, —C(═O)R^{A}, NH₂, or halogen;
X^{A} is —NR^{3A}R^{4A} or —OR^{4A};
R^{3A} is H, substituted or unsubstituted alkyl, or a bond with a substituent on an R^{4A} to form a heterocycle;
R^{4A} is aryl, heteroaryl, cycloalkyl, or heterocycloalkyl;
   wherein the aryl or heteroaryl of R^{4A} is optionally substituted with one or more halogen, —CN, —OR^{A}, —SR^{A}, —NO₂, —NR^{A}R^{A}, —NR^{A}S(═O)₂R^{A}, —C(═O)R^{A}, —OC(═O)R^{A}, —C(═O)C(═O)R^{A}, —C(═O)OR^{A}, —C(═O)NR^{A}OR^{A}, —OC(═O)OR^{A}, —C(═O)NR^{A}R^{A}, —OC(═O)NR^{A}R^{A}, —NR^{A}C(═O)NR^{A}R^{A}, —NR^{A}S(═O)₂NR^{A}R^{A}, —NR^{A}C(═O)R^{A}, —NR^{A}C(═O)OR^{A}, substituted or unsubstituted alkyl, substituted or unsubstituted cycloalkyl, substituted or unsubstituted heterocycloalkyl, substituted or unsubstituted aryl, or substituted or unsubstituted heteroaryl;
   wherein the cycloalkyl or heterocycloalkyl of R^{4A} is optionally substituted with one or more halogen, —CN, —OR^{A}, —SR^{A}, —S(═O)R^{A}, —S(═O)₂R^{A}, —NO₂, —NR^{A}R^{A}, —NR^{A}S(═O)₂R^{A}, —S(═O)₂NR^{A}R^{A}, —C(═O)R^{A}, —OC(═O)R^{A}, —C(═O)C(═O)R^{A}, —C(═O)OR^{A}, —C(═O)NR^{A}OR^{A}, —OC(═O)OR^{A}, —OC(═O)NR^{A}R^{A}, —NR^{A}C(═O)NR^{A}R^{A}, —NR^{A}S(═O)₂NR^{A}R^{A}, —NR^{A}C(═O)R^{A}, —NR^{A}C(═O)OR^{A}, substituted or unsubstituted alkyl, substituted or unsubstituted cycloalkyl, substituted or unsubstituted heterocycloalkyl, substituted or unsubstituted aryl, or substituted or unsubstituted heteroaryl;
each R^{5A} is independently halogen, —CN, —OR^{A}, —SR^{A}, —S(═O)R^{A}, —S(═O)₂R^{A}, —NO₂, —NR^{A}R^{A}, —NR^{A}S(═O)₂R^{A}, —S(═O)₂NR^{A}R^{A}, —C(═O)R^{A}, —OC(═O)R^{A}, —C(═O)C(═O)R^{A}, —C(═O)OR^{A}, —C(═O)NR^{A}OR^{A}, —OC(═O)OR^{A}, —C(═O)NR^{A}R^{A}, —OC(═O)NR^{A}R^{A}, —NR^{A}C(═O)NR^{A}R^{A}, —NR^{A}S(═O)₂NR^{A}R^{A}, —NR^{A}C(═O)R^{A}, —NR^{A}C(═O)OR^{A}, substituted or unsubstituted alkyl, substituted or unsubstituted cycloalkyl, substituted or unsubstituted heterocycloalkyl, substituted or unsubstituted aryl, or substituted or unsubstituted heteroaryl;
R^{6A} is H or substituted or unsubstituted alkyl;
R^{7A} is H, —S(═O)R^{A}, —S(═O)₂R^{A}, —S(═O)₂NR^{A}R^{A}, —C(═O)NR^{A}R^{A}, —C(═O)R^{71A}, —C(═O)C(═O)R^{A}, —C(═O)OR^{72A}, —C(═O)NR^{A}OR^{A}, substituted or unsubstituted alkyl, substituted or unsubstituted cycloalkyl, or substituted or unsubstituted heterocycloalkyl;
each R^{10A} and R^{11A} is independently H, substituted or unsubstituted alkyl, substituted or unsubstituted alkoxy, substituted or unsubstituted cycloalkyl, substituted or unsubstituted heterocycloalkyl, hydroxyl, halogen, or R^{10A} and R^{11A} on the same atom join to form a cycloalkyl or heterocycloalkyl, or R^{10A} and R^{11A} on the same atom are taken together to form an oxo;
R^{71A} is H, —CN, substituted or unsubstituted methyl, substituted or unsubstituted ethyl, substituted or unsubstituted C₃-C₁₀ alkyl, substituted or unsubstituted C₄-C₁₀ cycloalkyl, substituted or unsubstituted heterocycloalkyl, substituted or unsubstituted aryl, or substituted or unsubstituted;
R^{72A} is H, —CN, substituted or unsubstituted methyl, substituted or unsubstituted ethyl, linear C₃-C₅ alkyl, substituted or unsubstituted C₃-C₁₀ cycloalkyl, substituted or unsubstituted heterocycloalkyl, substituted or unsubstituted aryl, or substituted or unsubstituted heteroaryl;
each R^{A} is independently H, substituted or unsubstituted alkyl, substituted or unsubstituted heteroalkyl, substituted or unsubstituted aryl, or substituted or unsubstituted heteroaryl;
n^{A} is 1 or 2;
m^{A} is 1 or 2; wherein the sum of n and m is 2 or 3;
p^{A} is an integer from 0-3; and
the nitrogen in the fused ring system is optionally quaternized with C₁-C₆ alkyl,
or pharmaceutically acceptable salt thereof.

In some embodiments, R^{1A} is H, halogen, or C₁-C₆ alkyl. In some embodiments, R^{1A} is H or fluorine. In some embodiments, R^{1A} is H.

In some embodiments, R^{2A} is H, C₁-C₆ haloalkyl, or halogen. In some embodiments, R^{2A} is —CF₃, or halogen. In some embodiments, R^{2A} is —CF₃, —Cl or —Br. In some embodiments, R^{2A} is —CF₃. In some embodiments, R^{2A} is Br.

In some embodiments, X^{A} is —NR^{3A}R^{4A}. In some embodiments, R^{3A} is H or C₁-C₆ alkyl. In some embodiments, R^{3A} is H, or —CH₃. In some embodiments, R^{3A} is H.

In some embodiments, R^{4A} is aryl or heteroaryl, wherein the aryl or heteroaryl of R^{4A} is optionally substituted with one or more halogen, —CN, —OR^{A}, —SR^{A}, —NO₂, —NR^{A}R^{A}, —NR^{A}S(═O)₂R^{A}, —C(═O)R^{A}, —OC(═O)R^{A}, —C(═O)C(═O)R^{A}, —C(═O)OR^{A}, —C(═O)NR^{A}OR^{A}, —OC(═O)OR^{A}, —C(═O)NR^{A}R^{A}, —OC(═O)NR^{A}R^{A}, —NR^{A}C(═O)NR^{A}R^{A}, —NR^{A}S(═O)₂NR^{A}R^{A}, —NR^{A}C(═O)R^{A}, —NR^{A}C(═O)OR^{A}, substituted or unsubstituted alkyl, or substituted or unsubstituted cycloalkyl. In some embodiments, R^{4A} is aryl or heteroaryl wherein the aryl or heteroaryl of R^{4A} is optionally substituted with one or more halogen, —CN, —OR^{A}, —NR^{A}R^{A}, —C(═O)R^{A}, —OC(═O)R^{A}, —C(═O)C(═O)R^{A}, —C(═O)OR^{A}, —C(═O)NR^{A}OR^{A}, —OC(═O)OR^{A}, —C(═O)NR^{A}R^{A}, —OC(═O)NR^{A}R^{A}, —NR^{A}C(═O)NR^{A}R^{A}, —NR^{A}C(═O)R^{A}, —NR^{A}C(═O)OR^{A}, substituted or unsubstituted alkyl, or substituted or unsubstituted cycloalkyl. In some embodiments, R^{4A} is aryl or heteroaryl. In some embodiments, the aryl or heteroaryl of R^{4A} is optionally substituted with one or more halogen, —CN, —OR^{A}, —NR^{A}R^{A}, —C(═O)R^{A}, —OC(═O)R^{A}, —C(═O)OR^{A}, —C(═O)NR^{A}OR^{A}, —C(═O)NR^{A}R^{A}, —NR^{A}C(═O)R^{A}, substituted or unsubstituted alkyl, or substituted or unsubstituted cycloalkyl. In some embodiments, R^{4A} is aryl or heteroaryl wherein the aryl or heteroaryl of R^{4A} is optionally substituted with one or more halogen, —OR^{A}, —C(═O)R^{A}, —C(═O)NR^{A}R^{A}, —NR^{A}C(═O)R^{A}, or substituted or unsubstituted alkyl. In some embodiments, R^{4A} is aryl or heteroaryl wherein the aryl or heteroaryl of R^{4A} is optionally substituted with one or more halogen, —OR^{A}, —C(═O)NR^{A}R^{A}, or substituted or unsubstituted alkyl. In some embodiments, R^{4A} is 6-membered aryl or heteroaryl. In some embodiments, R^{4A} is phenyl, pyridyl, or pyrimidinyl. In some embodiments, R^{4A} is phenyl. In some embodiments, R^{4A} is phenyl substituted with

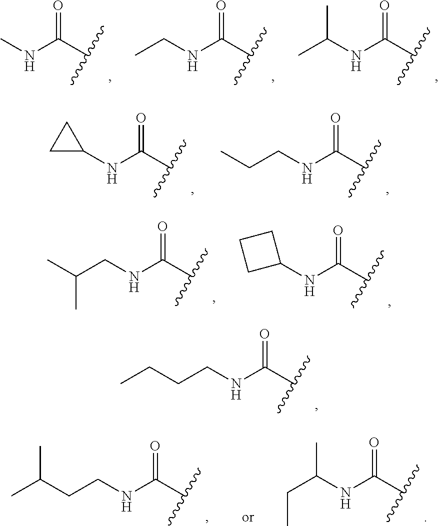

In some embodiments, R^{4A} is

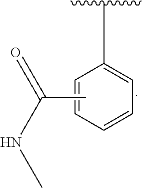

In some embodiments, R^{4A} is

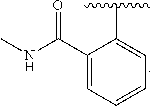 [SMILES: *c1ccccc1C(=O)NC]

In some embodiments, R^{4A} is cycloalkyl, or heterocycloalkyl optionally substituted with one or more halogen, —CN, —OR^{A}, —NR^{A}R^{A}, —C(═O)R^{A}, —OC(═O)R^{A}, —C(═O)C(═O)R^{A}, —C(═O)OR^{A}, —C(═O)NR^{A}OR^{A}, —OC(═O)OR^{A}, —C(═O)NR^{A}R^{A}, —OC(═O)NR^{A}R^{A}, —NR^{A}C(═O)NR^{A}R^{A}, —NR^{A}C(═O)R^{A}, —NR^{A}C(═O)OR^{A}, substituted or unsubstituted alkyl, or substituted or unsubstituted cycloalkyl. In some embodiments, R^{4A} is cycloalkyl optionally substituted with one or more halogen, —CN, —OR^{A}, —NR^{A}R^{A}, —C(═O)R^{A}, —OC(═O)R^{A}, —C(═O)C(═O)R^{A}, —C(═O)OR^{A}, —C(═O)NR^{A}OR^{A}, —OC(═O)OR^{A}, —C(═O)NR^{A}R^{A}, —OC(═O)NR^{A}R^{A}, —NR^{A}C(═O)NR^{A}R^{A}, —NR^{A}C(═O)R^{A}, —NR^{A}C(═O)OR^{A}, substituted or unsubstituted alkyl, or substituted or unsubstituted cycloalkyl. In some embodiments, R^{4A} is cyclopropyl, cyclobutyl, cyclopentyl, or cyclohexyl optionally substituted with one or more halogen, —CN, —OR^{A}, —NR^{A}R^{A}, —C(═O)R^{A}, —OC(═O)R^{A}, —C(═O)C(═O)R^{A}, —C(═O)OR^{A}, —C(═O)NR^{A}OR^{A}, —OC(═O)OR^{A}, —C(═O)NR^{A}R^{A}, —OC(═O)NR^{A}R^{A}, —NR^{A}C(═O)NR^{A}R^{A}, —NR^{A}C(═O)R^{A}, —NR^{A}C(═O)OR^{A}, substituted or unsubstituted alkyl, or substituted or unsubstituted cycloalkyl. In some embodiments, R^{4A} is cyclopropyl optionally substituted with one or more halogen, —CN, —OR^{A}, —NR^{A}R^{A}, —C(═O)R^{A}, —OC(═O)R^{A}, —C(═O)C(═O)R^{A}, —C(═O)OR^{A}, —C(═O)NR^{A}OR^{A}, —OC(═O)OR^{A}, —C(═O)NR^{A}R^{A}, —OC(═O)NR^{A}R^{A}, —NR^{A}C(═O)NR^{A}R^{A}, —NR^{A}C(═O)R^{A}, —NR^{A}C(═O)OR^{A}, substituted or unsubstituted alkyl, or substituted or unsubstituted cycloalkyl. In some embodiments, R^{4A} is cyclopropyl optionally substituted with one or more halogen, —OR^{A}, —C(═O)R^{A}, —C(═O)OR^{A}, —C(═O)NR^{A}R^{A}, or substituted or unsubstituted alkyl. In some embodiments, R^{4A} is cyclopropyl optionally substituted with one or more-OR^{A} or substituted or unsubstituted alkyl. In some embodiments, R^{4A} is cyclopropyl optionally substituted with OH or C₁-C₆ alkyl. In some embodiments, R^{4A} is unsubstituted cyclopropyl.

In some embodiments, each R^{5A} is independently halogen, —CN, —OR^{A}, —NR^{A}R^{A}, —C(═O)R^{A}, —OC(═O)R^{A}, —C(═O)OR^{A}, —C(═O)NR^{A}OR^{A}, —OC(═O)OR^{A}, —C(═O)NR^{A}R^{A}, —OC(═O)NR^{A}R^{A}, —NR^{A}C(═O)NR^{A}R^{A}, —NR^{A}S(═O)₂NR^{A}R^{A}, —NR^{A}C(═O)R^{A}, —NR^{A}C(═O)OR^{A}, substituted or unsubstituted alkyl, substituted or unsubstituted cycloalkyl, substituted or unsubstituted heterocycloalkyl, substituted or unsubstituted aryl, or substituted or unsubstituted heteroaryl. In some embodiments, each R^{5A} is independently halogen, —CN, —OR^{A}, —NR^{A}R^{A}, —C(═O)R^{A}, —OC(═O)R^{A}, —C(═O)OR^{A}, substituted or unsubstituted alkyl, or substituted or unsubstituted cycloalkyl. In some embodiments, each R^{5A} is independently halogen, —OR^{A}, —NR^{A}R^{A}, or unsubstituted C₁-C₆ alkyl.

In some embodiments, p^{A} is 0 or 1. In some embodiments, p^{A} is 0.

In some embodiments, R^{6A} is H or —CH₃. In some embodiments, R^{6A} is H.

In some embodiments, R^{7A} is —S(═O)R^{A}, —S(═O)₂R^{A}, —S(═O)₂NR^{A}R^{A}, —C(═O)NR^{A}R^{A}, —C(═O)R^{71A}, —C(═O)C(═O)R^{A}, —C(═O)OR^{72A}, —C(═O)NR^{A}OR^{A}, substituted or unsubstituted alkyl, substituted or unsubstituted cycloalkyl, or substituted or unsubstituted heterocycloalkyl. In some embodiments, R^{7A} is —S(═O)₂R^{A}, —S(═O)₂NR^{A}R^{A}, —C(═O)NR^{A}R^{A}, —C(═O)R^{71A}, —C(═O)OR^{72A}, —C(═O)NR^{A}OR^{A}, substituted or unsubstituted C₁-C₆ alkyl, substituted or unsubstituted C₃-C₈ cycloalkyl, or substituted or unsubstituted C₂-C₈ heterocycloalkyl. In some embodiments, R^{7A} is —S(═O)₂R^{A} S(═O)₂NR^{A}R^{A}, —C(═O)R^{71A}, substituted or unsubstituted C₁-C₆ alkyl, or substituted or unsubstituted C₂-C₈ heterocycloalkyl. In some embodiments, R^{7A} is —C(═O)R^{71A} or substituted or unsubstituted C₁-C₆ alkyl. In some embodiments, R^{7A} is C₁-C₆ alkyl optionally substituted with hydroxyl or alkoxy. In some embodiments, R^{7A} is H. In some embodiments, R^{7A} is

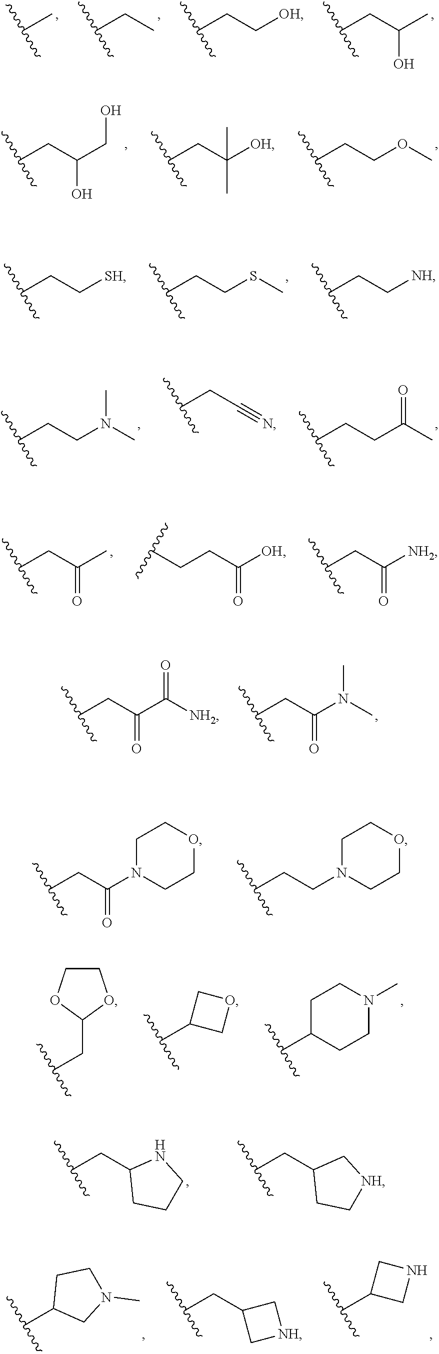

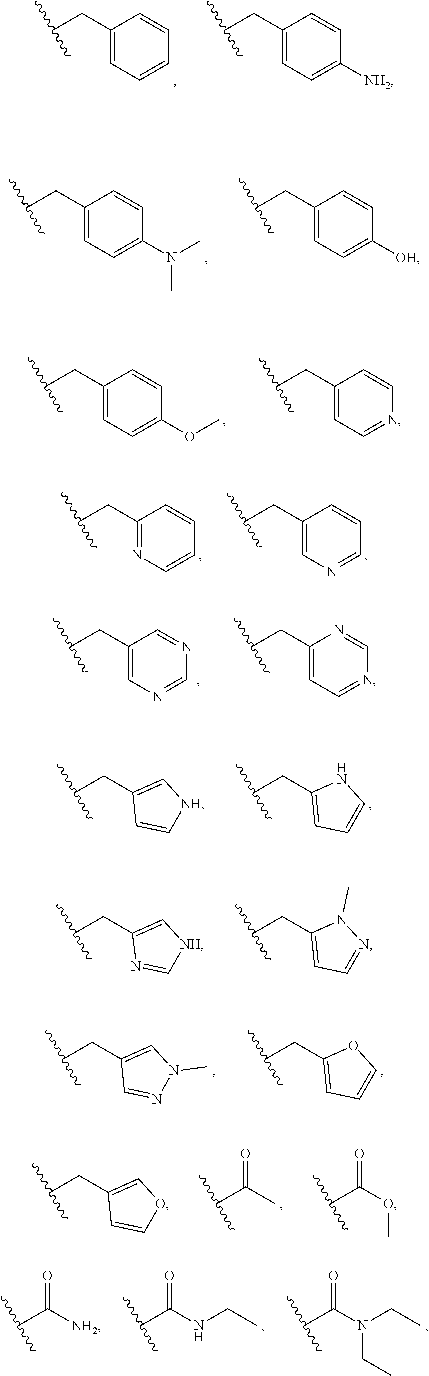

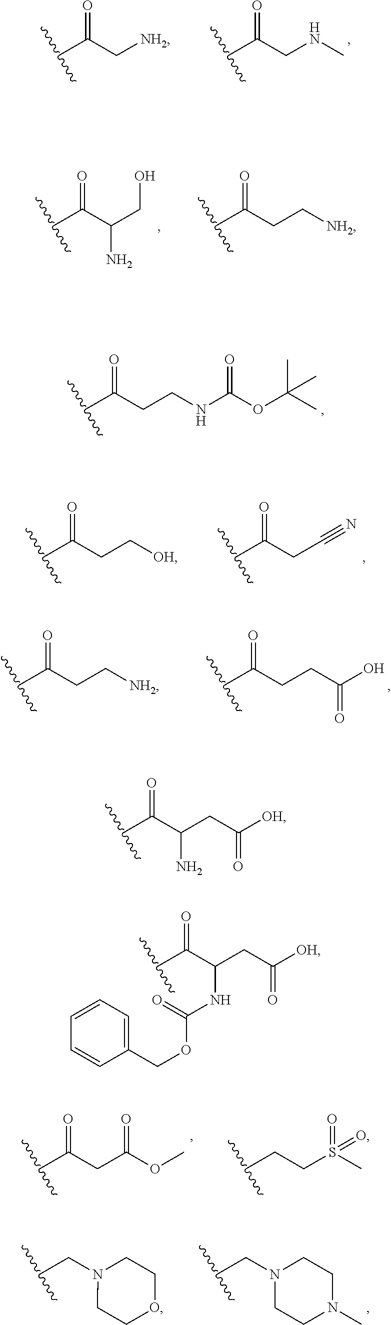

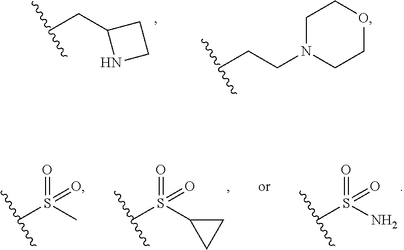

In some embodiments, each R^{10A} and R^{11A} is independently H or substituted or unsubstituted alkyl, or R^{10A} and R^{11A} on the same atom join to form a cycloalkyl, or R^{10A} and R^{11A} on the same atom are taken together to form an oxo. In some embodiments, each R^{10A} and R^{11A} is independently H or substituted or unsubstituted alkyl, or R^{10A} and R^{11A} on the same atom are taken together to form an oxo. In some embodiments, each R^{10A} and R^{11A} is independently H.

In some embodiments, n^{A} is 1 and m^{A} is 1. In some embodiments, n^{A} is 1 and m^{A} is 2. In some embodiments, n^{A} is 2 and m^{A} is 1.

In one aspect, provided herein, is a compound having a structure of Formula (IIB):

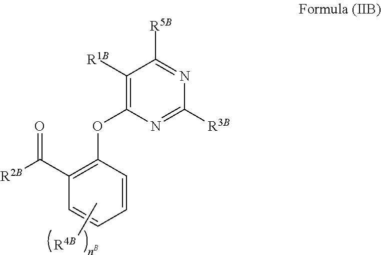

wherein:
R^{1B} is H, halogen, substituted or unsubstituted alkyl, or substituted or unsubstituted haloalkyl;
R^{2B} is substituted C₂ alkyl, substituted or unsubstituted C₃-C₁₀ alkyl, —NR^{21B}R^{22B}, or —OR^{23B};
R^{3B} is —OR^{31B}, —SR^{31B}, or —NR^{32B}R^{33B};
each R^{4B} is independently halogen, —CN, —OR^{B}, —SR^{B}, —S(═O)R^{B}, —S(═O)₂R^{B}, —NO₂, —NR^{B}R^{B}, —NR^{B}S(═O)₂R^{B}, —S(═O)₂NR^{B}R^{B}, —C(═O)R^{B}, —OC(═O)R^{B}, —C(═O)C(═O)R^{B}, —C(═O)OR^{B}, —C(═O)NR^{B}OR^{B}, —OC(═O)OR^{B}, —C(═O)NR^{B}R^{B}, —OC(═O)NR^{B}R^{B}, —NR^{B}C(═O)NR^{B}R^{B}, —NR^{B}S(═O)₂NR^{B}R^{B}, —NR^{B}C(═O)R^{B}, —NR^{B}C(═O)OR^{B}, substituted or unsubstituted alkyl, substituted or unsubstituted cycloalkyl, substituted or unsubstituted heterocycloalkyl, substituted or unsubstituted aryl, or substituted or unsubstituted heteroaryl;
R^{5B} is H or halogen;
R^{21B} is —OR^{26B}, NR^{27B}R^{28B}, substituted methyl, or substituted or unsubstituted C₂-C₁₀ alkyl;
   R^{22B} is H or substituted or unsubstituted alkyl; or
      R^{21B} and R^{22B} are taken together with the nitrogen atom to which they are attached to form a substituted or unsubstituted heterocycloalkyl containing at least one additional heteroatom selected from the group consisting of O, N, and S;
   R^{23B} is H or substituted or unsubstituted alkyl;
   R^{26B} is H or substituted or unsubstituted alkyl;
   R^{27B} and R^{28B} are each independently H or substituted or unsubstituted alkyl; or
      R^{27B} and R^{28B} are taken together with the nitrogen atom to which they are attached to form a substituted or unsubstituted heterocycloalkyl;
   R^{31B} is substituted or unsubstituted aryl or substituted or unsubstituted heteroaryl;
   R^{32B} is substituted or unsubstituted aryl or substituted or unsubstituted heteroaryl;
   R^{33B} is H or substituted or unsubstituted alkyl; or
      R^{32B} and R^{33B} are taken together with the nitrogen atom to which they are attached to form a substituted or unsubstituted heterocycloalkyl;
   each R^{B} is independently H, substituted or unsubstituted alkyl, substituted or unsubstituted heteroalkyl, substituted or unsubstituted aryl, or substituted or unsubstituted heteroaryl; and
   n^{B} is an integer from 0-4;
   or a pharmaceutically acceptable salt thereof.

In some embodiments, R^{1B} is halogen or C₁-C₆ haloalkyl. In some embodiments, R^{1B} is Cl, Br, or —CF₃. In some embodiments, R^{1B} is —CF₃. In some embodiments R^{1B} is Br.

In some embodiments, R^{2B} is —NR^{21B}R^{22B} or —OR^{23B}. In some embodiments, R^{2B} is —NR^{21B}R^{22B}. In some embodiments, R^{21B} is substituted methyl or substituted or unsubstituted C₂-C₆ alkyl. In some embodiments, R^{21B} is substituted methyl or substituted C₂-C₄ alkyl. In some embodiments, R^{21B} is

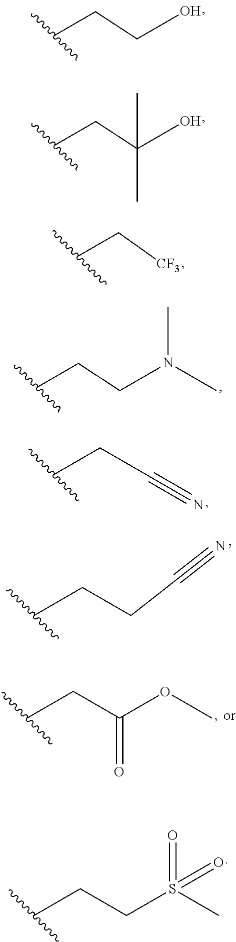

In some embodiments, R^{22B} is H or —CH₃. In some embodiments, R^{22B} is —CH₃. In some embodiments, R^{22B} is H.

In some embodiments, R^{2B} is —OR^{23B}. In some embodiments R^{23B} is H or —CH₃. In some embodiments, R^{23B} is —CH₃. In some embodiments, R^{3B} is —NR^{32B}R^{33B}. In some embodiments, R^{32B} is substituted or unsubstituted aryl or substituted or unsubstituted heteroaryl, wherein the aryl or heteroaryl is

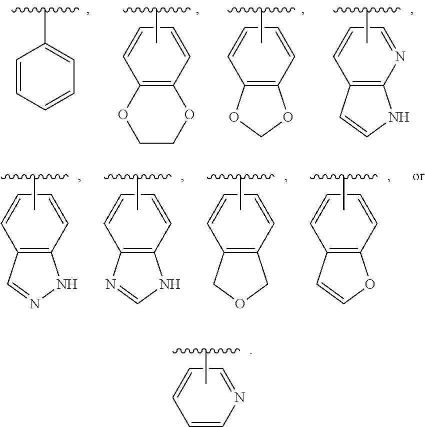

In some embodiments, R^{32B} is substituted or unsubstituted aryl or substituted or unsubstituted heteroaryl, wherein the aryl or heteroaryl is

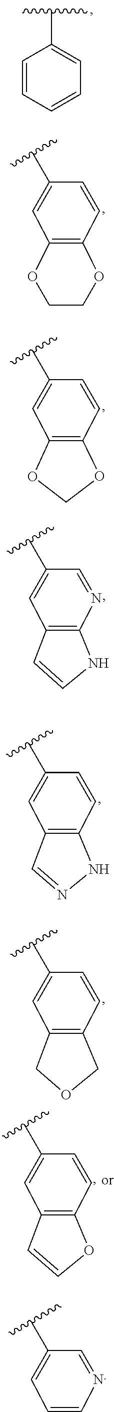

In some embodiments, R^{32B} is aryl optionally substituted with one or more halogen, —CN, —OR^{B}, —SR^{B}, —S(═O)R^{B}, —S(═O)₂R^{B}, —NO₂, —NR^{B}R^{B}, —NR^{B}S(═O)₂R^{B}, —S(═O)₂NR^{B}R^{B}, —C(═O)R^{B}, —OC(═O)R^{B}, —C(═O)C(═O)R^{B}, —C(═O)OR^{B}, —C(═O)NR^{B}OR^{B}, —OC(═O)OR^{B}, —C(═O)NR^{B}R^{B}, —OC(═O)NR^{B}R^{B}, —NR^{B}C(═O)NR^{B}R^{B}, —NR^{B}S(═O)₂NR^{B}R^{B}, —NR^{B}C(═O)R^{B}, —NR^{B}C(═O)OR^{B}, substituted or unsubstituted alkyl, substituted or unsubstituted cycloalkyl, or substituted or unsubstituted heterocycloalkyl. In some embodiments, R^{32B} is phenyl optionally substituted with one or more halogen, —CN, —OR^{B}, —SR^{B}, —S(═O)R^{B}, —S(═O)₂R^{B}, —NO₂, —NR^{B}R^{B}, —NR^{B}S(═O)₂R^{B}, —S(═O)₂NR^{B}R^{B}, —C(═O)R^{B}, —OC(═O)R^{B}, —C(═O)C(═O)R^{B}, —C(═O)OR^{B}, —C(═O)NR^{B}OR^{B}, —OC(═O)OR^{B}, —C(═O)NR^{B}R^{B}, —OC(═O)NR^{B}R^{B}, —NR^{B}C(═O)NR^{B}R^{B}, —NR^{B}S(═O)₂NR^{B}R^{B}, —NR^{B}C(═O)R^{B}, —NR^{B}C(═O)OR^{B}, substituted or unsubstituted alkyl, substituted or unsubstituted cycloalkyl, or substituted or unsubstituted heterocycloalkyl. In some embodiments, R^{32B} is

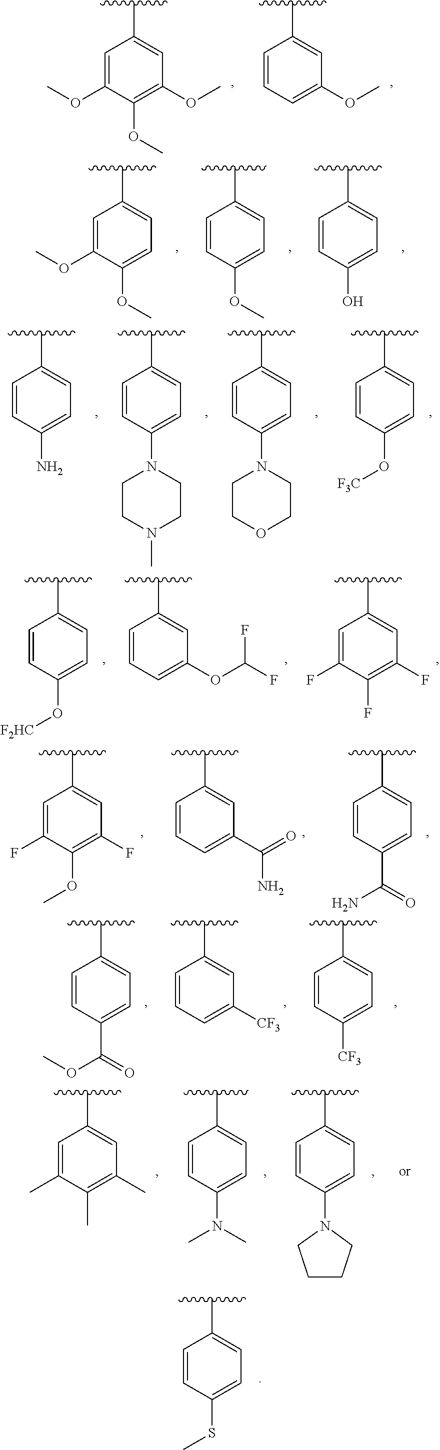

In some embodiments, R^{33B} H or C₁-C₆ alkyl. In some embodiments, R^{33B} is H or —CH₃. In some embodiments, R^{33B} is H. In some embodiments, R^{4B} is independently halogen, —CN, —OR^{B}, —C(═O)R^{B}, —OC(═O)R^{B}, —OC(═O)OR^{B}, —C(═O)NR^{B}R^{B}, —OC(═O)NR^{B}R^{B}, C₁-C₆ alkyl, or C₁-C₆ haloalkyl. In some embodiments, each R^{4B} is independently halogen, —OR^{B}, or C₁-C₆ alkyl. In some embodiments, each R^{4B} is independently —OR^{B}.

In some embodiments, n^{B} is 0, 1, or 2. In some embodiments, n^{B} is 0 or 1. In some embodiments, n^{B} is 0.

In some embodiments, R^{5B} is H or F. In some embodiments, R^{5B} is H.

In one aspect, provided herein, is a compound of Formula (IIIC):

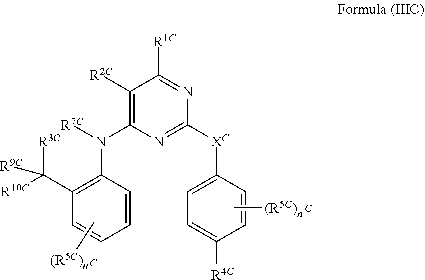

wherein:
R^{1C} is H, substituted or unsubstituted alkyl, or halogen;
R^{2C} is H, halogen, substituted or unsubstituted alkyl, or substituted or unsubstituted haloalkyl;
R^{3C} is —NR^{C}R^{C}, —OR^{C}, —O(C═O)R^{C}, —O(C═O)NR^{C}R^{C}, —NR^{C}(C═O)NR^{C}R^{C}, —NR^{C}(C═O)R^{C}, or —SR^{C};
R^{4C} is —NR^{41C}R^{42C}, —OR^{43C}, —C(═O)OR^{44C}, —C(═O)NR^{C}R^{C}, or —NR^{C}C(═O)R^{C};
each R^{5C} and R^{6C} is independently halogen, —CN, —OR^{C}, —SR^{C}, —S(═O)R^{C}, —S(═O)₂R^{C}, —NO₂, —NR^{C}R^{C}, —NR^{C}S(═O)₂R^{C}, —S(═O)₂NR^{C}R^{C}, —C(═O)R^{C}, —OC(═O)R^{C}, —C(═O)C(═O)R^{C}, —C(═O)OR^{C}, —C(═O)NR^{C}OR^{C}, —OC(═O)OR^{C}, —C(═O)NR^{C}R^{C}, —OC(═O)NR^{C}R^{C}, —NR^{C}C(═O)NR^{C}R^{C}, —NR^{C}S(═O)₂NR^{C}R^{C}, —NR^{C}C(═O)R^{C}, —NR^{C}C(═O)OR^{C}, substituted or unsubstituted alkyl, substituted or unsubstituted cycloalkyl, substituted or unsubstituted heterocycloalkyl, substituted or unsubstituted aryl, or substituted or unsubstituted heteroaryl;
R^{7C} is H or substituted or unsubstituted alkyl;
X^{C} is —O— or —NR^{8C}—;
R^{8C} is H or substituted or unsubstituted alkyl;
R^{9C} and R^{10C} are each independently H, substituted or unsubstituted alkyl, substituted or unsubstituted cycloalkyl, substituted or unsubstituted heterocycloalkyl, substituted or unsubstituted aryl, or substituted or unsubstituted heteroaryl;
or R^{9C} and R^{10C} are taken together with the carbon atom to which they are attached to form a substituted or unsubstituted cycloalkyl or substituted or unsubstituted heterocycloalkyl;
R^{41C} and R^{42C} are each independently hydrogen, alkyl, cycloalkyl, heterocycloalkyl, aryl, or heteroaryl; wherein the alkyl, cycloalkyl, heterocycloalkyl, aryl, and heteroaryl are independently optionally substituted with one or more R^{45C}.
or R^{41C} and R^{42C} are taken together with the nitrogen atom to which they are attached to form a substituted or unsubstituted heterocycloalkyl;
R^{43C} is hydrogen, —CN, alkyl, cycloalkyl, heterocycloalkyl, aryl, or heteroaryl; wherein the alkyl, cycloalkyl, heterocycloalkyl, aryl, and heteroaryl are independently optionally substituted with one or more R^{45C}.
R^{44C} is substituted or unsubstituted alkyl, substituted or unsubstituted cycloalkyl, substituted or unsubstituted heterocycloalkyl, substituted or unsubstituted aryl, or substituted or unsubstituted heteroaryl;
each R^{45C} is independently oxo, halogen, —CN, —OR^{C}, —S(═O)₂R^{C}, —S(═O)₂NR^{C}R^{C}, —C(═O)R^{C}, —OC(═O)R^{C}, —C(═O)OR^{C}, —OC(═O)OR^{C}, —C(═O)NR^{C}R^{C}, —OC(═O)NR^{C}R^{C}, —NR^{C}C(═O)NR^{C}R^{C}, —NR^{C}C(═O)R^{C}, alkyl, haloalkyl, or hydroxyalkyl;
each R^{C} is independently H, substituted or unsubstituted alkyl, substituted or unsubstituted heteroalkyl, substituted or unsubstituted aryl, or substituted or unsubstituted heteroaryl;
n^{C} is an integer from 0-4;
m^{C} is an integer from 0-4; and
with the proviso that when R^{4C} is —OMe and R^{2C} is halogen, then R^{3C} is not OH,
or pharmaceutically acceptable salt thereof.

In some embodiments, R^{1C} is H or halogen. In some embodiments, R^{1C} is H or F. In some embodiments, R^{1C} is H.

In some embodiments, R^{2C} is halogen or C₁-C₆ haloalkyl. In some embodiments, R^{2C} is Br, Cl, or —CF₃. In some embodiments, R^{2C} is Br.

In some embodiments, R^{3C} is —NR^{C}R^{C}, —OR^{C}, —O(C═O)R^{C}, or —O(C═O)NR^{C}R^{C}. In some embodiments, R^{3C} is —NR^{C}R^{C} or —O(C═O)NR^{C}R^{C}. In some embodiments, R^{3C} is —NR^{C}R^{C} and each R^{C} is independently hydrogen, substituted or unsubstituted C₁-C₆ alkyl, or substituted or unsubstituted cycloalkyl or both R^{C}s are taken together with the nitrogen atom to which they are attached to form a substituted or unsubstituted heterocycloalkyl. In some embodiments, R^{3C} is —NR^{C}R^{C} each R^{C} is independently selected from H,

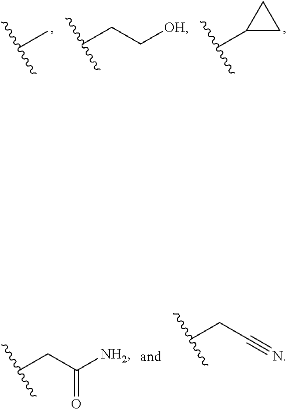

In some embodiments, R^{3C} is —NR^{C}R^{C} and both R^{C}s are taken together to form a heterocycloalkyl selected from

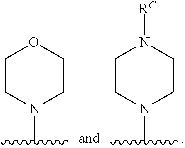

In some embodiments, R^{3C} is selected from

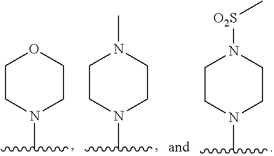

In some embodiments, R^{3C} is —OR^{C} or —O(C═O)R^{C}. In some embodiments, R^{3C} is OR^{C} and the R^{C} of R^{3C} is hydrogen, substituted or unsubstituted C₁-C₆ alkyl, substituted or unsubstituted cycloalkyl, substituted or unsubstituted heterocycloalkyl, substituted or unsubstituted aryl, or substituted or unsubstituted heteroaryl. In some embodiments, R^{3C} is —OR^{C} or —O(C═O)R^{C} and the R^{C} of R^{3C} is

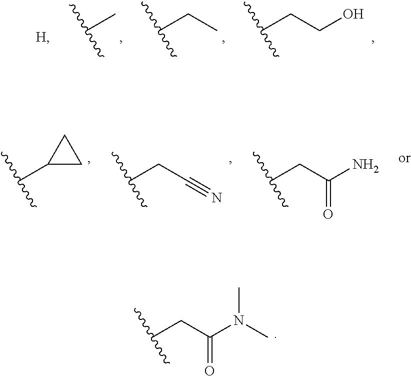

In some embodiments, R^{3C} is —OR^{C} or —O(C═O)R^{C} and the R^{C} of R^{3C} is

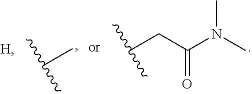

In some embodiments, R^{3C} is

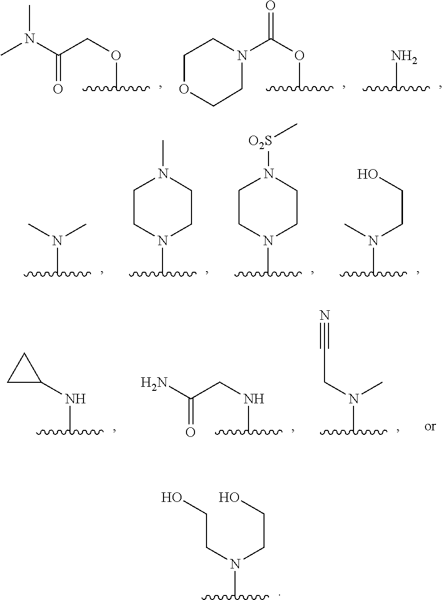

In some embodiments, R^{4C} is —NR^{41C}R^{42C}, —OR^{43C}, —C(═O)NR^{C}R^{C}, or —NR^{C}C(═O)R^{C}.

In some embodiments, R^{4C} is —NR^{41C}R^{42C}. In some embodiments, R^{41C} and R^{42C} are each independently hydrogen, alkyl, or cycloalkyl, wherein the alkyl or cycloalkyl is optionally substituted with one or more R^{45C} or R^{41C} and R^{42C} are taken together with the nitrogen atom to which they are attached to form a substituted or unsubstituted heterocycloalkyl. In some embodiments, R^{41C} and R^{42C} is independently H,

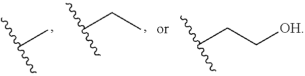

In some embodiments, R^{41C} and R^{42C} are taken together with the nitrogen atom to which they are attached to form a heterocycloalkyl, wherein the heterocycloalkyl is

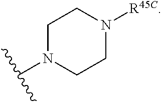

In some embodiments, R^{41C} and R^{42C} are taken together with the nitrogen atom to which they are attached to form

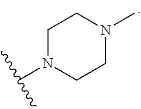 [SMILES: *N1CCN(C)CC1]

In some embodiments, R^{4C} is —OR^{43C}. In some embodiments, R^{43C} is hydrogen or C₁-C₆alkyl optionally substituted with one or more R^{45C}. In some embodiments, R^{43C} is H, —CH₃, —CH₂CH₃, CH₂F, —CHF₂, or CF₃.

In some embodiments, R^{4C} is —C(═O)NR^{41C}R^{42C}. In some embodiments, R^{41C} and R^{42C} are each independently hydrogen, substituted or unsubstituted C₁-C₆ alkyl, or cycloalkyl; wherein each alkyl or cycloalkyl is independently optionally substituted with one or more R^{45C}. In some embodiments, R^{41C} and R^{42C} are each independently H, —CH₃, or —CH₂CH₃.

In some embodiments, R^{4C} is —NR^{C}C(═O)R^{C}. In some embodiments, R^{4C} is —NR^{C}C(═O)R^{C} and one R^{C} of R^{4C} is H or —CH₃; and the other R^{C} of R^{4C} is substituted or unsubstituted alkyl or substituted or unsubstituted cycloalkyl. In some embodiments, R^{4C} is

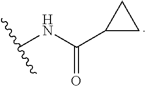 [SMILES: *NC(=O)C1CC1]

In some embodiments, R^{4C} is

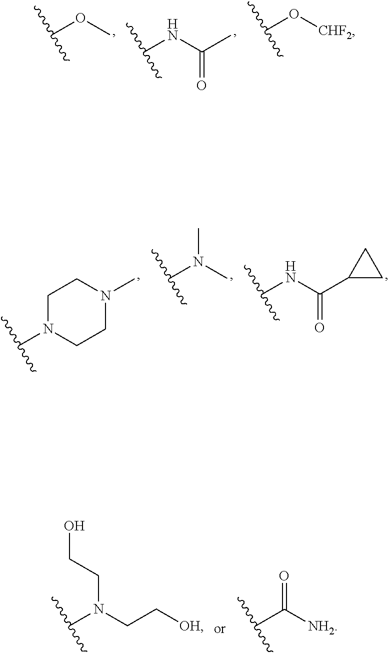

In some embodiments, each R^{5C} is independently halogen, —CN, —OR^{C}, —NR^{C}R^{C}, —NR^{C}S(═O)₂R^{C}, —S(═O)₂NR^{C}R^{C}, —OC(═O)OR^{C}, —C(═O)NR^{C}R^{C}, —OC(═O)NR^{C}R^{C}, —NR^{C}C(═O)R^{C}, or substituted or unsubstituted alkyl. In some embodiments, each R^{5C} is independently halogen, —CN, —OR^{C}, or substituted or unsubstituted alkyl. In some embodiments, each R^{5C} is independently halogen or —OR^{C}. In some embodiments, each R^{5C} is independently —O(C₁-C₆ alkyl). In some embodiments, each R^{5C} is independently —OCH₃.

In some embodiments, n^{C} is 0, 1, or 2. In some embodiments, n^{C} is 0 or 1. In some embodiments, n^{C} is 0.

In some embodiments, each R^{6C} is independently halogen, —CN, —OR^{C}, —C(═O)R^{C}, —OC(═O)R^{C}—C(═O)OR^{C}, —OC(═O)OR^{C}, —OC(═O)NR^{C}R^{C}, or substituted or unsubstituted alkyl. In some embodiments, each R^{6C} is independently halogen or —OR^{C}. In some embodiments, each R^{6C} is —O(C₁-C₆ alkyl).

In some embodiments, m^{C} is 0, 1, or 2. In some embodiments, m^{C} is 2. In some embodiments, m^{C} is 2 and R^{6C} is —OCH₃. In some embodiments, m^{C} is 0 or 1. In some embodiments, m^{C} is 0.

In some embodiments, R^{7C} is H or —CH₃. In some embodiments, R^{7C} is H.

In some embodiments, X^{C} is —NR⁸—. In some embodiments, R^{8C} is H or —CH₃. In some embodiments, R^{8C} is H.

In some embodiments, R^{9C} and R^{10C} are each independently H or substituted or unsubstituted alkyl. In some embodiments, R^{9C} and R^{10C} are each independently H or C₁-C₆ alkyl. In some embodiments, R^{9C} is —CH₃ and R^{10C} is H. In some embodiments, R^{9C} and R^{10C} are each H.

In one aspect, provided herein, is a compound having a structure of Formula (IVD)

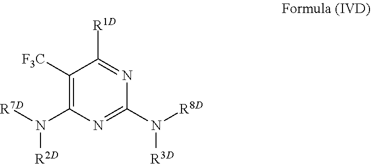

wherein:
R^{1D} is H or halogen;
R^{2D} is

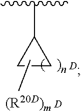

m^{D} is an integer from 1 to 3;
n^{D} is an integer from 1 to 6;
R^{3D} is

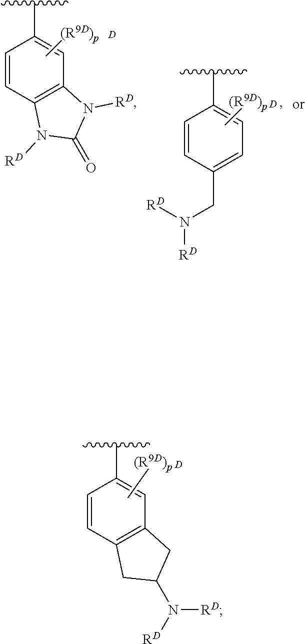

R^{7D} and R^{8D} are each independently H or substituted or unsubstituted alkyl;
each R^{9D} is independently halogen, —CN, —OR^{D}, S(═O)₂R^{D}, —NR^{D}R^{D}, —S(═O)₂NR^{D}R^{D}, —C(═O)R^{D}, —OC(═O)R^{D}, —C(═O)OR^{D}, —OC(═O)OR^{D}, —C(═O)NR^{D}R^{D}, —OC(═O)NR^{D}R^{D}, —NR^{D}C(═O)NR^{D}R^{D}, —NR^{D}C(═O)R^{D}, alkyl, haloalkyl, or hydroxyalkyl;
p^{D} is an integer from 0 to 2;
each R^{20D} is independently halogen, —CN, —OR^{D}, S(═O)₂R^{D}, —S(═O)₂NR^{D}R^{D}, —C(═O)R^{D}, —OC(═O)R^{D}, —C(═O)OR^{D}, —OC(═O)OR^{D}, —OC(═O)NR^{D}R^{D}, —NR^{D}C(═O)NR^{D}R^{D}, —NR^{D}C(═O)R^{D}, alkyl, haloalkyl, or hydroxyalkyl; and
each R^{D} is independently H, substituted or unsubstituted alkyl, substituted or unsubstituted heteroalkyl, substituted or unsubstituted aryl, or substituted or unsubstituted heteroaryl
or a pharmaceutically acceptable salt thereof.

In some embodiments, R^{1D} is H or fluorine. In some embodiments, R^{1D} is H.

In some embodiments, R^{2D} is

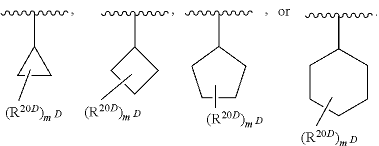

In some embodiments, R² is

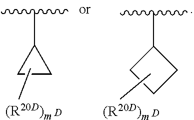

In some embodiments, R² is

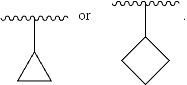

In some embodiments, each R^{20D} is independently halogen, —CN, —OR^{D}, —C(═O)R^{D}, —OC(═O)R^{D}, —OC(═O)OR^{D}, —OC(═O)NR^{D}R^{D}, —NR^{D}C(═O)R^{D}, or C₁-C₆ alkyl. In some embodiments, each R^{20D} is independently halogen, —CN, —OR^{D}, or C₁-C₆ alkyl.

In some embodiments, m^{D} is 0 or 1. In some embodiments, m^{D} is 0.

In some embodiments, R^{3D} is

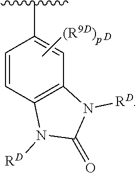

In some embodiments, each R^{D} is independently H or —CH₃. In some embodiments, each R^{D} is independently H.

In some embodiments, R^{3D} is

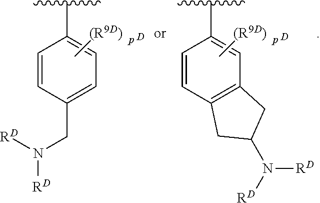

In some embodiments, each R^{D} is independently hydrogen, —C(═O)C₁-C₆ alkyl, —C(═O)OC₁-C₆ alkyl, or C₁-C₆ alkyl, wherein each alkyl of each R^{D} is substituted or unsubstituted. In some embodiments, each R^{D} and R^{D} is independently H or —CH₃. In some embodiments, one R^{D} is H and one R^{D} is

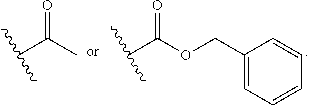

In some embodiments, R^{7D} and R^{8D} are each independently H or —CH₃. In some embodiments, R^{7D} and R^{8D} are each independently H.

In some embodiments, each R^{9D} is independently halogen, —CN, —OR^{D}, —NR^{D}R^{D}, —C(═O)R^{D}, —OC(═O)R^{D}, —OC(═O)OR^{D}, —C(═O)NR^{D}R^{D}, —NR^{D}C(═O)R^{D}, or C₁-C₆ alkyl. In some embodiments, each R^{9D} is independently halogen, —CN, —OR^{D}, or C₁-C₆ alkyl.

In some embodiments, p^{D} is 0 or 1. In some embodiments, p^{D} is 0.

In one aspect, provided herein, is a compound having a structure of Formula (VE):

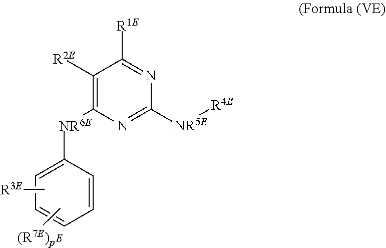

wherein:
R^{1E} is H, nitrile, or halogen;
R^{2E} is halogen, nitirile, methyl, cyclopropyl, or —CF₃;
R^{3E} is halogen,

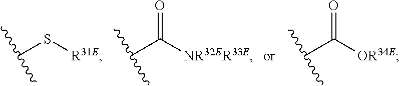

R^{4E} is aryl substituted with one or more —OR^{35E}, substituted or unsubstituted cycloalkyl, or substituted or unsubstituted heterocycloalkyl,

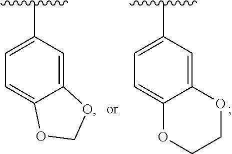 [SMILES: *c1ccc2c(c1)OCO2 ; *c1ccc2c(c1)OCCO2]

R^{5E} and R^{6E} are each independently H or C₁-C₆ alkyl;
each R^{7E} is independently halogen, —CN, —OR^{E}, —S(═O)₂R^{E}, —NR^{E}R^{E}, —S(═O)₂NR^{E}R^{E}, —C(═O)R^{E}, —OC(═O)R^{E}, —C(═O)OR^{E}, —OC(═O)OR^{E}, —C(═O)NR^{E}R^{E}, —OC(═O)NR^{E}R^{E}, —NR^{E}C(═O)NR^{E}R^{E}, —NR^{E}C(═O)R^{E}, C₁-C₆ alkyl, C₁-C₆ haloalkyl, or C₁-C₆ hydroxyalkyl;
p^{E} is an integer from 0 to 3;
R^{31E} is H, C₁-C₆ alkyl, or cycloalkyl;
   R^{32E} and R^{33E} are each independently H, substituted or unsubstituted C₁-C₆ alkyl, or cycloalkyl;
R^{34E} is H, C₁-C₆ alkyl, or cycloalkyl;
each R^{35E} is independently substituted or unsubstituted alkyl, or substituted or unsubstituted heteroalkyl;
each R^{E} is independently hydrogen, C₁-C₆ alkyl, cycloalkyl, heterocycloalkyl, aryl, or heteroaryl; wherein the alkyl, cycloalkyl, heterocycloalkyl, aryl, and heteroaryl are independently optionally substituted with one or more halogen, —OH, —NH₂, substituted amino, cycloalkyl, oxo, or C₁-C₆ alkyl;
wherein when R^{3E} is

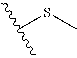 [SMILES: *SC]

then R^{2E} is not Br; and
wherein when R^{3E} is

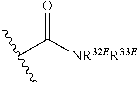

then R^{2E} is not Cl and R^{4E} is not

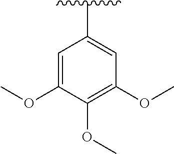

or a pharmaceutically acceptable salt thereof.

In some embodiments, R^{1E} is H or F. In some embodiments, R^{1E} is H.

In some embodiments, R^{2E} is Cl, Br, or —CF₃. In some embodiments, R^{2E} is Br or —CF₃. In some embodiments, R^{3E} is —SR^{31E}. In some embodiments, R^{3E} is —SH, —SCH₃, or —SCH₂CH₃. In some embodiments, R^{3E} is —SCH₃.

In some embodiments, R^{3E} is

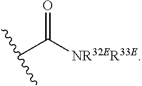

In some embodiments, R^{3E} is

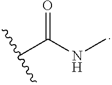 [SMILES: *C(=O)NC]

In some embodiments, R^{3E} is

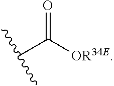

In some embodiments, R^{3E} is

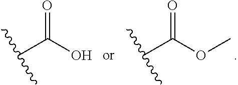

In some embodiments, R^{3E} is

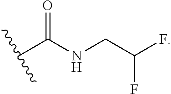 [SMILES: *C(=O)NCC(F)F]

In some embodiments, R^{3E} is

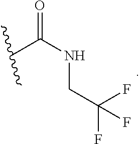 [SMILES: *C(=O)NCC(F)(F)F]

In some embodiments, R^{4E} is

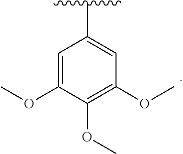

In some embodiments, R^{4E} is

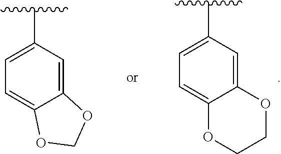 [SMILES: *c1ccc2c(c1)OCO2 ; *c1ccc2c(c1)OCCO2]

In some embodiments, R^{4E} is

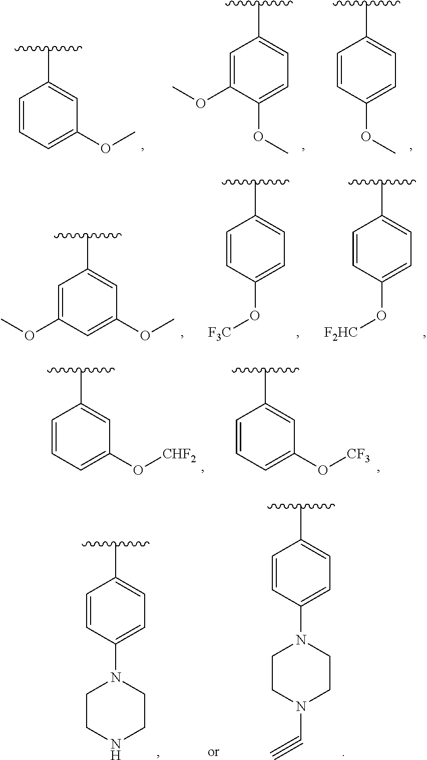

In some embodiments, R^{5E} and R^{6E} are each independently H or —CH₃. In some embodiments, R^{5E} and R^{6E} are each independently H.

In some embodiments, each R^{7E} is independently halogen, —CN, —OR^{E}, —NR^{E}R^{E}, —C(═O)R^{E}, —OC(═O)R^{E}, —C(═O)OR^{E}, —C(═O)NR^{E}R^{E}, or C₁-C₆ alkyl. In some embodiments, each R^{7E} is independently halogen, —OR^{E}, —OC(═O)R^{E}, or C₁-C₆ alkyl. In some embodiments, R^{7E} is independently halogen or —OCH₃. In some embodiments, R^{7E} is

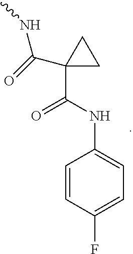

In some embodiments, p^{E} is 0 or 1. In some embodiments, p^{E} is 0.

In one aspect, provided herein, is a pharmaceutical composition comprising the compound or pharmaceutically acceptable salt thereof of any one of the compounds provided herein and a pharmaceutically acceptable carrier. In some embodiments, the pharmaceutical composition is formulated for intravenous or intraperitoneal injection.

In one aspect, provided herein, is a method of treating a ULK1 or ULK2 mediated disease in a subject in need thereof, the method comprising administering to the subject a compound or pharmaceutical composition of any one of the compounds provided herein. In some embodiments, the ULK1 or ULK2 mediated disease is characterized by abnormal autophagy. In some embodiments, the abnormal autophagy has been therapeutically induced.

In some embodiments, the disease is cancer. In some embodiments, the cancer is lung cancer or pancreatic cancer. In some embodiments, the lung cancer is non-small cell lung cancer (NSCLC). In some embodiments, the cancer is pancreatic cancer. In some embodiments, the pancreatic cancer is pancreatic ductal adenocarcinoma (PDAC). In some embodiments, the cancer is breast cancer. In some embodiments, the breast cancer is triple negative breast cancer (TNBC).

In some embodiments, the disease is Tuberous Sclerosis Complex (TSC) or lymphangioleiomyomatosis (LAM).

In some embodiments, the compound is co-administered with an additional therapeutic agent. In some embodiments, the additional therapeutic agent is an mTOR inhibitor. In some embodiments, the additional therapeutic agent is carboplatin. In some embodiments, the additional therapeutic agent is an MEK inhibitor. In some embodiments, the additional therapeutic agent is trametinib. In some embodiments, the additional therapeutic agent is a PARP inhibitor. In some embodiments, the additional therapeutic agent is olaparib. In some embodiments, the additional therapeutic agent is a standard of care therapy.

In some embodiments, administering the compound degrades ATG13 in the subject.

### DETAILED DESCRIPTION OF THE INVENTION

### Definitions

Unless defined otherwise, all technical and scientific terms used herein have the same meaning as is commonly understood by one of skill in the art to which this invention belongs. All patents and publications referred to herein are incorporated by reference.

As used in the specification and claims, the singular form “a”, “an” and “the” includes plural references unless the context clearly dictates otherwise.

“Alkyl” refers to a straight or branched hydrocarbon chain radical consisting solely of carbon and hydrogen atoms, which may optionally be unsaturated with one or more double or triple bonds, and preferably having from one to fifteen carbon atoms (i.e., C₁-C₁₅ alkyl). In certain embodiments, an alkyl comprises one to six carbon atoms (i.e., C₁-C₆ alkyl). In other embodiments, an alkyl comprises one to three carbon atoms (i.e., C₁-C₃ alkyl). In certain embodiments, the alkyl group is selected from methyl, ethyl, 1-propyl (n-propyl), 1-methylethyl (iso-propyl), 1-butyl (n-butyl), 1-methylpropyl (sec-butyl), 2-methylpropyl (iso-butyl), 1,1-dimethylethyl (tert-butyl), 1-pentyl (n-pentyl). The alkyl is attached to the rest of the molecule by a single bond. Unless otherwise specified, the term “alkyl” and its equivalents encompass linear, branched, and/or cyclic alkyl groups. In some instances, an “alkyl” comprises both cyclic and acyclic (linear and/or branched) alkyl components. When an alkyl group is described as “linear,” the referenced alkyl group is not substituted with additional alkyl groups and is unbranched. When an alkyl group is described as “saturated,” the referenced alkyl group does not contain any double or triple carbon-carbon bonds (e.g. alkene or alkyne).

“Alkylene” or “alkylene chain” refers to a divalent alkyl group.

“Aryl” refers to an aromatic monocyclic or aromatic multicyclic hydrocarbon ring system. The aromatic monocyclic or aromatic multicyclic hydrocarbon ring system contains only hydrogen and carbon and from five to eighteen carbon atoms, where at least one of the rings in the ring system is aromatic, i.e., it contains a cyclic, delocalized (4n+2) π-electron system in accordance with the Hückel theory. The ring system from which aryl groups are derived include, but are not limited to, groups such as benzene, fluorene, indane, indene, tetralin and naphthalene.

The term “C_{x-y}” or “Cₓ-C_{y}” when used in conjunction with a chemical moiety, such as alkyl, alkenyl, or alkynyl is meant to include groups that contain from x to y carbons in the chain. For example, the term “C_{x-y}alkyl” refers to saturated or unsaturated hydrocarbon groups, including straight-chain alkyl and branched-chain alkyl groups that contain from x to y carbons in the chain. The terms “C_{x-y}alkenyl” and “C_{x-y}alkynyl” refer to unsaturated aliphatic groups analogous in length and possible substitution to the alkyls described above, but that contain at least one double or triple bond respectively.

“Cycloalkyl” refers to a saturated ring in which each atom of the ring is carbon. Cycloalkyl may include monocyclic and polycyclic rings such as 3- to 10-membered monocyclic rings, 6- to 12-membered fused bicyclic rings, 6- to 12-membered spirocyclic rings, and 6- to 12-membered bridged rings. In certain embodiments, a cycloalkyl comprises three to ten carbon atoms. In other embodiments, a cycloalkyl comprises five to seven carbon atoms. The cycloalkyl may be attached to the rest of the molecule by a single bond. Examples of monocyclic cycloalkyls include, e.g., cyclopropyl, cyclobutyl, cyclopentyl, cyclohexyl, cycloheptyl, and cyclooctyl. Polycyclic cycloalkyl radicals include, for example, adamantyl, norbornyl (i.e., bicyclo[2.2.1]heptanyl), norbornenyl, decalinyl, 7,7-dimethyl-bicyclo[2.2.1]heptanyl, and the like.

“Halo” or, alternatively, “halogen” or “halide,” means fluoro, chloro, bromo or iodo. In some embodiments, halo is fluoro, chloro, or bromo.

“Haloalkyl” refers to an alkyl radical, as defined above, that is substituted by one or more halo radicals, for example, trifluoromethyl, dichloromethyl, bromomethyl, 2,2,2-trifluoroethyl, 1-chloromethyl-2-fluoroethyl, and the like. In some embodiments, the alkyl part of the haloalkyl radical is optionally substituted as described herein.

“Heteroalkyl” refers to an alkyl group wherein one or more of the carbons of the alkyl group is replaced with a heteroatom. Exemplary heteroatoms include N, O, Si, P, B, and S atoms, preferably N, O and S. Note that valency of heteroatoms may not be identical to that of a carbon atom, so, for example, a methylene (CH₂) of an alkyl may be replaced with an NH group, S group, O group, or the like in a heteroalkyl.

“Heteroalkylene” refers to an alkylene group wherein one or more of the carbons of the alkylene group is replaced with a heteroatom. Exemplary heteroatoms include N, O, Si, P, B, and S atoms, preferably N, O and S.

“Heterocycloalkyl” refers to a saturated or unsaturated (e.g., non-aromatic) ring with carbon atoms and at least one heteroatom (e.g., a cycloalkyl wherein one or more of the carbon groups is substituted with a heteroatom). Exemplary heteroatoms include N, O, Si, P, B, and S atoms. Heterocycloalkyl may include monocyclic and polycyclic rings such as 3- to 10-membered monocyclic rings, 6- to 12-membered fused bicyclic rings, 6- to 12-membered spirocyclic rings, and 6- to 12-membered bridged rings. The heteroatoms in the heterocycloalkyl radical are optionally oxidized. One or more nitrogen atoms, if present, are optionally quaternized. The heterocycloalkyl is attached to the rest of the molecule through any atom of the heterocycloalkyl, valence permitting, such as any carbon or nitrogen atoms of the heterocycloalkyl. Examples of heterocycloalkyl radicals include, but are not limited to, dioxolanyl, thienyl[1,3]dithianyl, decahydroisoquinolyl, imidazolinyl, imidazolidinyl, isothiazolidinyl, isoxazolidinyl, morpholinyl, octahydroindolyl, octahydroisoindolyl, 2-oxopiperazinyl, 2-oxopiperidinyl, 2-oxopyrrolidinyl, oxazolidinyl, piperidinyl, piperazinyl, 4-piperidonyl, pyrrolidinyl, pyrazolidinyl, quinuclidinyl, thiazolidinyl, tetrahydrofuryl, trithianyl, tetrahydropyranyl, thiomorpholinyl, thiamorpholinyl, 1-oxo-thiomorpholinyl, and 1,1-dioxo-thiomorpholinyl.

“Heteroaryl” refers to an aromatic ring comprising carbon atoms and one or more heteroatoms. Exemplary heteroatoms include N, O, Si, P, B, and S atoms. As used herein, the heteroaryl ring may be selected from monocyclic or bicyclic and fused or bridged ring systems rings wherein at least one of the rings in the ring system is aromatic, i.e., it contains a cyclic, delocalized (4n+2) π-electron system in accordance with the Hückel theory. The heteroatom(s) in the heteroaryl radical may be optionally oxidized. One or more nitrogen atoms, if present, are optionally quaternized. The heteroaryl may be attached to the rest of the molecule through any atom of the heteroaryl, valence permitting, such as a carbon or nitrogen atom of the heteroaryl. Examples of heteroaryls include, but are not limited to, azepinyl, acridinyl, benzimidazolyl, benzindolyl, 1,3-benzodioxolyl, benzofuranyl, benzooxazolyl, benzo[d]thiazolyl, benzothiadiazolyl, benzo[b][1,4]dioxepinyl, benzo[b][1,4]oxazinyl, 1,4-benzodioxanyl, benzonaphthofuranyl, benzoxazolyl, benzodioxolyl, benzodioxinyl, benzopyranyl, benzopyranonyl, benzofuranyl, benzofuranonyl, benzothienyl (benzothiophenyl), benzothieno[3,2-d]pyrimidinyl, benzotriazolyl, benzo[4,6]imidazo[1,2-a]pyridinyl, carbazolyl, cinnolinyl, cyclopenta[d]pyrimidinyl, 6,7-dihydro-5H-cyclopenta[4,5]thieno[2,3-d]pyrimidinyl, 5,6-dihydrobenzo[h]quinazolinyl, 5,6-dihydrobenzo[h]cinnolinyl, 6,7-dihydro-5H-benzo[6,7]cyclohepta[1,2-c]pyridazinyl, dibenzofuranyl, dibenzothiophenyl, furanyl, furanonyl, furo[3,2-c]pyridinyl, 5,6,7,8,9,10-hexahydrocycloocta[d]pyrimidinyl, 5,6,7,8,9,10-hexahydrocycloocta[d]pyridazinyl, 5,6,7,8,9,10-hexahydrocycloocta[d]pyridinyl, isothiazolyl, imidazolyl, indazolyl, indolyl, indazolyl, isoindolyl, indolinyl, isoindolinyl, isoquinolyl, indolizinyl, isoxazolyl, 5,8-methano-5,6,7,8-tetrahydroquinazolinyl, naphthyridinyl, 1,6-naphthyridinonyl, oxadiazolyl, 2-oxoazepinyl, oxazolyl, oxiranyl, 5,6,6a,7,8,9,10,10a-octahydrobenzo[h]quinazolinyl, 1-phenyl-1H-pyrrolyl, phenazinyl, phenothiazinyl, phenoxazinyl, phthalazinyl, pteridinyl, purinyl, pyrrolyl, pyrazolyl, pyrazolo[3,4-d]pyrimidinyl, pyridinyl, pyrido[3,2-d]pyrimidinyl, pyrido[3,4-d]pyrimidinyl, pyrazinyl, pyrimidinyl, pyridazinyl, pyrrolyl, quinazolinyl, quinoxalinyl, quinolinyl, isoquinolinyl, tetrahydroquinolinyl, 5,6,7,8-tetrahydroquinazolinyl, 5,6,7,8-tetrahydrobenzo[4,5]thieno[2,3-d]pyrimidinyl, 6,7,8,9-tetrahydro-5H-cyclohepta[4,5]thieno[2,3-d]pyrimidinyl, 5,6,7,8-tetrahydropyrido[4,5-c]pyridazinyl, thiazolyl, thiadiazolyl, triazolyl, tetrazolyl, triazinyl, thieno[2,3-d]pyrimidinyl, thieno[3,2-d]pyrimidinyl, thieno[2,3-c]pridinyl, and thiophenyl (i.e. thienyl).

The term “salt” or “pharmaceutically acceptable salt” refers to salts derived from a variety of organic and inorganic counter ions well known in the art. Pharmaceutically acceptable acid addition salts may be formed with inorganic acids and organic acids. Inorganic acids from which salts are derived include, for example, hydrochloric acid, hydrobromic acid, sulfuric acid, nitric acid, phosphoric acid, and the like. Organic acids from which salts are derived include, for example, acetic acid, propionic acid, glycolic acid, pyruvic acid, oxalic acid, maleic acid, malonic acid, succinic acid, fumaric acid, tartaric acid, citric acid, benzoic acid, cinnamic acid, mandelic acid, methanesulfonic acid, ethanesulfonic acid, p-toluenesulfonic acid, salicylic acid, and the like. Pharmaceutically acceptable base addition salts may be formed with inorganic and organic bases. Inorganic bases from which salts are derived include, for example, sodium, potassium, lithium, ammonium, calcium, magnesium, iron, zinc, copper, manganese, aluminum, and the like. Organic bases from which salts are derived include, for example, primary, secondary, and tertiary amines, substituted amines including naturally occurring substituted amines, cyclic amines, basic ion exchange resins, and the like, specifically such as isopropylamine, trimethylamine, diethylamine, triethylamine, tripropylamine, and ethanolamine. In some embodiments, the pharmaceutically acceptable base addition salt is chosen from ammonium, potassium, sodium, calcium, and magnesium salts.

The phrase “pharmaceutically acceptable” is employed herein to refer to those compounds, materials, compositions, and/or dosage forms which are, within the scope of sound medical judgment, suitable for use in contact with the tissues of human beings and animals without excessive toxicity, irritation, allergic response, or other problem or complication, commensurate with a reasonable benefit/risk ratio.

The phrase “pharmaceutically acceptable excipient” or “pharmaceutically acceptable carrier” as used herein means a pharmaceutically acceptable material, composition or vehicle, such as a liquid or solid filler, diluent, excipient, solvent or encapsulating material. Each carrier is “acceptable” in the sense of being compatible with the other ingredients of the formulation and not injurious to the patient. Some examples of materials which serve as pharmaceutically acceptable carriers include: (1) sugars, such as lactose, glucose and sucrose; (2) starches, such as corn starch and potato starch; (3) cellulose, and its derivatives, such as sodium carboxymethyl cellulose, ethyl cellulose and cellulose acetate; (4) powdered tragacanth; (5) malt; (6) gelatin; (7) talc; (8) excipients, such as cocoa butter and suppository waxes; (9) oils, such as peanut oil, cottonseed oil, safflower oil, sesame oil, olive oil, corn oil and soybean oil; (10) glycols, such as propylene glycol; (11) polyols, such as glycerin, sorbitol, mannitol and polyethylene glycol; (12) esters, such as ethyl oleate and ethyl laurate; (13) agar; (14) buffering agents, such as magnesium hydroxide and aluminum hydroxide; (15) alginic acid; (16) pyrogen-free water; (17) isotonic saline; (18) Ringer's solution; (19) ethyl alcohol; (20) phosphate buffer solutions; and (21) other non-toxic compatible substances employed in pharmaceutical formulations.

In certain embodiments, the term “prevent” or “preventing” as related to a disease or disorder may refer to a compound that, in a statistical sample, reduces the occurrence of the disorder or condition in the treated sample relative to an untreated control sample, or delays the onset or reduces the severity of one or more symptoms of the disorder or condition relative to the untreated control sample.

The term “substituted” refers to moieties having substituents replacing a hydrogen on one or more carbons or heteroatoms of the structure. It will be understood that “substitution” or “substituted with” includes the implicit proviso that such substitution is in accordance with permitted valence of the substituted atom and the substituent, and that the substitution results in a stable compound, e.g., which does not spontaneously undergo transformation such as by rearrangement, cyclization, elimination, etc. As used herein, the term “substituted” is contemplated to include all permissible substituents of organic compounds. In a broad aspect, the permissible substituents include acyclic and cyclic, branched and unbranched, carbocyclic and heterocyclic, aromatic and non-aromatic substituents of organic compounds. The permissible substituents may be one or more and the same or different for appropriate organic compounds. For purposes of this disclosure, the heteroatoms such as nitrogen may have hydrogen substituents and/or any permissible substituents of organic compounds described herein which satisfy the valences of the heteroatoms.

Substituents may include any substituents described herein, for example, a halogen, a hydroxyl, a carbonyl (such as a carboxyl, an alkoxycarbonyl, a formyl, or an acyl), a thiocarbonyl (such as a thioester, a thioacetate, or a thioformate), an alkoxyl, a phosphoryl, a phosphate, a phosphonate, a phosphinate, an amino, an amido, an amidine, an imine, a cyano, a nitro, an azido, a sulfhydryl, an alkylthio, a sulfate, a sulfonate, a sulfamoyl, a sulfonamido, a sulfonyl, an aralkyl, a carbocycle, a heterocycle, a cycloalkyl, a heterocycloalkyl, an aromatic and heteroaromatic moiety. In some embodiments, substituents may include any substituents described herein, for example: halogen, hydroxy, oxo (═O), thioxo (═S), cyano (—CN), nitro (—NO₂), imino (═N—H), oximo (═N—OH), hydrazino (═N—NH₂), —R^{b}—OR^{a}, —R^{b}—OC(O)—R^{a}, —R^{b}—OC(O)—OR^{a}, —R^{b}—OC(O)—N(R^{a})₂, —R^{b}—N(R^{a})₂, —R^{b}—C(O)R^{a}, —R^{b}—C(O)OR^{a}, —R^{b}—C(O)N(R^{a})₂, —R^{b}—O—R^{c}—C(O)N(R^{a})₂, —R^{b}—N(R^{a})C(O)OR^{a}, —R^{b}—N(R^{a})C(O)R^{a}, —R^{b}—N(R^{a})S(O)ₜR^{a} (where t is 1 or 2), —R^{b}—S(O)ₜR^{a} (where t is 1 or 2), —R^{b}—S(O)ₜOR^{a} (where t is 1 or 2), and —R^{b}—S(O)ₜN(R^{a})₂ (where t is 1 or 2); and alkyl, alkenyl, alkynyl, aryl, aralkyl, aralkenyl, aralkynyl, cycloalkyl, cycloalkylalkyl, heterocycloalkyl, heterocycloalkylalkyl, heteroaryl, and heteroarylalkyl any of which may be optionally substituted by alkyl, alkenyl, alkynyl, halogen, hydroxy, haloalkyl, haloalkenyl, haloalkynyl, oxo (═O), thioxo (═S), cyano (—CN), nitro (—NO₂), imino (═N—H), oximo (═N—OH), hydrazine (═N—NH₂), —R^{b}—OR^{a}, —R^{b}—OC(O)—R^{a}, —R^{b}—OC(O)—OR^{a}, —R^{b}—OC(O)—N(R^{a})₂, —R^{b}—N(R^{a})₂, —R^{b}—C(O)R^{a}, —R^{b}—C(O)OR^{a}, —R^{b}—C(O)N(R^{a})₂, —R^{b}—O—R^{c}—C(O)N(R^{a})₂, —R^{b}—N(R^{a})C(O)OR^{a}, —R^{b}—N(R^{a})C(O)R^{a}, —R^{b}—N(R^{a})S(O)ₜR^{a} (where t is 1 or 2), —R^{b}—S(O)ₜR^{a} (where t is 1 or 2), —R^{b}—S(O)ₜOR^{a} (where t is 1 or 2) and —R^{b}—S(O)ₜN(R^{a})₂ (where t is 1 or 2); wherein each R^{a} is independently selected from hydrogen, alkyl, cycloalkyl, cycloalkylalkyl, aryl, aralkyl, heterocycloalkyl, heterocycloalkylalkyl, heteroaryl, or heteroarylalkyl, wherein each R^{a}, valence permitting, may be optionally substituted with alkyl, alkenyl, alkynyl, halogen, haloalkyl, haloalkenyl, haloalkynyl, oxo (═O), thioxo (═S), cyano (—CN), nitro (—NO₂), imino (═N—H), oximo (═N—OH), hydrazine (═N—NH₂), —R^{b}—OR^{a}, —R^{b}—OC(O)—R^{a}, —R^{b}—OC(O)—OR^{a}, —R^{b}—OC(O)—N(R^{a})₂, —R^{b}—N(R^{a})₂, —R^{b}—C(O)R^{a}, —R^{b}—C(O)OR^{a}, —R^{b}—C(O)N(R^{a})₂, —R^{b}—O—R^{c}—C(O)N(R^{a})₂, —R^{b}—N(R^{a})C(O)OR^{a}, —R^{b}—N(R^{a})C(O)R^{a}, —R^{b}—N(R^{a})S(O)ₜR^{a} (where t is 1 or 2), —R^{b}—S(O)ₜR^{a} (where t is 1 or 2), —R^{b}—S(O)ₜOR^{a} (where t is 1 or 2) and —R^{b}—S(O)ₜN(R^{a})₂ (where t is 1 or 2); and wherein each R^{b} is independently selected from a direct bond or a straight or branched alkylene, alkenylene, or alkynylene chain, and each R^{c} is a straight or branched alkylene, alkenylene or alkynylene chain.

The terms “treat,” “treating” or “treatment,” as used herein, may include alleviating, abating or ameliorating a disease or condition symptoms, preventing additional symptoms, ameliorating or preventing the underlying causes of symptoms, inhibiting the disease or condition, e.g., arresting the development of the disease or condition, relieving the disease or condition, causing regression of the disease or condition, relieving a condition caused by the disease or condition, or stopping the symptoms of the disease or condition either prophylactically and/or therapeutically.

Compounds of the present invention also include crystalline and amorphous forms of those compounds, pharmaceutically acceptable salts, and active metabolites of these compounds having the same type of activity, including, for example, polymorphs, pseudopolymorphs, solvates, hydrates, unsolvated polymorphs (including anhydrates), conformational polymorphs, and amorphous forms of the compounds, as well as mixtures thereof.

### Autophagy

In certain instances, autophagy is a cellular response to loss of nutrients in which cells catabolize various proteins and organelles to provide building blocks and critical metabolites needed for cell survival. In some instances, autophagy plays an important homeostatic role in many tissues by removing protein aggregates and defective organelles that accumulate with cellular damage over time. While genetics first defined the core components of autophagy conserved across all eukaryotes, the molecular details of how the different autophagy complexes regulate one another and the precise temporal and spatial ordering of biochemical events involved in autophagy induction are typically considered to be poorly understood currently.

In healthy individuals, normal autophagy is, in certain instances, an important process for balancing sources of energy at critical times in development and in response to nutrient stress. In certain instances, autophagy also plays a housekeeping role in removing misfolded or aggregated proteins, clearing damaged organelles, such as mitochondria, endoplasmic reticulum and peroxisomes, as well as eliminating intracellular pathogens. Thus, autophagy is often thought of as a survival mechanism. In various instances, autophagy is either non-selective or selective in the removal of specific organelles, ribosomes and protein aggregates. In addition to elimination of intracellular aggregates and damaged organelles, in certain instances, autophagy promotes cellular senescence and cell surface antigen presentation, protects against genome instability and prevents or inhibits necrosis, giving it an important role in preventing, treating, or inhibiting diseases such as cancer, neurodegeneration, cardiomyopathy, diabetes, liver disease, autoimmune diseases and infections.

In some instances, defects in autophagy pathways are associated with a number of human pathologies, including infectious diseases, neurodegenerative disorders, and cancer. In some instances, the role of autophagy differs in different stages of cancer development; for example, in some instances, initially, autophagy has a preventive effect against cancer, but once a tumor develops, the cancer cells, in certain instances, utilize autophagy for their own cytoprotection. In some cancers, the mutations that cause uncontrolled cell growth which results in the formation of tumors or other cancerous tissue also effectuates changes in autophagy. In some instances, these changes in the autophagic pathways in the cancer cells results in increased survivability and durability of cancer cells. In some instances, this leads to the cells resisting apoptosis and cell death in response to standard cancer treatments, thus reducing the efficacy of cancer therapeutics. In certain instances, rather than killing the cancer cells, the therapeutics merely have the effect of arresting cancer tissue growth, with the cancer tissue entering a cystostatic phase upon treatment. Consequently, in some instances, the cancerous tissue is not killed during treatment, the growth is simply arrested. Upon cessation of treatment, the cancerous tissue is able to resume growth, thus increasing symptoms and complications for the patient. In light of this, in some instances, the addition of a therapeutic that disrupts autophagy has the effect of converting the cytostatic response of the cancer cells to cancer cell death.

In certain cancers, the changes in autophagy caused by the cancer are important for the survival of the cancer cells. As the mutations that cause cancer result in uncontrolled cell growth, in some instances, these cells rely on autophagy to properly regulate the consumption of nutrients to ensure the survival of the cells in conditions that would cause the death of a healthy cell. Thus, methods of inhibiting autophagy in cells present, in certain instances, a method of treating cancer without the need of an additional cancer therapeutic.

### ULK1 and ULK2

In many instances, ULK1 and/or ULK 2 are important protein in regulating autophagy in mammalian cells. In certain instances, ULK1 and/or ULK2 are activated under conditions of nutrient deprivation by several upstream signals, which is followed by the initiation of autophagy. The requirement for ULK1 and/or ULK2 in autophagy initiation has been studied in the context of nutrient deprivation.

In certain instances, ULK1 complex, combining ULK1, ATG (autophagy-related protein) 13 (ATG13), FIP200 (focal adhesion kinase family interacting protein of 200 kDa), and ATG101 is one of the first protein complexes that comes in to play in the initiation and formation of autophagosomes when an autophagic response is initiated. Additionally, ULK1 is considered to be unique as a core conserved component of the autophagy pathway which is a serine/threonine kinase, making it a particularly unique target of opportunity for development of compounds to control autophagy. Equally importantly for a clinical therapeutic index for agents inhibiting ULK1, mice genetically engineered to completely lack ULK1 are viable with significant pathology. Thus, in many instances, a ULK1 selective kinase inhibitor is well tolerated by normal tissues, but not by tumor cells that have become reliant on ULK1 mediated autophagy for survival.

In some instances, ULK2 takes over the functional role of ULK1 when ULK1 function has been inhibited. Thus, in some cases, an inhibitor that is effective for both ULK1 and ULK2 is desirable to mitigate this effect.

### Compounds

The present disclosure provides compounds and salts, and formulations thereof, for use in treating various diseases. In some embodiments, the compounds are ULK inhibitors. In some embodiments, the compounds of the present disclosure are ULK1 inhibitors. In some embodiments, the compounds of the present disclosure are specific ULK1 inhibitors. In some embodiments, the compounds are inhibitors of both ULK1 and ULK2.

In one aspect, the present disclosure provides a compound having a structure of Formula (IA):

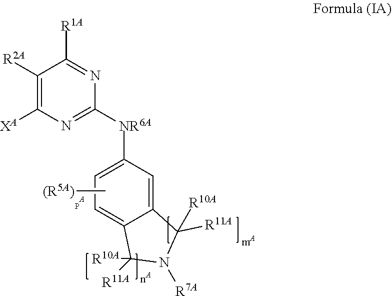

wherein;
R^{1A} is H, halogen, or substituted or unsubstituted alkyl;
R^{2A} is H, haloalkyl, —C(═O)R^{A}, NH₂, or halogen;
X^{A} is —NR^{3A}R^{4A} or —OR^{4A};
R^{3A} is H, substituted or unsubstituted alkyl, or a bond with a substituent on an R^{4A} to form a heterocycle;
R^{4A} is aryl, heteroaryl, cycloalkyl, or heterocycloalkyl;
   wherein the aryl or heteroaryl of R^{4A} is optionally substituted with one or more halogen, —CN, —OR^{A}, —SR^{A}, —NO₂, —NR^{A}R^{A}, —NR^{A}S(═O)₂R^{A}, —C(═O)R^{A}, —OC(═O)R^{A}, —C(═O)C(═O)R^{A}, —C(═O)OR^{A}, —C(═O)NR^{A}OR^{A}, —OC(═O)OR^{A}, —C(═O)NR^{A}R^{A}, —OC(═O)NR^{A}R^{A}, —NR^{A}C(═O)NR^{A}R^{A}, —NR^{A}S(═O)₂NR^{A}R^{A}, —NR^{A}C(═O)R^{A}, —NR^{A}C(═O)OR^{A}, substituted or unsubstituted alkyl, substituted or unsubstituted cycloalkyl, substituted or unsubstituted heterocycloalkyl, substituted or unsubstituted aryl, or substituted or unsubstituted heteroaryl;
   wherein the cycloalkyl or heterocycloalkyl of R^{4A} is optionally substituted with one or more halogen, —CN, —OR^{A}, —SR^{A}, —S(═O)R^{A}, —S(═O)₂R^{A}, —NO₂, —NR^{A}R^{A}, —NR^{A}S(═O)₂R^{A}, —S(═O)₂NR^{A}R^{A}, —C(═O)R^{A}, —OC(═O)R^{A}, —C(═O)C(═O)R^{A}, —C(═O)OR^{A}, —C(═O)NR^{A}OR^{A}, —OC(═O)OR^{A}, —OC(═O)NR^{A}R^{A}, —NR^{A}C(═O)NR^{A}R^{A}, —NR^{A}S(═O)₂NR^{A}R^{A}, —NR^{A}C(═O)R^{A}, —NR^{A}C(═O)OR^{A}, substituted or unsubstituted alkyl, substituted or unsubstituted cycloalkyl, substituted or unsubstituted heterocycloalkyl, substituted or unsubstituted aryl, or substituted or unsubstituted heteroaryl;
each R^{5A} is independently halogen, —CN, —OR^{A}, —SR^{A}, —S(═O)R^{A}, —S(═O)₂R^{A}, —NO₂, —NR^{A}R^{A}, —NR^{A}S(═O)₂R^{A}, —S(═O)₂NR^{A}R^{A}, —C(═O)R^{A}, —OC(═O)R^{A}, —C(═O)C(═O)R^{A}, —C(═O)OR^{A}, —C(═O)NR^{A}OR^{A}, —OC(═O)OR^{A}, —C(═O)NR^{A}R^{A}, —OC(═O)NR^{A}R^{A}, —NR^{A}C(═O)NR^{A}R^{A}, —NR^{A}S(═O)₂NR^{A}R^{A}, —NR^{A}C(═O)R^{A}, —NR^{A}C(═O)OR^{A}, substituted or unsubstituted alkyl, substituted or unsubstituted cycloalkyl, substituted or unsubstituted heterocycloalkyl, substituted or unsubstituted aryl, or substituted or unsubstituted heteroaryl;
R^{6A} is H or substituted or unsubstituted alkyl;
R^{7A} is —S(═O)R^{A}, —S(═O)₂R^{A}, —S(═O)₂NR^{A}R^{A}, —C(═O)NR^{A}R^{A}, —C(═O)R^{71A}, —C(═O)C(═O)R^{A}, —C(═O)OR^{72A}, —C(═O)NR^{A}OR^{A}, substituted or unsubstituted alkyl, substituted or unsubstituted cycloalkyl, or substituted or unsubstituted heterocycloalkyl;
each R^{10A} and R^{11A} is independently H, substituted or unsubstituted alkyl, substituted or unsubstituted alkoxy, substituted or unsubstituted cycloalkyl, substituted or unsubstituted heterocycloalkyl, hydroxyl, halogen, or R^{10A} and R^{11A} on the same atom join to form a cycloalkyl or heterocycloalkyl, or R^{10A} and R^{11A} on the same atom are taken together to form an oxo;
R^{71A} is H, —CN, substituted or unsubstituted methyl, substituted or unsubstituted ethyl, substituted or unsubstituted C₃-C₁₀ alkyl, substituted or unsubstituted C₄-C₁₀ cycloalkyl, substituted or unsubstituted heterocycloalkyl, substituted or unsubstituted aryl, or substituted or unsubstituted;
R^{72A} is H, —CN, substituted or unsubstituted methyl, substituted or unsubstituted ethyl, linear C₃-C₅ alkyl, substituted or unsubstituted C₃-C₁₀ cycloalkyl, substituted or unsubstituted heterocycloalkyl, substituted or unsubstituted aryl, or substituted or unsubstituted heteroaryl;
each R^{A} is independently H, substituted or unsubstituted alkyl, substituted or unsubstituted heteroalkyl, substituted or unsubstituted aryl, or substituted or unsubstituted heteroaryl;
n^{A} is 1 or 2;
m^{A} is 1 or 2; wherein the sum of n and m is 2 or 3;
p^{A} is an integer from 0-3, and
the nitrogen in the fused ring system is optionally quaternized with C₁-C₆ alkyl,
or pharmaceutically acceptable salt thereof.

In some embodiments, R^{1A} is H, halogen, or C₁-C₆ alkyl. In some embodiments, R^{1A} is H or fluorine. In some embodiments, R^{1A} is H. In some embodiments, R^{1A} is fluorine.

In some embodiments, R^{2A} is H, C₁-C₆ haloalkyl, or halogen. In some embodiments, R^{2A} is —CF₃, or halogen. In some embodiments, R^{2A} is —CF₃, —Cl, or —Br. In some embodiments, R^{2A} is —CF₃. In some embodiments, R^{2A} is Br. In some embodiments, R^{2A} is Cl. In some embodiments, R^{2A} is halogen.

In some embodiments, X^{A} is —NR^{3A}R^{4A}. In some embodiments, R^{3A} is H or C₁-C₆ alkyl. In some embodiments, R^{3A} is H, or —CH₃. In some embodiments, R^{3A} is H. In some embodiments, R^{3A} is H or C₁-C₃ alkyl. In some embodiments, R^{3A} is H, methyl, or ethyl.

In some embodiments, R^{4A} is aryl or heteroaryl, wherein the aryl or heteroaryl of R^{4A} is optionally substituted with one or more halogen, —CN, —OR^{A}, —SR^{A}, —NO₂, —NR^{A}R^{A}, —NR^{A}S(═O)₂R^{A}, —C(═O)R^{A}, —OC(═O)R^{A}, —C(═O)C(═O)R^{A}, —C(═O)OR^{A}, —C(═O)NR^{A}OR^{A}, —OC(═O)OR^{A}, —C(═O)NR^{A}R^{A}, —OC(═O)NR^{A}R^{A}, —NR^{A}C(═O)NR^{A}R^{A}, —NR^{A}S(═O)₂NR^{A}R^{A}, —NR^{A}C(═O)R^{A}, —NR^{A}C(═O)OR^{A}, substituted or unsubstituted alkyl, or substituted or unsubstituted cycloalkyl. In some embodiments, R^{4A} is aryl or heteroaryl wherein the aryl or heteroaryl of R^{4A} is optionally substituted with one or more halogen, —CN, —OR^{A}, —NR^{A}R^{A}, —C(═O)R^{A}, —OC(═O)R^{A}, —C(═O)C(═O)R^{A}, —C(═O)OR^{A}, —C(═O)NR^{A}OR^{A}, —OC(═O)OR^{A}, —C(═O)NR^{A}R^{A}, —OC(═O)NR^{A}R^{A}, —NR^{A}C(═O)NR^{A}R^{A}, —NR^{A}C(═O)R^{A}, —NR^{A}C(═O)OR^{A}, substituted or unsubstituted alkyl, or substituted or unsubstituted cycloalkyl. In some embodiments, R^{4A} is aryl or heteroaryl. In some embodiments, the aryl or heteroaryl of R^{4A} is optionally substituted with one or more halogen, —CN, —OR^{A}, —NR^{A}R^{A}, —C(═O)R^{A}, —OC(═O)R^{A}, —C(═O)OR^{A}, —C(═O)NR^{A}OR^{A}, —C(═O)NR^{A}R^{A}, —NR^{A}C(═O)R^{A}, substituted or unsubstituted alkyl, or substituted or unsubstituted cycloalkyl. In some embodiments, R^{4A} is aryl or heteroaryl, wherein the aryl or heteroaryl of R^{4A} is optionally substituted with one or more halogen, —OR^{A}, —C(═O)R^{A}, —C(═O)NR^{A}R^{A}, —NR^{A}C(═O)R^{A}, or substituted or unsubstituted alkyl. In some embodiments, R^{4A} is aryl or heteroaryl, wherein the aryl or heteroaryl of R^{4A} is optionally substituted with one or more halogen, —OR^{A}, —C(═O)NR^{A}R^{A}, or substituted or unsubstituted alkyl. In some embodiments, R^{4A} is 6-membered aryl or heteroaryl. In some embodiments, R^{4A} is 6-membered aryl or 6-membered heteroaryl. In some embodiments, R^{4A} is phenyl, pyridyl, or pyrimidinyl. In some embodiments, R^{4A} is phenyl. In some embodiments, R^{4A} is phenyl substituted with

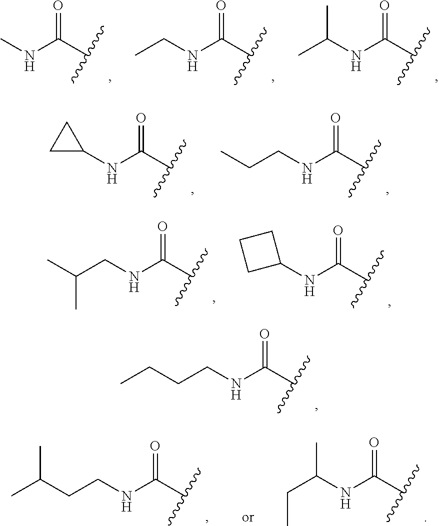

In some embodiments, R^{4A} is

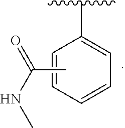

In some embodiments, R^{4A} is

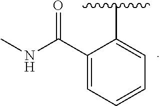 [SMILES: *c1ccccc1C(=O)NC]

In some embodiments, R^{4A} is cycloalkyl, or heterocycloalkyl optionally substituted with one or more halogen, —CN, —OR^{A}, —NR^{A}R^{A}, —C(═O)R^{A}, —OC(═O)R^{A}, —C(═O)C(═O)R^{A}, —C(═O)OR^{A}, —C(═O)NR^{A}OR^{A}, —OC(═O)OR^{A}, —C(═O)NR^{A}R^{A}, —OC(═O)NR^{A}R^{A}, —NR^{A}C(═O)NR^{A}R^{A}, —NR^{A}C(═O)R^{A}, —NR^{A}C(═O)OR^{A}, substituted or unsubstituted alkyl, or substituted or unsubstituted cycloalkyl. In some embodiments, R^{4A} is cycloalkyl optionally substituted with one or more halogen, —CN, —OR^{A}, —NR^{A}R^{A}, —C(═O)R^{A}, —OC(═O)R^{A}, —C(═O)C(═O)R^{A}, —C(═O)OR^{A}, —C(═O)NR^{A}OR^{A}, —OC(═O)OR^{A}, —C(═O)NR^{A}R^{A}, —OC(═O)NR^{A}R^{A}, —NR^{A}C(═O)NR^{A}R^{A}, —NR^{A}C(═O)R^{A}, —NR^{A}C(═O)OR^{A}, substituted or unsubstituted alkyl, or substituted or unsubstituted cycloalkyl. In some embodiments, R^{4A} is cyclopropyl, cyclobutyl, cyclopentyl, or cyclohexyl optionally substituted with one or more halogen, —CN, —OR^{A}, —NR^{A}R^{A}, —C(═O)R^{A}, —OC(═O)R^{A}, —C(═O)C(═O)R^{A}, —C(═O)OR^{A}, —C(═O)NR^{A}OR^{A}, —OC(═O)OR^{A}, —C(═O)NR^{A}R^{A}, —OC(═O)NR^{A}R^{A}, —NR^{A}C(═O)NR^{A}R^{A}, —NR^{A}C(═O)R^{A}, —NR^{A}C(═O)OR^{A}, substituted or unsubstituted alkyl, or substituted or unsubstituted cycloalkyl. In some embodiments, R^{4A} is cyclopropyl optionally substituted with one or more halogen, —CN, —OR^{A}, —NR^{A}R^{A}, —C(═O)R^{A}, —OC(═O)R^{A}, —C(═O)C(═O)R^{A}, —C(═O)OR^{A}, —C(═O)NR^{A}OR^{A}, —OC(═O)OR^{A}, —C(═O)NR^{A}R^{A}, —OC(═O)NR^{A}R^{A}, —NR^{A}C(═O)NR^{A}R^{A}, —NR^{A}C(═O)R^{A}, —NR^{A}C(═O)OR^{A}, substituted or unsubstituted alkyl, or substituted or unsubstituted cycloalkyl. In some embodiments, R^{4A} is cyclopropyl or cyclobutyl optionally substituted with one or more halogen, —OR^{A}, —C(═O)R^{A}, —C(═O)OR^{A}, —C(═O)NR^{A}R^{A}, or substituted or unsubstituted alkyl. In some embodiments, R^{4A} is cyclopropyl optionally substituted with one or more halogen, —OR^{A}, —C(═O)R^{A}, —C(═O)OR^{A}, —C(═O)NR^{A}R^{A}, or substituted or unsubstituted alkyl. In some embodiments, R^{4A} is cyclopropyl or cyclobutyl optionally substituted with one or more-OR^{A} or substituted or unsubstituted alkyl. In some embodiments, R^{4A} is cyclopropyl optionally substituted with one or more-OR^{A} or substituted or unsubstituted alkyl. In some embodiments, R^{4A} is cyclopropyl optionally substituted with OH or C₁-C₆ alkyl. In some embodiments, R^{4A} is unsubstituted cyclopropyl. In some embodiments, R^{4A} is unsubstituted cyclobutyl.

In some embodiments, each R^{5A} is independently halogen, —CN, —OR^{A}, —NR^{A}R^{A}, —C(═O)R^{A}, —OC(═O)R^{A}, —C(═O)OR^{A}, —C(═O)NR^{A}OR^{A}, —OC(═O)OR^{A}, —C(═O)NR^{A}R^{A}, —OC(═O)NR^{A}R^{A}, —NR^{A}C(═O)NR^{A}R^{A}, —NR^{A}S(═O)₂NR^{A}R^{A}, —NR^{A}C(═O)R^{A}, —NR^{A}C(═O)OR^{A}, substituted or unsubstituted alkyl, substituted or unsubstituted cycloalkyl, substituted or unsubstituted heterocycloalkyl, substituted or unsubstituted aryl, or substituted or unsubstituted heteroaryl. In some embodiments, each R^{5A} is independently halogen, —CN, —OR^{A}, —NR^{A}R^{A}, —C(═O)R^{A}, —OC(═O)R^{A}, —C(═O)OR^{A}, substituted or unsubstituted alkyl, or substituted or unsubstituted cycloalkyl. In some embodiments, each R^{5A} is independently halogen, —OR^{A}, —NR^{A}R^{A}, or unsubstituted C₁-C₆ alkyl.

In some embodiments, p^{A} is 0 or 1. In some embodiments, p^{A} is 0. In some embodiments, p^{A} is 2. In some embodiments, p^{A} is 1. In some embodiments, p^{A} is 1 or 2.

In some embodiments, R^{6A} is H or —CH₃. In some embodiments, R^{6A} is H. In some embodiments, R^{6A} is H or C₁-C₃ alkyl. In some embodiments, R^{6A} is C₁-C₃ alkyl. In some embodiments, R^{6A} is —CH₃.

In some embodiments, R^{7A} is —S(═O)₂R^{A}, —S(═O)₂NR^{A}R^{A}, —C(═O)NR^{A}R^{A}, —C(═O)R^{71A}, —C(═O)OR^{72A}, —C(═O)NR^{A}OR^{A}, substituted or unsubstituted C₁-C₆ alkyl, substituted or unsubstituted C₃-C₈ cycloalkyl, or substituted or unsubstituted C₂-C₈ heterocycloalkyl. In some embodiments, R^{7A} is —S(═O)₂R^{A} S(═O)₂NR^{A}R^{A}, —C(═O)R^{71A}, substituted or unsubstituted C₁-C₆ alkyl, or substituted or unsubstituted C₂-C₈ heterocycloalkyl. In some embodiments, R^{7A} is —S(═O)₂R^{A}, —S(═O)₂NR^{A}R^{A}, —C(═O)NR^{A}R^{A}, —C(═O)R^{71A}, —C(═O)OR^{72A}, —C(═O)NR^{A}OR^{A}, substituted or unsubstituted saturated C₁-C₆ alkyl, substituted or unsubstituted C₃-C₈ cycloalkyl, or substituted or unsubstituted C₂-C₈ heterocycloalkyl. In some embodiments, R^{7A} is —S(═O)₂R^{A} S(═O)₂NR^{A}R^{A}, —C(═O)R^{71A}, substituted or unsubstituted C₁-C₆ alkyl, or substituted or unsubstituted C₂-C₈ heterocycloalkyl. In some embodiments, R^{7A} is —S(═O)₂R^{A} S(═O)₂NR^{A}R^{A}, —C(═O)R^{71A}, substituted or unsubstituted saturated C₁-C₆ alkyl, or substituted or unsubstituted C₂-C₈ heterocycloalkyl. In some embodiments, R^{7A} is substituted or unsubstituted saturated C₁-C₆ alkyl. In some embodiments, R^{7A} is substituted or unsubstituted C₂-C₈ heterocycloalkyl. In some embodiments, R^{7A} is —C(═O)R^{71A} or substituted or unsubstituted C₁-C₆ alkyl. In some embodiments, R^{7A} is

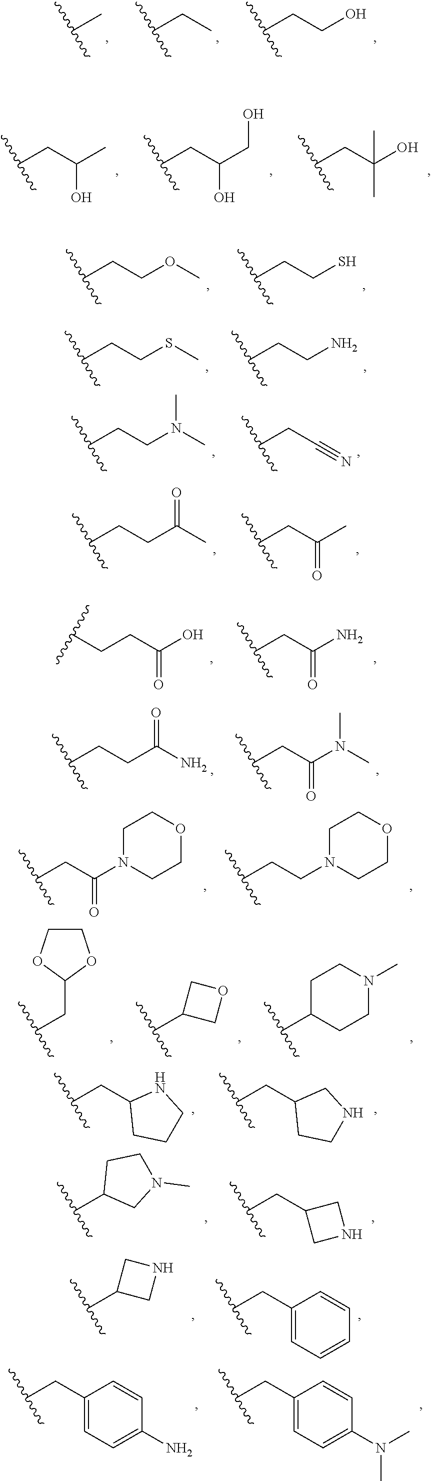

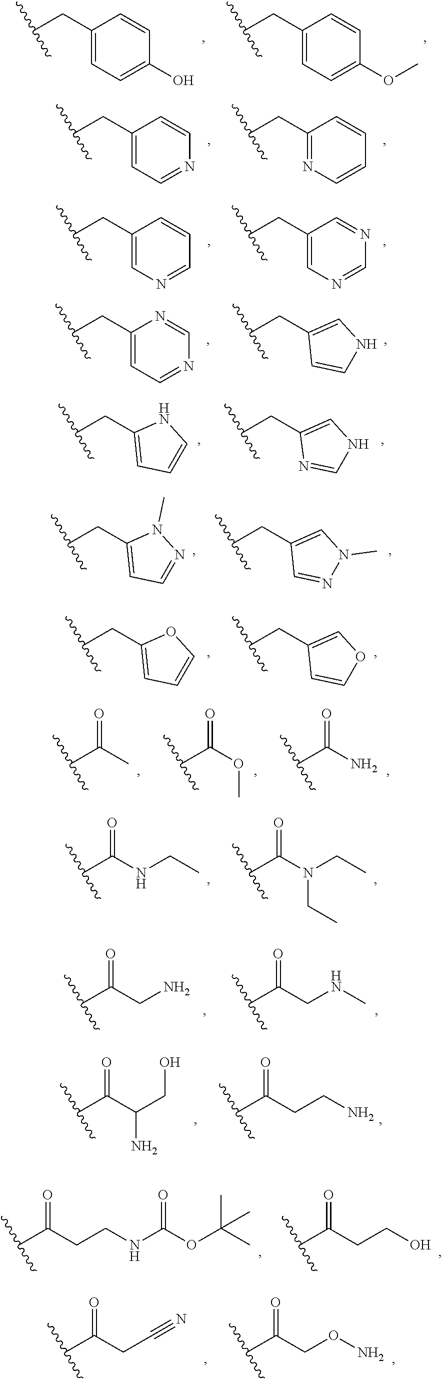

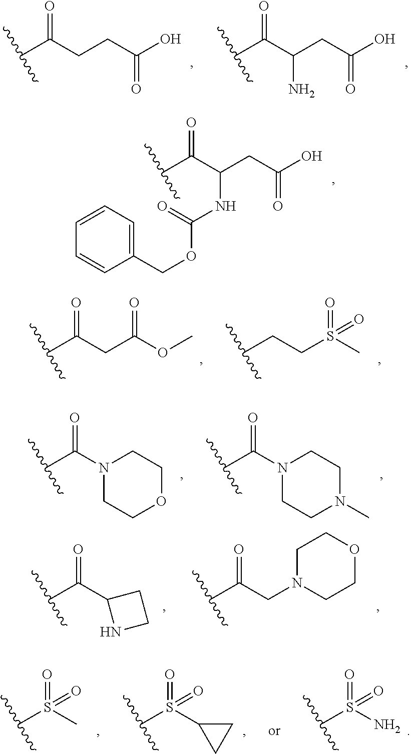

In some embodiments, each R^{10A} and R^{11A} is independently H or substituted or unsubstituted alkyl, or R^{10A} and R^{11A} on the same atom join to form a cycloalkyl, or R^{10A} and R^{11A} on the same atom are taken together to form an oxo. In some embodiments, each R^{10A} and R^{11A} is independently H or substituted or unsubstituted alkyl, or R^{10A} and R^{11A} on the same atom are taken together to form an oxo. In some embodiments, each R^{10A} and R^{11A} is independently H or methyl. In some embodiments, each R^{10A} and R^{11A} is independently H.

In some embodiments, n^{A} is 1 and m^{A} is 1. In some embodiments, n^{A} is 1 and m^{A} is 2. In some embodiments, n^{A} is 2 and m^{A} is 1

Any combination of the groups described above for the various variables is contemplated herein. Throughout the specification, groups and substituents thereof are chosen by one skilled in the field to provide stable moieties and compounds.

Illustrative compounds of Formula I and related analogs are shown in Table 1 (along with their respective IC₅₀ values for ULK1 inhibition assays). For ADP Glo assay, IC₅₀s are represented nM, with A representing IC₅₀<1 nM, B representing 10 nM>IC₅₀>1 nM, and C representing IC₅₀>10 nM. NT indicates the compound was not tested. ULK1 inhibition assays were performed in a 5 uL reaction volume containing 2 ug/mL recombinant human ULK1 protein (1-649, SignalChem #U01-11G) and 80 ug/mL myelin basic protein (MBP, Sigma-Aldrich #M1891) in the presence of 25 uM ATP (Sigma-Aldrich A7699). ULK 1 inhibition was assessed after one hour. Compounds were tested in triplicate in a 16-dose IC₅₀ mode with 3-fold serial dilution and a starting dose of 30 uM. Staurosporine, a non-selective protein kinase inhibitor, was used in the assay as a positive control.

IC₅₀s were also measured by ULK1 NanoBRET assay according to the following protocol: Human embryonic kidney cells (HEK293T) were transfected with NanoLuc®-ULK1 Fusion Vector (Promega #NV2211) using jetPRIME transfection reagent (Polyplus Transfection #114-15). Following 24 h, cells were trypsinized and resuspended in Opti-MEM® I (1×), Reduced Serum Medium (Gibco, #11058-021). Approximately, 7,000 cells per well (in 34 μL total volume) were replated into non-binding surface 384 well plates. Complete NanoBRET 20× Tracer K-5 reagent was prepared according to the manufacturer's directions and 2 μL were added to each well of the 384 plate (assay plate). The assay plate was mixed on an orbital shaker for 15 seconds at 700 rpm. Compounds were serially diluted at 200× final concentration in 100% DMSO, then diluted to 10× final concentration in assay media (Opti-MEM® I, Reduced Serum Medium). Next, 4 μL 10× test compounds were added to each well of the assay plate, followed by mixing at 700 rpm for 15 seconds. The assay plate was incubated for 2 h in a 37 C incubator with 5% CO2 and then equilibrated to RT for 15 min. The 3× Complete Substrate plus Inhibitor Solution was prepared according to the manufacturer's directions with a concentration of Extracellular NanoLuc® Inhibitor of 60 μM to be used at a working concentration of 20 PM. The 3× Complete Substrate plus Inhibitor Solution was mixed and 20 μL per well was added to the assay plate and incubated at RT for 2-3 min. Donor emission wavelength (450 nm) and acceptor emission wavelength (610 nm) were measured using an assay compatible luminometer (see manufacturer's specifications).

TABLE 1

| | | ULK1 IC₅₀ | ULK1 IC₅₀ |
|---|---|---|---|
| | | ADP Glo (A < 1 | NanoBRET |
| Com- | | nM, 1 nM < B < | (A < 100 |
| pound | | 10 nM, C > | nM, B > 100 |
| Number | Structure | 10 nM) | nM) |
| A1 | 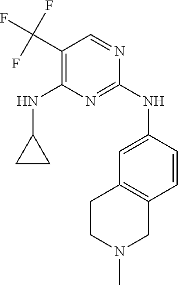 [SMILES: CN1CCc2cc(Nc3ncc(C(F)(F)F)c(NC4CC4)n3)ccc2C1] | A | |
| A2 | 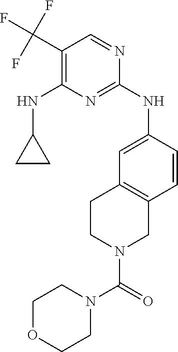 [SMILES: O=C(N1CCOCC1)N1CCc2cc(Nc3ncc(C(F)(F)F)c(NC4CC4)n3)ccc2C1] | B | |
| A3 | 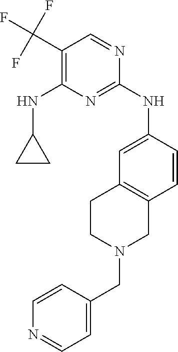 [SMILES: FC(F)(F)c1cnc(Nc2ccc3c(c2)CCN(Cc2ccncc2)C3)nc1NC1CC1] | A | |
| A4 | 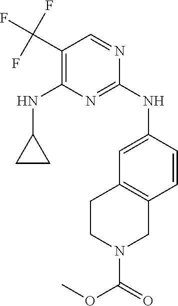 [SMILES: COC(=O)N1CCc2cc(Nc3ncc(C(F)(F)F)c(NC4CC4)n3)ccc2C1] | B | |
| A5 | 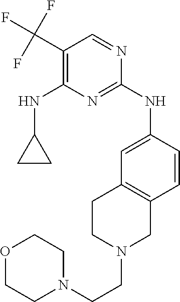 [SMILES: FC(F)(F)c1cnc(Nc2ccc3c(c2)CCN(CCN2CCOCC2)C3)nc1NC1CC1] | A | |
| A6 | 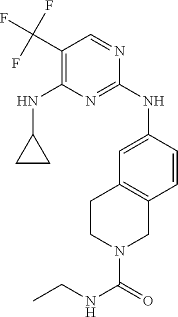 [SMILES: CCNC(=O)N1CCc2cc(Nc3ncc(C(F)(F)F)c(NC4CC4)n3)ccc2C1] | B | |
| A7 | 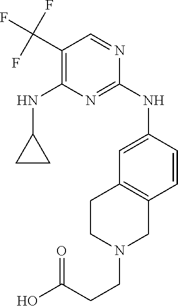 [SMILES: O=C(O)CCN1CCc2cc(Nc3ncc(C(F)(F)F)c(NC4CC4)n3)ccc2C1] | A | |
| A8 | 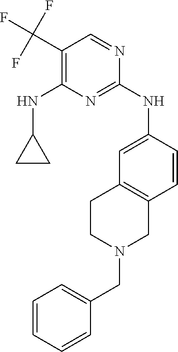 [SMILES: FC(F)(F)c1cnc(Nc2ccc3c(c2)CCN(Cc2ccccc2)C3)nc1NC1CC1] | A | |
| A9 | 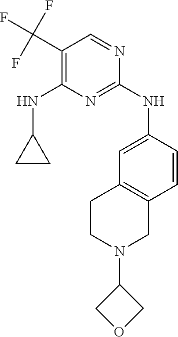 [SMILES: FC(F)(F)c1cnc(Nc2ccc3c(c2)CCN(C2COC2)C3)nc1NC1CC1] | B | |
| A10 | 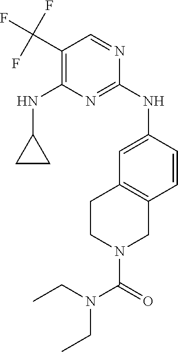 [SMILES: CCN(CC)C(=O)N1CCc2cc(Nc3ncc(C(F)(F)F)c(NC4CC4)n3)ccc2C1] | B | |
| A11 | 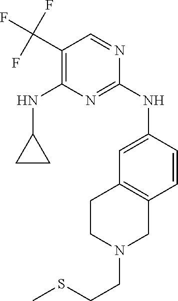 [SMILES: CSCCN1CCc2cc(Nc3ncc(C(F)(F)F)c(NC4CC4)n3)ccc2C1] | A | |
| A12 | 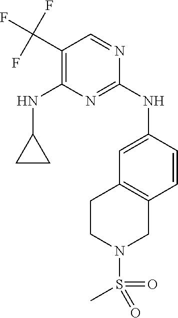 [SMILES: CS(=O)(=O)N1CCc2cc(Nc3ncc(C(F)(F)F)c(NC4CC4)n3)ccc2C1] | B | |
| A13 | 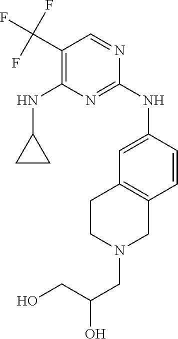 [SMILES: OCC(O)CN1CCc2cc(Nc3ncc(C(F)(F)F)c(NC4CC4)n3)ccc2C1] | C | |
| A14 | 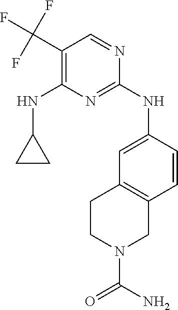 [SMILES: NC(=O)N1CCc2cc(Nc3ncc(C(F)(F)F)c(NC4CC4)n3)ccc2C1] | B | |
| A15 | 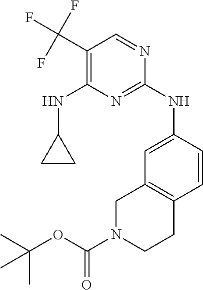 [SMILES: CC(C)(C)OC(=O)N1CCc2ccc(Nc3ncc(C(F)(F)F)c(NC4CC4)n3)cc2C1] | B | |
| A16 | 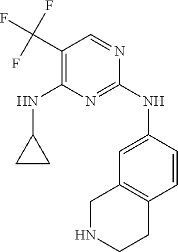 [SMILES: FC(F)(F)c1cnc(Nc2ccc3c(c2)CNCC3)nc1NC1CC1] | A | |
| A17 | 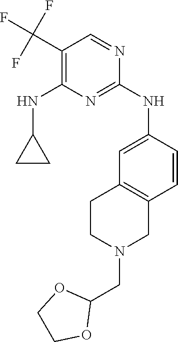 [SMILES: FC(F)(F)c1cnc(Nc2ccc3c(c2)CCN(CC2OCCO2)C3)nc1NC1CC1] | A | |
| A18 | 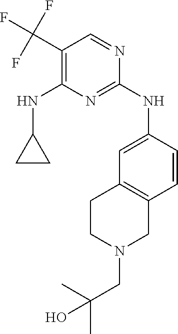 [SMILES: CC(C)(O)CN1CCc2cc(Nc3ncc(C(F)(F)F)c(NC4CC4)n3)ccc2C1] | A | |
| A19 | 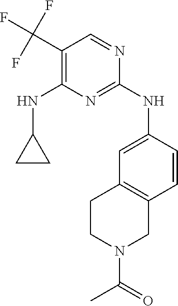 [SMILES: CC(=O)N1CCc2cc(Nc3ncc(C(F)(F)F)c(NC4CC4)n3)ccc2C1] | A | A |
| A20 | 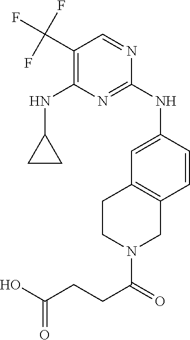 [SMILES: O=C(O)CCC(=O)N1CCc2cc(Nc3ncc(C(F)(F)F)c(NC4CC4)n3)ccc2C1] | A | |
| A21 | 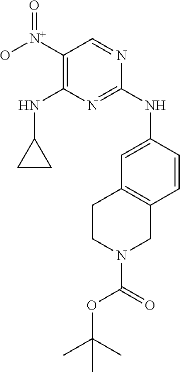 | C | |
| A22 | 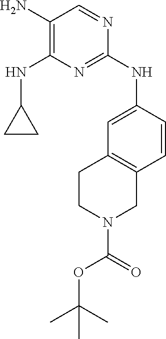 [SMILES: CC(C)(C)OC(=O)N1CCc2cc(Nc3ncc(N)c(NC4CC4)n3)ccc2C1] | C | |
| A23 | 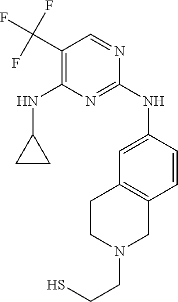 [SMILES: FC(F)(F)c1cnc(Nc2ccc3c(c2)CCN(CCS)C3)nc1NC1CC1] | A | |
| A24 | 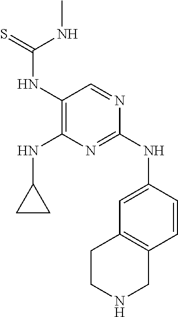 [SMILES: CNC(=S)Nc1cnc(Nc2ccc3c(c2)CCNC3)nc1NC1CC1] | C | |
| A25 | 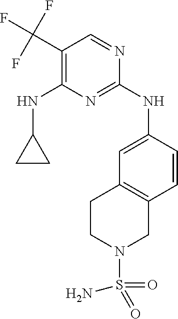 [SMILES: NS(=O)(=O)N1CCc2cc(Nc3ncc(C(F)(F)F)c(NC4CC4)n3)ccc2C1] | A | |
| A26 | 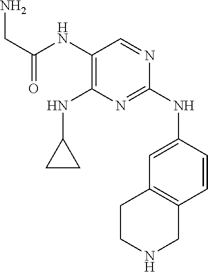 [SMILES: NCC(=O)Nc1cnc(Nc2ccc3c(c2)CCNC3)nc1NC1CC1] | C | |
| A27 | 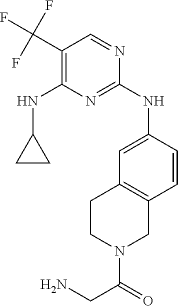 [SMILES: NCC(=O)N1CCc2cc(Nc3ncc(C(F)(F)F)c(NC4CC4)n3)ccc2C1] | A | |
| A28 | 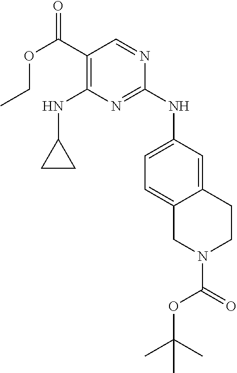 [SMILES: CCOC(=O)c1cnc(Nc2ccc3c(c2)CCN(C(=O)OC(C)(C)C)C3)nc1NC1CC1] | C | |
| A29 | 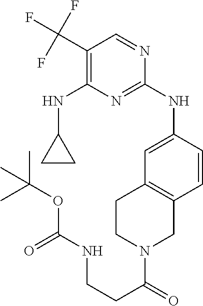 [SMILES: CC(C)(C)OC(=O)NCCC(=O)N1CCc2cc(Nc3ncc(C(F)(F)F)c(NC4CC4)n3)ccc2C1] | A | |
| A30 | 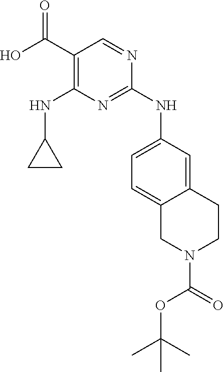 [SMILES: CC(C)(C)OC(=O)N1CCc2cc(Nc3ncc(C(=O)O)c(NC4CC4)n3)ccc2C1] | C | |
| A31 | 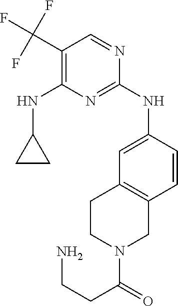 [SMILES: NCCC(=O)N1CCc2cc(Nc3ncc(C(F)(F)F)c(NC4CC4)n3)ccc2C1] | A | |
| A32 | 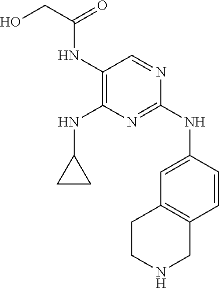 [SMILES: O=C(CO)Nc1cnc(Nc2ccc3c(c2)CCNC3)nc1NC1CC1] | | |
| A33 | 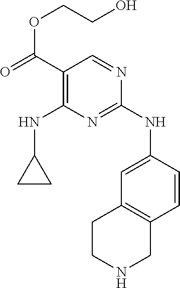 [SMILES: O=C(OCCO)c1cnc(Nc2ccc3c(c2)CCNC3)nc1NC1CC1] | C | |
| A34 | 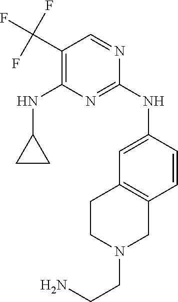 [SMILES: NCCN1CCc2cc(Nc3ncc(C(F)(F)F)c(NC4CC4)n3)ccc2C1] | A | |
| A35 | 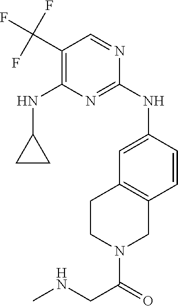 [SMILES: CNCC(=O)N1CCc2cc(Nc3ncc(C(F)(F)F)c(NC4CC4)n3)ccc2C1] | A | |
| A36 | 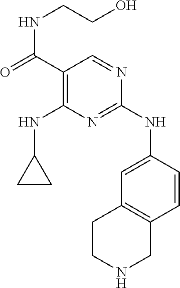 [SMILES: O=C(NCCO)c1cnc(Nc2ccc3c(c2)CCNC3)nc1NC1CC1] | C | |
| A37 | 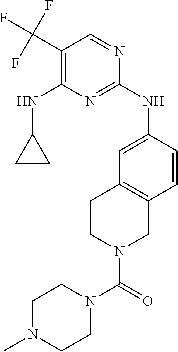 [SMILES: CN1CCN(C(=O)N2CCc3cc(Nc4ncc(C(F)(F)F)c(NC5CC5)n4)ccc3C2)CC1] | A | |
| A38 | 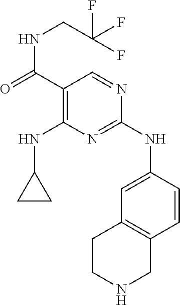 [SMILES: O=C(NCC(F)(F)F)c1cnc(Nc2ccc3c(c2)CCNC3)nc1NC1CC1] | C | |
| A39 | 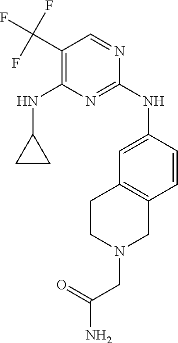 [SMILES: NC(=O)CN1CCc2cc(Nc3ncc(C(F)(F)F)c(NC4CC4)n3)ccc2C1] | C | |
| A40 | 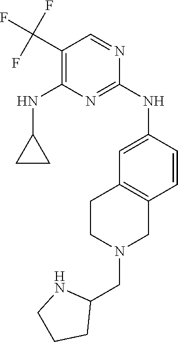 [SMILES: FC(F)(F)c1cnc(Nc2ccc3c(c2)CCN(CC2CCCN2)C3)nc1NC1CC1] | B | |
| A41 | 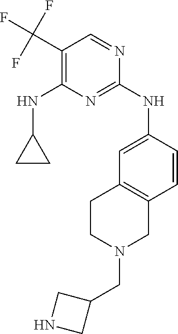 [SMILES: FC(F)(F)c1cnc(Nc2ccc3c(c2)CCN(CC2CNC2)C3)nc1NC1CC1] | A | |
| A42 | 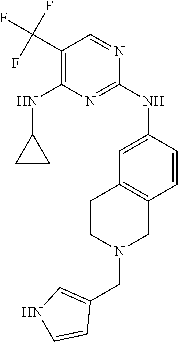 [SMILES: FC(F)(F)c1cnc(Nc2ccc3c(c2)CCN(Cc2cc[nH]c2)C3)nc1NC1CC1] | A | |
| A43 | 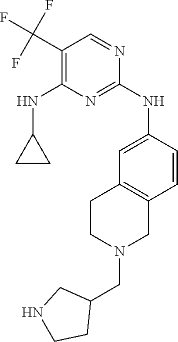 [SMILES: FC(F)(F)c1cnc(Nc2ccc3c(c2)CCN(CC2CCNC2)C3)nc1NC1CC1] | A | |
| A44 | 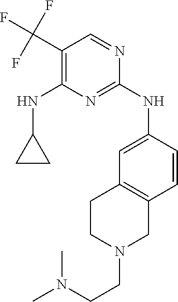 [SMILES: CN(C)CCN1CCc2cc(Nc3ncc(C(F)(F)F)c(NC4CC4)n3)ccc2C1] | A | |
| A45 | 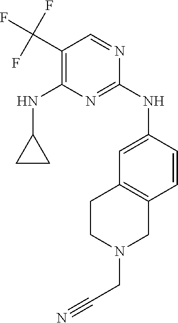 [SMILES: N#CCN1CCc2cc(Nc3ncc(C(F)(F)F)c(NC4CC4)n3)ccc2C1] | A | |
| A46 | 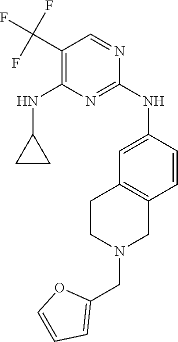 [SMILES: FC(F)(F)c1cnc(Nc2ccc3c(c2)CCN(Cc2ccco2)C3)nc1NC1CC1] | A | |
| A47 | 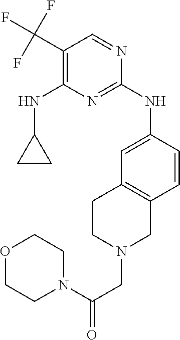 [SMILES: O=C(CN1CCc2cc(Nc3ncc(C(F)(F)F)c(NC4CC4)n3)ccc2C1)N1CCOCC1] | A | A |
| A48 | 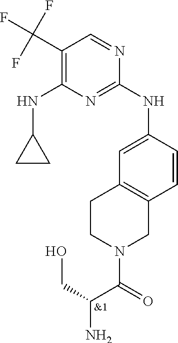 [SMILES: N[C@H](CO)C(=O)N1CCc2cc(Nc3ncc(C(F)(F)F)c(NC4CC4)n3)ccc2C1] | A | |
| A49 | 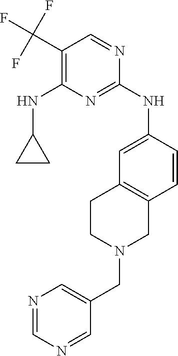 [SMILES: FC(F)(F)c1cnc(Nc2ccc3c(c2)CCN(Cc2cncnc2)C3)nc1NC1CC1] | A | |
| A50 | 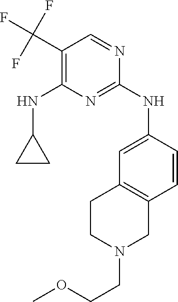 [SMILES: COCCN1CCc2cc(Nc3ncc(C(F)(F)F)c(NC4CC4)n3)ccc2C1] | A | |
| A51 | 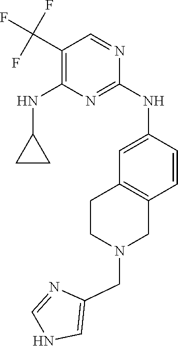 [SMILES: FC(F)(F)c1cnc(Nc2ccc3c(c2)CCN(Cc2c[nH]cn2)C3)nc1NC1CC1] | A | A |
| A52 | 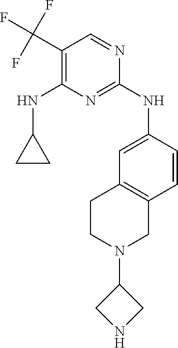 [SMILES: FC(F)(F)c1cnc(Nc2ccc3c(c2)CCN(C2CNC2)C3)nc1NC1CC1] | A | |
| A53 | 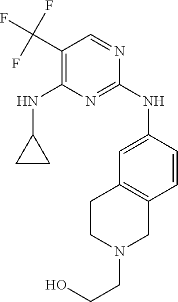 [SMILES: OCCN1CCc2cc(Nc3ncc(C(F)(F)F)c(NC4CC4)n3)ccc2C1] | A | |
| A54 | 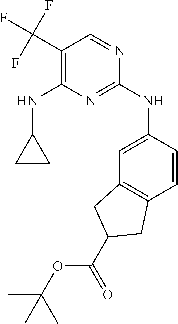 [SMILES: CC(C)(C)OC(=O)C1Cc2ccc(Nc3ncc(C(F)(F)F)c(NC4CC4)n3)cc2C1] | B | |
| A55 | 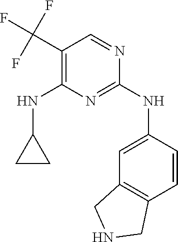 [SMILES: FC(F)(F)c1cnc(Nc2ccc3c(c2)CNC3)nc1NC1CC1] | A | A |
| A56 | 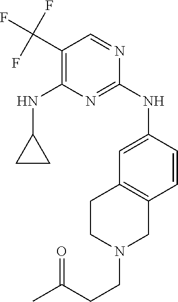 [SMILES: CC(=O)CCN1CCc2cc(Nc3ncc(C(F)(F)F)c(NC4CC4)n3)ccc2C1] | A | |
| A57 | 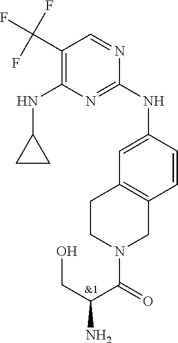 [SMILES: N[C@@H](CO)C(=O)N1CCc2cc(Nc3ncc(C(F)(F)F)c(NC4CC4)n3)ccc2C1] | B | |
| A58 | 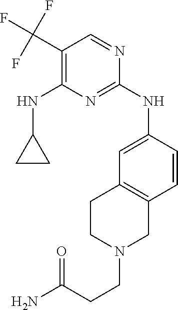 [SMILES: NC(=O)CCN1CCc2cc(Nc3ncc(C(F)(F)F)c(NC4CC4)n3)ccc2C1] | A | |
| A59 | 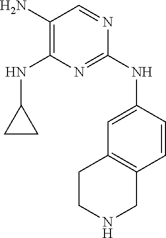 [SMILES: Nc1cnc(Nc2ccc3c(c2)CCNC3)nc1NC1CC1] | C | |
| A60 | 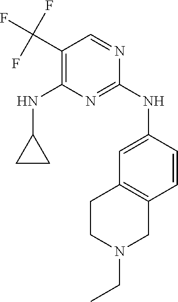 [SMILES: CCN1CCc2cc(Nc3ncc(C(F)(F)F)c(NC4CC4)n3)ccc2C1] | B | |
| A61 | 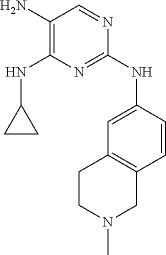 [SMILES: CN1CCc2cc(Nc3ncc(N)c(NC4CC4)n3)ccc2C1] | C | |
| A62 | 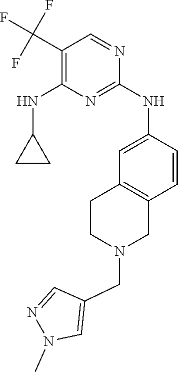 [SMILES: Cn1cc(CN2CCc3cc(Nc4ncc(C(F)(F)F)c(NC5CC5)n4)ccc3C2)cn1] | A | |
| A63 | 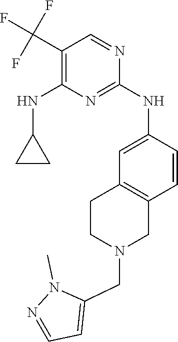 [SMILES: Cn1nccc1CN1CCc2cc(Nc3ncc(C(F)(F)F)c(NC4CC4)n3)ccc2C1] | A | |
| A64 | 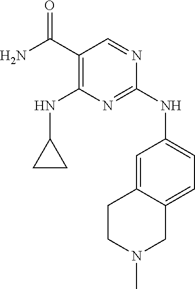 [SMILES: CN1CCc2cc(Nc3ncc(C(N)=O)c(NC4CC4)n3)ccc2C1] | A | |
| A65 | 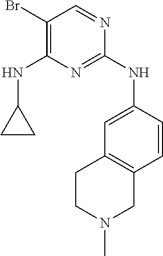 [SMILES: CN1CCc2cc(Nc3ncc(Br)c(NC4CC4)n3)ccc2C1] | A | |
| A66 | 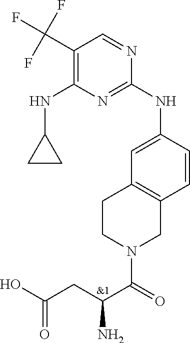 [SMILES: N[C@@H](CC(=O)O)C(=O)N1CCc2cc(Nc3ncc(C(F)(F)F)c(NC4CC4)n3)ccc2C1] | A | |
| A67 | 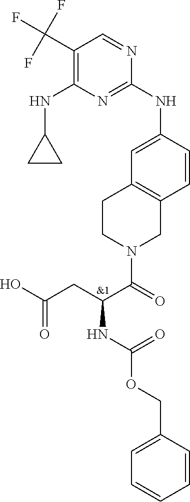 [SMILES: O=C(O)C[C@H](NC(=O)OCc1ccccc1)C(=O)N1CCc2cc(Nc3ncc(C(F)(F)F)c(NC4CC4)n3)ccc2C1] | A | |
| A68 | 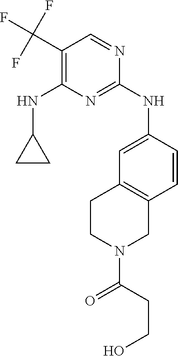 [SMILES: O=C(CCO)N1CCc2cc(Nc3ncc(C(F)(F)F)c(NC4CC4)n3)ccc2C1] | A | |
| A69 | 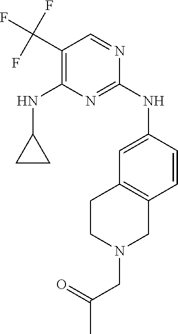 [SMILES: CC(=O)CN1CCc2cc(Nc3ncc(C(F)(F)F)c(NC4CC4)n3)ccc2C1] | A | |
| A70 | 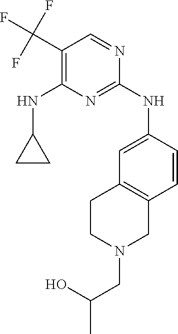 [SMILES: CC(O)CN1CCc2cc(Nc3ncc(C(F)(F)F)c(NC4CC4)n3)ccc2C1] | A | |
| A71 | 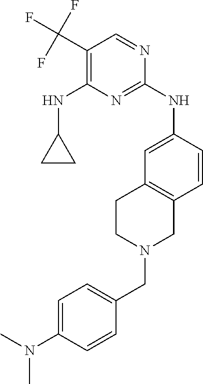 [SMILES: CN(C)c1ccc(CN2CCc3cc(Nc4ncc(C(F)(F)F)c(NC5CC5)n4)ccc3C2)cc1] | A | |
| A72 | 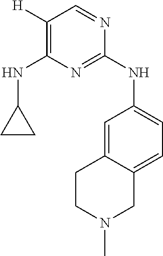 [SMILES: CN1CCc2cc(Nc3nccc(NC4CC4)n3)ccc2C1] | C | |
| A73 | 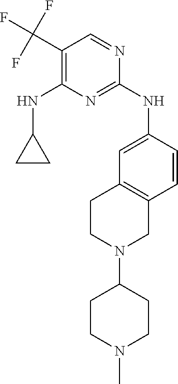 [SMILES: CN1CCC(N2CCc3cc(Nc4ncc(C(F)(F)F)c(NC5CC5)n4)ccc3C2)CC1] | A | |
| A74 | 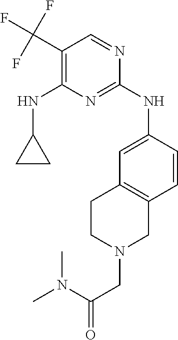 [SMILES: CN(C)C(=O)CN1CCc2cc(Nc3ncc(C(F)(F)F)c(NC4CC4)n3)ccc2C1] | A | |
| A75 | 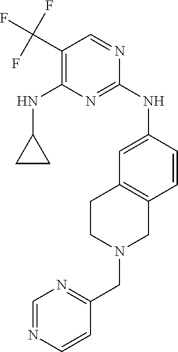 [SMILES: FC(F)(F)c1cnc(Nc2ccc3c(c2)CCN(Cc2ccncn2)C3)nc1NC1CC1] | A | |
| A76 | 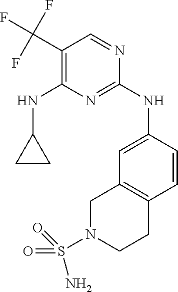 [SMILES: NS(=O)(=O)N1CCc2ccc(Nc3ncc(C(F)(F)F)c(NC4CC4)n3)cc2C1] | B | |
| A77 | 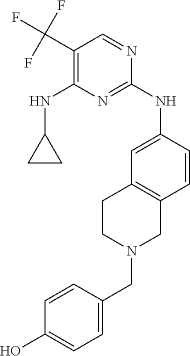 [SMILES: Oc1ccc(CN2CCc3cc(Nc4ncc(C(F)(F)F)c(NC5CC5)n4)ccc3C2)cc1] | A | |
| A78 | 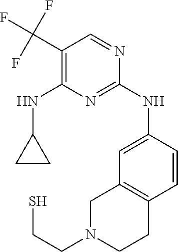 [SMILES: FC(F)(F)c1cnc(Nc2ccc3c(c2)CN(CCS)CC3)nc1NC1CC1] | A | |
| A79 | 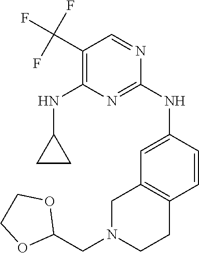 [SMILES: FC(F)(F)c1cnc(Nc2ccc3c(c2)CN(CC2OCCO2)CC3)nc1NC1CC1] | B | |
| A80 | 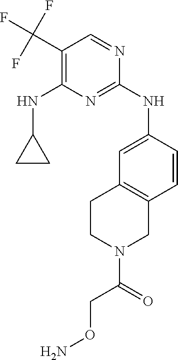 [SMILES: NOCC(=O)N1CCc2cc(Nc3ncc(C(F)(F)F)c(NC4CC4)n3)ccc2C1] | B | |
| A81 | 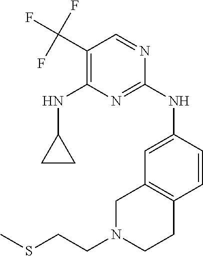 [SMILES: CSCCN1CCc2ccc(Nc3ncc(C(F)(F)F)c(NC4CC4)n3)cc2C1] | A | |
| A82 | 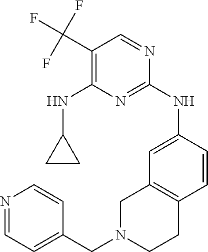 [SMILES: FC(F)(F)c1cnc(Nc2ccc3c(c2)CN(Cc2ccncc2)CC3)nc1NC1CC1] | A | |
| A83 | 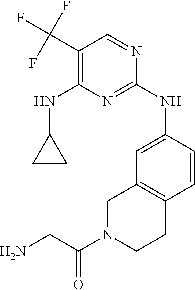 [SMILES: NCC(=O)N1CCc2ccc(Nc3ncc(C(F)(F)F)c(NC4CC4)n3)cc2C1] | B | |
| A84 | 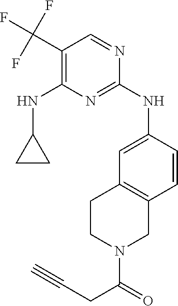 [SMILES: C#CCC(=O)N1CCc2cc(Nc3ncc(C(F)(F)F)c(NC4CC4)n3)ccc2C1] | B | B |
| A85 | 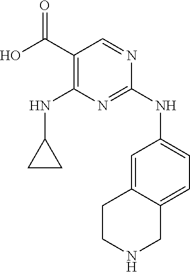 [SMILES: O=C(O)c1cnc(Nc2ccc3c(c2)CCNC3)nc1NC1CC1] | C | |
| A86 | 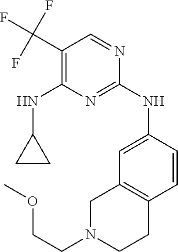 [SMILES: COCCN1CCc2ccc(Nc3ncc(C(F)(F)F)c(NC4CC4)n3)cc2C1] | A | |
| A87 | 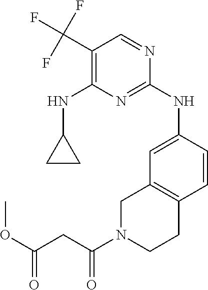 [SMILES: COC(=O)CC(=O)N1CCc2ccc(Nc3ncc(C(F)(F)F)c(NC4CC4)n3)cc2C1] | B | |
| A88 | 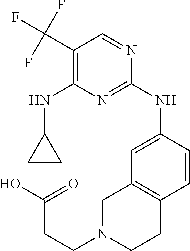 [SMILES: O=C(O)CCN1CCc2ccc(Nc3ncc(C(F)(F)F)c(NC4CC4)n3)cc2C1] | B | |
| A89 | 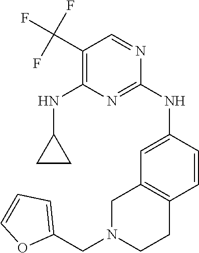 [SMILES: FC(F)(F)c1cnc(Nc2ccc3c(c2)CN(Cc2ccco2)CC3)nc1NC1CC1] | B | |
| A90 | 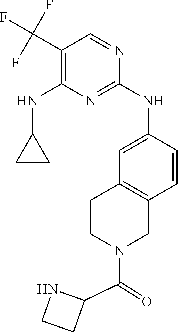 [SMILES: O=C(C1CCN1)N1CCc2cc(Nc3ncc(C(F)(F)F)c(NC4CC4)n3)ccc2C1] | B | |
| A91 | 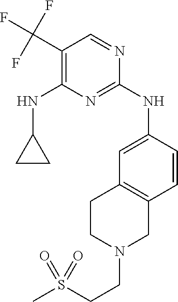 [SMILES: CS(=O)(=O)CCN1CCc2cc(Nc3ncc(C(F)(F)F)c(NC4CC4)n3)ccc2C1] | B | |
| A92 | 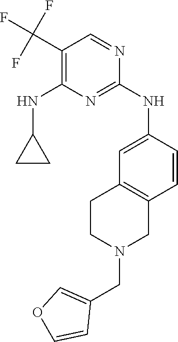 [SMILES: FC(F)(F)c1cnc(Nc2ccc3c(c2)CCN(Cc2ccoc2)C3)nc1NC1CC1] | B | |
| A93 | 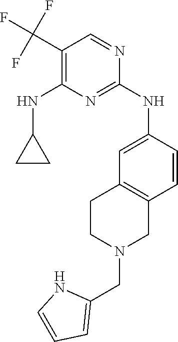 [SMILES: FC(F)(F)c1cnc(Nc2ccc3c(c2)CCN(Cc2ccc[nH]2)C3)nc1NC1CC1] | B | |
| A94 | 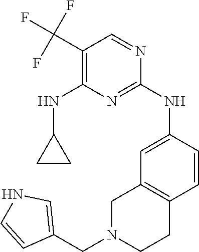 [SMILES: FC(F)(F)c1cnc(Nc2ccc3c(c2)CN(Cc2cc[nH]c2)CC3)nc1NC1CC1] | B | |
| A95 | 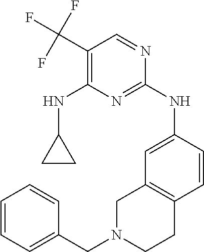 [SMILES: FC(F)(F)c1cnc(Nc2ccc3c(c2)CN(Cc2ccccc2)CC3)nc1NC1CC1] | C | |
| A96 | 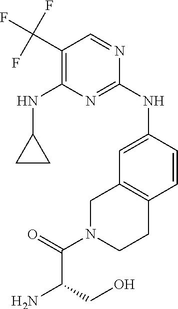 [SMILES: N[C@@H](CO)C(=O)N1CCc2ccc(Nc3ncc(C(F)(F)F)c(NC4CC4)n3)cc2C1] | C | |
| A97 | 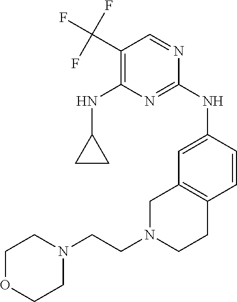 [SMILES: FC(F)(F)c1cnc(Nc2ccc3c(c2)CN(CCN2CCOCC2)CC3)nc1NC1CC1] | B | |
| A98 | 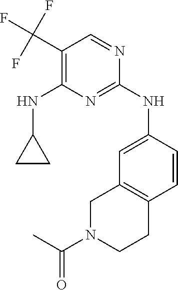 [SMILES: CC(=O)N1CCc2ccc(Nc3ncc(C(F)(F)F)c(NC4CC4)n3)cc2C1] | B | |
| A99 | 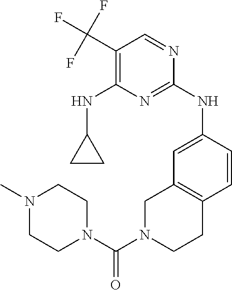 [SMILES: CN1CCN(C(=O)N2CCc3ccc(Nc4ncc(C(F)(F)F)c(NC5CC5)n4)cc3C2)CC1] | B | |
| A100 | 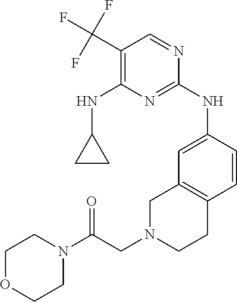 [SMILES: O=C(CN1CCc2ccc(Nc3ncc(C(F)(F)F)c(NC4CC4)n3)cc2C1)N1CCOCC1] | A | |
| A101 | 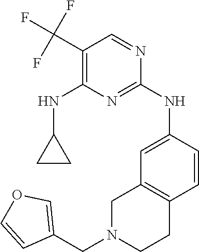 [SMILES: FC(F)(F)c1cnc(Nc2ccc3c(c2)CN(Cc2ccoc2)CC3)nc1NC1CC1] | A | |
| A102 | 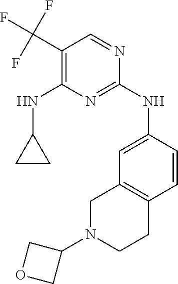 [SMILES: FC(F)(F)c1cnc(Nc2ccc3c(c2)CN(C2COC2)CC3)nc1NC1CC1] | B | |
| A103 | 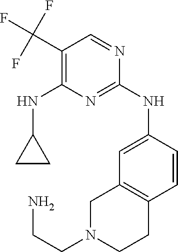 [SMILES: NCCN1CCc2ccc(Nc3ncc(C(F)(F)F)c(NC4CC4)n3)cc2C1] | B | |
| A104 | 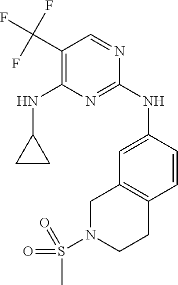 [SMILES: CS(=O)(=O)N1CCc2ccc(Nc3ncc(C(F)(F)F)c(NC4CC4)n3)cc2C1] | B | |
| A105 | 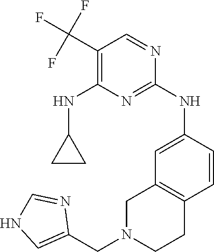 [SMILES: FC(F)(F)c1cnc(Nc2ccc3c(c2)CN(Cc2c[nH]cn2)CC3)nc1NC1CC1] | B | |
| A106 | 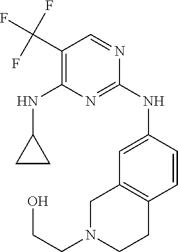 [SMILES: OCCN1CCc2ccc(Nc3ncc(C(F)(F)F)c(NC4CC4)n3)cc2C1] | B | |
| A107 | 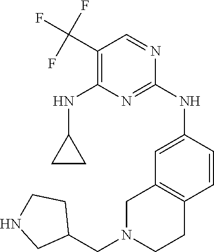 [SMILES: FC(F)(F)c1cnc(Nc2ccc3c(c2)CN(CC2CCNC2)CC3)nc1NC1CC1] | C | |
| A108 | 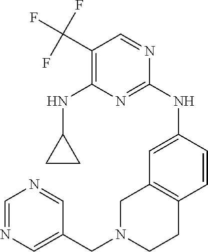 [SMILES: FC(F)(F)c1cnc(Nc2ccc3c(c2)CN(Cc2cncnc2)CC3)nc1NC1CC1] | A | |
| A109 | 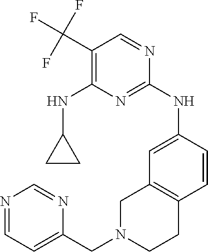 [SMILES: FC(F)(F)c1cnc(Nc2ccc3c(c2)CN(Cc2ccncn2)CC3)nc1NC1CC1] | A | |
| A110 | 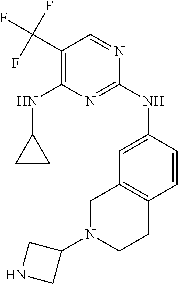 [SMILES: FC(F)(F)c1cnc(Nc2ccc3c(c2)CN(C2CNC2)CC3)nc1NC1CC1] | A | |
| A111 | 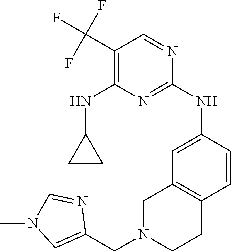 [SMILES: Cn1cnc(CN2CCc3ccc(Nc4ncc(C(F)(F)F)c(NC5CC5)n4)cc3C2)c1] | B | |
| A112 | 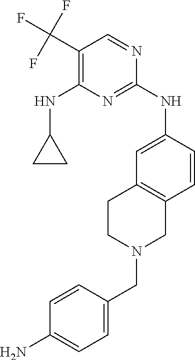 [SMILES: Nc1ccc(CN2CCc3cc(Nc4ncc(C(F)(F)F)c(NC5CC5)n4)ccc3C2)cc1] | C | |
| A113 | 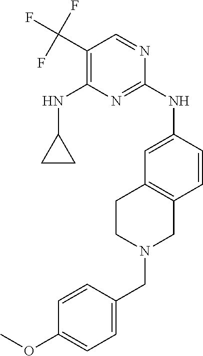 [SMILES: COc1ccc(CN2CCc3cc(Nc4ncc(C(F)(F)F)c(NC5CC5)n4)ccc3C2)cc1] | A | |
| A114 | 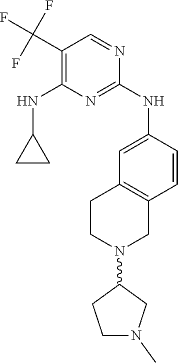 [SMILES: CN1CCC(N2CCc3cc(Nc4ncc(C(F)(F)F)c(NC5CC5)n4)ccc3C2)C1] | A | |
| A115 | 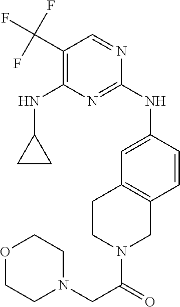 [SMILES: O=C(CN1CCOCC1)N1CCc2cc(Nc3ncc(C(F)(F)F)c(NC4CC4)n3)ccc2C1] | B | |
| A116 | 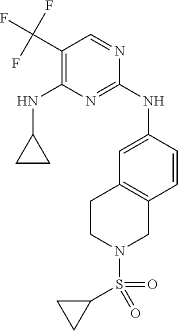 [SMILES: O=S(=O)(C1CC1)N1CCc2cc(Nc3ncc(C(F)(F)F)c(NC4CC4)n3)ccc2C1] | B | |
| A117 | 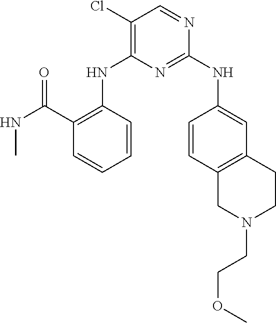 [SMILES: CNC(=O)c1ccccc1Nc1nc(Nc2ccc3c(c2)CCN(CCOC)C3)ncc1Cl] | B | A |
| A118 | 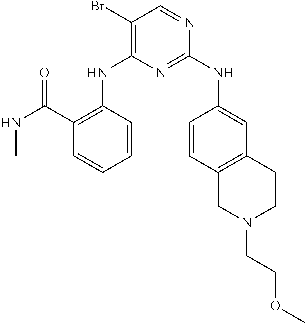 [SMILES: CNC(=O)c1ccccc1Nc1nc(Nc2ccc3c(c2)CCN(CCOC)C3)ncc1Br] | B | A |
| A119 | 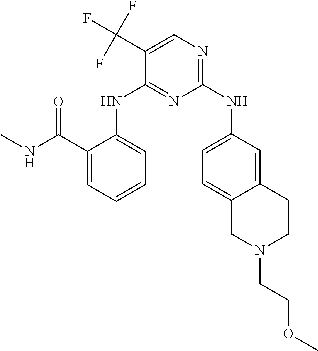 | B | A |
| A120 | 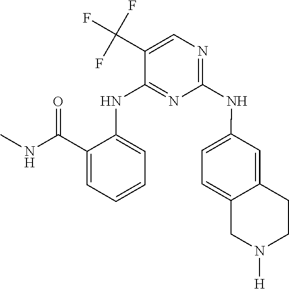 [SMILES: CNC(=O)c1ccccc1Nc1nc(Nc2ccc3c(c2)CCNC3)ncc1C(F)(F)F] | B | A |
| A121 | 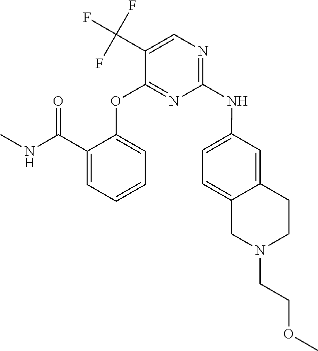 | C | |
| A122 | 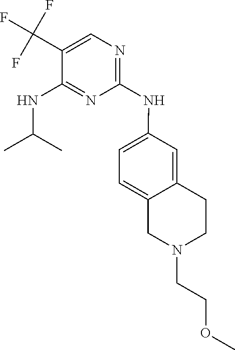 [SMILES: COCCN1CCc2cc(Nc3ncc(C(F)(F)F)c(NC(C)C)n3)ccc2C1] | B | A |
| A123 | 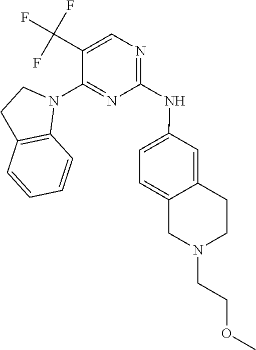 [SMILES: COCCN1CCc2cc(Nc3ncc(C(F)(F)F)c(N4CCc5ccccc54)n3)ccc2C1] | C | |
| A124 | 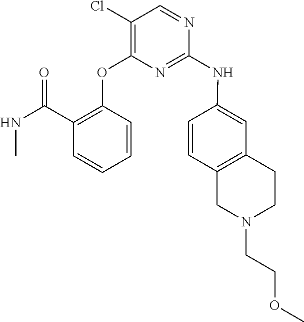 [SMILES: CNC(=O)c1ccccc1Oc1nc(Nc2ccc3c(c2)CCN(CCOC)C3)ncc1Cl] | C | |
| A125 | 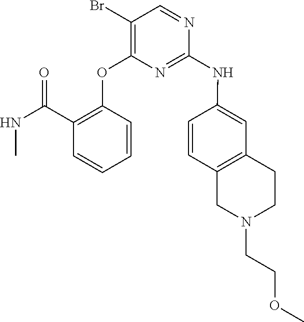 [SMILES: CNC(=O)c1ccccc1Oc1nc(Nc2ccc3c(c2)CCN(CCOC)C3)ncc1Br] | C | |
| A126 | 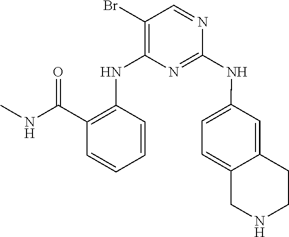 [SMILES: CNC(=O)c1ccccc1Nc1nc(Nc2ccc3c(c2)CCNC3)ncc1Br] | C | |
| A127 | 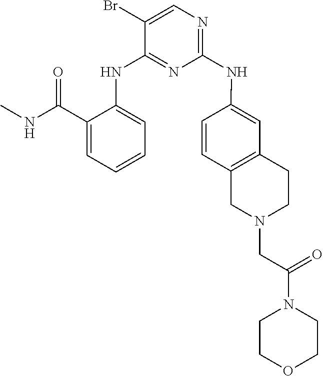 [SMILES: CNC(=O)c1ccccc1Nc1nc(Nc2ccc3c(c2)CCN(CC(=O)N2CCOCC2)C3)ncc1Br] | C | |
| A128 | 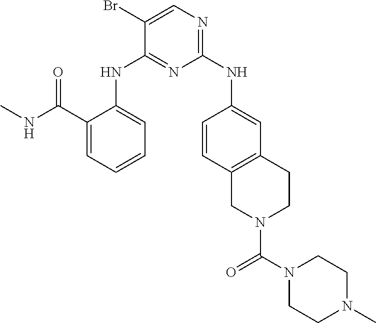 | C | |
| A129 | 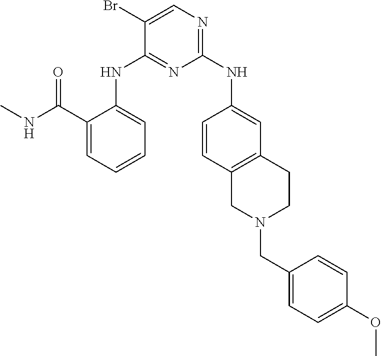 | C | |
| A130 | 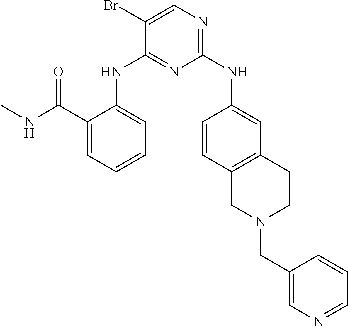 [SMILES: CNC(=O)c1ccccc1Nc1nc(Nc2ccc3c(c2)CCN(Cc2cccnc2)C3)ncc1Br] | C | |
| A131 | 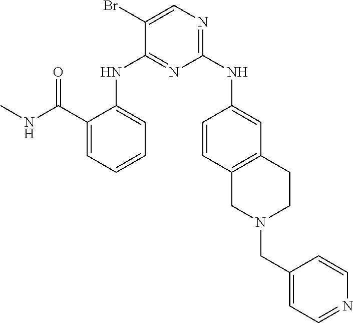 [SMILES: CNC(=O)c1ccccc1Nc1nc(Nc2ccc3c(c2)CCN(Cc2ccncc2)C3)ncc1Br] | C | |
| A132 | 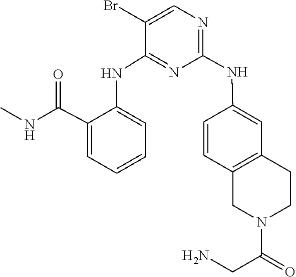 [SMILES: CNC(=O)c1ccccc1Nc1nc(Nc2ccc3c(c2)CCN(C(=O)CN)C3)ncc1Br] | C | |
| A133 | 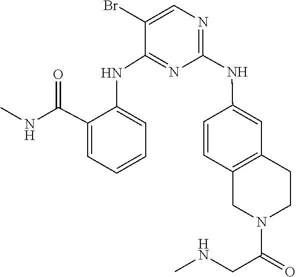 [SMILES: CNCC(=O)N1CCc2cc(Nc3ncc(Br)c(Nc4ccccc4C(=O)NC)n3)ccc2C1] | C | |
| A134 | 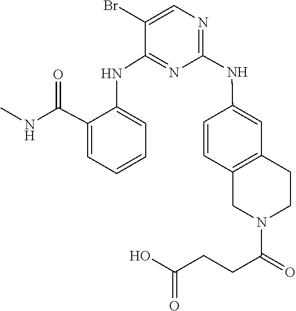 [SMILES: CNC(=O)c1ccccc1Nc1nc(Nc2ccc3c(c2)CCN(C(=O)CCC(=O)O)C3)ncc1Br] | C | |
| A135 | 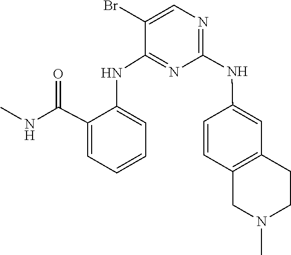 [SMILES: CNC(=O)c1ccccc1Nc1nc(Nc2ccc3c(c2)CCN(C)C3)ncc1Br] | C | |
| A136 | 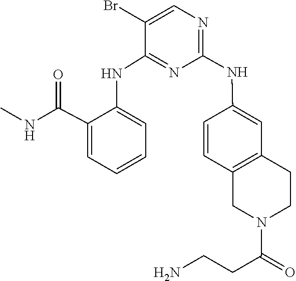 [SMILES: CNC(=O)c1ccccc1Nc1nc(Nc2ccc3c(c2)CCN(C(=O)CCN)C3)ncc1Br] | C | |
| A137 | 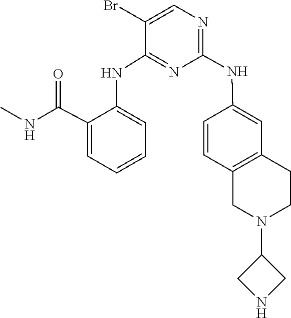 [SMILES: CNC(=O)c1ccccc1Nc1nc(Nc2ccc3c(c2)CCN(C2CNC2)C3)ncc1Br] | C | |
| A138 | 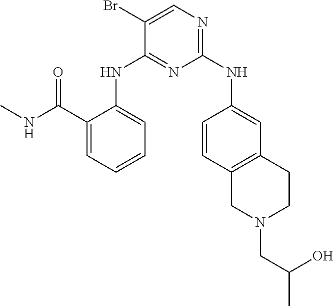 [SMILES: CNC(=O)c1ccccc1Nc1nc(Nc2ccc3c(c2)CCN(CC(C)O)C3)ncc1Br] | C | |
| A139 | 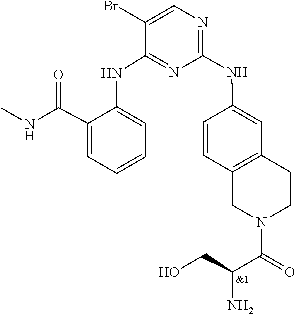 [SMILES: CNC(=O)c1ccccc1Nc1nc(Nc2ccc3c(c2)CCN(C(=O)[C@@H](N)CO)C3)ncc1Br] | C | |
| A140 | 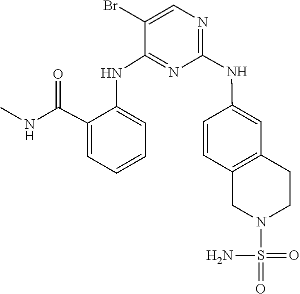 [SMILES: CNC(=O)c1ccccc1Nc1nc(Nc2ccc3c(c2)CCN(S(N)(=O)=O)C3)ncc1Br] | C | |
| A141 | 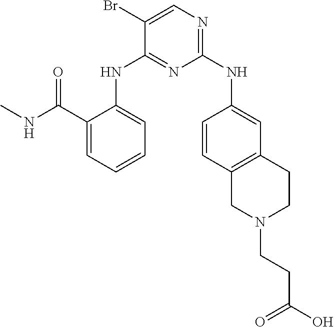 [SMILES: CNC(=O)c1ccccc1Nc1nc(Nc2ccc3c(c2)CCN(CCC(=O)O)C3)ncc1Br] | C | |
| A142 | 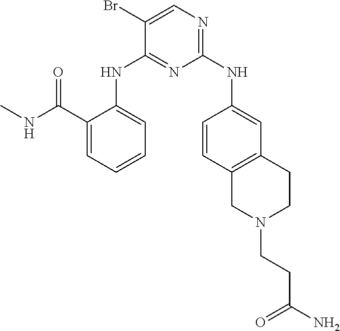 [SMILES: CNC(=O)c1ccccc1Nc1nc(Nc2ccc3c(c2)CCN(CCC(N)=O)C3)ncc1Br] | C | |
| A143 | 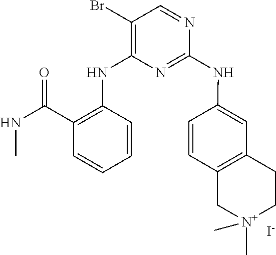 [SMILES: CNC(=O)c1ccccc1Nc1nc(Nc2ccc3c(c2)CC[N+](C)(C)C3)ncc1Br ; [I-]] | C | |
| A144 | 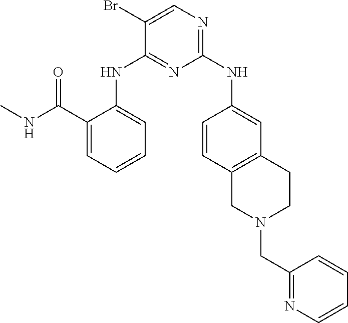 [SMILES: CNC(=O)c1ccccc1Nc1nc(Nc2ccc3c(c2)CCN(Cc2ccccn2)C3)ncc1Br] | C | |
| A145 | 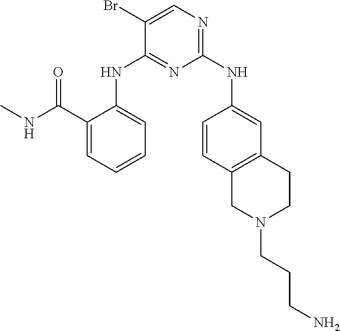 [SMILES: CNC(=O)c1ccccc1Nc1nc(Nc2ccc3c(c2)CCN(CCCN)C3)ncc1Br] | C | |
| A146 | 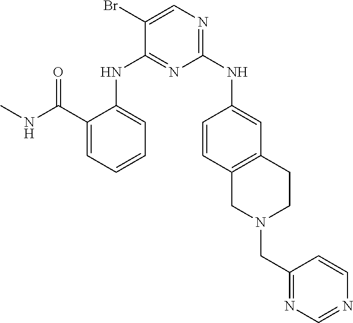 [SMILES: CNC(=O)c1ccccc1Nc1nc(Nc2ccc3c(c2)CCN(Cc2ccncn2)C3)ncc1Br] | C | |
| A147 | 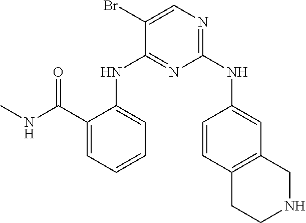 [SMILES: CNC(=O)c1ccccc1Nc1nc(Nc2ccc3c(c2)CNCC3)ncc1Br] | C | |
| A148 | 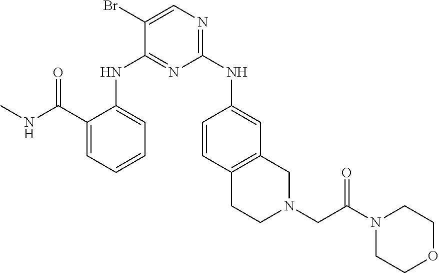 [SMILES: CNC(=O)c1ccccc1Nc1nc(Nc2ccc3c(c2)CN(CC(=O)N2CCOCC2)CC3)ncc1Br] | C | |
| A149 | 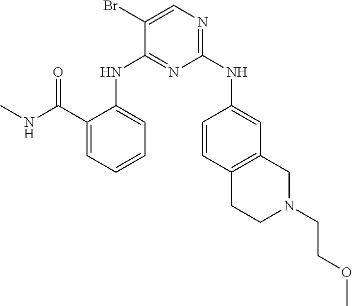 | C | |
| A150 | 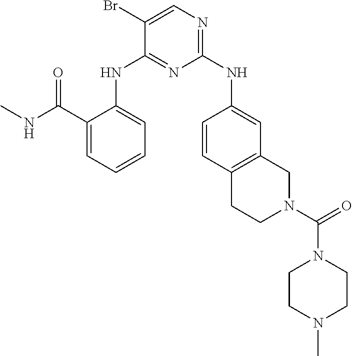 [SMILES: CNC(=O)c1ccccc1Nc1nc(Nc2ccc3c(c2)CN(C(=O)N2CCN(C)CC2)CC3)ncc1Br] | C | |
| A151 | 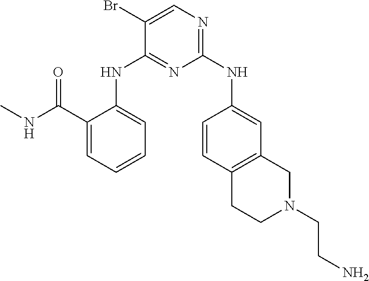 [SMILES: CNC(=O)c1ccccc1Nc1nc(Nc2ccc3c(c2)CN(CCN)CC3)ncc1Br] | C | |
| A152 | 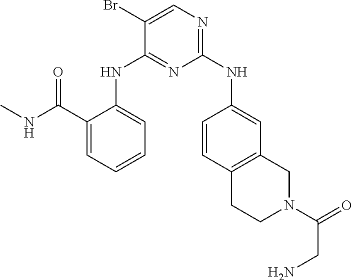 [SMILES: CNC(=O)c1ccccc1Nc1nc(Nc2ccc3c(c2)CN(C(=O)CN)CC3)ncc1Br] | C | |
| A153 | 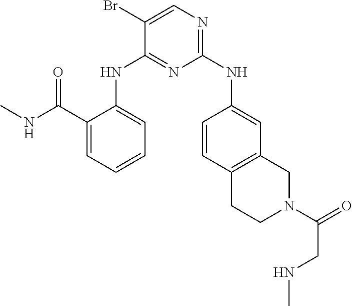 [SMILES: CNCC(=O)N1CCc2ccc(Nc3ncc(Br)c(Nc4ccccc4C(=O)NC)n3)cc2C1] | C | |
| A154 | 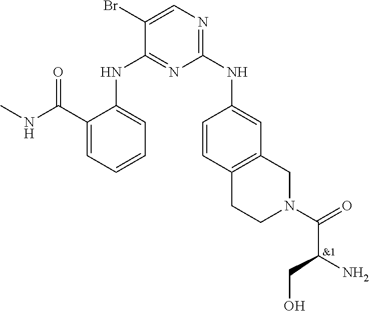 [SMILES: CNC(=O)c1ccccc1Nc1nc(Nc2ccc3c(c2)CN(C(=O)[C@@H](N)CO)CC3)ncc1Br] | C | |
| A155 | 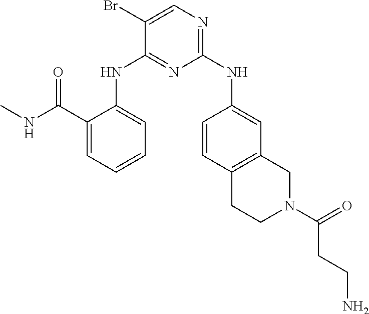 [SMILES: CNC(=O)c1ccccc1Nc1nc(Nc2ccc3c(c2)CN(C(=O)CCN)CC3)ncc1Br] | C | |
| A156 | 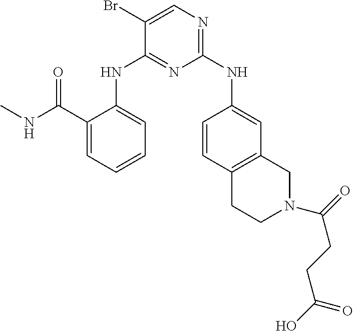 [SMILES: CNC(=O)c1ccccc1Nc1nc(Nc2ccc3c(c2)CN(C(=O)CCC(=O)O)CC3)ncc1Br] | C | |
| A157 | 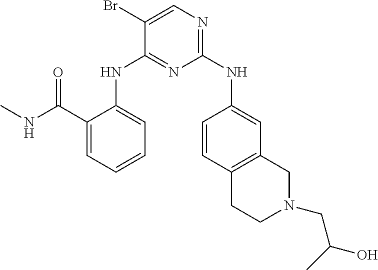 [SMILES: CNC(=O)c1ccccc1Nc1nc(Nc2ccc3c(c2)CN(CC(C)O)CC3)ncc1Br] | C | |
| A158 | 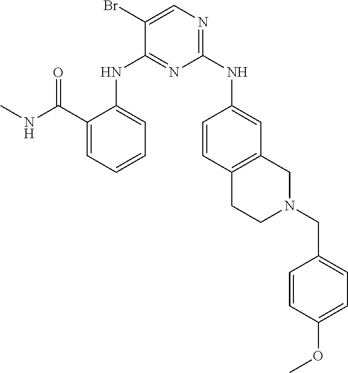 [SMILES: CNC(=O)c1ccccc1Nc1nc(Nc2ccc3c(c2)CN(Cc2ccc(OC)cc2)CC3)ncc1Br] | C | |
| A159 | 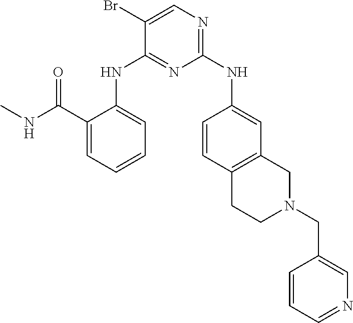 [SMILES: CNC(=O)c1ccccc1Nc1nc(Nc2ccc3c(c2)CN(Cc2cccnc2)CC3)ncc1Br] | C | |
| A160 | 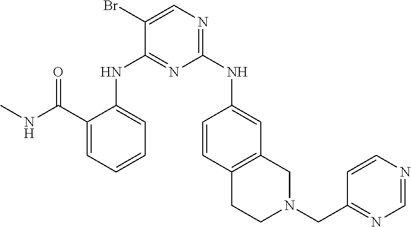 [SMILES: CNC(=O)c1ccccc1Nc1nc(Nc2ccc3c(c2)CN(Cc2ccncn2)CC3)ncc1Br] | C | |
| A161 | 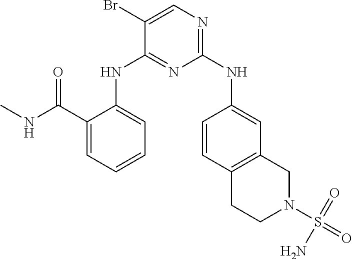 [SMILES: CNC(=O)c1ccccc1Nc1nc(Nc2ccc3c(c2)CN(S(N)(=O)=O)CC3)ncc1Br] | C | |
| A162 | 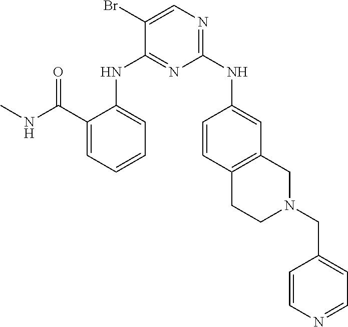 [SMILES: CNC(=O)c1ccccc1Nc1nc(Nc2ccc3c(c2)CN(Cc2ccncc2)CC3)ncc1Br] | C | |
| A163 | 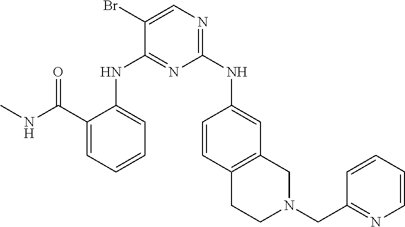 [SMILES: CNC(=O)c1ccccc1Nc1nc(Nc2ccc3c(c2)CN(Cc2ccccn2)CC3)ncc1Br] | C | |
| A164 | 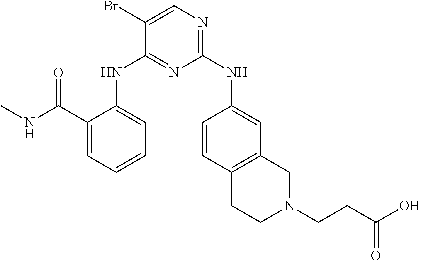 [SMILES: CNC(=O)c1ccccc1Nc1nc(Nc2ccc3c(c2)CN(CCC(=O)O)CC3)ncc1Br] | C | |
| A165 | 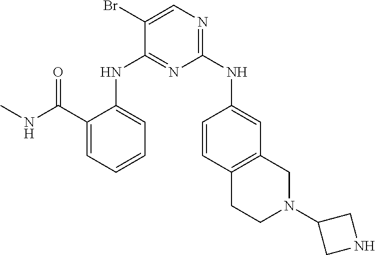 [SMILES: CNC(=O)c1ccccc1Nc1nc(Nc2ccc3c(c2)CN(C2CNC2)CC3)ncc1Br] | C | |
| A166 | 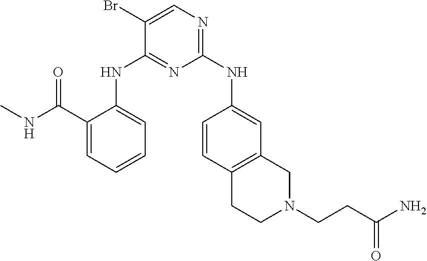 [SMILES: CNC(=O)c1ccccc1Nc1nc(Nc2ccc3c(c2)CN(CCC(N)=O)CC3)ncc1Br] | C | |
| A167 | 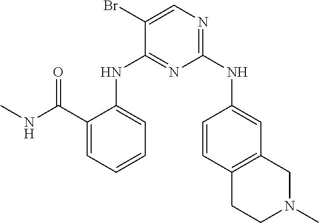 | C | |
| A168 | 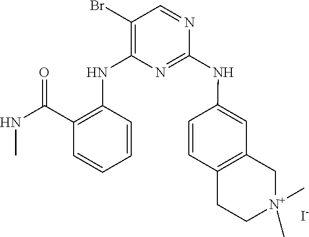 [SMILES: CNC(=O)c1ccccc1Nc1nc(Nc2ccc3c(c2)C[N+](C)(C)CC3)ncc1Br ; [I-]] | C | |

In one aspect, provided herein, is a compound having a structure of Formula (IIB):

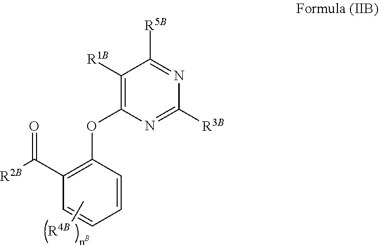

wherein:
R^{1B} is H, halogen, substituted or unsubstituted alkyl, or substituted or unsubstituted haloalkyl;
R^{2B} is substituted C₂ alkyl, substituted or unsubstituted C₃-C₁₀ alkyl, —NR^{21B}R^{22B}, or —OR²³B.
R^{3B} is —OR^{31B}, —SR^{31B}, or —NR^{32B}R^{33B};
each R^{4B} is independently halogen, —CN, —OR^{B}, —SR^{B}, —S(═O)R^{B}, —S(═O)₂R^{B}, —NO₂, —NR^{B}R^{B}, —NR^{B}S(═O)₂R^{B}, —S(═O)₂NR^{B}R^{B}, —C(═O)R^{B}, —OC(═O)R^{B}, —C(═O)C(═O)R^{B}, —C(═O)OR^{B}, —C(═O)NR^{B}OR^{B}, —OC(═O)OR^{B}, —C(═O)NR^{B}R^{B}, —OC(═O)NR^{B}R^{B}, —NR^{B}C(═O)NR^{B}R^{B}, —NR^{B}S(═O)₂NR^{B}R^{B}, —NR^{B}C(═O)R^{B}, —NR^{B}C(═O)OR^{B}, substituted or unsubstituted alkyl, substituted or unsubstituted cycloalkyl, substituted or unsubstituted heterocycloalkyl, substituted or unsubstituted aryl, or substituted or unsubstituted heteroaryl;
R^{5B} is H or halogen;
R^{21B} is —OR^{26B}, NR^{27B}R^{28B}, substituted methyl, or substituted or unsubstituted C₂-C₁₀ alkyl;
   R^{22B} is H or substituted or unsubstituted alkyl; or
      R^{21B} and R^{22B} are taken together with the nitrogen atom to which they are attached to form a substituted or unsubstituted heterocycloalkyl containing at least one additional heteroatom selected from the group consisting of O, N, and S;
   R^{23B} is H or substituted or unsubstituted alkyl;
   R^{26B} is H or substituted or unsubstituted alkyl;
   R^{27B} and R^{28B} are each independently H or substituted or unsubstituted alkyl; or
   R^{27B} and R^{28B} are taken together with the nitrogen atom to which they are attached to form a substituted or unsubstituted heterocycloalkyl;
   R^{31B} is substituted or unsubstituted aryl or substituted or unsubstituted heteroaryl;
   R^{32B} is substituted or unsubstituted aryl or substituted or unsubstituted heteroaryl;
   R^{33B} is H or substituted or unsubstituted alkyl; or
   R^{32B} and R^{33B} are taken together with the nitrogen atom to which they are attached to form a substituted or unsubstituted heterocycloalkyl;
   each R^{B} is independently H, substituted or unsubstituted alkyl, substituted or unsubstituted heteroalkyl, substituted or unsubstituted aryl, or substituted or unsubstituted heteroaryl; and
   n^{B} is an integer from 0-4;
   or a pharmaceutically acceptable salt thereof.

In some embodiments, R^{1B} is halogen or C₁-C₆ haloalkyl. In some embodiments, R^{1B} is Cl, Br, or —CF₃. In some embodiments, R^{1B} is —CF₃. In some embodiments R^{1B} is Br. In some embodiments, R^{1B} is Cl.

In some embodiments, R^{2B} is —NR^{21B}R^{22B} or —OR^{23B}. In some embodiments, R^{2B} is —NR^{21B}R^{22B}. In some embodiments, R^{21B} is substituted methyl or substituted or unsubstituted C₂-C₆ alkyl. In some embodiments, R^{21B} is substituted methyl or substituted C₂-C₄ alkyl. In some embodiments, R^{21B} is substituted C₂-C₄ alkyl. In some embodiments, R^{21B} is

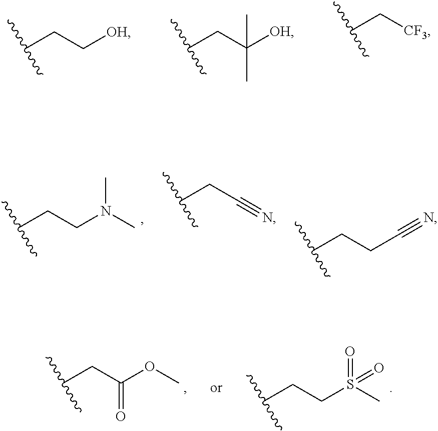

In some embodiments, R^{22B} is H or —CH₃. In some embodiments, R^{22B} is —CH₃. In some embodiments, R^{22B} is H. In some embodiments, R^{22B} is H, —CH₃, —CH₂CH₃, or —CH₂CH₂CH₃.

In some embodiments, R^{2B} is —OR^{23B}. In some embodiments R^{23B} is H or —CH₃. In some embodiments, R^{23B} is —CH₃. In some embodiments R^{23B} is H. In some embodiments, R^{23B} is H, —CH₃, —CH₂CH₃, or —CH₂CH₂CH₃.

In some embodiments, R^{3B} is —NR^{32B}R^{33B}. In some embodiments, R^{32B} is substituted or unsubstituted aryl fused with a 5- or 6-membered ring. In some embodiments, R^{32B} is substituted or unsubstituted heteroaryl fused with a 5- or 6-membered ring. In some embodiments, R^{32B} is substituted or unsubstituted aryl or substituted or unsubstituted heteroaryl, wherein the aryl or heteroaryl is

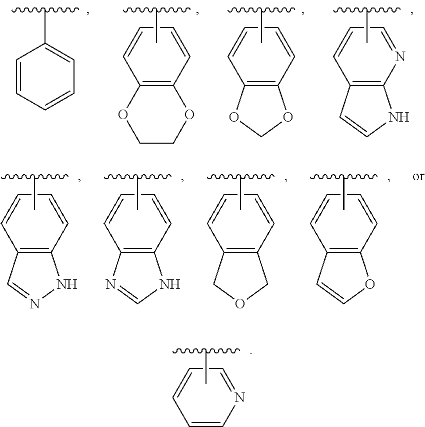

In some embodiments, R^{32B} is substituted or unsubstituted aryl or substituted or unsubstituted heteroaryl, wherein the aryl or heteroaryl is

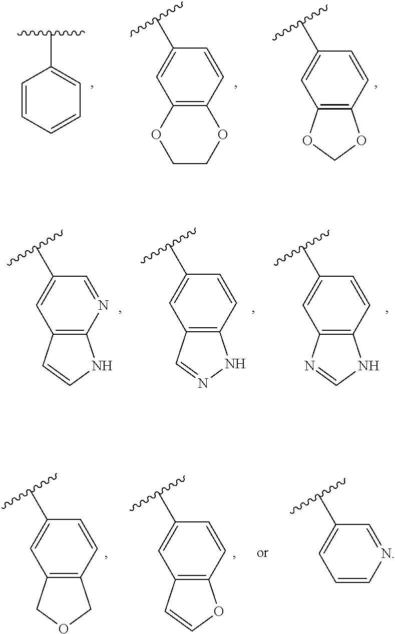

In some embodiments, R^{32B} is unsubstituted aryl or heteroaryl.

In some embodiments, R^{32B} is aryl optionally substituted with one or more halogen, —CN, —OR^{B}, —SR^{B}, —S(═O)R^{B}, S(═O)₂R^{B}, —NO₂, —NR^{B}R^{B}, —NR^{B}S(═O)₂R^{B}, S(═O)₂NR^{B}R^{B}, —C(═O)R^{B}, —OC(═O)R^{B}, —C(═O)C(═O)R^{B}, —C(═O)OR^{B}, —C(═O)NR^{B}OR^{B}, —OC(═O)OR^{B}, —C(═O)NR^{B}R^{B}, —OC(═O)NR^{B}R^{B}, —NR^{B}C(═O)NR^{B}R^{B}, —NR^{B}S(═O)₂NR^{B}R^{B}, —NR^{B}C(═O)R^{B}, —NR^{B}C(═O)OR^{B}, substituted or unsubstituted alkyl, substituted or unsubstituted cycloalkyl, or substituted or unsubstituted heterocycloalkyl. In some embodiments, R^{32B} is phenyl optionally substituted with one or more halogen, —CN, —OR^{B}, —SR^{B}, —S(═O)R^{B}, —S(═O)₂R^{B}, —NO₂, —NR^{B}R^{B}, —NR^{B}S(═O)₂R^{B}, —S(═O)₂NR^{B}R^{B}, —C(═O)R^{B}, —OC(═O)R^{B}, —C(═O)C(═O)R^{B}, —C(═O)OR^{B}, —C(═O)NR^{B}OR^{B}, —OC(═O)OR^{B}, —C(═O)NR^{B}R^{B}, —OC(═O)NR^{B}R^{B}, —NR^{B}C(═O)NR^{B}R^{B}, —NR^{B}S(═O)₂NR^{B}R^{B}, —NR^{B}C(═O)R^{B}, —NR^{B}C(═O)OR^{B}, substituted or unsubstituted alkyl, substituted or unsubstituted cycloalkyl, or substituted or unsubstituted heterocycloalkyl. In some embodiments, R^{32B} is

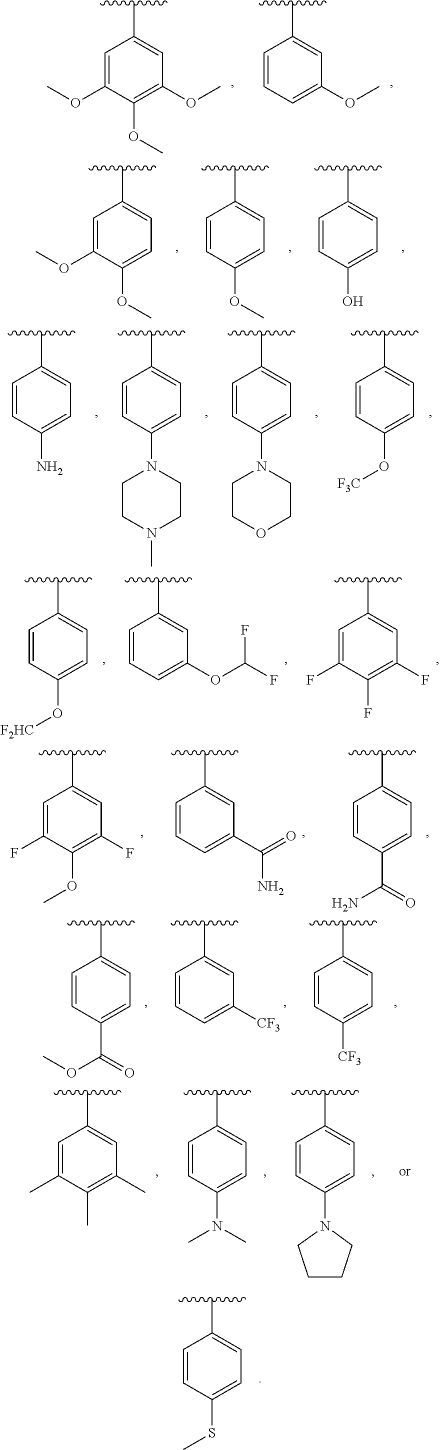

In some embodiments, R^{33B} H or C₁-C₆ alkyl. In some embodiments, R^{33B} H or C₁-C₃ alkyl. In some embodiments, R^{33B} is H or —CH₃. In some embodiments, R^{33B} is H. In some embodiments, R^{33B} is —CH₃.

In some embodiments, R^{4B} is independently halogen, —CN, —OR^{B}, —C(═O)R^{B}, —OC(═O)R^{B}, —OC(═O)OR^{B}, —C(═O)NR^{B}R^{B}, —OC(═O)NR^{B}R^{B}, C₁-C₆ alkyl, or C₁-C₆ haloalkyl. In some embodiments, each R^{4B} is independently halogen, —OR^{B}, or C₁-C₆ alkyl. In some embodiments, each R^{4B} is independently —OR^{B}.

In some embodiments, n^{B} is 0, 1, or 2. In some embodiments, n^{B} is 0 or 1. In some embodiments, n^{B} is 0. In some embodiments, n^{B} is 1. In some embodiments, n^{B} is 2. In some embodiments, n^{B} is 3. In some embodiments, n^{B} is 4.

In some embodiments, R^{5B} is H or F. In some embodiments, R^{5B} is H. In some embodiments, R^{5B} is F.

Any combination of the groups described above for the various variables is contemplated herein. Throughout the specification, groups and substituents thereof are chosen by one skilled in the field to provide stable moieties and compounds.

Illustrative compounds of Formula II and related analogs are shown in Table 2 (along with their respective IC₅₀ values for ULK1 inhibition assay). IC₅₀s are represented nM, with A representing IC₅₀<100 nM, B representing 1000 nM>IC₅₀>20 nM, and C representing IC₅₀>1000 nM. NT indicates the compound was not tested.

TABLE 2

| | | ULK1 IC50 ADP-Glo |
|---|---|---|
| | | (A < 100 nM, 100 nM |
| Compound | | < B < 1000 nM, C > |
| Number | Structure | 1000 nM) |
| B1 | 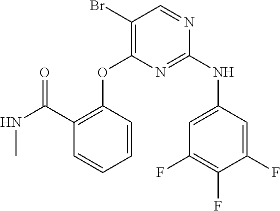 [SMILES: CNC(=O)c1ccccc1Oc1nc(Nc2cc(F)c(F)c(F)c2)ncc1Br] | C |
| B2 | 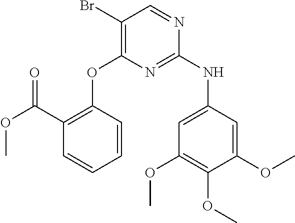 [SMILES: COC(=O)c1ccccc1Oc1nc(Nc2cc(OC)c(OC)c(OC)c2)ncc1Br] | A |
| B3 | 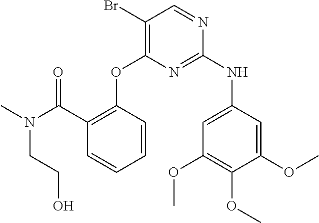 | B |
| B4 | 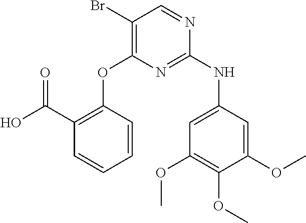 [SMILES: COc1cc(Nc2ncc(Br)c(Oc3ccccc3C(=O)O)n2)cc(OC)c1OC] | B |
| B5 | 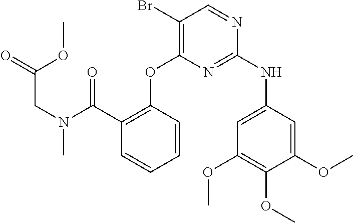 [SMILES: COC(=O)CN(C)C(=O)c1ccccc1Oc1nc(Nc2cc(OC)c(OC)c(OC)c2)ncc1Br] | B |
| B6 | 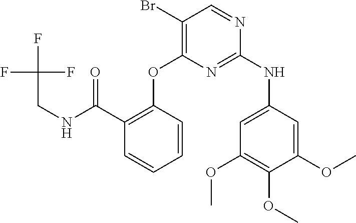 [SMILES: COc1cc(Nc2ncc(Br)c(Oc3ccccc3C(=O)NCC(F)(F)F)n2)cc(OC)c1OC] | B |
| B7 | 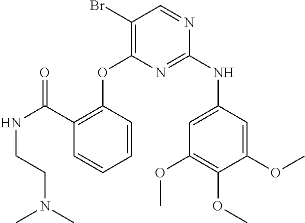 [SMILES: COc1cc(Nc2ncc(Br)c(Oc3ccccc3C(=O)NCCN(C)C)n2)cc(OC)c1OC] | C |
| B8 | 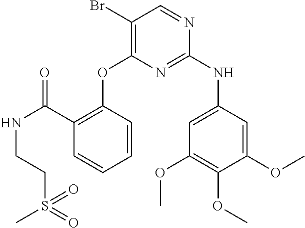 [SMILES: COc1cc(Nc2ncc(Br)c(Oc3ccccc3C(=O)NCCS(C)(=O)=O)n2)cc(OC)c1OC] | C |
| B9 | 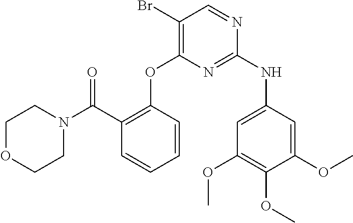 [SMILES: COc1cc(Nc2ncc(Br)c(Oc3ccccc3C(=O)N3CCOCC3)n2)cc(OC)c1OC] | C |
| B10 | 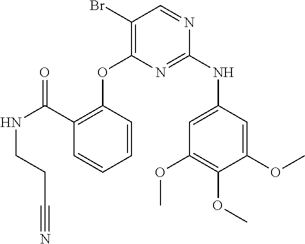 [SMILES: COc1cc(Nc2ncc(Br)c(Oc3ccccc3C(=O)NCCC#N)n2)cc(OC)c1OC] | C |
| B11 | 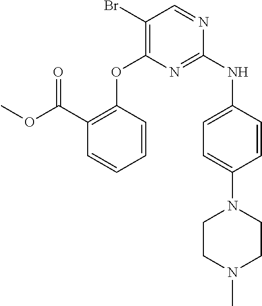 [SMILES: COC(=O)c1ccccc1Oc1nc(Nc2ccc(N3CCN(C)CC3)cc2)ncc1Br] | A |
| B12 | 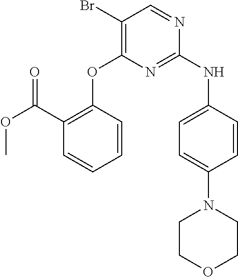 [SMILES: COC(=O)c1ccccc1Oc1nc(Nc2ccc(N3CCOCC3)cc2)ncc1Br] | C |
| B13 | 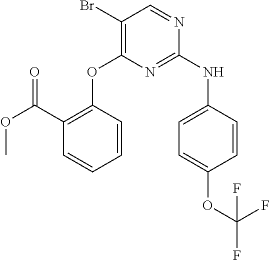 [SMILES: COC(=O)c1ccccc1Oc1nc(Nc2ccc(OC(F)(F)F)cc2)ncc1Br] | C |
| B14 | 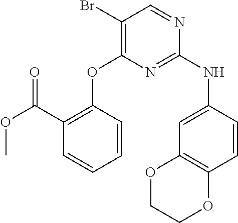 [SMILES: COC(=O)c1ccccc1Oc1nc(Nc2ccc3c(c2)OCCO3)ncc1Br] | C |
| B15 | 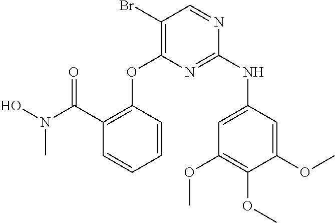 [SMILES: COc1cc(Nc2ncc(Br)c(Oc3ccccc3C(=O)N(C)O)n2)cc(OC)c1OC] | A |
| B16 | 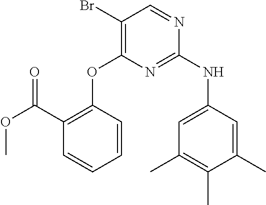 [SMILES: COC(=O)c1ccccc1Oc1nc(Nc2cc(C)c(C)c(C)c2)ncc1Br] | C |
| B17 | 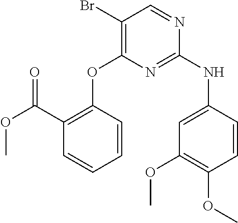 [SMILES: COC(=O)c1ccccc1Oc1nc(Nc2ccc(OC)c(OC)c2)ncc1Br] | C |
| B18 | 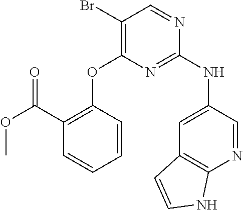 [SMILES: COC(=O)c1ccccc1Oc1nc(Nc2cnc3[nH]ccc3c2)ncc1Br] | C |
| B19 | 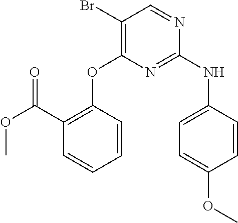 [SMILES: COC(=O)c1ccccc1Oc1nc(Nc2ccc(OC)cc2)ncc1Br] | C |
| B20 | 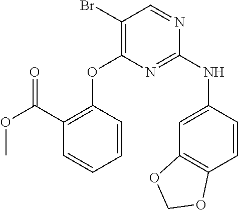 [SMILES: COC(=O)c1ccccc1Oc1nc(Nc2ccc3c(c2)OCO3)ncc1Br] | C |
| B21 | 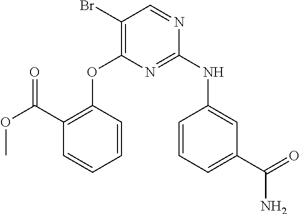 [SMILES: COC(=O)c1ccccc1Oc1nc(Nc2cccc(C(N)=O)c2)ncc1Br] | C |
| B22 | 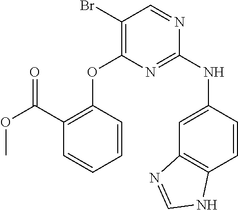 [SMILES: COC(=O)c1ccccc1Oc1nc(Nc2ccc3[nH]cnc3c2)ncc1Br] | C |
| B23 | 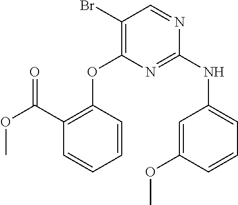 [SMILES: COC(=O)c1ccccc1Oc1nc(Nc2cccc(OC)c2)ncc1Br] | C |
| B24 | 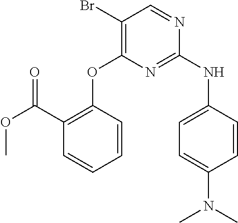 [SMILES: COC(=O)c1ccccc1Oc1nc(Nc2ccc(N(C)C)cc2)ncc1Br] | B |
| B25 | 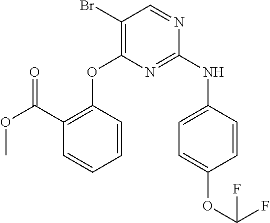 [SMILES: COC(=O)c1ccccc1Oc1nc(Nc2ccc(OC(F)F)cc2)ncc1Br] | C |
| B26 | 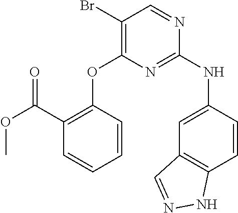 [SMILES: COC(=O)c1ccccc1Oc1nc(Nc2ccc3[nH]ncc3c2)ncc1Br] | B |
| B27 | 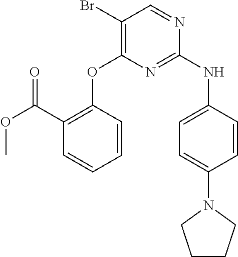 [SMILES: COC(=O)c1ccccc1Oc1nc(Nc2ccc(N3CCCC3)cc2)ncc1Br] | C |
| B28 | 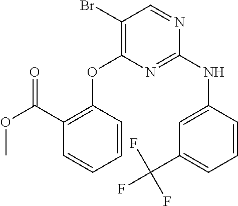 [SMILES: COC(=O)c1ccccc1Oc1nc(Nc2cccc(C(F)(F)F)c2)ncc1Br] | C |
| B29 | 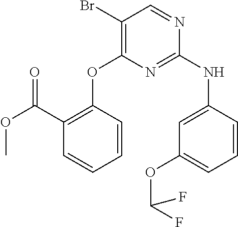 [SMILES: COC(=O)c1ccccc1Oc1nc(Nc2cccc(OC(F)F)c2)ncc1Br] | C |
| B30 | 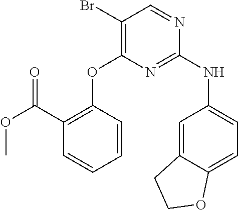 [SMILES: COC(=O)c1ccccc1Oc1nc(Nc2ccc3c(c2)CCO3)ncc1Br] | C |
| B31 | 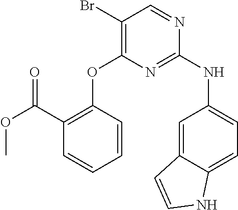 [SMILES: COC(=O)c1ccccc1Oc1nc(Nc2ccc3[nH]ccc3c2)ncc1Br] | C |
| B32 | 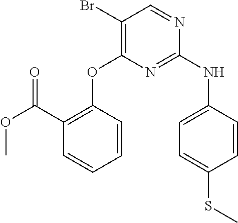 [SMILES: COC(=O)c1ccccc1Oc1nc(Nc2ccc(SC)cc2)ncc1Br] | C |
| B33 | 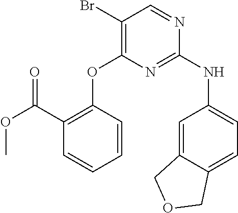 [SMILES: COC(=O)c1ccccc1Oc1nc(Nc2ccc3c(c2)COC3)ncc1Br] | C |
| B34 | 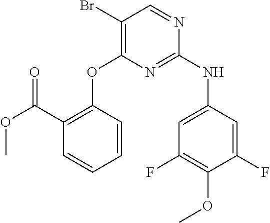 [SMILES: COC(=O)c1ccccc1Oc1nc(Nc2cc(F)c(OC)c(F)c2)ncc1Br] | C |
| B35 | 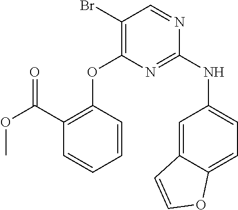 [SMILES: COC(=O)c1ccccc1Oc1nc(Nc2ccc3occc3c2)ncc1Br] | C |
| B36 | 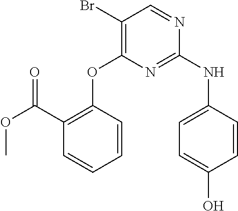 [SMILES: COC(=O)c1ccccc1Oc1nc(Nc2ccc(O)cc2)ncc1Br] | C |
| B37 | 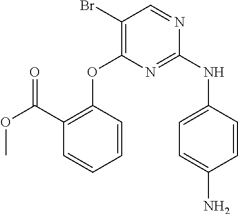 [SMILES: COC(=O)c1ccccc1Oc1nc(Nc2ccc(N)cc2)ncc1Br] | B |
| B38 | 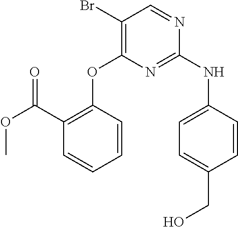 [SMILES: COC(=O)c1ccccc1Oc1nc(Nc2ccc(CO)cc2)ncc1Br] | C |
| B39 | 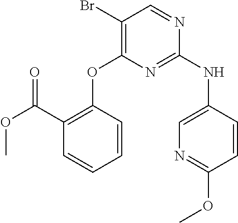 [SMILES: COC(=O)c1ccccc1Oc1nc(Nc2ccc(OC)nc2)ncc1Br] | C |
| B40 | 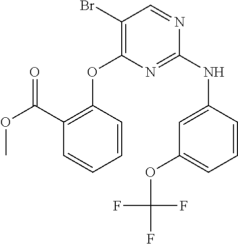 [SMILES: COC(=O)c1ccccc1Oc1nc(Nc2cccc(OC(F)(F)F)c2)ncc1Br] | C |
| B41 | 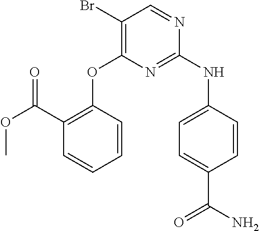 [SMILES: COC(=O)c1ccccc1Oc1nc(Nc2ccc(C(N)=O)cc2)ncc1Br] | C |
| B42 | 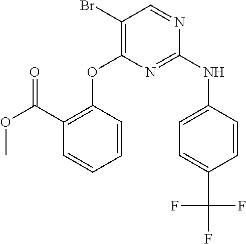 [SMILES: COC(=O)c1ccccc1Oc1nc(Nc2ccc(C(F)(F)F)cc2)ncc1Br] | C |
| B43 | 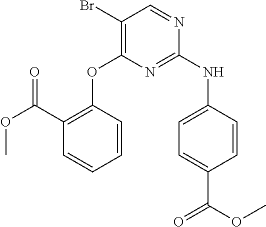 [SMILES: COC(=O)c1ccc(Nc2ncc(Br)c(Oc3ccccc3C(=O)OC)n2)cc1] | C |
| B44 | 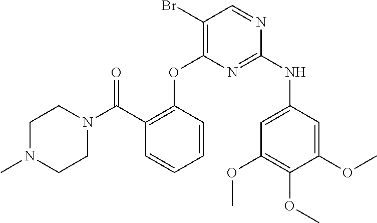 | C |
| B45 | 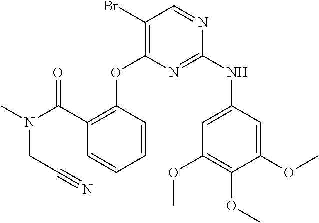 | B |
| B46 | 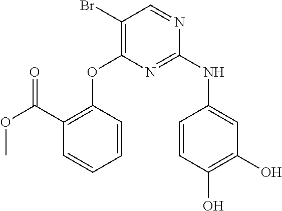 [SMILES: COC(=O)c1ccccc1Oc1nc(Nc2ccc(O)c(O)c2)ncc1Br] | C |
| B47 | 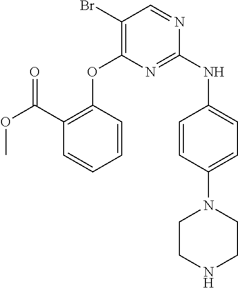 [SMILES: COC(=O)c1ccccc1Oc1nc(Nc2ccc(N3CCNCC3)cc2)ncc1Br] | C |
| B48 | 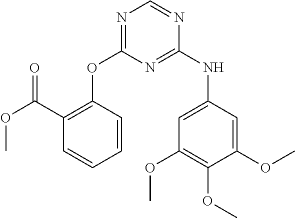 [SMILES: COC(=O)c1ccccc1Oc1ncnc(Nc2cc(OC)c(OC)c(OC)c2)n1] | C |
| B49 | 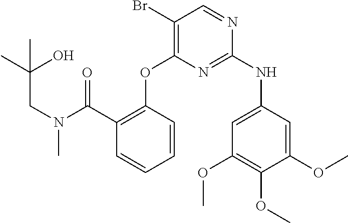 [SMILES: COc1cc(Nc2ncc(Br)c(Oc3ccccc3C(=O)N(C)CC(C)(C)O)n2)cc(OC)c1OC] | C |
| B50 | 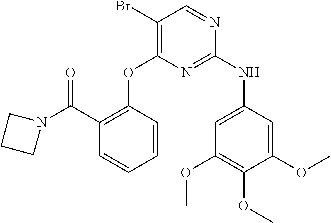 [SMILES: COc1cc(Nc2ncc(Br)c(Oc3ccccc3C(=O)N3CCC3)n2)cc(OC)c1OC] | C |
| B51 | 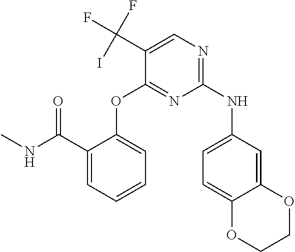 [SMILES: CNC(=O)c1ccccc1Oc1nc(Nc2ccc3c(c2)OCCO3)ncc1C(F)(F)I] | C |
| B52 | 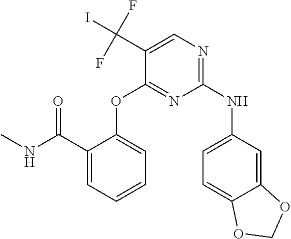 [SMILES: CNC(=O)c1ccccc1Oc1nc(Nc2ccc3c(c2)OCO3)ncc1C(F)(F)I] | C |
| B53 | 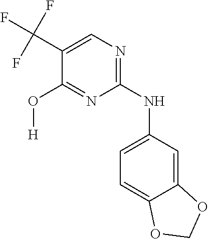 [SMILES: Oc1nc(Nc2ccc3c(c2)OCO3)ncc1C(F)(F)F] | C |

In one aspect, provided herein, is a compound of Formula (IIIC):

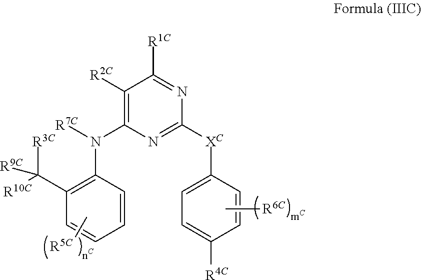

wherein:
R^{1C} is H, substituted or unsubstituted alkyl, or halogen;
R^{2C} is H, halogen, substituted or unsubstituted alkyl, or substituted or unsubstituted haloalkyl;
R^{3C} is —NR^{C}R^{C}, —OR^{C}, —O(C═O)R^{C}, —O(C═O)NR^{C}R^{C}, —NR^{C}(C═O)NR^{C}R^{C}, —NR^{C}(C═O)R^{C}, or —SR^{C};
R^{4C} is —NR^{41C}R^{42C}, —OR^{43C}, —C(═O)OR^{44C}, —C(═O)NR^{C}R^{C}, or —NR^{C}C(═O)R^{C};
each R^{5C} and R^{6C} is independently halogen, —CN, —OR^{C}, —SR^{C}, —S(═O)R^{C}, —S(═O)₂R^{C}, —NO₂, —NR^{C}R^{C}, —NR^{C}S(═O)₂R^{C}, —S(═O)₂NR^{C}R^{C}, —C(═O)R^{C}, —OC(═O)R^{C}, —C(═O)C(═O)R^{C}, —C(═O)OR^{C}, —C(═O)NR^{C}OR^{C}, —OC(═O)OR^{C}, —C(═O)NR^{C}R^{C}, —OC(═O)NR^{C}R^{C}, —NR^{C}C(═O)NR^{C}R^{C}, —NR^{C}S(═O)₂NR^{C}R^{C}, —NR^{C}C(═O)R^{C}, —NR^{C}C(═O)OR^{C}, substituted or unsubstituted alkyl, substituted or unsubstituted cycloalkyl, substituted or unsubstituted heterocycloalkyl, substituted or unsubstituted aryl, or substituted or unsubstituted heteroaryl;
R^{7C} is H or substituted or unsubstituted alkyl;
X^{C} is —O— or —NR^{8C}—;
R^{8C} is H or substituted or unsubstituted alkyl;
R^{9C} and R^{10C} are each independently H, substituted or unsubstituted alkyl, substituted or unsubstituted cycloalkyl, substituted or unsubstituted heterocycloalkyl, substituted or unsubstituted aryl, or substituted or unsubstituted heteroaryl;
or R^{9C} and R^{10C} are taken together with the carbon atom to which they are attached to form a substituted or unsubstituted cycloalkyl or substituted or unsubstituted heterocycloalkyl;
R^{41C} and R^{42C} are each independently hydrogen, alkyl, cycloalkyl, heterocycloalkyl, aryl, or heteroaryl; wherein the alkyl, cycloalkyl, heterocycloalkyl, aryl, and heteroaryl are independently optionally substituted with one or more R^{45C};
or R^{41C} and R^{42C} are taken together with the nitrogen atom to which they are attached to form a substituted or unsubstituted heterocycloalkyl;
R^{43C} is hydrogen, —CN, alkyl, cycloalkyl, heterocycloalkyl, aryl, or heteroaryl; wherein the alkyl, cycloalkyl, heterocycloalkyl, aryl, and heteroaryl are independently optionally substituted with one or more R^{45C};
R^{44C} is substituted or unsubstituted alkyl, substituted or unsubstituted cycloalkyl, substituted or unsubstituted heterocycloalkyl, substituted or unsubstituted aryl, or substituted or unsubstituted heteroaryl;
each R^{45C} is independently oxo, halogen, —CN, —OR^{C}, —S(═O)₂R^{C}, —S(═O)₂NR^{C}R^{C}, —C(═O)R^{C}, —OC(═O)R^{C}, —C(═O)OR^{C}, —OC(═O)OR^{C}, —C(═O)NR^{C}R^{C}, —OC(═O)NR^{C}R^{C}, —NR^{C}C(═O)NR^{C}R^{C}, —NR^{C}C(═O)R^{C}, alkyl, haloalkyl, or hydroxyalkyl;
each R^{C} is independently H, substituted or unsubstituted alkyl, substituted or unsubstituted heteroalkyl, substituted or unsubstituted aryl, or substituted or unsubstituted heteroaryl;
n^{C} is an integer from 0-4;
m^{C} is an integer from 0-4; and
with the proviso that when R^{4C} is —OMe and R^{2C} is halogen, then R^{3C} is not OH,
or pharmaceutically acceptable salt thereof.

In some embodiments, R^{1C} is H or halogen. In some embodiments, R^{1C} is H or F. In some embodiments, R^{1C} is H. In some embodiments, R^{1C} is F.

In some embodiments, R^{2C} is halogen or C₁-C₆ haloalkyl. In some embodiments, R^{2C} is Br, Cl, or —CF₃. In some embodiments, R^{2C} is Br. In some embodiments, R^{2C} is Cl. In some embodiments, R^{2C} is —CF₃.

In some embodiments, R^{3C} is —NR^{C}R^{C}, —OR^{C}, —O(C═O)R^{C}, or —O(C═O)NR^{C}R^{C}. In some embodiments, R^{3C} is —NR^{C}R^{C} or —O(C═O)NR^{C}R^{C}. In some embodiments, R^{3C} is —NR^{C}R^{C} and each R^{C} is independently hydrogen, substituted or unsubstituted C₁-C₆ alkyl, or substituted or unsubstituted cycloalkyl or both R^{C}s are taken together with the nitrogen atom to which they are attached to form a substituted or unsubstituted heterocycloalkyl. In some embodiments, R^{3C} is —NR^{C}R^{C} each R^{C} is independently selected from H,

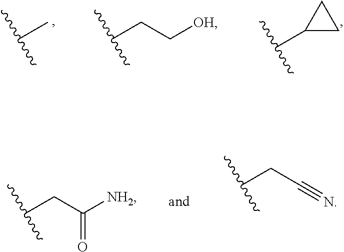

In some embodiments, R^{3C} is —NR^{C}R^{C} and both R^{C}s are taken together to form a heterocycloalkyl selected from

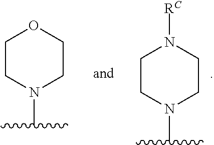

In some embodiments, R^{3C} is selected from

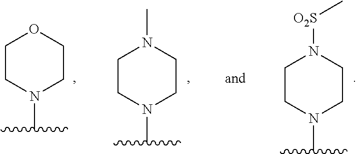

In some embodiments, R^{3C} is —OR^{C} or —O(C═O)R^{C}. In some embodiments, R^{3C} is OR^{C} and the R^{C} of R^{3C} is hydrogen, substituted or unsubstituted C₁-C₆ alkyl, substituted or unsubstituted cycloalkyl, substituted or unsubstituted heterocycloalkyl, substituted or unsubstituted aryl, or substituted or unsubstituted heteroaryl. In some embodiments, R^{3C} is —OR^{C} or —O(C═O)R^{C} and the R^{C} of R^{3C} is

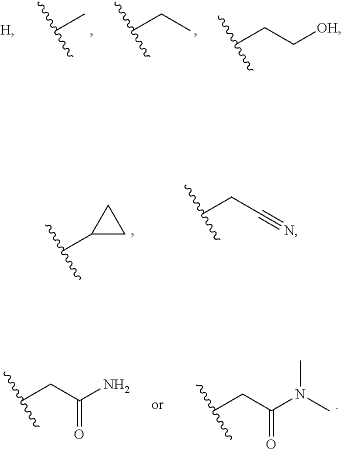

In some embodiments, R^{3C} is —OR^{C} or —O(C═O)R^{C} and the R^{C} of R^{3C} is

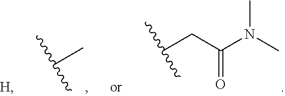

In some embodiments, R^{3C} is

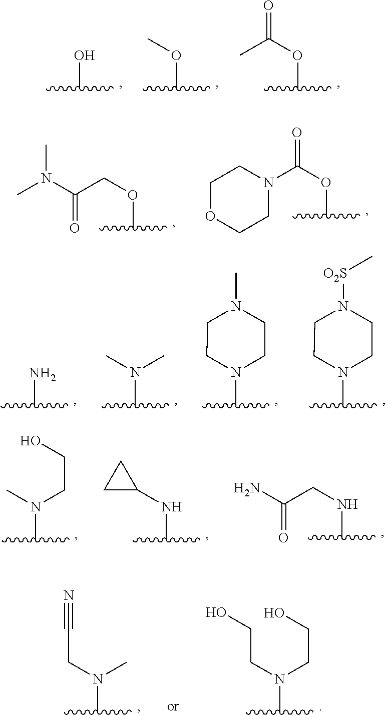

In some embodiments, R^{4C} is —NR^{41C}R^{42C}, —OR^{43C}, —C(═O)NR^{C}R^{C}, or —NR^{C}C(═O)R^{C}.

In some embodiments, R^{4C} is —NR^{41C}R^{42C}. In some embodiments, R^{41C} and R^{42C} are each independently hydrogen, alkyl, or cycloalkyl, wherein the alkyl or cycloalkyl is optionally substituted with one or more R^{45C} or R^{41C} and R^{42C} are taken together with the nitrogen atom to which they are attached to form a substituted or unsubstituted heterocycloalkyl. In some embodiments, R^{41C} and R^{42C} is independently H,

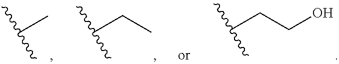

In some embodiments, R^{41C} and R^{42C} are taken together with the nitrogen atom to which they are attached to form a heterocycloalkyl, wherein the heterocycloalkyl is

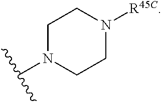

In some embodiments, R^{41C} and R^{42C} are taken together with the nitrogen atom to which they are attached to form

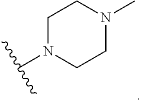 [SMILES: *N1CCN(C)CC1]

In some embodiments, R^{4C} is —OR^{43C}. In some embodiments, R^{43C} is hydrogen or C₁-C₆alkyl optionally substituted with one or more R^{45C}. In some embodiments, R^{43C} is H, —CH₃, —CH₂CH₃, CH₂F, —CHF₂, or CF₃.

In some embodiments, R^{4C} is —C(═O)NR^{41C}R^{42C}. In some embodiments, R^{41C} and R^{42C} are each independently hydrogen, substituted or unsubstituted C₁-C₆ alkyl, or cycloalkyl; wherein each alkyl or cycloalkyl is independently optionally substituted with one or more R^{45C}. In some embodiments, R^{41C} and R^{42C} are each independently H, —CH₃, or —CH₂CH₃.

In some embodiments, R^{4C} is —NR^{C}C(═O)R^{C}. In some embodiments, R^{4C} is —NR^{C}C(═O)R^{C} and one R^{C} of R^{4C} is H or —CH₃; and the other R^{C} of R^{4C} is substituted or unsubstituted alkyl or substituted or unsubstituted cycloalkyl. In some embodiments, R^{4C} is

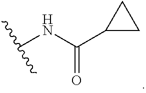 [SMILES: *NC(=O)C1CC1]

In some embodiments, R^{4C} is

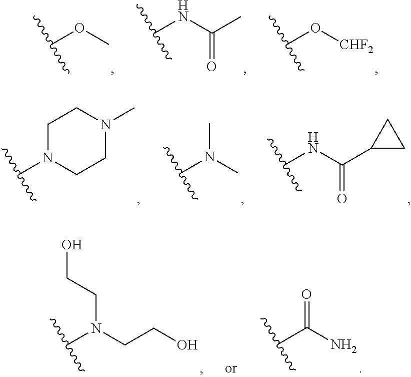

In some embodiments, each R^{5C} is independently halogen, —CN, —OR^{C}, —NR^{C}R^{C}, —NR^{C}S(═O)₂R^{C}, —S(═O)₂NR^{C}R^{C}, —OC(═O)OR^{C}, —C(═O)NR^{C}R^{C}, —OC(═O)NR^{C}R^{C}, —NR^{C}C(═O)R^{C}, or substituted or unsubstituted alkyl. In some embodiments, each R^{5C} is independently halogen, —CN, —OR^{C}, or substituted or unsubstituted alkyl. In some embodiments, each R^{5C} is independently halogen or —OR^{C}. In some embodiments, each R^{5C} is independently —O(C₁-C₆ alkyl). In some embodiments, each R^{5C} is independently —OCH₃.

In some embodiments, n^{C} is 0, 1, or 2. In some embodiments, n^{C} is 0 or 1. In some embodiments, n^{C} is 0. In some embodiments, n^{C} is 1. In some embodiments, n^{C} is 2. In some embodiments, n^{C} is 3.

In some embodiments, each R^{6C} is independently halogen, —CN, —OR^{C}, —C(═O)R^{C}, —OC(═O)R^{C}—C(═O)OR^{C}, —OC(═O)OR^{C}, —OC(═O)NR^{C}R^{C}, or substituted or unsubstituted alkyl. In some embodiments, each R^{6C} is independently halogen or —OR^{C}. In some embodiments, each R^{6C} is —O(C₁-C₆ alkyl). In some embodiments, each R^{6C} is —OCH₃.

In some embodiments, m^{C} is 0, 1, or 2. In some embodiments, m^{C} is 2. In some embodiments, m^{C} is 2 and each R^{6C} is —OCH₃. In some embodiments, m^{C} is 0 or 1. In some embodiments, m^{C} is 0. In some embodiments, m^{C} is 1. In some embodiments, m^{C} is 3.

In some embodiments, R^{7C} is H or —CH₃. In some embodiments, R^{7C} is H. In some embodiments, R^{7C} is —CH₃. In some embodiments, R^{7C} is H or C₁-C₃ alkyl.

In some embodiments, X^{C} is —NR⁸—. In some embodiments, R^{8C} is H or —CH₃. In some embodiments, R^{8C} is H. In some embodiments, R^{8C} is —CH₃. In some embodiments, R^{8C} is H or C₁-C₃ alkyl.

In some embodiments, R^{9C} and R^{10C} are each independently H or substituted or unsubstituted alkyl. In some embodiments, R^{9C} and R^{10C} are each independently H or C₁-C₆ alkyl. In some embodiments, R^{9C} is —CH₃ and R^{10C} is H. In some embodiments, R^{9C} and R^{10C} are each H. In some embodiments, R^{9C} and R^{10C} are each independently H or —CH₃.

In one aspect, provided herein, is a compound having a structure of Formula (IVD)

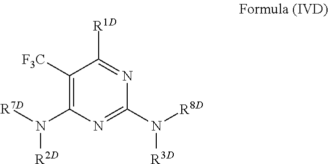

wherein:
R^{1D} is H or halogen;
R^{2D} is

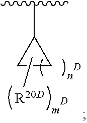

m^{D} is an integer from 1 to 3;
n^{D} is an integer from 1 to 6;
R^{3D} is

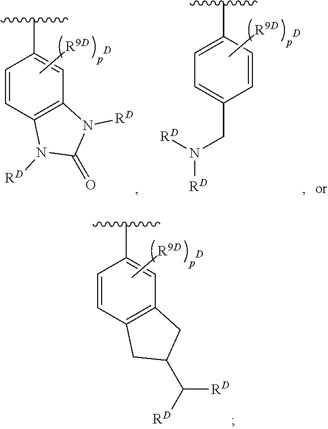

R^{7D} and R^{8D} are each independently H or substituted or unsubstituted alkyl;
each R^{9D} is independently halogen, —CN, —OR^{D}, S(═O)₂R^{D}, —NR^{D}R^{D}, —S(═O)₂NR^{D}R^{D}, —C(═O)R^{D}, —OC(═O)R^{D}, —C(═O)OR^{D}, —OC(═O)OR^{D}, —C(═O)NR^{D}R^{D}, —OC(═O)NR^{D}R^{D}, —NR^{D}C(═O)NR^{D}R^{D}, —NR^{D}C(═O)R^{D}, alkyl, haloalkyl, or hydroxyalkyl;
p^{D} is an integer from 0 to 2;
each R^{20D} is independently halogen, —CN, —OR^{D}, S(═O)₂R^{D}, —S(═O)₂NR^{D}R^{D}, —C(═O)R^{D}, —OC(═O)R^{D}, —C(═O)OR^{D}, —OC(═O)OR^{D}, —OC(═O)NR^{D}R^{D}, —NR^{D}C(═O)NR^{D}R^{D}, —NR^{D}C(═O)R^{D}, alkyl, haloalkyl, or hydroxyalkyl; and
each R^{D} is independently H, substituted or unsubstituted alkyl, substituted or unsubstituted heteroalkyl, substituted or unsubstituted aryl, or substituted or unsubstituted heteroaryl
or a pharmaceutically acceptable salt thereof.

In some embodiments, R^{1D} is H or fluorine. In some embodiments, R^{1D} is H. In some embodiments, R^{1D} is fluorine.

In some embodiments, R^{2D} is

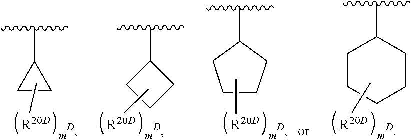

In some embodiments, R² is

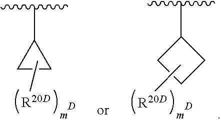

In some embodiments, R² is

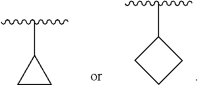

In some embodiments, each R^{20D} is independently halogen, —CN, —OR^{D}, —C(═O)R^{D}, —OC(═O)R^{D}, —OC(═O)OR^{D}, —OC(═O)NR^{D}R^{D}, —NR^{D}C(═O)R^{D}, or substituted or unsubstituted C₁-C₆alkyl. In some embodiments, each R^{20D} is independently halogen, —CN, —OR^{D}, —C(═O)R^{D}, —OC(═O)R^{D}, —OC(═O)OR^{D}, —OC(═O)NR^{D}R^{D}, —NR^{D}C(═O)R^{D}, or C₁-C₆ alkyl. In some embodiments, each R^{20D} is independently halogen, —CN, —OR^{D}, or substituted or unsubstituted C₁-C₆ alkyl. In some embodiments, each R^{20D} is independently halogen, —CN, —OR^{D}, or C₁-C₆ alkyl.

In some embodiments, m^{D} is 0 or 1. In some embodiments, m^{D} is 0. In some embodiments, m^{D} is 1.

In some embodiments, R^{3D} is

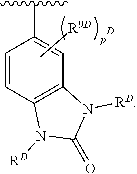

In some embodiments, each R^{D} is independently H or —CH₃. In some embodiments, each R^{D} is independently H. In some embodiments, each R^{D} of R^{3D} is independently H or —CH₃. In some embodiments, each R^{D} of R^{3D} is independently H.

In some embodiments, R^{3D} is

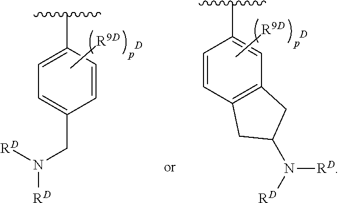

In some embodiments, each R^{D} is independently hydrogen, —C(═O)C₁-C₆ alkyl, —C(═O)OC₁-C₆ alkyl, or C₁-C₆ alkyl, wherein each alkyl of each R^{D} is substituted or unsubstituted. In some embodiments, each R^{D} is independently H or —CH₃. In some embodiments, each R^{D} of R^{3D} is independently hydrogen, —C(═O)C₁-C₆ alkyl, —C(═O)OC₁-C₆ alkyl, or C₁-C₆ alkyl, wherein each alkyl of each R^{D} is substituted or unsubstituted. In some embodiments, each R^{D} of R^{3D} is independently H or —CH₃. In some embodiments, one R^{D} is of R^{3D} H and one R^{D} of R^{3D} is

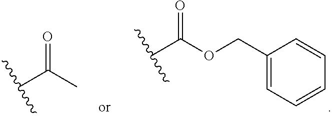

In some embodiments, R^{7D} and R^{8D} are each independently H or —CH₃. In some embodiments, R^{7D} and R^{8D} are each independently H.

In some embodiments, each R^{9D} is independently halogen, —CN, —OR^{D}, —NR^{D}R^{D}, —C(═O)R^{D}, —OC(═O)R^{D}, —OC(═O)OR^{D}, —C(═O)NR^{D}R^{D}, —NR^{D}C(═O)R^{D}, or C₁-C₆ alkyl. In some embodiments, each R^{9D} is independently halogen, —CN, —OR^{D}, or C₁-C₆ alkyl.

In some embodiments, p^{D} is 0 or 1. In some embodiments, p^{D} is 0. In some embodiments, p^{D} is 1.

In one aspect, provided herein, is a compound having a structure of Formula (VE):

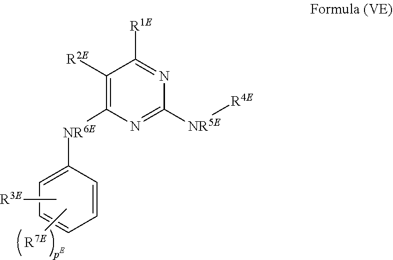

wherein:
R^{1E} is H, nitrile, or halogen;
R^{2E} is halogen, nitrile, methyl, cyclopropyl, or —CF₃;
R^{3E} is halogen,

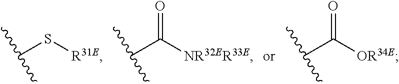

R^{4E} is aryl substituted with one or more —OR^{35E}, substituted or unsubstituted cycloalkyl, or substituted or unsubstituted heterocycloalkyl,

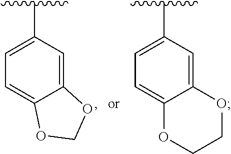 [SMILES: *c1ccc2c(c1)OCO2 ; *c1ccc2c(c1)OCCO2]

R^{5E} and R^{6E} are each independently H or C₁-C₆ alkyl;
each R^{7E} is independently halogen, —CN, —OR^{E}, —S(═O)₂R^{E}, —NR^{E}R^{E}, —S(═O)₂NR^{E}R^{E}, —C(═O)R^{E}, —OC(═O)R^{E}, —C(═O)OR^{E}, —OC(═O)OR^{E}, —C(═O)NR^{E}R^{E}, —OC(═O)NR^{E}R^{E}, —NR^{E}C(═O)NR^{E}R^{E}, —NR^{E}C(═O)R^{E}, C₁-C₆ alkyl, C₁-C₆ haloalkyl, or C₁-C₆ hydroxyalkyl;
p^{E} is an integer from 0 to 3;
R^{31E} is H, C₁-C₆ alkyl, or cycloalkyl;
R^{32E} and R^{33E} are each independently H, substituted or unsubstituted C₁-C₆ alkyl, or cycloalkyl;
R^{34E} is H, C₁-C₆ alkyl, or cycloalkyl;
each R^{35E} is independently substituted or unsubstituted alkyl, or substituted or unsubstituted heteroalkyl;
each R^{E} is independently hydrogen, C₁-C₆ alkyl, cycloalkyl, heterocycloalkyl, aryl, or heteroaryl; wherein the alkyl, cycloalkyl, heterocycloalkyl, aryl, and heteroaryl are independently optionally substituted with one or more halogen, —OH, —NH₂, substituted amino, cycloalkyl, oxo, or C₁-C₆ alkyl;
wherein when R^{3E} is

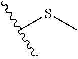 [SMILES: *SC]

then R^{2E} is not Br; and
wherein when R^{3E} is

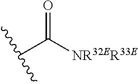

then R^{2E} is not Cl and R^{4E} is not

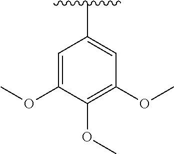

or a pharmaceutically acceptable salt thereof.

In some embodiments, R^{1E} is H or F. In some embodiments, R^{1E} is H. In some embodiments, R^{1E} is nitrile.

In some embodiments, R^{2E} is Cl, Br, or —CF₃. In some embodiments, R^{2E} is Br or —CF₃. In some embodiments, R^{2E} is F. In some embodiments, R^{2E} is I. In some embodiments, R^{2E} is nitrile. In some embodiments, R^{2E} is methyl. In some embodiments, R^{2E} is cyclopropyl.

In some embodiments, R^{3E} is —SR^{31E}. In some embodiments, R^{3E} is —SH, —SCH₃, or —SCH₂CH₃. In some embodiments, R^{3E} is —SCH₃.

In some embodiments, R^{3E} is

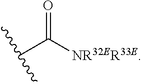

In some embodiments, R^{3E} is

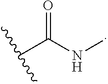

In some embodiments, R^{3E} is

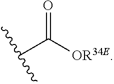

In some embodiments, R^{3E} is

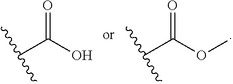

In some embodiments, R^{3E} is

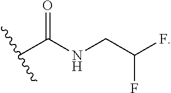 [SMILES: *C(=O)NCC(F)F]

In some embodiments, R^{3E} is

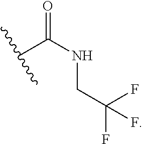 [SMILES: *C(=O)NCC(F)(F)F]

In some embodiments, R^{4E} is

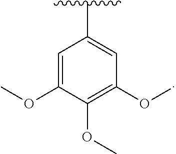

In some embodiments, R^{4E} is

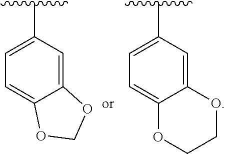 [SMILES: *c1ccc2c(c1)OCO2 ; *c1ccc2c(c1)OCCO2]

In some embodiments, R^{4E} is

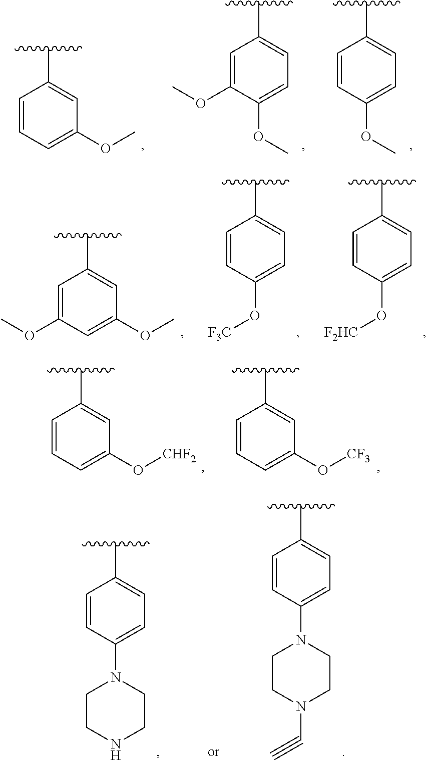

In some embodiments, R^{5E} and R^{6E} are each independently H or —CH₃. In some embodiments, R^{5E} and R^{6E} are each independently H.

In some embodiments, each R^{7E} is independently halogen, —CN, —OR^{E}, —NR^{E}R^{E}, —C(═O)R^{E}, —OC(═O)R^{E}, —C(═O)OR^{E}, —C(═O)NR^{E}R^{E}, or C₁-C₆ alkyl. In some embodiments, each R^{7E} is independently halogen, —OR^{E}, —OC(═O)R^{E}, or C₁-C₆ alkyl. In some embodiments, R^{7E} is independently halogen or —OCH₃. In some embodiments, R^{7E} is

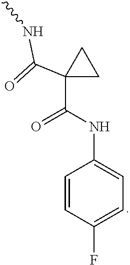

In some embodiments, p^{E} is 0 or 1. In some embodiments, p^{E} is 0. In some embodiments, p^{E} is 1.

Any combination of the groups described above for the various variables is contemplated herein. Throughout the specification, groups and substituents thereof are chosen by one skilled in the field to provide stable moieties and compounds.

Illustrative compounds of Formula III and related analogs (C1-C27), compounds of Formula IV and related analogs (D1-D6), and compounds of Formula V and related analogs (E1-E13) are shown in Table 3 (along with their respective IC₅₀ values for ULK1 inhibition assay). IC₅₀s for ADP Glo assay are represented in nM, with A representing IC₅₀<5 nM, B representing 5 nM>IC₅₀>100 nM, and C representing IC₅₀>100 nM. IC₅₀s or nanoBRET assay are represented in nM, with A<100 nM and B>100 nM. NT indicates the compound was not tested.

TABLE 3

| | | ULK1 IC50 | |
|---|---|---|---|
| | | ADP-Glo (A < 5 | ULK1 IC50 |
| | | nM, 5 nM < B < | NanoBRET (A < |
| Compound | | 100 nM, C > 100 | 100 nM, B > 100 |
| Number | Structure | nM) | nM) |
| C1 | 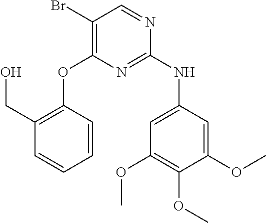 [SMILES: COc1cc(Nc2ncc(Br)c(Oc3ccccc3CO)n2)cc(OC)c1OC] | C | |
| C2 | 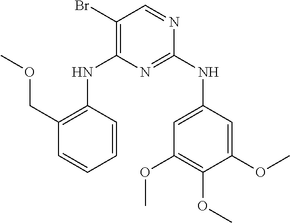 | A | |
| C3 | 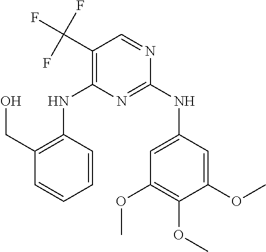 [SMILES: COc1cc(Nc2ncc(C(F)(F)F)c(Nc3ccccc3CO)n2)cc(OC)c1OC] | A | |
| C4 | 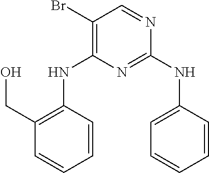 [SMILES: OCc1ccccc1Nc1nc(Nc2ccccc2)ncc1Br] | C | |
| C5 | 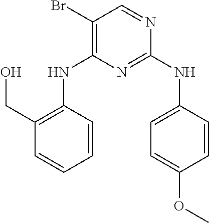 [SMILES: COc1ccc(Nc2ncc(Br)c(Nc3ccccc3CO)n2)cc1] | B | |
| C6 | 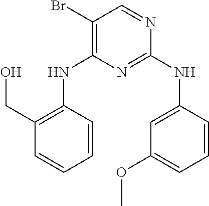 [SMILES: COc1cccc(Nc2ncc(Br)c(Nc3ccccc3CO)n2)c1] | A | |
| C7 | 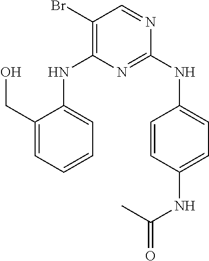 [SMILES: CC(=O)Nc1ccc(Nc2ncc(Br)c(Nc3ccccc3CO)n2)cc1] | B | |
| C8 | 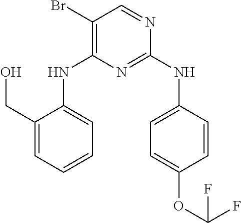 [SMILES: OCc1ccccc1Nc1nc(Nc2ccc(OC(F)F)cc2)ncc1Br] | B | |
| C9 | 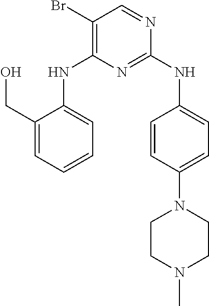 [SMILES: CN1CCN(c2ccc(Nc3ncc(Br)c(Nc4ccccc4CO)n3)cc2)CC1] | A | |
| C10 | 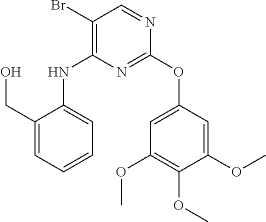 [SMILES: COc1cc(Oc2ncc(Br)c(Nc3ccccc3CO)n2)cc(OC)c1OC] | C | |
| C11 | 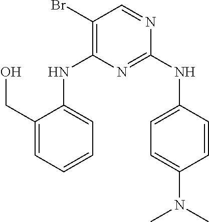 [SMILES: CN(C)c1ccc(Nc2ncc(Br)c(Nc3ccccc3CO)n2)cc1] | A | |
| C12 | 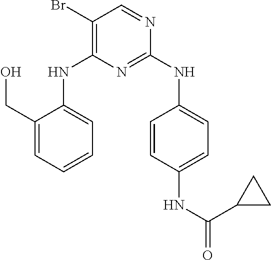 [SMILES: O=C(Nc1ccc(Nc2ncc(Br)c(Nc3ccccc3CO)n2)cc1)C1CC1] | A | |
| C13 | 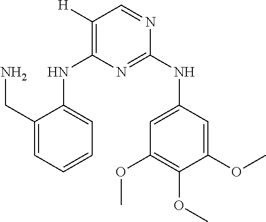 [SMILES: COc1cc(Nc2nccc(Nc3ccccc3CN)n2)cc(OC)c1OC] | C | |
| C14 | 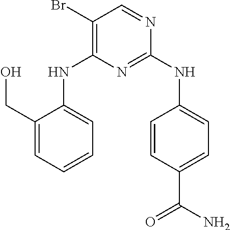 [SMILES: NC(=O)c1ccc(Nc2ncc(Br)c(Nc3ccccc3CO)n2)cc1] | B | |
| C15 | 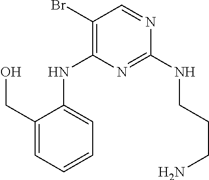 [SMILES: NCCCNc1ncc(Br)c(Nc2ccccc2CO)n1] | C | |
| C16 | 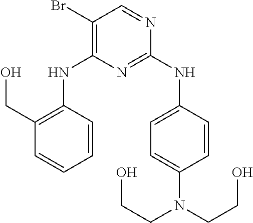 [SMILES: OCCN(CCO)c1ccc(Nc2ncc(Br)c(Nc3ccccc3CO)n2)cc1] | A | |
| C17 | 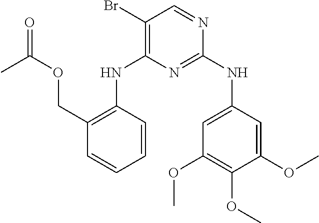 [SMILES: COc1cc(Nc2ncc(Br)c(Nc3ccccc3COC(C)=O)n2)cc(OC)c1OC] | B | |
| C18 | 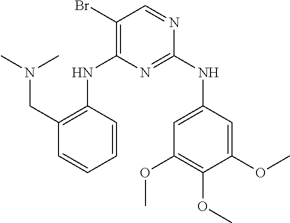 | B | |
| C19 | 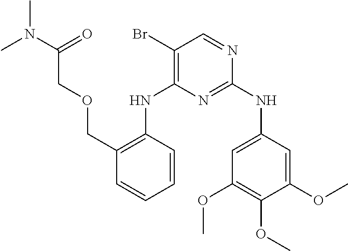 | C | |
| C20 | 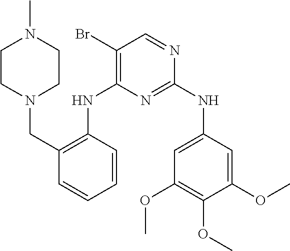 [SMILES: COc1cc(Nc2ncc(Br)c(Nc3ccccc3CN3CCN(C)CC3)n2)cc(OC)c1OC] | C | |
| C21 | 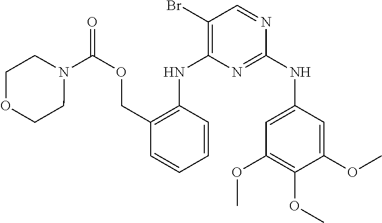 [SMILES: COc1cc(Nc2ncc(Br)c(Nc3ccccc3COC(=O)N3CCOCC3)n2)cc(OC)c1OC] | C | |
| C22 | 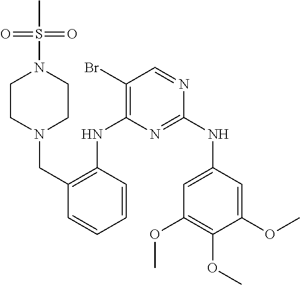 [SMILES: COc1cc(Nc2ncc(Br)c(Nc3ccccc3CN3CCN(S(C)(=O)=O)CC3)n2)cc(OC)c1OC] | C | |
| C23 | 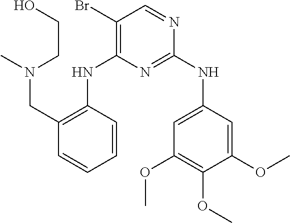 | C | |
| C24 | 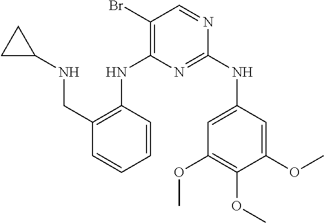 [SMILES: COc1cc(Nc2ncc(Br)c(Nc3ccccc3CNC3CC3)n2)cc(OC)c1OC] | C | |
| C25 | 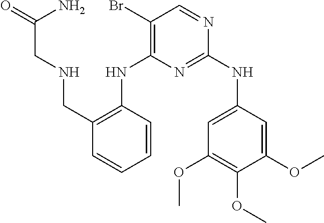 [SMILES: COc1cc(Nc2ncc(Br)c(Nc3ccccc3CNCC(N)=O)n2)cc(OC)c1OC] | B | |
| C26 | 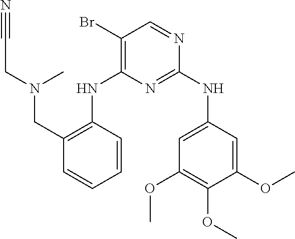 [SMILES: COc1cc(Nc2ncc(Br)c(Nc3ccccc3CN(C)CC#N)n2)cc(OC)c1OC] | B | |
| C27 | 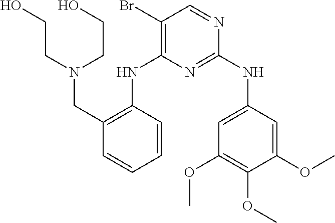 [SMILES: COc1cc(Nc2ncc(Br)c(Nc3ccccc3CN(CCO)CCO)n2)cc(OC)c1OC] | B | |
| C28 | 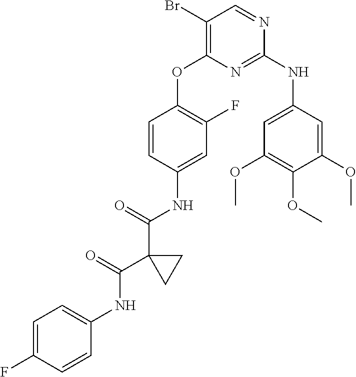 [SMILES: COc1cc(Nc2ncc(Br)c(Oc3ccc(NC(=O)C4(C(=O)Nc5ccc(F)cc5)CC4)cc3F)n2)cc(OC)c1OC] | C | |
| C29 | 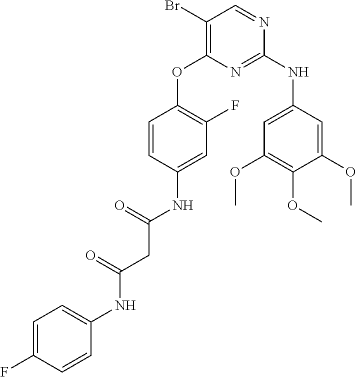 [SMILES: COc1cc(Nc2ncc(Br)c(Oc3ccc(NC(=O)CC(=O)Nc4ccc(F)cc4)cc3F)n2)cc(OC)c1OC] | C | |
| D1 | 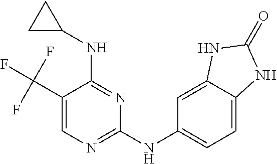 [SMILES: O=c1[nH]c2ccc(Nc3ncc(C(F)(F)F)c(NC4CC4)n3)cc2[nH]1] | B | |
| D2 | 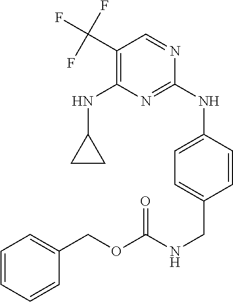 [SMILES: O=C(NCc1ccc(Nc2ncc(C(F)(F)F)c(NC3CC3)n2)cc1)OCc1ccccc1] | A | |
| D3 | 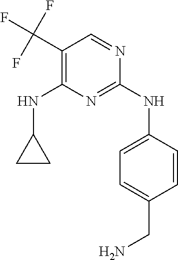 [SMILES: NCc1ccc(Nc2ncc(C(F)(F)F)c(NC3CC3)n2)cc1] | A | |
| D4 | 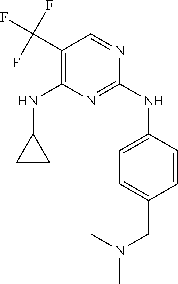 [SMILES: CN(C)Cc1ccc(Nc2ncc(C(F)(F)F)c(NC3CC3)n2)cc1] | A | |
| D5 | 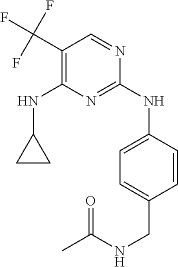 [SMILES: CC(=O)NCc1ccc(Nc2ncc(C(F)(F)F)c(NC3CC3)n2)cc1] | B | |
| D6 | 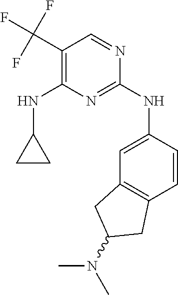 [SMILES: CN(C)C1Cc2ccc(Nc3ncc(C(F)(F)F)c(NC4CC4)n3)cc2C1] | A | |
| E1 | 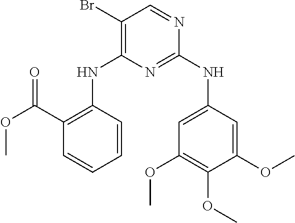 [SMILES: COC(=O)c1ccccc1Nc1nc(Nc2cc(OC)c(OC)c(OC)c2)ncc1Br] | A | |
| E2 | 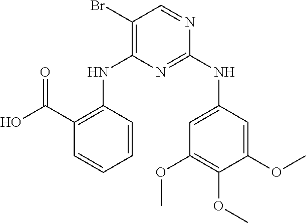 [SMILES: COc1cc(Nc2ncc(Br)c(Nc3ccccc3C(=O)O)n2)cc(OC)c1OC] | A | |
| E3 | 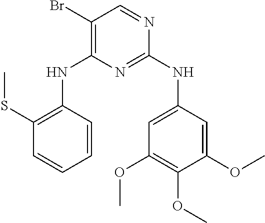 [SMILES: COc1cc(Nc2ncc(Br)c(Nc3ccccc3SC)n2)cc(OC)c1OC] | B | |
| E4 | 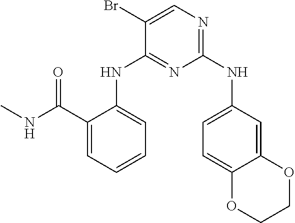 [SMILES: CNC(=O)c1ccccc1Nc1nc(Nc2ccc3c(c2)OCCO3)ncc1Br] | B | B |
| E5 | 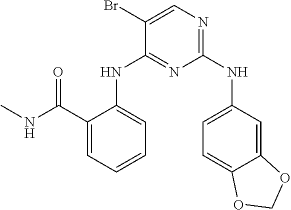 [SMILES: CNC(=O)c1ccccc1Nc1nc(Nc2ccc3c(c2)OCO3)ncc1Br] | B | B |
| E6 | 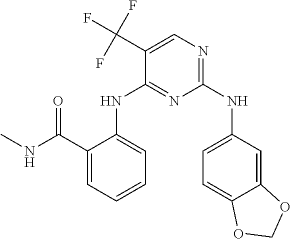 [SMILES: CNC(=O)c1ccccc1Nc1nc(Nc2ccc3c(c2)OCO3)ncc1C(F)(F)F] | B | B |
| E7 | 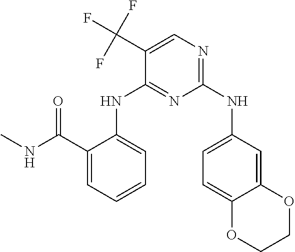 [SMILES: CNC(=O)c1ccccc1Nc1nc(Nc2ccc3c(c2)OCCO3)ncc1C(F)(F)F] | B | B |
| E8 | 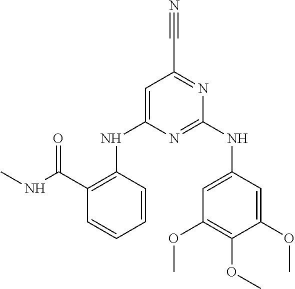 [SMILES: CNC(=O)c1ccccc1Nc1cc(C#N)nc(Nc2cc(OC)c(OC)c(OC)c2)n1] | C | |
| E9 | 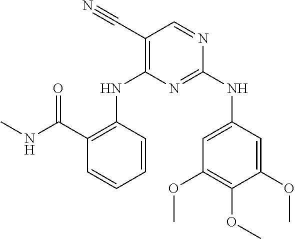 [SMILES: CNC(=O)c1ccccc1Nc1nc(Nc2cc(OC)c(OC)c(OC)c2)ncc1C#N] | | A |
| E10 | 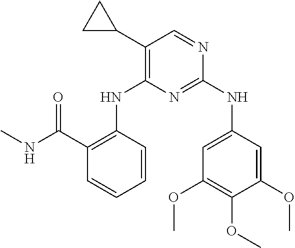 [SMILES: CNC(=O)c1ccccc1Nc1nc(Nc2cc(OC)c(OC)c(OC)c2)ncc1C1CC1] | | B |
| E11 | 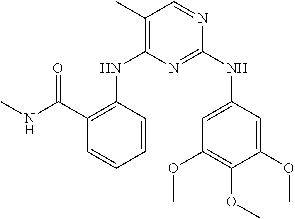 [SMILES: CNC(=O)c1ccccc1Nc1nc(Nc2cc(OC)c(OC)c(OC)c2)ncc1C] | | B |
| E12 | 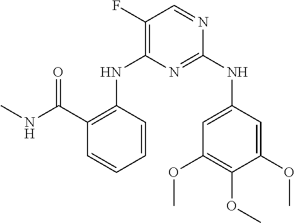 [SMILES: CNC(=O)c1ccccc1Nc1nc(Nc2cc(OC)c(OC)c(OC)c2)ncc1F] | | B |
| E13 | 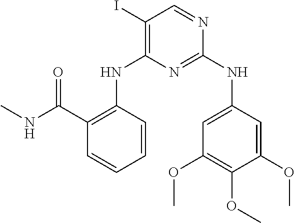 [SMILES: CNC(=O)c1ccccc1Nc1nc(Nc2cc(OC)c(OC)c(OC)c2)ncc1I] | | A |
| E14 | 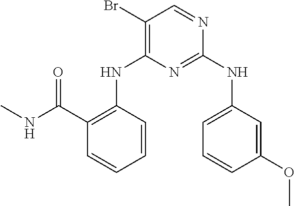 [SMILES: CNC(=O)c1ccccc1Nc1nc(Nc2cccc(OC)c2)ncc1Br] | B | |
| E15 | 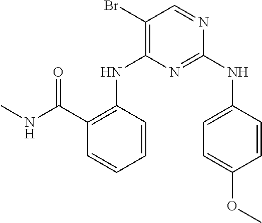 | C | |
| E16 | 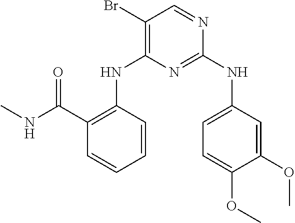 | B | |
| E17 | 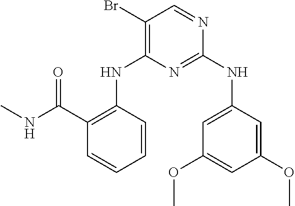 | B | |
| E18 | 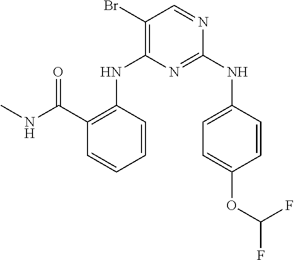 [SMILES: CNC(=O)c1ccccc1Nc1nc(Nc2ccc(OC(F)F)cc2)ncc1Br] | C | |
| E19 | 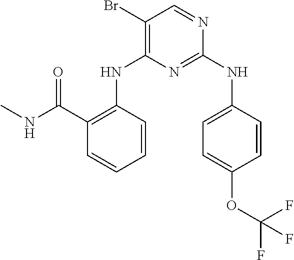 [SMILES: CNC(=O)c1ccccc1Nc1nc(Nc2ccc(OC(F)(F)F)cc2)ncc1Br] | C | |
| E20 | 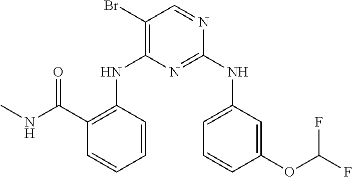 [SMILES: CNC(=O)c1ccccc1Nc1nc(Nc2cccc(OC(F)F)c2)ncc1Br] | C | |
| E21 | 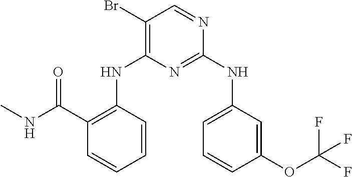 [SMILES: CNC(=O)c1ccccc1Nc1nc(Nc2cccc(OC(F)(F)F)c2)ncc1Br] | B | |
| E22 | 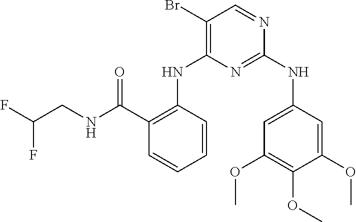 [SMILES: COc1cc(Nc2ncc(Br)c(Nc3ccccc3C(=O)NCC(F)F)n2)cc(OC)c1OC] | B | |
| E23 | 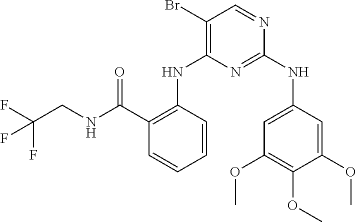 [SMILES: COc1cc(Nc2ncc(Br)c(Nc3ccccc3C(=O)NCC(F)(F)F)n2)cc(OC)c1OC] | B | |
| E24 | 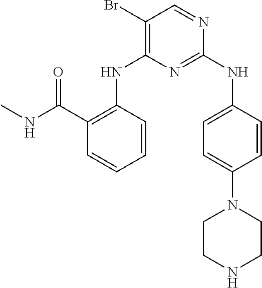 [SMILES: CNC(=O)c1ccccc1Nc1nc(Nc2ccc(N3CCNCC3)cc2)ncc1Br] | B | |
| E25 | 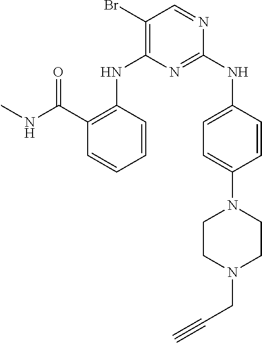 [SMILES: C#CCN1CCN(c2ccc(Nc3ncc(Br)c(Nc4ccccc4C(=O)NC)n3)cc2)CC1] | B | |
| E26 | 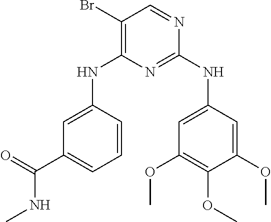 [SMILES: CNC(=O)c1cccc(Nc2nc(Nc3cc(OC)c(OC)c(OC)c3)ncc2Br)c1] | C | |
| E27 | 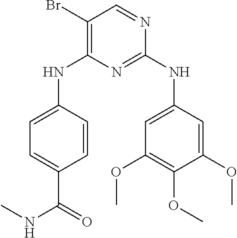 | C | |
| E28 | 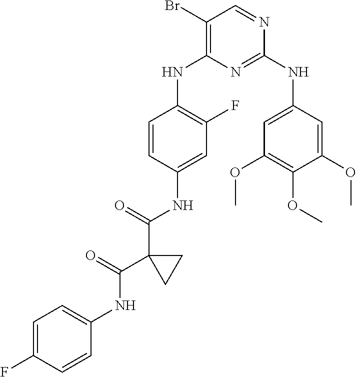 [SMILES: COc1cc(Nc2ncc(Br)c(Nc3ccc(NC(=O)C4(C(=O)Nc5ccc(F)cc5)CC4)cc3F)n2)cc(OC)c1OC] | C | |

The invention provides salts of any one of the compounds described herein. Pharmaceutically-acceptable salts include, for example, acid-addition salts and base-addition salts. In some embodiments, the acid that is added to the compound to form an acid-addition salt can be an organic acid or an inorganic acid. In some embodiments, a base that is added to the compound to form a base-addition salt is an organic base or an inorganic base. In some embodiments, a pharmaceutically-acceptable salt is a metal salt.

In some embodiments, metal salts arise from the addition of an inorganic base to a compound of the invention. The inorganic base consists of a metal cation paired with a basic counterion, such as, for example, hydroxide, carbonate, bicarbonate, or phosphate. In some embodiments, the metal is an alkali metal, alkaline earth metal, transition metal, or main group metal. In some embodiments, the metal is lithium, sodium, potassium, cesium, cerium, magnesium, manganese, iron, calcium, strontium, cobalt, titanium, aluminum, copper, cadmium, or zinc.

In some embodiments, a metal salt is a lithium salt, a sodium salt, a potassium salt, a cesium salt, a cerium salt, a magnesium salt, a manganese salt, an iron salt, a calcium salt, a strontium salt, a cobalt salt, a titanium salt, an aluminum salt, a copper salt, a cadmium salt, or a zinc salt.

In some embodiments, ammonium salts arise from the addition of ammonia or an organic amine to a compound of the invention. In some embodiments, the organic amine is triethyl amine, diisopropyl amine, ethanol amine, diethanol amine, triethanol amine, morpholine, N-methylmorpholine, piperidine, N-methylpiperidine, N-ethylpiperidine, dibenzylamine, piperazine, pyridine, pyrrazole, pipyrrazole, imidazole, pyrazine, or pipyrazine.

In some embodiments, an ammonium salt is a triethyl amine salt, a diisopropyl amine salt, an ethanol amine salt, a diethanol amine salt, a triethanol amine salt, a morpholine salt, an N-methylmorpholine salt, a piperidine salt, an N-methylpiperidine salt, an N-ethylpiperidine salt, a dibenzylamine salt, a piperazine salt, a pyridine salt, a pyrrazole salt, an imidazole salt, or a pyrazine salt.

In some embodiments, acid addition salts arise from the addition of an acid to a compound of the invention. In some embodiments, the acid is organic. In some embodiments, the acid is inorganic. In some embodiments, the acid is hydrochloric acid, hydrobromic acid, hydroiodic acid, nitric acid, nitrous acid, sulfuric acid, sulfurous acid, a phosphoric acid, isonicotinic acid, lactic acid, salicylic acid, tartaric acid, ascorbic acid, gentisinic acid, gluconic acid, glucaronic acid, saccaric acid, formic acid, benzoic acid, glutamic acid, pantothenic acid, acetic acid, propionic acid, butyric acid, fumaric acid, succinic acid, methanesulfonic acid, ethanesulfonic acid, benzenesulfonic acid, p-toluenesulfonic acid, citric acid, oxalic acid, or maleic acid.

In some embodiments, the salt is a hydrochloride salt, a hydrobromide salt, a hydroiodide salt, a nitrate salt, a nitrite salt, a sulfate salt, a sulfite salt, a phosphate salt, isonicotinate salt, a lactate salt, a salicylate salt, a tartrate salt, an ascorbate salt, a gentisinate salt, a gluconate salt, a glucaronate salt, a saccarate salt, a formate salt, a benzoate salt, a glutamate salt, a pantothenate salt, an acetate salt, a propionate salt, a butyrate salt, a fumarate salt, a succinate salt, a methanesulfonate (mesylate) salt, an ethanesulfonate salt, a benzenesulfonate salt, a p-toluenesulfonate salt, a citrate salt, an oxalate salt, or a maleate salt.

The compounds described herein may in some cases exist as diastereomers, enantiomers, or other stereoisomeric forms. The compounds and salts presented herein include all diastereomeric, enantiomeric, and epimeric forms as well as the appropriate mixtures thereof. Separation of stereoisomers may be performed by chromatography or by forming diastereomers and separating by recrystallization, or chromatography, or any combination thereof. (Jean Jacques, Andre Collet, Samuel H. Wilen, “Enantiomers, Racemates and Resolutions”, John Wiley And Sons, Inc., 1981, herein incorporated by reference for this disclosure). Stereoisomers may also be obtained by stereoselective synthesis.

According to another embodiment, the present disclosure provides methods of producing the above-defined compounds. The compounds may be synthesized using any suitable techniques. Advantageously, these compounds are conveniently synthesized from readily available starting materials. Synthetic chemistry transformations and methodologies useful in synthesizing the compounds described herein are known in the art and include, for example, those described in R. Larock, Comprehensive Organic Transformations (1989); T. W. Greene and P. G. M. Wuts, Protective Groups in Organic Synthesis, 2d. Ed. (1991); L. Fieser and M. Fieser, Fieser and Fieser's Reagents for Organic Synthesis (1994); and L. Paquette, ed., Encyclopedia of Reagents for Organic Synthesis (1995).). In general, synthesis and measurements of ULK1 inhibitory activity of the compounds described herein was performed using method analogous to those previously described in PCT International Application No. PCT/US2015/046777 which is hereby incorporated by reference in its entirety.

### Pharmaceutical Formulations

The compounds of the present invention may be administered in various forms, including those detailed herein. The treatment with the compound may be a component of a combination therapy or an adjunct therapy, i.e. the subject or patient in need of the drug is treated or given another drug for the disease in conjunction with one or more of the instant compounds. In some embodiments, this combination therapy is sequential therapy where the patient is treated first with one drug and then the other or the two drugs are given simultaneously. In some embodiments, these are administered independently by the same route or by two or more different routes of administration depending on the dosage forms employed.

As used herein, a “pharmaceutically acceptable carrier” is a pharmaceutically acceptable solvent, suspending agent or vehicle, for delivering the instant compounds to the animal or human. The carrier may be liquid or solid and is selected with the planned manner of administration in mind. Liposomes are also a pharmaceutically acceptable carrier.

The dosage of the compounds administered in treatment will vary depending upon factors such as the pharmacodynamic characteristics of a specific chemotherapeutic agent and its mode and route of administration; the age, sex, metabolic rate, absorptive efficiency, health and weight of the recipient; the nature and extent of the symptoms; the kind of concurrent treatment being administered; the frequency of treatment with; and the desired therapeutic effect.

A dosage unit of the compounds used in the method of the present invention may comprise a single compound or mixtures thereof with additional agents. In some embodiments, the compounds are administered in oral dosage forms as tablets, capsules, pills, powders, granules, elixirs, tinctures, suspensions, syrups, and emulsions. The compounds may also be administered in intravenous (bolus or infusion), intraperitoneal, subcutaneous, or intramuscular form, or introduced directly, e.g. by injection, topical application, or other methods, into or onto a site of infection, all using dosage forms well known to those of ordinary skill in the pharmaceutical arts.

The compounds used in the method of the present invention may be administered in admixture with suitable pharmaceutical diluents, extenders, excipients, or carriers (collectively referred to herein as a pharmaceutically acceptable carrier) suitably selected with respect to the intended form of administration and as consistent with conventional pharmaceutical practices. The unit will be in a form suitable for oral, rectal, topical, intravenous or direct injection or parenteral administration. In some embodiments, the compounds are administered alone or mixed with a pharmaceutically acceptable carrier. In some embodiments, this carrier is a solid or liquid, and the type of carrier is generally chosen based on the type of administration being used. In some embodiments, the active agent is co-administered in the form of a tablet or capsule, liposome, as an agglomerated powder or in a liquid form. Examples of suitable solid carriers include lactose, sucrose, gelatin and agar. Capsule or tablets are easily formulated and made easy to swallow or chew; other solid forms include granules, and bulk powders. Tablets may contain suitable binders, lubricants, diluents, disintegrating agents, coloring agents, flavoring agents, flow-inducing agents, and melting agents. Examples of suitable liquid dosage forms include solutions or suspensions in water, pharmaceutically acceptable fats and oils, alcohols or other organic solvents, including esters, emulsions, syrups or elixirs, suspensions, solutions and/or suspensions reconstituted from non-effervescent granules and effervescent preparations reconstituted from effervescent granules. Such liquid dosage forms may contain, for example, suitable solvents, preservatives, emulsifying agents, suspending agents, diluents, sweeteners, thickeners, and melting agents. Oral dosage forms optionally contain flavorants and coloring agents. Parenteral and intravenous forms may also include minerals and other materials to make them compatible with the type of injection or delivery system chosen.

Aspects of the invention include articles of manufacture, or kits, comprising the active agents described herein, and formulations thereof, as well as instructions for use. An article of manufacture, or kit, can further contain at least one additional reagent, e.g., a chemotherapeutic drug, etc. Articles of manufacture and kits typically include a label indicating the intended use of their contents. The term “label” as used herein includes any writing, or recorded material supplied on or with a kit, or which otherwise accompanies a kit.

Techniques and compositions for making dosage forms useful in the present invention are described in the following references: 7 Modern Pharmaceutics, Chapters 9 and 10 (Banker & Rhodes, Editors, 1979); Pharmaceutical Dosage Forms: Tablets (Lieberman et al., 1981); Ansel, Introduction to Pharmaceutical Dosage Forms 2nd Edition (1976); Remington's Pharmaceutical Sciences, 17th ed. (Mack Publishing Company, Easton, Pa., 1985); Advances in Pharmaceutical Sciences (David Ganderton, Trevor Jones, Eds., 1992); Advances in Pharmaceutical Sciences Vol. 7. (David Ganderton, Trevor Jones, James McGinity, Eds., 1995); Aqueous Polymeric Coatings for Pharmaceutical Dosage Forms (Drugs and the Pharmaceutical Sciences, Series 36 (James McGinity, Ed., 1989); Pharmaceutical Particulate Carriers: Therapeutic Applications: Drugs and the Pharmaceutical Sciences, Vol 61 (Alain Rolland, Ed., 1993); Drug Delivery to the Gastrointestinal Tract (Ellis Horwood Books in the Biological Sciences. Series in Pharmaceutical Technology; J. G. Hardy, S. S. Davis, Clive G. Wilson, Eds.); Modem Pharmaceutics Drugs and the Pharmaceutical Sciences, Vol 40 (Gilbert S. Banker, Christopher T. Rhodes, Eds.). All of the aforementioned publications are incorporated by reference herein.

The compounds used in the method of the present invention may also be administered in the form of liposome delivery systems, such as small unilamellar vesicles, large unilamallar vesicles, and multilamellar vesicles. Liposomes may be formed from a variety of phospholipids, such as cholesterol, stearylamine, or phosphatidylcholines. The compounds may be administered as components of tissue-targeted emulsions.

The compounds used in the method of the present invention may also be coupled to soluble polymers as targetable drug carriers or as a prodrug. Such polymers include polyvinylpyrrolidone, pyran copolymer, polyhydroxylpropylmethacrylamide-phenol, polyhydroxyethylasparta-midephenol, or polyethyleneoxide-polylysine substituted with palmitoyl residues. Furthermore, the compounds may be coupled to a class of biodegradable polymers useful in achieving controlled release of a drug, for example, polylactic acid, polyglycolic acid, copolymers of polylactic and polyglycolic acid, polyepsilon caprolactone, polyhydroxy butyric acid, polyorthoesters, polyacetals, polydihydropyrans, polycyanoacylates, and crosslinked or amphipathic block copolymers of hydrogels.

Parenteral and intravenous forms may also include minerals and other materials to make them compatible with the type of injection or delivery system chosen.

Each embodiment disclosed herein is contemplated as being applicable to each of the other disclosed embodiments. Thus, all combinations of the various elements described herein are within the scope of the invention.

### Methods of the Disclosure

In some instances, ULK inhibitors are used and/or useful in the treatment of cancer and/or ULK mediated disorders. Surprisingly, in certain instances, ULK inhibitors are efficacious as a monotherapy. In some instances, the ULK inhibitor inhibits ULK1. In some instances, the ULK inhibitor is a ULK1 specific inhibitor. In some instances, the ULK inhibitor inhibits both ULK1 and ULK2. In other instances, it is also surprising that ULK inhibitors are used/useful in augmenting or improving standard of care therapies.

Monotherapy

In one aspect, provided herein, is a method of treating a disease or disorder with a ULK inhibitor. In various embodiments, the ULK inhibitor is administered alone to treat a disease or disorder. In some embodiments, the method comprises administering to a subject in need thereof a therapeutically effective amount of a ULK inhibitor. In some instances, the ULK inhibitor inhibits ULK1. In some instances, the ULK inhibitor is a ULK1 specific inhibitor. In some instances, the ULK inhibitor inhibits both ULK1 and ULK2.

In some embodiments, the ULK inhibitor is administered as a monotherapy. In some embodiments, the ULK inhibitor is the sole therapeutic agent administered to the patient for the treatment of the disease or disorder. In some embodiments, the ULK inhibitor is the sole anti-cancer agent administered to the patient. In some embodiments, the ULK inhibitor is administered as a monotherapy with additional inactive ingredients as part of a pharmaceutical formulation. In some instances, the ULK inhibitor inhibits ULK1. In some instances, the ULK inhibitor is a ULK1 specific inhibitor. In some instances, the ULK inhibitor inhibits both ULK1 and ULK2.

In some embodiments, the disease or disorder is characterized by abnormal autophagy. In some embodiments, the abnormal autophagy is therapeutically induced. In some embodiments, the disease or disorder is refractory. In some embodiments, the disease or disorder is refractory to treatment with a non-ULK inhibitor therapeutic agent. In embodiments, the disease or disorder is resistant to treatment with a non-ULK inhibitor therapeutic agent.

In some embodiments, the disease or disorder treated with a ULK inhibitor as a monotherapy is cancer. In some instances, the ULK inhibitor inhibits ULK1. In some instances, the ULK inhibitor is a ULK1 specific inhibitor. In some instances, the ULK inhibitor inhibits both ULK1 and ULK2. In some embodiments, the cancer is lung cancer. In specific embodiments, the lung cancer is non-small cell lung cancer. In some embodiments, the cancer is an advanced stage non-small cell lung cancer. In some embodiments, the cancer comprises a tumor. In some embodiments, the non-small cell lung cancer comprises a tumor. In some embodiments, the non-small cell lung cancer is characterized by abnormal autophagy. In some embodiments, the lung cancer is refractory. In some embodiments, the lung cancer is refractory to treatment with carboplatin. In some embodiments, the non-small cell lung cancer is refractory. In some embodiments, the non-small cell lung cancer is refractory to treatment with carboplatin. In some embodiments, the lung cancer is characterized by cytostasis.

In some embodiments, the cancer is pancreatic cancer. In some embodiments, the pancreatic cancer comprises a tumor. In some embodiments, the pancreatic cancer is characterized by abnormal autophagy. In some embodiments, the pancreatic cancer is refractory. In some embodiments, the pancreatic cancer is characterized by cytostasis. In some embodiments, the pancreatic cancer is pancreatic ductal adenocarcinoma (PDAC).

In some embodiments, the cancer is breast cancer. In some embodiments, the breast cancer comprises a tumor. In some embodiments, the breast cancer is characterized by abnormal autophagy. In some embodiments, the breast cancer is refractory. In some embodiments, the breast cancer is characterized by cytostasis. In some embodiments, the breast cancer is triple negative breast cancer.

In some embodiments, the disease or disorder treated with a ULK inhibitor as a monotherapy is lymphoangiomyoleiomatosis. In some embodiments, the disease or disorder treated with a ULK inhibitor as a monotherapy is tuberous sclerosis complex. In some instances, the ULK inhibitor inhibits ULK1. In some instances, the ULK inhibitor is a ULK1 specific inhibitor. In some instances, the ULK inhibitor inhibits both ULK1 and ULK2.

In some embodiments, administering a ULK inhibitor slows progression of the disease or disorder. In some embodiments, administering a ULK inhibitor slows progression of the disease or disorder by at least 10%, at least 20%, at least 30%, at least 40%, at least 50%, at least 60%, at least 70%, at least 80%, at least 90%, or at least 95%. In some embodiments, progression is measured by tumor growth. In some embodiments, administering a ULK inhibitor arrests cancer cell growth. In some embodiments, administering a ULK inhibitor reduces tumor volume. In some instances, the ULK inhibitor inhibits ULK1. In some instances, the ULK inhibitor is a ULK1 specific inhibitor. In some instances, the ULK inhibitor inhibits both ULK1 and ULK2.

In some embodiments, the method of treatment comprises decreasing phosphorylation of ATG13 in the subject. In some embodiments, the method comprises degrading ATG13 in diseased tissue of the subject. In some embodiments, administering the ULK inhibitor degrades ATG13.

In some embodiments, the subject comprises a mutation in at least one of KRAS, PTEN, TSC1, TSC2, PIk3CA, P53, STK11 (a.k.a. LKB1), KEAP1, NRF2, ALK4, GNAS, or EGFR. In some embodiments, the subject comprises a mutation in at least one of SMAD4, p16/CDKM2A, or BRCA2.

Combination Therapy

The compounds, or the pharmaceutically acceptable salts thereof, provided herein may be administered in combination with one or more therapeutic agents.

Also described herein are combination therapies. In some instances, the combination therapies of the present invention comprise a ULK inhibitor and an additional therapeutic agent. In some instances, the ULK inhibitor inhibits ULK1. In some instances, the ULK inhibitor is a ULK1 specific inhibitor. In some instances, the ULK inhibitor inhibits both ULK1 and ULK2. In some embodiments, there is an additional therapeutic benefit when compared to treatment with the additional therapeutic agent alone. In some instances, the combination of the ULK inhibitor and the additional therapeutic agent shut down pathways of autophagy. This allows for enhanced cell death in diseased tissue, as the diseased cells will not be able to rely on autophagic processes for survival once the pathway is shut off with a ULK inhibitor. In some embodiments, the addition of a ULK inhibitor allows for successful treatment of a disease that is otherwise refractory to treatment of the additional therapeutic agent by itself. In some embodiments, the addition of the ULK inhibitor enhances the efficacy of the additional therapeutic agent. In some embodiments, the addition of the ULK inhibitor has a synergistic effect with the additional therapeutic agent. In some embodiments, the additional therapeutic agent is a standard of care therapy.

In one aspect, provided herein, is a method of treating a disease or disorder with a ULK inhibitor and an additional therapeutic agent. In some embodiments, the method comprises administering to a subject in need thereof a therapeutically effective amount of a ULK inhibitor. In some embodiments, the method comprises administering to a subject in need thereof a therapeutically effective amount of a ULK inhibitor and a therapeutically effective amount of an additional therapeutic agent. In some instances, the ULK inhibitor inhibits ULK1. In some instances, the ULK inhibitor is a ULK1 specific inhibitor. In some instances, the ULK inhibitor inhibits both ULK1 and ULK2.

In some embodiments, the disease or disorder is lymphoangiomyoleiomatosis. In some embodiments, the disease or disorder is tuberous sclerosis complex.

In some embodiments, the disease or disorder is cancer. In some embodiments, the disease or disorder is refractory cancer. In some embodiments, the cancer comprises a tumor. In some embodiments, the cancer is refractory to treatment with carboplatin. In some embodiments, the cancer is refractory to trametinib. In some embodiments, the cancer is refractory to an MEK inhibitor. In some embodiments, cancer is pancreatic cancer. In some embodiments, the cancer is lung cancer. In some embodiments, the lung cancer is non-small cell lung cancer. In some embodiments, the cancer is refractory to an mTOR inhibitor. In some embodiments, the cancer is refractory to rapamycin. In some embodiments, the cancer is refractory to treatment with a rapamycin analog.

In some embodiments, the cancer is pancreatic cancer and the additional therapeutic agent is trametinb. In some embodiments, the cancer is pancreatic cancer and the additional therapeutic agent is an MEK inhibitor. In some embodiments, the MEK inhibitor is trametinib, cobimetinib, binimetinib, or selumetinib. In some embodiments, the cancer is pancreatic cancer and the additional therapeutic agent is gemcitabine. In some embodiments, the cancer is pancreatic cancer and the additional therapeutic agent is a nucleoside analog. In some embodiments, the cancer is pancreatic cancer and the additional therapeutic agent is gemcitabine, everolimus, erlotinib, or sunitinib. In some embodiments, the additional therapeutic agent is FOLFIRINOX (5-fluorouracil, leucovorin, irinotecan, and oxaliplatin), gemcitabine, or gemcitabine/abraxane. In some embodiments, the additional therapeutic agent is capeditabine, leucovorin, nab-paclitaxel, nanoliposomal irinotecan, gemcitabine/nab-paclitaxel, pembrolizumab, or cisplatin. In some embodiments, the additional therapeutic agent is capeditabine, leucovorin, nab-paclitaxel, nanoliposomal irinotecan, gemcitabine/nab-paclitaxel, pembrolizumab, or cisplatin. In some embodiments, the pancreatic cancer is pancreatic ductal adenocarcinoma (PDAC). In some embodiments, the subject with pancreatic cancer comprises a mutation in at least one of SMAD4, p16/CDKM2A, or BRCA2. In some embodiments, the cancer is pancreatic cancer and the additional therapeutic agent is a standard of care therapy.

In some embodiments, the cancer is breast cancer. In some embodiments, the cancer is breast cancer and the additional therapeutic agent is a standard of care therapy. In some embodiments, the cancer is breast cancer and the additional therapeutic agent is anastrozole, exemestane, letrozole, or tamoxifen. In some embodiments, the cancer is breast cancer and the additional therapeutic agent is a poly ADP ribose polymerase (PARP) inhibitor. In some embodiments, the PARP inhibitor is olaparib, rucaparib, niraparib, or talazoparib. In some embodiments, the breast cancer is triple negative breast cancer (TNBC).

In some embodiments, the cancer is lung cancer and the additional therapeutic agent is carboplatin. In some embodiments, the cancer is lung cancer and the additional therapeutic agent is a carboplatin analog. In some embodiments, the cancer is non-small cell lung cancer and the additional therapeutic agent is carboplatin. In some embodiments, the cancer is non-small cell lung cancer and the additional therapeutic agent is a carboplatin analog. In some embodiments, the carboplatin analog is cisplatin or dicycloplatin. In some embodiments, the cancer is lung cancer and the additional therapeutic agent is erlotinib, gefitinib, osimertinib, or crizotinib. In some embodiments, the cancer is non-small cell lung cancer and the additional therapeutic agent is erlotinib, gefitinib, osimertinib, or crizotinib. In some embodiments, the cancer is lung cancer and the additional therapeutic agent is pemetrexed, docetaxol, or pembroluzimab. In some embodiments, the cancer is non-small cell lung cancer and the additional therapeutic agent is pemetrexed, docetaxol, or pembroluzimab. In some embodiments, the cancer is lung cancer and the additional therapeutic agent is gemcitabine, bortexomib, trastuzumab, vinorelbine, doxorubicin, irinotecan, temsirolimus, sunitinib, nivolumab, or bevacizumab. In some embodiments, the cancer is lung cancer and the additional therapeutic agent is carboplatin/gemcitabine, carboplatin/paclitaxel/cetuximua, cisplatin/pemetrexed, cisplatin/docetaxel, cisplatin/docetaxel/bevacizumab, everolimus/nab-paclitaxel, or tremelimumab/durvalumab. In some embodiments, the cancer is non-small cell lung cancer and the additional therapeutic agent is gemcitabine, bortexomib, trastuzumab, vinorelbine, doxorubicin, irinotecan, temsirolimus, sunitinib, nivolumab, or bevacizumab. In some embodiments, the cancer is non-small cell lung cancer and the additional therapeutic agent is carboplatin/gemcitabine, carboplatin/paclitaxel/cetuximua, cisplatin/pemetrexed, cisplatin/docetaxel, cisplatin/docetaxel/bevacizumab, everolimus/nab-paclitaxel, or tremelimumab/durvalumab. In some embodiments, the subject with lung cancer comprises a mutation in KRAS, PTEN, TSC1, TSC2, PIk3CA, P53, STK11 (a.k.a. LKB1), KEAP1, NRF2, ALK4, GNAS or EGFR.

In some embodiments, the additional therapeutic agent is carboplatin. In some embodiments, the additional therapeutic agent is carboplatin or a carboplatin analog. In some embodiments, the carboplatin analog is cisplatin or dicycloplatin.

In some embodiments, the additional therapeutic agent is erlotinib, gefitinib, osimertinib, or crizotinib. In some embodiments, the additional therapeutic agent is pemetrexed, docetaxol, or pembroluzimab. In some embodiments, the additional therapeutic agent is carboplatin/gemcitabine, carboplatin/paclitaxel/cetuximua, cisplatin/pemetrexed, cisplatin/docetaxel, cisplatin/docetaxel/bevacizumab, everolimus/nab-paclitaxel, or tremelimumab/durvalumab.

In some embodiments, the additional therapeutic agent is anastrozole, exemestane, letrozole, or tamoxifen. In some embodiments, the additional therapeutic agent is a poly ADP ribose polymerase (PARP) inhibitor. In some embodiments, the PARP inhibitor is olaparib, rucaparib, niraparib, or talazoparib.

In some embodiments, the additional therapeutic agent is gemcitabine, everolimus, erlotinib, or sunitinib. In some embodiments, the additional therapeutic agent is a nucleoside analog. In some embodiments, is FOLFIRINOX (5-fluorouracil, leucovorin, irinotecan, and oxaliplatin), gemcitabine, or gemcitabine/abraxane. In some embodiments, the additional therapeutic agent is capeditabine, leucovorin, nab-paclitaxel, nanoliposomal irinotecan, gemcitabine/nab-paclitaxel, pembrolizumab, or cisplatin.

In some embodiments, the additional therapeutic agent is an MEK inhibitor. In some embodiments, the additional therapeutic agent is trametinib. In some embodiments, the MEK inhibitor is trametinib, cobimetinib, binimetinib, or selumetinib.

In some embodiments, the additional therapeutic agent is gemcitabine. In some embodiments, the additional therapeutic agent is a nucleoside analog.

In some embodiments, the additional therapeutic agent is an mTOR inhibitor. In some embodiments, the additional therapeutic agent is rapamycin. In some embodiments, mTOR inhibitor is rapamycin, sirolimus, temsirolimus, everolimus, ridaforolimus, NVPBEZ235, BGT226, XL765, GDC0980, SF1 126, PK1587, PF04691502, GSK2126458, INK128, TORKiCC223, OSI027, AZD8055, AZD2014, and Palomid 529, metformin, or AICAR (5-amino-1-P-D-ribofuranosyl-imidazole-4-carboxamide). In some embodiments, the additional therapeutic agent is a rapamycin analog.

In some embodiments, the disease or disorder is lymphoangiomyoleiomatosis and the additional therapeutic agent is an mTOR inhibitor. In some embodiments, the disease or disorder is tuberous sclerosis complex and the additional therapeutic agent is an mTOR inhibitor.

In some embodiments, the additional therapeutic agent was previously administered to the subject without a ULK inhibitor. In some embodiments, the additional therapeutic agent induces a cytostatic response. In some embodiments, the additional therapeutic agent induces a cytostatic response when administered without a ULK inhibitor. In some embodiments, the additional therapeutic agent induces a cytostatic response in disease tissue. In some embodiments, the additional therapeutic agent induces a cytostatic response in the diseased tissue when the additional therapeutic agent was administered without a ULK inhibitor. In some instances, the ULK inhibitor inhibits ULK1. In some instances, the ULK inhibitor is a ULK1 specific inhibitor. In some instances, the ULK inhibitor inhibits both ULK1 and ULK2

In some embodiments, the subject is treated with the additional therapeutic agent prior to treatment with the ULK inhibitor. In some embodiments, treatment with the additional therapeutic agent is ceased prior to administration of the ULK inhibitor. In some embodiments, treatment with the additional therapeutic agent produces a cytostatic response in diseased tissue.

In some embodiments, the ULK inhibitor and the additional therapeutic agent are administered concomitantly. In some embodiments, the ULK inhibitor and the additional therapeutic agent are administered together at the start of treatment. In some instances, the ULK inhibitor inhibits ULK1. In some instances, the ULK inhibitor is a ULK1 specific inhibitor. In some instances, the ULK inhibitor inhibits both ULK1 and ULK2.

In some embodiments, the disease or disorder is characterized by abnormal autophagy. In some embodiments, the abnormal autophagy is therapeutically induced. In some embodiments, the disease or disorder is refractory. In some embodiments, the disease or disorder is refractory to treatment with an additional therapeutic agent. In embodiments, the disease or disorder is resistant to treatment with an additional therapeutic agent.

In some embodiments, administering a ULK inhibitor slows progression of the disease or disorder. In some embodiments, administering a ULK inhibitor slows progression of the disease or disorder when compared to administration of the additional therapeutic agent with the ULK inhibitor. In some embodiments, administering a ULK inhibitor slows progression of the disease or disorder by at least 10%, at least 20%, at least 30%, at least 40%, at least 50%, at least 60%, at least 70%, at least 80%, at least 90%, or at least 95%. In some embodiments, administering a ULK slows the progression of the disease or disorder by at least 10%, at least 20%, at least 30%, at least 40%, at least 50%, at least 60%, at least 70%, at least 80%, at least 90%, or at least 95% when compared to administration of the additional therapeutic agent with the ULK inhibitor. In some embodiments, progression of the disease or disorder comprises growth of a tumor. In some embodiments, progression is measured by tumor growth. In some embodiments, administering a ULK inhibitor arrests cancer cell growth. In some embodiments, administering a ULK inhibitor reduces tumor volume. In some instances, the ULK inhibitor inhibits ULK1. In some instances, the ULK inhibitor is a ULK1 specific inhibitor. In some instances, the ULK inhibitor inhibits both ULK1 and ULK2.

In some embodiments, administering a ULK inhibitor enhances the efficacy of the additional therapeutic agent by at least 10%, at least 20%, at least 30%, at least 40%, at least 50%, at least 60%, at least 70%, at least 80%, at least 90%, or at least 95%. In some embodiments, administering a ULK inhibitor enhances the efficacy of the additional therapeutic agent by at least 10%, at least 20%, at least 30%, at least 40%, at least 50%, at least 60%, at least 70%, at least 80%, at least 90%, or at least 95% when compared to administration of the additional therapeutic agent with the ULK inhibitor. In some embodiments, the efficacy is measured by a change in the rate of tumor growth. In some embodiments, efficacy is measured by reduction of tumor volume. In some instances, the ULK inhibitor inhibits ULK1. In some instances, the ULK inhibitor is a ULK1 specific inhibitor. In some instances, the ULK inhibitor inhibits both ULK1 and ULK2.

In some embodiments, the method of treatment comprises decreasing phosphorylation of ATG13 in the subject. In some embodiments, the method comprises degrading ATG13 in diseased tissue of the subject. In some embodiments, administering a ULK inhibitor causes degradation of ATG13.

In some embodiments, the subject comprises a mutation in at least one of KRAS, PTEN, TSC1, TSC2, PIk3CA, P53, STK11 (a.k.a. LKB1), KEAP1, NRF2, ALK4, GNAS, or EGFR. In some embodiments, the subject comprises a mutation in at least one of SMAD4, p16/CDKM2A, or BRCA2.

Additional indications for which ULK1 inhibitors are useful are described in PCT International Application No. PCT/US2015/046777, which is hereby incorporated by reference in its entirety.

### EXAMPLES

### Chemical Synthesis

Reactions conducted under microwave irradiation were performed in a CEM Discover microwave reactor using either CEM 10 mL reaction vessels or a ChemGlass heavy wall pressure vessel (100 mL, 38 mm×190 mm). Reaction progress was monitored by reverse-phase HPLC and/or thin-layer chromatography (TLC). Liquid chromatography-mass spectrometry was performed using either Waters or Shimadzu 2010EV LCMS instruments using water and acetonitrile or methanol doped with 0.1% formic acid. TLC was performed using silica gel 60 F254 pre-coated plates (0.25 mm). Flash chromatography was performed using silica gel (32-63 m particle size) or aluminum oxide (activated, basic, ^{˜}150 mesh size). Automated chromatographic purification was carried out using pre-packed silica or C18 cartridges (from RediSep and Luknova) and eluted using an ISCO Companion system. Reverse phase purifications were conducted using water and acetonitrile or methanol doped with 0.1% formic acid. All final product compounds were purified using one of these two chromotographic methods. Purity and characterization of compounds was established by a combination of TLC, liquid chromatography-mass spectroscopy (LC-MS) and Nuclear Magnetic Resonance (NMR) analytical techniques. ¹H and ¹³C NMR spectra were obtained on a Joel 400 spectrometer at 400 MHz and 101 MHz, respectively. Chemical shifts are reported in δ (ppm) and were internally referenced to deuterated solvent signals.

### LC-MS Conditions

In Examples 31-73, HPLC-MS analyses were performed on a Waters ACQUITY UPLC with SQ mass detector and PDA eλ detector. The column used was a Phenomenex Kinetex C18 column (1.7 um, 2.1×50 mm). The mobile phase consisted of eluent A (water, 0.05% TFA) and eluent B (CH₃CN, 0.05% TFA), and the elution proceeded at 0.5 mL/min. The initial conditions were 90% A, then 90% A to 10% A linearly decreased within 1.75 min, then from 10% A to 90% A within 0.25 min. The total run time is 2 minutes.

Abbreviations used: mass spectrometry (MS), palladium on carbon (Pd—C), acetonitrile (MeCN), dichloromethane (DCM), diethyl ether (Et₂O), ethyl acetate (EtOAc), ethanol (EtOH), methanol (MeOH), tetrahydrofuran (THF), and trifluoroacetic acid (TFA).

The following abbreviations and terms have the indicated meanings throughout:

BOC or Boc=tert-butoxycarbonyl

DCM=dichloromethane

DIPEA or DIEA=N,N-diisopropylethylamine

EDCI.HCl=1-ethyl-3-(3-dimethylaminopropyl) carbodiimide hydrochloride

eq=equivalent(s)

Et=ethyl

EtOAc or EA=ethyl acetate

EtOH=ethanol

g=gram

h or hr(s)=hour

HOBt=hydroxybenzotriazole

HPLC=high pressure liquid chromatography

kg or Kg=kilogram

L or l=liter

LC/MS=LCMS=liquid chromatography-mass spectrometry

LRMS=low resolution mass spectrometry

m/z=mass-to-charge ratio

Me=methyl

MeOH=methanol

mg=milligram

min(s)=minute(s)

mL=milliliter

mmol=millimole

RP-HPLC=reverse phase-high pressure liquid chromatography

rt or RT=room temperature

THF=tetrahydrofuran

TLC=thin layer chromatography

UV=ultraviolet

### Example 1: Synthesis of Compound of Formula (IA)

Compounds of Formula (IA) may be synthesized according to the general Scheme 1 described below.

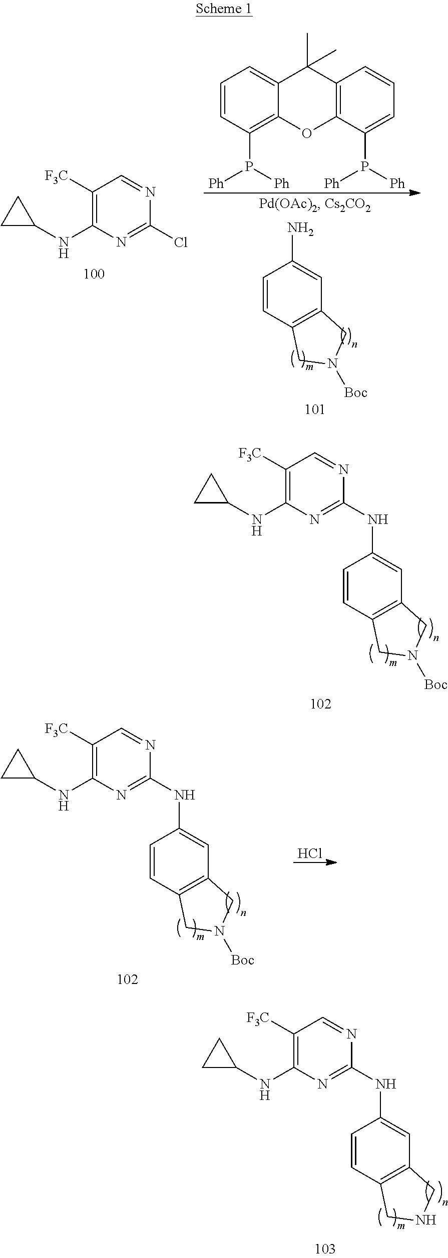

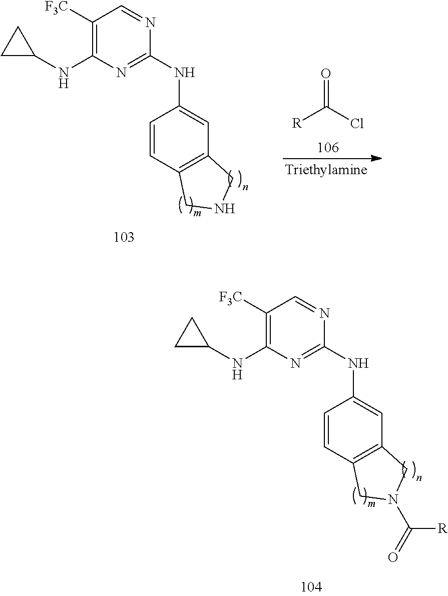

Flame dried flask and stir bar. Bubbled nitrogen through reagents and solvents prior to heating. 2-chloro-N-cyclopropyl-5-(trifluoromethyl)pyrimidin-4-amine 100 (0.100 g, 0.421 mmol), diacetoxypalladium (2.83 mg, 0.013 mmol), Boc-protected alkyl-amino aniline 101 0.463 mmol) and cesium carbonate (0.178 g, 0.547 mmol) are mixed in 1,4-dioxane (2 ml). The mixture is microwaved at 130° C. for 20 min. Filtered through Celite with MeOH and then concentrated. Add acetone and filter the solid; product is in the filtrate, which is concentrated. Product 102 is recovered after purification by flash chromatography on silica gel (DCM-EtOAc).

Intermediate 102 and hydrogen chloride in water (0.489 ml, 1.468 mmol, 3M in water) are mixed in methanol (1 ml). Heated to 60° C. for 16 h and then concentrated. The solid is washed with DCM to give product 103.

To yield acylated compounds 104, compounds 103, acyl chloride 106, and triethylamine (0.026 ml, 0.187 mmol) are mixed in DMF (3 ml). Heated to 60° C. for 8 h. Add MeOH and concentrate. Product is recovered after flash chromatography on silica gel (DCM-EtOAc).

The remaining compounds not covered by the synthetic route of Scheme 1 were prepared by analogous methods.

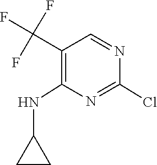 [SMILES: FC(F)(F)c1cnc(Cl)nc1NC1CC1]

2-Chloro-N-cyclopropyl-5-(trifluoromethyl)pyrimidin-4-amine. A solution of 2,4-dichloro-5-(trifluoromethyl)pyrimidine (1.00 g, 4.61 mmol), cyclopropylamine (0.32 mL, 4.61 mmol) and N,N-diisopropylethylamine (0.80 mL, 4.61 mmol) in acetonitrile (15 mL) was microwaved at 70° C. for 10 minutes in a 38 mL pressure vessel. The reaction mixture was then concentrated in vacuo and purified by automated reverse phase chromatography (water-acetonitrile eluent). Tan solid (0.349 g, 32% yield). LC-MS (ESI) calculated for C₈H₈ClF₃N₃[M+H]⁺: 238.04; found 238.30. ¹H NMR (400 MHz, DMSO-d6): δ 8.39 (s, 1H), 7.93 (d, J=3.6 Hz, 1H, 1H), 2.88 (dq, J=7.2, 3.6 Hz, 1H), 0.79-0.72 (m, 2H), 0.70-0.64 (m, 2H). ¹³C NMR (101 MHz, DMSO-d6): δ 162.66, 159.73, 155.42, 123.34 (q, J=271 Hz), 105.31 (q, J=32 Hz), 24.72, 6.30.

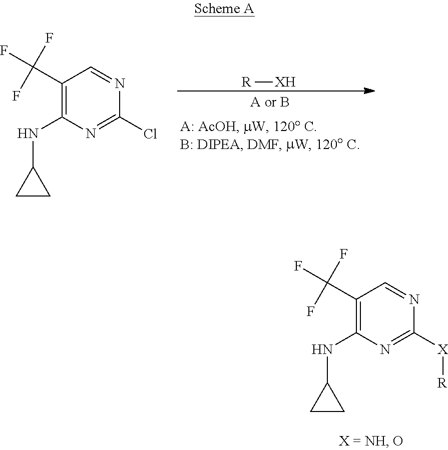

### General Method for Compounds in Scheme A.

A solution of 2-chloro-N-cyclopropyl-5-(trifluoromethyl)pyrimidin-4-amine (1.0 equiv.) and the appropriate aniline or phenol (1.0 equiv.) in acetic acid (2 mL) was microwaved at 120° C. for 10 minutes and then concentrated in vacuo. The crude product was purified by automated reverse phase chromatography to afford the title compound (Method 1A). To a solution of 2-chloro-N-cyclopropyl-5-(trifluoromethyl)pyrimidin-4-amine (1.0 equiv.), the appropriate aniline or phenol (1.2 equiv.), and N,N-diisopropylethylamine (1.2 equiv.) in DMF was microwaved at 120° C. for 10 min. The reaction mixture was concentrated in vacuo and the crude product was purified by automated reverse phase chromatography to afford the title compound (Method 1B).

### Example 2: Preparation cyclopropyl(6-((4-(cyclopropylamino)-5-(trifluoromethyl) pyrimidin-2-yl)amino)-3,4-dihydroisoquinolin-2(1H)-yl)methanone

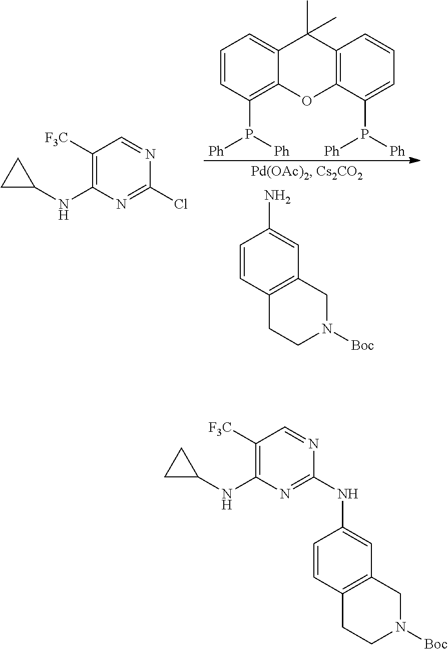

Flame dried flask and stir bar. Bubbled nitrogen through reagents and solvents prior to heating. 2-chloro-N-cyclopropyl-5-(trifluoromethyl)pyrimidin-4-amine (0.100 g, 0.421 mmol), diacetoxypalladium (2.83 mg, 0.013 mmol), tert-butyl 6-amino-3,4-dihydroisoquinoline-2(1H)-carboxylate (0.115 g, 0.463 mmol) and cesium carbonate (0.178 g, 0.547 mmol) were mixed in 1,4-dioxane (2 ml). The mixture was microwaved at 130° C. for 20 min. Filtered through Celite with MeOH and then concentrated. Added acetone and filtered the solid; product is in the filtrate, which was concentrated. 166 mg of product were isolated after flash chromatography on silica gel (DCM-EtOAc). MS calcd for [C₂₂H₂₆F₃N₅O₂+H]⁺: 450.21, found 450.55.

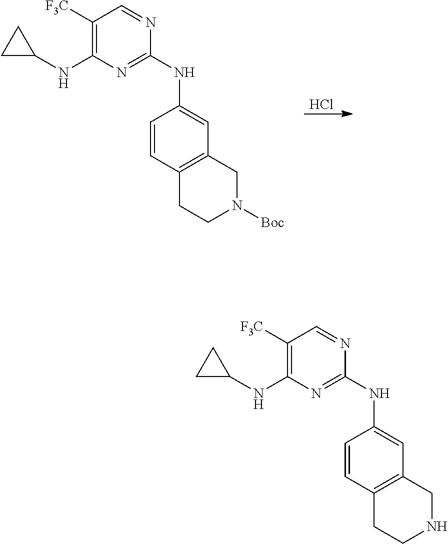 [SMILES: CC(C)(C)OC(=O)N1CCc2ccc(Nc3ncc(C(F)(F)F)c(NC4CC4)n3)cc2C1 ; Cl ; FC(F)(F)c1cnc(Nc2ccc3c(c2)CNCC3)nc1NC1CC1]

tert-Butyl 6-((4-(cyclopropylamino)-5-(trifluoromethyl)pyrimidin-2-yl)amino)-3,4-dihydroisoquinoline-2(1H)-carboxylate (0.132 g, 0.294 mmol) and hydrogen chloride in water (0.489 ml, 1.468 mmol, 3M in water) were mixed in methanol (1 ml). Heated to 60° C. for 16 h and then concentrated. The solid was washed with DCM to give 109 mg of product. MS calcd for [C₁₇H₁₈F₃N₅+H]⁺: 350.16, found 350.05.

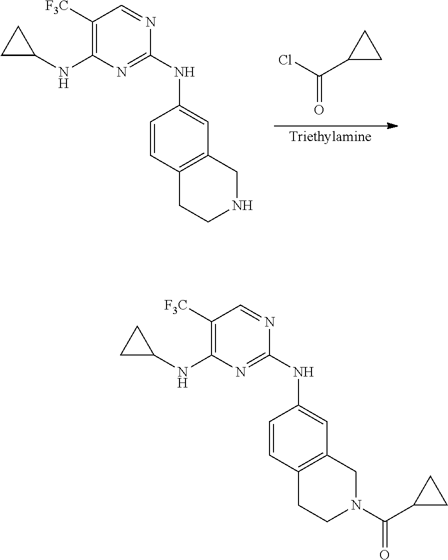

N4-Cyclopropyl-N2-(1,2,3,4-tetrahydroisoquinolin-6-yl)-5-(trifluoromethyl)pyrimidine-2,4-diamine.HCl (0.018 g, 0.047 mmol), cyclopropanecarbonyl chloride (4.23 μl, 0.047 mmol) and triethylamine (0.026 ml, 0.187 mmol) were mixed in DMF (3 ml). Heated to 60° C. for 8 h. Added MeOH and concentrated. 8 mg of product was recovered after flash chromatography on silica gel (DCM-EtOAc). MS calcd for [C₂₁H₂₂F₃N₅O+H]⁺: 418.19, found 418.00.

### Example 3: Synthesis of Compounds of Formula (IIB)

Compounds of Formula (IIB) may be synthesized according to the general Scheme 2 described below.

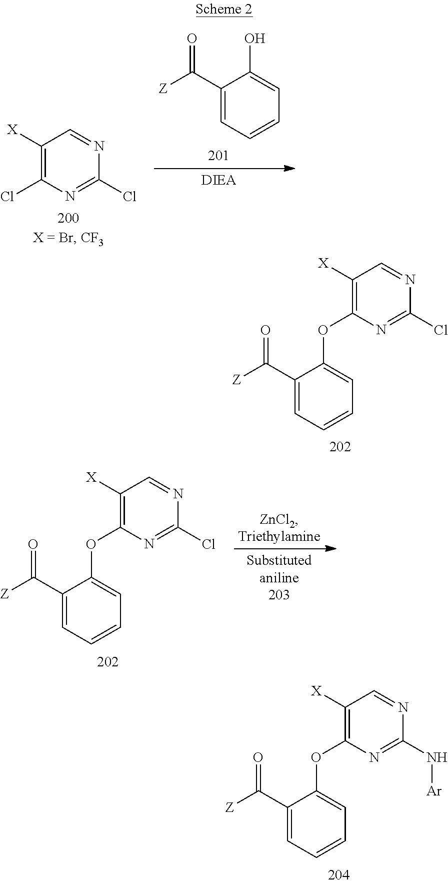

Dichloro pyrmidine 200 (0.65 mmol), substituted phenoxy compound 201 (0.65 mmol), and triethylamine (0.72 mmol) are mixed in acetonitrile (3 mL). the mixture is microwaved at 100° C. for 10 min, then concentrated to yield crude intermediate 202, which is used as is.

Intermediate 202 (0.6 mmol) and zinc (II) chloride (0.6 mmol) are mixed in 1,2-dichloroethane (3 mL) and t-butanol (0.5 mL). Triethylamine (0.66 mmol) and substituted aniline 203 (0.6 mmol) are added. The mixture is microwaved at 120° C. for 20 min and then concentrated. The product 204 is then purified by automated reverse phase chromatography and collected.

### Example 4: Preparation of 2-((5-bromo-2-((3,4,5-trimethoxyphenyl)amino)pyrimidin-4-yl)oxy)-N-cyclopropylbenzamide

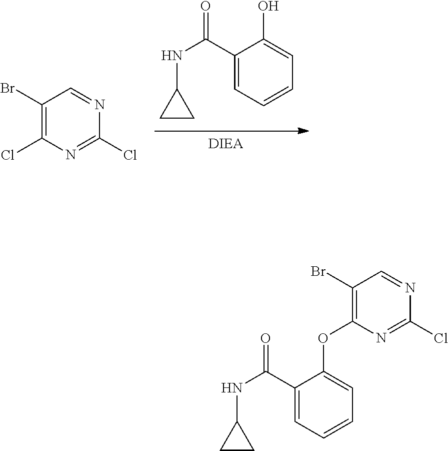 [SMILES: Clc1ncc(Br)c(Cl)n1 ; O=C(NC1CC1)c1ccccc1O ; O=C(NC1CC1)c1ccccc1Oc1nc(Cl)ncc1Br]

5-Bromo-2,4-dichloropyrimidine (0.150 g, 0.658 mmol), N-cyclopropyl-2-hydroxy benzamide (0.117 g, 0.658 mmol) and N-ethyl-N-isopropylpropan-2-amine (0.126 ml, 0.724 mmol) were mixed in acetonitrile (3 ml). The mixture was microwaved at 100° C. for 10 min and then concentrated and used as-is. MS calcd for [C₁₄H₁₁BrClN₃O₂+H]⁺: 367.98 found 367.70.

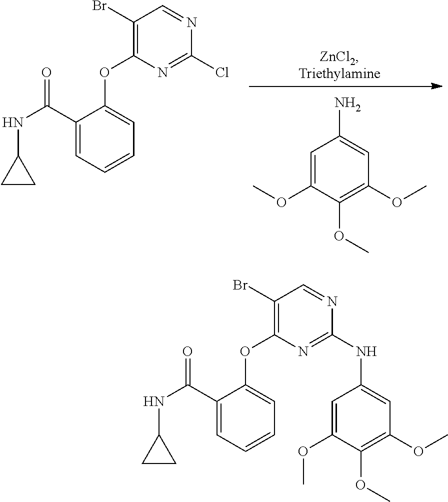

2-((5-Bromo-2-chloropyrimidin-4-yl)oxy)-N-cyclopropylbenzamide (0.230 g, 0.624 mmol) and zinc(II) chloride (0.085 g, 0.624 mmol) were mixed in 1,2-dichloroethane (3 ml) and t-butanol (0.5 ml). triethylamine (0.096 ml, 0.686 mmol) and 3,4,5-trimethoxyaniline (0.114 g, 0.624 mmol) were added. The mixture was microwaved at 120° C. for 20 min and then concentrated. 67 mg of product was recovered after automated reverse phase chromatography (water-MeCN). MS calcd for [C₂₃H₂₃BrN₄O₅+H]⁺: 515.10 found 515.05.

### Example 5: Synthesis of Compounds of Formula (IIIC)

Compounds of Formula (IIIC) may be synthesized according to the general Scheme 3 described below.

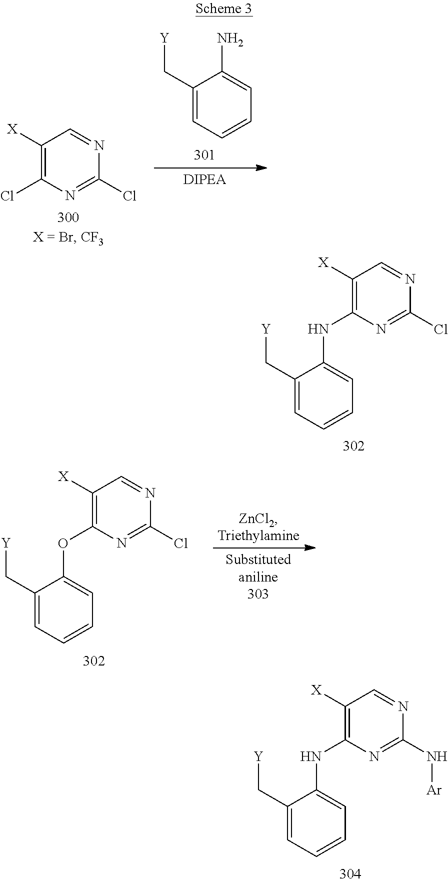

5-X-2,4-dichloropyrimidine 300 (0.439 mmol), substituted aniline 301 (0.439 mmol) and N-ethyl-N-isopropylpropan-2-amine (0.084 ml, 0.483 mmol) are mixed in acetonitrile (2 ml). The mixture is microwaved at 100° C. for 20 min and then concentrated to yield crude intermediate 302, which is used in the next step as is.

Intermediate 302 (0.4 mmol) and zinc(II) chloride (0.065 g, 0.477 mmol) are mixed in 1,2-dichloroethane (2 ml). After 30 min, triethylamine (0.072 ml, 0.517 mmol) and substituted aniline 303 (0.4 mmol) are added. The mixture is microwaved at 140° C. for 20 min and then concentrated. The material is purified using automated reverse phase chromatography (water-10% THF in MeCN) to give product 304.

### Example 6 Preparation of (2-((5-bromo-2-((3,4,5-trimethoxyphenyl)amino)pyrimidin-4-yl)amino)phenyl)methanol

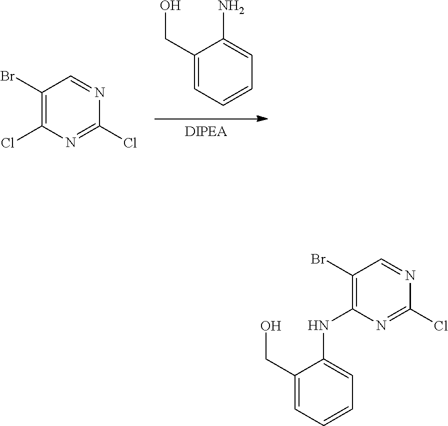 [SMILES: Clc1ncc(Br)c(Cl)n1 ; Nc1ccccc1CO ; OCc1ccccc1Nc1nc(Cl)ncc1Br]

5-Bromo-2,4-dichloropyrimidine (0.100 g, 0.439 mmol), (2-aminophenyl)methanol (0.054 g, 0.439 mmol) and N-ethyl-N-isopropylpropan-2-amine (0.084 ml, 0.483 mmol) were mixed in acetonitrile (2 ml). The mixture was microwaved at 100° C. for 20 min and then concentrated and used as-is. MS calcd for [Cn H₉BrClN₃O+H]⁺: 313.97, found 313.60.

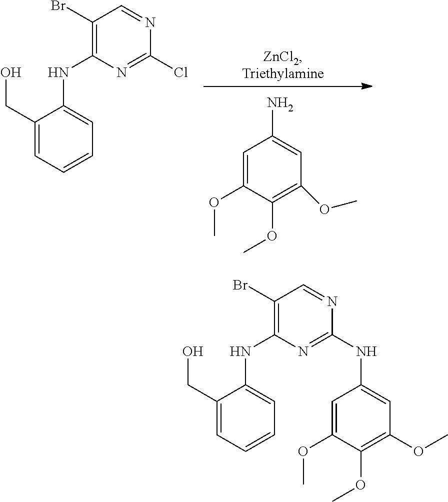

(2-((5-Bromo-2-chloropyrimidin-4-yl)amino)phenyl)methanol (0.125 g, 0.397 mmol) and zinc(II) chloride (0.065 g, 0.477 mmol) were mixed in 1,2-dichloroethane (2 ml). After 30 min, triethylamine (0.072 ml, 0.517 mmol) and 3,4,5-trimethoxyaniline (0.073 g, 0.397 mmol) were added. The mixture was microwaved at 140° C. for 20 min and then concentrated. The material was purified using automated reverse phase chromatography (water-10% THF in MeCN) to give semipure material. It was further purified by flash chromatography on silica gel (DCM-EtOAc) to give 22 mg of product. MS calcd for [C₂₀H₂₁BrN₄O₄+H]⁺: 461.08, found 460.90.

### Example 7 General Synthesis Schemes

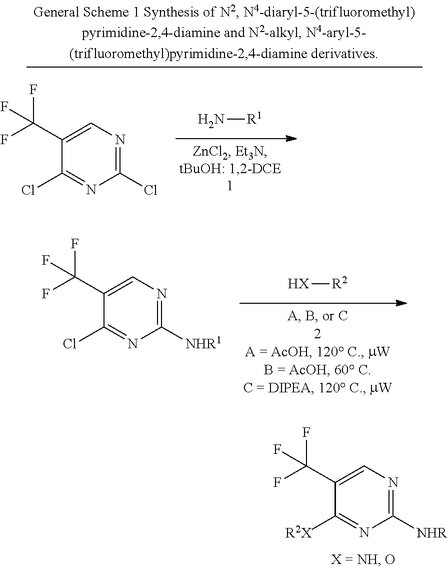

Method 1—General procedure for the synthesis of 4-chloro-5-trifluoromethyl-N-arylpyrimidin-2-amine derivatives (shown in General Scheme 1). To a solution of 2,4-dichloro-5-(trifluoromethyl)pyrimidine (1.0 equiv.) in 1,2-dichloroethane:t-butanol (1:1) was added zinc chloride (1.2 equiv.) at 0° C. After 1 hour, the appropriate aniline (1.0 equiv.) and triethylamine (1.2 equiv.) in 1,2-dichloroethane:t-butanol (1:1, 10 mL) was added to the reaction mixture. After 3 hours, the reaction mixture was concentrated in vacuo to obtain the crude product. The crude product was purified by automated normal phase chromatography to afford the desired 4-chloro-5-trifluoromethyl-N-arylpyrimidin-2-amine derivative.

Method 2—General procedure for the synthesis of N²,N⁴-diaryl-5-(trifluoromethyl) pyrimidine-2,4-diamine and N²-alkyl,N⁴-aryl-5-(trifluoromethyl)pyrimidine-2,4-diamine derivatives (using reaction conditions A, B or C, shown in General Scheme 1). To a solution of 4-chloro-5-trifluoromethyl-N-arylpyrimidin-2-amine derivative (1.0 equiv.) and the appropriate aniline or phenol (1.1 equiv.) in acetic acid (2 mL) was microwaved at 120° C. for 10 minutes and then concentrated in vacuo. The crude product was purified by automated chromatography (Method 2a). To a solution of 4-chloro-5-trifluoromethyl-N-arylpyrimidin-2-amine derivative (1.0 equiv.) and the appropriate aniline or phenol (1.1 equiv.) in acetic acid was heated at 60° C. The reaction mixture was then concentrated in vacuo and the crude product was purified by automated chromatography to afford the desired N²,N⁴-diaryl-5-(trifluoromethyl)pyrimidine-2,4-diamine derivative (Method 2b). To a solution of 4-chloro-5-trifluoromethyl-N-arylpyrimidin-2-amine derivative (1.0 equiv.), the appropriate aniline or phenol (1.2 equiv.), and N,N-diisopropylethylamine (1.2 equiv.) in DMF (2 mL) was microwaved at 120° C. and then concentrated in vacuo. The crude product was purified by automated chromatography (Method 2c).

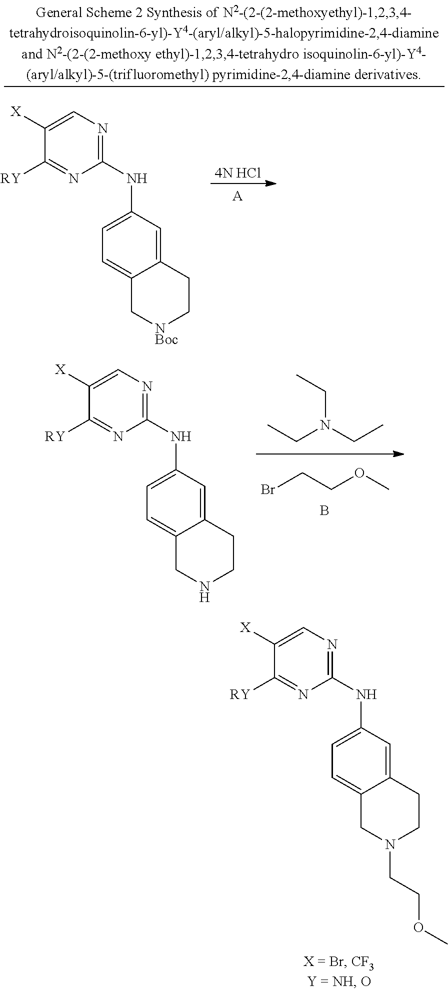

Method 3—General Procedure for the Synthesis of N²-(2-(2-methoxyethyl)-1,2,3,4-tetra hydroisoquinolin-6-yl)-Y⁴-(aryl/alkyl)-5-halopyrimidine-2,4-diamine and N²-(2-(2-methoxy ethyl)-1,2,3,4-tetrahydroisoquinolin-6-yl)-Y⁴-(aryl/alkyl)-5-(trifluoromethyl) pyrimidine-2,4-diamine derivatives (Using Reaction Conditions A and B Shown in General Scheme 2).

The intermediate tert-butyl 6-((N⁴-(aryl/alkyl)-5-(trifluoromethyl)pyrimidin-2-yl)amino)-3,4-dihydroisoquinoline-2(1H)-carboxylate (1.0 equiv.) was treated with hydrochloride solution in organic solvent (methanol or dioxane, 1-4M) for 2 hours and the reaction mixture was concentrated in vacuo (Method 3a). The crude salt was used subsequently in the next step or the HCl salt was neutralized and purified to afford the free amine N²-(1,2,3,4-tetrahydroisoquinolin-6-yl)-Y⁴-(aryl/alkyl)-5-(trifluoromethyl/halo) pyrimidine-2,4-diamine.

To a solution of N²-(1,2,3,4-tetrahydroisoquinolin-6-yl)-Y⁴-(aryl/alkyl)-5-(trifluoromethyl/halo) pyrimidine-2,4-diamine (1.0 equiv.), 1-bromo-2-methoxyethane (1.2 equiv.) and triethylamine (4.0 equiv.) in DMF (5 mL) was heated at 60° C. for 4 hours. The reaction mixture was then concentrated in vacuo and the crude product was purified by automated normal phase chromatography to afford the desired pyrimidine-2,4-diamine derivative (Method 3b).

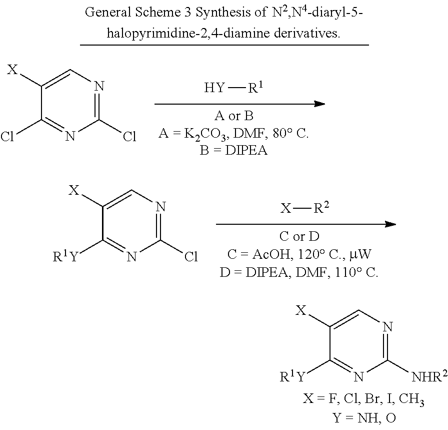

Method 4—General Procedure for the Synthesis of 2-chloro-5-halo-Y⁴-arylpyrimidin-4-amine and 2-chloro-5-methyl-Y⁴-arylpyrimidin-4-amine (Using Reaction Conditions A or B Shown in General Scheme 3).

To a solution of appropriate aniline or phenol (1.0 equiv.), 2,4-dichloro-5-(X) pyrimidine (1.3 equiv.) and potassium carbonate (1.3 equiv.) in DMF was heated at 80° C. for 4 hours. The reaction mixture was cooled to room temperature, concentrated in vacuo and titrated in water. The resulting precipitate was collected by filtration provide the desired 2-chloro-arylpyrimidine derivative. The crude product was used for next step without further purification (Method 4a). To a solution of appropriate aniline or phenol (1.0 equiv.), 2,4-dichloro-5-(X) pyrimidine (1.1 equiv.) and N,N-diisopropylethylamine (1.5 equiv.) in organic solvent (DMF, ethanol, or ⁿbutanol) was heated at 80° C. The reaction mixture was cooled to room temperature and concentrated in vacuo. The crude solid was dissolved in ethyl acetate (20 mL) and washed with water (3×5 mL). The organic fraction was dried over sodium sulfate and concentrated to afford the crude product which was used in the subsequent step without further purification (Method 4b)

Method 5—General Procedure for the Synthesis of 5-halo-N², Y⁴-diarylpyrimidine 2,4-diamine and N²,Y⁴-diaryl-5-methyl-pyrimidine 2,4-diamine derivatives (Using Reaction Conditions C or D Shown in General Scheme 3).

To a solution of 2-chloro-5-(halo/methyl)-Y⁴-arylpyrimidin-4-amine (1.0 equiv) and the appropriate aniline (1.1 equiv.) in acetic acid (2 mL) was microwaved at 120° C. for 10 minutes and then concentrated in vacuo. The crude product was purified by automated chromatography (Method 5a). To a solution of 2-chloro-5-(halo/methyl)-Y⁴-arylpyrimidin-4-amine (1.0 equiv.), the appropriate aniline (1.1 equiv.), and N,N-diisopropylethylamine (1.1 equiv.) in DMF was heated at 110° C. The reaction mixture was then concentrated in vacuo and the crude product was purified by automated chromatography to afford the desired pyrimidine 2,4-diamine derivatives derivative (Method 5b).

### Example 8 Synthesis of Compound E6

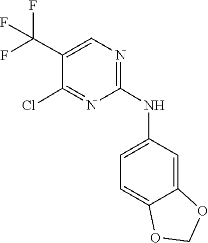 [SMILES: FC(F)(F)c1cnc(Nc2ccc3c(c2)OCO3)nc1Cl]

N-(Benzo[d][1,3]dioxol-5-yl)-4-chloro-5-(trifluoromethyl)pyrimidin-2-amine. The title compound was prepared by reaction of 2,4-dichloro-5-(trifluoromethyl)pyrimidine (1.00 g, 4.61 mmol), benzo[d][1,3]dioxol-5-amine (664 mg, 4.84 mmol), zinc chloride (754 mg, 5.53 mmol), and triethylamine (0.77 mL, 5.6 mmol) in 1,2-dichloroethane:t-butanol (1:1, 40 mL) according to Method 1 to provide the title compound as an off white solid (1.03 g, 70%). LC-MS (ESI) calcd. for C₁₂H₈ClF₃N₃O₂ [M+H]⁺: 318.03; found: 317.90.

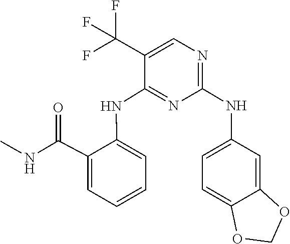 [SMILES: CNC(=O)c1ccccc1Nc1nc(Nc2ccc3c(c2)OCO3)ncc1C(F)(F)F]

2-((2-(Benzo[d][1,3]dioxol-5-ylamino)-5-(trifluoromethyl)pyrimidin-4-yl)amino)-N-methyl benzamide (Compound E6). The title compound was prepared by reaction of N-(benzo[d][1,3]dioxol-5-yl)-4-chloro-5-(trifluoromethyl)pyrimidin-2-amine (250 mg, 0.79 mmol) and 2-amino-N-methylbenzamide (130 mg, 0.87 mmol) according to Method 2a. The crude product was purified by automated reverse phase chromatography to afford the title compound as a white solid (95 mg, 28%). LC-MS (ESI) calcd. for C₂₀H₁₇F₃N₅O₃[M+H]⁺: 432.13; found: 432.95.

### Example 9 Synthesis of Compound B52

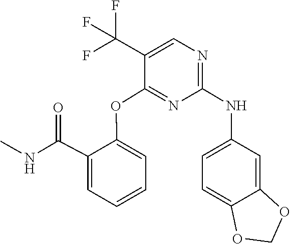 [SMILES: CNC(=O)c1ccccc1Oc1nc(Nc2ccc3c(c2)OCO3)ncc1C(F)(F)F]

2-((2-(Benzo[d][1,3]dioxol-5-ylamino)-5-(trifluoromethyl)pyrimidin-4-yl)oxy)-N-methyl benzamide (B52). The title compound was prepared by reaction of N-(benzo[d][1,3]dioxol-5-yl)-4-chloro-5-(trifluoromethyl)pyrimidin-2-amine (250 mg, 0.79 mmol), 2-hydroxy-N-methylbenzamide (144 mg, 0.94 mmol), and N,N-diisopropylethylamine (0.16 mL, 0.94 mmol) at 120° C. for 80 minutes according to Method 2c. The crude product was purified by automated reverse phase chromatography to afford the title compound as a white solid (71 mg, 21%). LC-MS (ESI) calcd. for C₂₀H₁₆F₃N₄O₄[M+H]⁺: 433.11; found: 433.50.

### Example 10 Synthesis of Compound B53

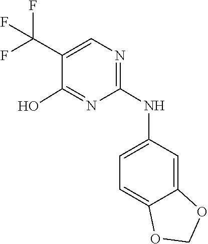 [SMILES: Oc1nc(Nc2ccc3c(c2)OCO3)ncc1C(F)(F)F]

2-(Benzo[d][1,3]dioxol-5-ylamino)-5-(trifluoromethyl)pyrimidin-4-ol (B53). The title compound was prepared by reaction of N-(benzo[d][1,3]dioxol-5-yl)-4-chloro-5-(trifluoromethyl)pyrimidin-2-amine (252 mg, 0.79 mmol) and 2-hydroxy-N-methylbenzamide (132 mg, 0.87 mmol) at 120° C. for 40 minutes according to Method 2a. The crude product was purified by automated reverse phase chromatography to afford the title compound as a white solid (76 mg, 32%). LC-MS (ESI) calcd. for C₁₂H₉F₃N₃O₃ [M+H]⁺: 300.06; found: 300.40.

### Example 11 Synthesis of Compound E7

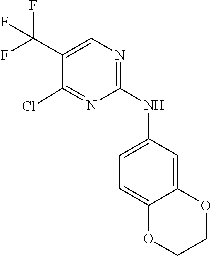 [SMILES: FC(F)(F)c1cnc(Nc2ccc3c(c2)OCCO3)nc1Cl]

4-Chloro-N-(2,3-dihydrobenzo[b][1,4]dioxin-6-yl)-5-(trifluoromethyl)pyrimidin-2-amine. The title compound was prepared by reaction of 2,4-dichloro-5-(trifluoromethyl)pyrimidine (400 mg, 1.84 mmol), 2,3-dihydrobenzo[b][1,4]dioxin-6-amine (293 mg, 1.94 mmol), zinc chloride (301 mg, 2.21 mmol), and triethylamine (0.31 mL, 2.21 mmol) in 1,2-dichloroethane:t-butanol (1:1, 20 mL) according to Method 1 to provide the title compound as an yellow solid (582 mg, 95%). LC-MS (ESI) calcd. for C₁₃H₁₀ClF₃N₃O₂ [M+H]⁺: 332.04; found: 331.95.

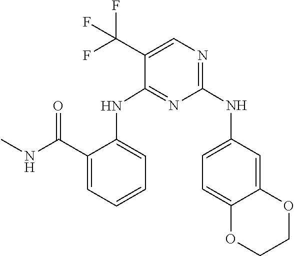 [SMILES: CNC(=O)c1ccccc1Nc1nc(Nc2ccc3c(c2)OCCO3)ncc1C(F)(F)F]

2-((2-((2,3-Dihydrobenzo[b][1,4]dioxin-6-yl)amino)-5-(trifluoromethyl)pyrimidin-4-yl) amino)-N-methylbenzamide (E7). The title compound was prepared by reaction of 4-chloro-N-(2,3-dihydrobenzo[b][1,4]dioxin-6-yl)-5-(trifluoromethyl)pyrimidin-2-amine (250 mg, 0.75 mmol) and 2-amino-N-methylbenzamide (125 mg, 0.83 mmol) according to Method 2a. The crude product was purified by automated reverse phase chromatography to afford the title compound as a brown solid (153 mg, 46%). LC-MS (ESI) calcd. for C₂₁H₁₉F₃N₅O₃[M+H]⁺: 446.14; found: 447.65.

### Example 12 Synthesis of Compound B51

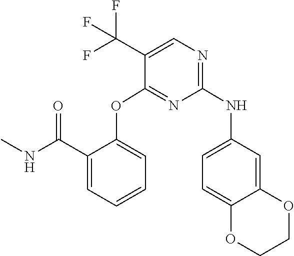 [SMILES: CNC(=O)c1ccccc1Oc1nc(Nc2ccc3c(c2)OCCO3)ncc1C(F)(F)F]

2-((2-((2,3-Dihydrobenzo[b][1,4]dioxin-6-yl)amino)-5-(trifluoromethyl)pyrimidin-4-yl)oxy)-N-methylbenzamide (B51). The title compound was prepared by reaction of 4-chloro-N-(2,3-dihydrobenzo[b][1,4]dioxin-6-yl)-5-(trifluoromethyl)pyrimidin-2-amine (200 mg, 0.60 mmol), 2-hydroxy-N-methylbenzamide (110 mg, 0.73 mmol), and N,N-diisopropylethylamine (0.13 mL, 0.73 mmol) at 120° C. for 30 minutes according to Method 2c. The crude product was purified by automated reverse phase chromatography to afford the title compound as a white solid (79 mg, 29%). LC-MS (ESI) calcd. for C₂₁H₁₈F₃N₄O₄[M+H]⁺: 447.13; found: 447.90.

### Example 13 Synthesis of Compound A120

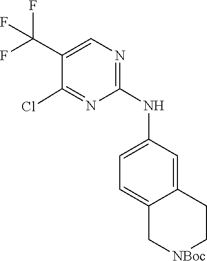 [SMILES: CC(C)(C)OC(=O)N1CCc2cc(Nc3ncc(C(F)(F)F)c(Cl)n3)ccc2C1]

tert-Butyl 6-((4-chloro-5-(trifluoromethyl)pyrimidin-2-yl)amino)-3,4-dihydroisoquinoline-2(1H)-carboxylate. The title compound was prepared by reaction of 2,4-dichloro-5-(trifluoromethyl)pyrimidine (2.00 g, 9.22 mmol), tert-butyl 6-amino-3,4-dihydroisoquinoline-2(1H)-carboxylate (2.29 g, 9.22 mmol), zinc chloride (1.51 g, 11 mmol), and triethylamine (1.5 mL, 11 mmol) in 1,2-dichloroethane:t-butanol (1:1, 90 mL) according to Method 1 to provide the title compound as a white solid (3.86 g, 98%). LC-MS (ESI) calcd. for C₁₉H₂₁ClF₃N₄O₂ [M-C₄H₇]⁺: 373.07; found: 373.40.

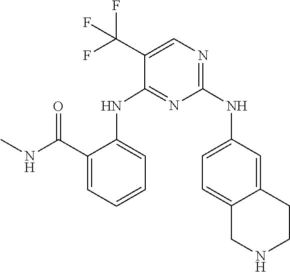 [SMILES: CNC(=O)c1ccccc1Nc1nc(Nc2ccc3c(c2)CCNC3)ncc1C(F)(F)F]

N-Methyl-2-((2-((1,2,3,4-tetrahydroisoquinolin-6-yl)amino)-5-(trifluoromethyl)pyrimidin-4-yl)amino)benzamide (A120). The title compound was prepared by reaction of tert-butyl 6-((4-chloro-5-(trifluoromethyl)pyrimidin-2-yl)amino)-3,4-dihydroisoquinoline-2(1H)-carboxylate (320 mg, 0.75 mmol), and 2-amino-N-methylbenzamide (123 mg, 0.82 mmol) according to Method 2a. The crude product was purified by automated reverse phase chromatography to afford the title compound as a white solid (156 mg, 47%). LC-MS (ESI) calcd. for C₂₂H₂₂F₃N₆O [M+H]⁺: 443.18; found: 443.65.

### Example 14 Synthesis of Compound A119

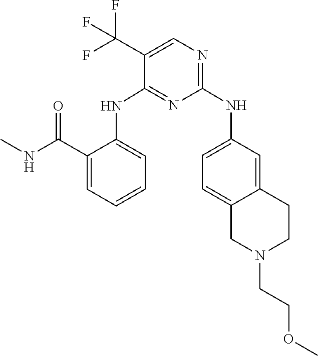

2-((2-((2-(2-Methoxyethyl)-1,2,3,4-tetrahydroisoquinolin-6-yl)amino)-5-(trifluoromethyl) pyrimidin-4-yl)amino)-N-methylbenzamide (A119). The title compound was prepared by reaction of N-methyl-2-((2-((1,2,3,4-tetrahydroisoquinolin-6-yl)amino)-5-(trifluoromethyl) pyrimidin-4-yl)amino)benzamide (154 mg, 0.349 mmol), 1-bromo-2-methoxyethane (40 μL, 0.43 mmol), and triethylamine (0.19 mL, 1.40 mmol) according to Method 3b. The crude product was purified by automated reverse phase chromatography to afford the title compound as a yellow solid (36 mg, 21%). LC-MS (ESI) calcd. for C₂₅H₂₈F₃N₆O₂ [M+H]⁺: 501.22; found: 501.30.

### Example 15 Synthesis of Compound A121

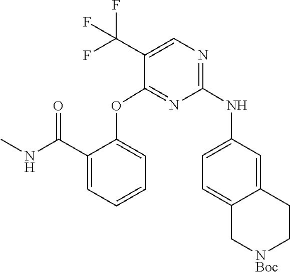 [SMILES: CNC(=O)c1ccccc1Oc1nc(Nc2ccc3c(c2)CCN(C(=O)OC(C)(C)C)C3)ncc1C(F)(F)F]

tert-Butyl 6-((4-(2-(methylcarbamoyl)phenoxy)-5-(trifluoromethyl)pyrimidin-2-yl)amino)-3,4-dihydroisoquinoline-2(1H)-carboxylate. The title compound was prepared by reaction of tert-butyl 6-((4-chloro-5-(trifluoromethyl)pyrimidin-2-yl)amino)-3,4-dihydroisoquinoline-2(1H)-carboxylate (320 mg, 0.746 mmol), 2-hydroxy-N-methylbenzamide (124 mg, 0.821 mmol), and N,N-diisopropylethylamine (0.16 mL, 0.895 mmol) at 120° C. for 60 minutes according to Method 2c to afford the title compound as a colored solid (311 mg, 77%). LC-MS (ESI) calcd. for C₂₇H₂₉F₃N₅O₄ [M+H]⁺: 544.22; found: 544.70.

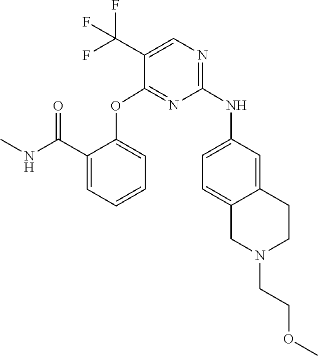

2-((2-((2-(2-Methoxyethyl)-1,2,3,4-tetrahydroisoquinolin-6-yl)amino)-5-(trifluoromethyl) pyrimidin-4-yl)oxy)-N-methylbenzamide. The title compound was prepared by reaction of tert-butyl 6-((4-(2-(methylcarbamoyl)phenoxy)-5-(trifluoromethyl)pyrimidin-2-yl) amino)-3,4-dihydroisoquinoline-2(1H)-carboxylate (310 mg, 0.57 mmol), 1-bromo-2-methoxyethane (64 μL, 0.68 mmol), and triethylamine (0.32 mL, 2.28 mmol) was heated at 80° C. for 18 hours similar to Method 3b. The crude product was purified by automated reverse phase chromatography to afford the title compound as a yellow solid (132 mg, 46%). LC-MS (ESI) calcd. for C₂₅H₂₇F₃N₅O₃ [M+H]⁺: 502.21; found: 502.60.

### Example 16 Synthesis of Compound A122

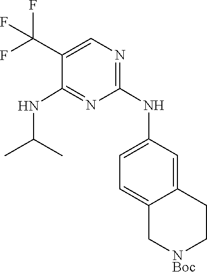 [SMILES: CC(C)Nc1nc(Nc2ccc3c(c2)CCN(C(=O)OC(C)(C)C)C3)ncc1C(F)(F)F]

tert-Butyl 6-((4-(isopropylamino)-5-(trifluoromethyl)pyrimidin-2-yl)amino)-3,4-dihydroisoquinoline-2(1H)-carboxylate. The title compound was prepared by reaction of tert-butyl 6-((4-chloro-5-(trifluoromethyl)pyrimidin-2-yl)amino)-3,4-dihydroisoquinoline-2(1H)-carboxylate (300 mg, 0.70 mmol), propan-2-amine (72 μL, 0.84 mmol), and N,N-diisopropylethylamine (0.15 mL, 0.84 mmol) at 120° C. for 10 minutes according to Method 2c. The crude product was purified by automated reverse phase chromatography to afford the title compound as a brown solid (309 mg, 98%). LC-MS (ESI) calcd. for C₂₂H₂₉F₃N₅O₂ [M+H]⁺: 452.23; found: 453.05.

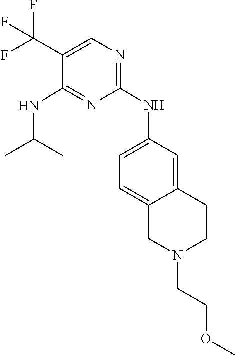 [SMILES: COCCN1CCc2cc(Nc3ncc(C(F)(F)F)c(NC(C)C)n3)ccc2C1]

N⁴-Isopropyl-N²-(2-(2-methoxyethyl)-1,2,3,4-tetrahydroisoquinolin-6-yl)-5-(trifluoromethyl)pyrimidine-2,4-diamine (A122). Tert-butyl 6-((4-(isopropylamino)-5-(trifluoromethyl)pyrimidin-2-yl)amino)-3,4-dihydroisoquinoline-2(1H)-carboxylate (309 mg, 0.68 mmol) was treated with hydrochloric acid in methanol (1M, 3 mL, 3.4 mmol) according to Method 3a to afford the corresponding HCl salt used crude in the next step. The title compound was prepared by reaction of the crude salt, 1-bromo-2-methoxyethane (77 μL, 0.82 mmol), and triethylamine (0.38 mL, 2.74 mmol) heated at 60° C. for 8 hours similar to Method 3b. The crude product was purified by automated reverse phase chromatography to afford the title compound as a yellow solid (22 mg, 8%). LC-MS (ESI) calcd. for C₂₀H₂₇F₃N₅O [M+H]⁺: 410.22; found: 410.25.

### Example 17 Synthesis of Compound A123

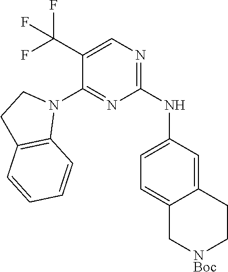 [SMILES: CC(C)(C)OC(=O)N1CCc2cc(Nc3ncc(C(F)(F)F)c(N4CCc5ccccc54)n3)ccc2C1]

tert-Butyl 6-((4-(indolin-1-yl)-5-(trifluoromethyl)pyrimidin-2-yl)amino)-3,4-dihydroiso-quinoline-2(1H)-carboxylate. The title compound was prepared by reaction of tert-butyl 6-((4-chloro-5-(trifluoromethyl)pyrimidin-2-yl)amino)-3,4-dihydroisoquinoline-2(1H)-carboxylate (320 mg, 0.75 mmol), indoline (0.10 mL, 0.89 mmol), and N,N-diisopropylethylamine (0.16 mL, 0.90 mmol) at 120° C. for 20 minutes according to Method 2c. The crude product was purified by automated reverse phase chromatography to afford the title compound as a colored solid (371 mg, 97%). LC-MS (ESI) calcd. for C₂₇H₂₉F₃N₅O₂ [M+H]⁺: 512.23; found: 512.75.

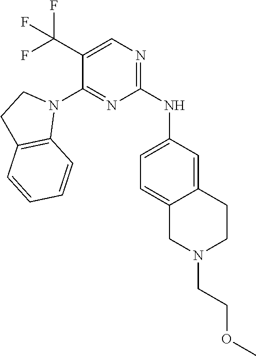 [SMILES: COCCN1CCc2cc(Nc3ncc(C(F)(F)F)c(N4CCc5ccccc54)n3)ccc2C1]

N-(4-(Indolin-1-yl)-5-(trifluoromethyl)pyrimidin-2-yl)-2-(2-methoxyethyl)-1,2,3,4-tetra-hydroisoquinolin-6-amine (A123). Tert-butyl 6-((4-(indolin-1-yl)-5-(trifluoromethyl) pyrimidin-2-yl)amino)-3,4-dihydroisoquinoline-2(1H)-carboxylate (371 mg, 0.73 mmol) was treated with hydrochloric acid in dioxane (4 M, 2 mL, 7.3 mmol) according to Method 3a to afford the corresponding HCl salt used crude in the next step. The title compound was prepared by reaction of the crude salt, 1-bromo-2-methoxyethane (82 μL, 0.87 mmol), and triethylamine (0.40 mL, 2.9 mmol) heated at 80° C. for 18 hours similar to Method 3b. The crude product was purified by automated reverse phase chromatography to afford the title compound as an orange solid (224 mg, 66%). LC-MS (ESI) calcd. for C₂₅H₂₇F₃N₅O [M+H]⁺: 470.22; found: 470.65.

### Example 18 Synthesis of Compound E4

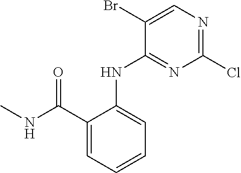 [SMILES: CNC(=O)c1ccccc1Nc1nc(Cl)ncc1Br]

2-((5-Bromo-2-chloropyrimidin-4-yl)amino)-N-methylbenzamide. The title compound was prepared by reaction of 5-bromo-2,4-dichloropyrimidine (5.88 g, 25.8 mmol), 2-amino-N-methylbenzamide (3.10 g, 20.6 mmol), and potassium carbonate (3.71 g, 26.8 mmol) in DMF (60 mL) heated at 80° C. for 4 hours according to Method 4a to afford the title compound as a yellow solid (6.66 g, 95%). LC-MS (ESI) calcd. for C₁₂H₁₁BrClN₄O [M+H]⁺: 340.98; found: 340.95.

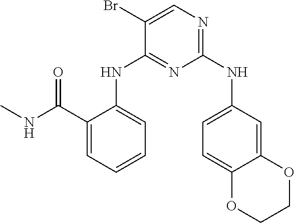 [SMILES: CNC(=O)c1ccccc1Nc1nc(Nc2ccc3c(c2)OCCO3)ncc1Br]

2-((5-Bromo-2-((2,3-dihydrobenzo[b][1,4]dioxin-6-yl)amino)pyrimidin-4-yl)amino)-N-methylbenzamide (E4). The title compound was prepared by reaction of 2-((5-bromo-2-chloropyrimidin-4-yl)amino)-N-methylbenzamide (252 mg, 0.74 mmol), 2,3-dihydrobenzo[b][1,4]dioxin-6-amine (123 mg, 0.81 mmol), and DIPEA (0.14 mL, 0.81 mmol) in DMF (5 mL) heated at 110° C. for 18 hours according to Method 5b. The crude product was purified by automated reverse phase chromatography to afford the title compound as a brown solid (29 mg, 9%). LC-MS (ESI) calcd. for C₂₀H₁₉BrN₅O₃ [M+H]⁺: 456.07; found: 458.05.

### Example 19 Synthesis of Compound E5

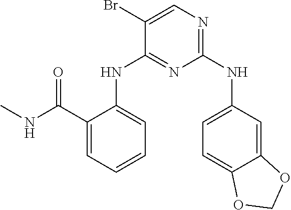 [SMILES: CNC(=O)c1ccccc1Nc1nc(Nc2ccc3c(c2)OCO3)ncc1Br]

2-((2-(Benzo[d][1,3]dioxol-5-ylamino)-5-bromopyrimidin-4-yl)amino)-N-methylbenzamide (E5). The title compound was prepared by reaction of 2-((5-bromo-2-chloro pyrimidin-4-yl)amino)-N-methylbenzamide (252 mg, 0.74 mmol) and benzo[d][1,3]dioxol-5-amine (111 mg, 0.81 mmol) according to Method 5a. The crude product was purified by automated reverse phase chromatography to afford the title compound as a brown solid (32 mg, 10%). LC-MS (ESI) calcd. for C₁₉H₁₇BrN₅O₃ [M+H]⁺: 442.05; found: 443.45.

### Example 20 Synthesis of Compound E9

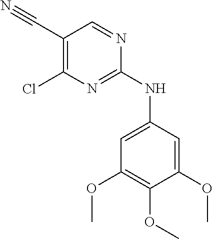 [SMILES: COc1cc(Nc2ncc(C#N)c(Cl)n2)cc(OC)c1OC]

4-Chloro-2-((3,4,5-trimethoxyphenyl)amino)pyrimidine-5-carbonitrile. The title compound was prepared by reaction of 2,4-dichloropyrimidine-5-carbonitrile (400 mg, 2.30 mmol), 3,4,5-trimethoxyaniline (421 mg, 2.30 mmol), zinc chloride (376 mg, 2.76 mmol), and triethylamine (0.38 mL, 2.76 mmol) in 1,2-dichloroethane:t-butanol (1:1, 20 mL) for 4 hours according to Method 1 to provide the title compound as a yellow solid (493 mg, 67%). LC-MS (ESI) calcd. for C₁₄H₁₄ClN₄O₃[M+H]⁺: 321.08; found: 321.35.

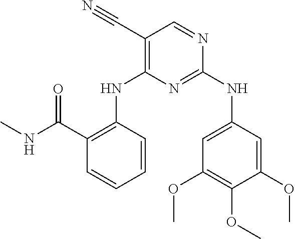

2-((5-cyano-2-((3,4,5-trimethoxyphenyl)amino)pyrimidin-4-yl)amino)-N-methylbenzamide Compound E9. To a solution of 4-chloro-2-((3,4,5-trimethoxyphenyl)amino)pyrimidine-5-carbonitrile (492 mg, 1.53 mmol), 2-amino-N-methylbenzamide (277 mg, 1.84 mmol), and DIPEA (0.32 mL, 1.84 mmol) in DMF (10 mL) was heated at 120° C. for 6 hours. The reaction mixture was then concentrated in vacuo and the crude product was purified by automated normal phase chromatography to afford the title compound as a white solid (89 mg, 13%). LC-MS (ESI) calcd. for C₂₂H₂₃N₆O₄ [M+H]⁺: 435.18; found: 435.60.

### Example 21 Synthesis of Compound E8

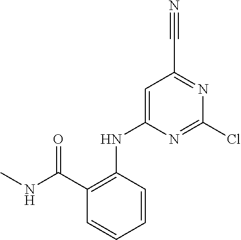 [SMILES: CNC(=O)c1ccccc1Nc1cc(C#N)nc(Cl)n1]

2-((2-chloro-6-cyanopyrimidin-4-yl)amino)-N-methylbenzamide. The title compound was prepared by reaction of 2,6-dichloropyrimidine-4-carbonitrile (280 mg, 1.61 mmol), 2-amino-N-methylbenzamide (220 mg, 1.46 mmol), and potassium carbonate (263 mg, 1.90 mmol) in DMF (5 mL) heated at 80° C. for 2 hours according to Method 4a, filtered, and concentrated in vacuo. The crude product was purified by automated normal phase chromatography to afford the title compound as a colored solid (147 mg, 35%). LC-MS (ESI) calcd. for C₁₃H₁₁ClN₅O [M+H]⁺: 288.07; found: 288.00.

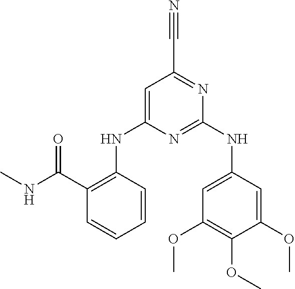

2-((6-Cyano-2-((3,4,5-trimethoxyphenyl)amino)pyrimidin-4-yl)amino)-N-methylbenzamide (Compound E8). The title compound was prepared by reaction of 2-((2-chloro-6-cyanopyrimidin-4-yl)amino)-N-methylbenzamide (147 mg, 510 mmol), 3,4,5-trimethoxyaniline (140 mg, 765 mmol), and DIPEA (0.13 mL, 765 mmol) in DMF (5 mL) at 120° C. for 5 hours according to Method 5b. The crude product was purified by automated reverse phase chromatography to afford the title compound as an orange solid (27 mg, 12%). LC-MS (ESI) calcd. for C₂₂H₂₃N₆O₄ [M+H]⁺: 435.18; found: 435.55.

### Example 22 Synthesis of Compound E10

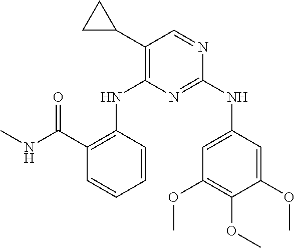

2-((5-Cyclopropyl-2-((3,4,5-trimethoxyphenyl)amino)pyrimidin-4-yl)amino)-N-methyl-benzamide (Compound E10). To a solution of 2-((5-bromo-2-((3,4,5-trimethoxyphenyl)amino) pyrimidin-4-yl)amino)-N-methylbenzamide (250 mg, 0.51 mmol), cyclopropylboronic acid (66 mg, 0.77 mmol), potassium phosphate (312 mg, 1.79 mmol), and tricyclohexylphosphine (14 mg, 0.05 mmol) in DME (5 mL) and water (0.5 mL) was added palladium(II) acetate (12 mg, 0.05 mmol). The reaction mixture was heated at 95° C. for 48 hours. The mixture was cooled to room temperature, diluted in methanol, filtered through celite, and concentrated in vacuo. The crude product was purified by automated normal phase chromatography to afford the title compound as a grey solid (55 mg, 24%). LC-MS (ESI) calcd. for C₂₄H₂₈N₅O₄ [M+H]⁺: 450.21; found: 450.25.

### Example 23 Synthesis of Compound E11

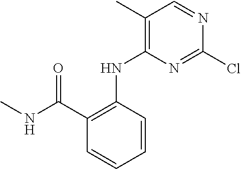 [SMILES: CNC(=O)c1ccccc1Nc1nc(Cl)ncc1C]

2-((2-Chloro-5-methylpyrimidin-4-yl)amino)-N-methylbenzamide. The title compound was prepared by reaction of 2,4-dichloro-5-methylpyrimidine (501 mg, 3.08 mmol), 2-amino-N-methylbenzamide (420 mg, 2.80 mmol), and DIPEA (0.73 mL, 4.19 mmol) in DMF (20 mL) heated at 80° C. for 5 hours according to Method 4b. The crude product was recrystallized in acetone to afford the title compound as a white solid (411 mg, 53%). LC-MS (ESI) calcd. for C₁₃H₁₄ClN₄O [M+H]⁺: 277.09; found: 277.05.

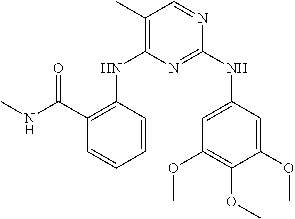

N-Methyl-2-((5-methyl-2-((3,4,5-trimethoxyphenyl)amino)pyrimidin-4-yl)amino)benzamide (Compound E11). To a solution of 2-((2-chloro-5-methylpyrimidin-4-yl)amino)-N-methyl-benzamide (190 mg, 0.69 mmol) and 3,4,5-trimethoxyaniline (252 mg, 1.37 mmol) in n-butanol (5 mL) and acetic acid (1 mL) was heated at 120° C. for 3 hours. The reaction mixture was then concentrated in vacuo and the crude product was purified by automated normal phase chromatography to afford the title compound a tan solid (88 mg, 30%). LC-MS (ESI) calcd. for C₂₂H₂₆N₅O₄ [M+H]⁺: 424.20; found: 424.20.

### Example 24 Synthesis of Compound E12

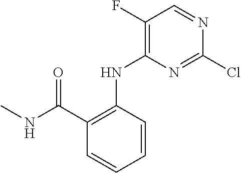 [SMILES: CNC(=O)c1ccccc1Nc1nc(Cl)ncc1F]

2-((2-Chloro-5-fluoropyrimidin-4-yl)amino)-N-methylbenzamide. The title compound was prepared by reaction of 2,4-dichloro-5-fluoropyrimidine (550 mg, 3.30 mmol), 2-amino-N-methylbenzamide (450 mg, 3.00 mmol), and DIPEA (1.0 mL, 5.99 mmol) in ethanol (10 mL) according to Method 4b at 60° C. for 6 hours to afford the title compound as a pale yellow solid (359 mg, 43%). LC-MS (ESI) calcd. for C₁₂H₁₁ClFN₄O [M+H]⁺: 281.06; found: 281.35.

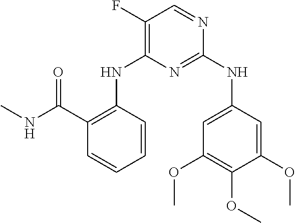

2-((5-Fluoro-2-((3,4,5-trimethoxyphenyl)amino)pyrimidin-4-yl)amino)-N-methylbenzamide (Compound E12). The title compound was prepared by reaction of 2-((2-chloro-5-fluoropyrimidin-4-yl)amino)-N-methylbenzamide (250 mg, 0.89 mmol) and 3,4,5-trimethoxyaniline (196 mg, 1.07 mmol) in ethanol (2 mL) microwaved at 150° C. for 60 minutes. The reaction mixture was concentrated in vacuo and the crude product was purified by automated normal phase chromatography to afford the title compound a grey solid (80 mg, 21%). LC-MS (ESI) calcd. for C₂₁H₂₃FN₅O₄[M+H]⁺: 428.17; found: 428.55.

### Example 25 Synthesis of Compound E13

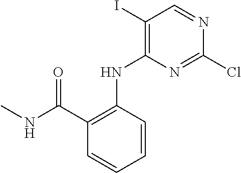 [SMILES: CNC(=O)c1ccccc1Nc1nc(Cl)ncc1I]

2-((2-Chloro-5-iodopyrimidin-4-yl)amino)-N-methylbenzamide. The title compound was prepared by reaction of 2,4-dichloro-5-iodopyrimidine (554 mg, 2.01 mmol), 2-amino-N-methylbenzamide (275 mg, 1.83 mmol), and DIPEA (0.35 mL, 2.01 mmol) in ⁿbutanol (15 mL) according to Method 4b at 120° C. for 7 hours to afford the title compound as yellow solid (566 mg, 80%). LC-MS (ESI) calcd. for C₁₂H₁₁ClIN₄O [M+H]⁺: 388.97; found: 389.30.

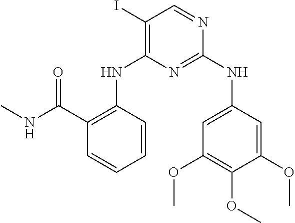

2-((5-iodo-2-((3,4,5-trimethoxyphenyl)amino)pyrimidin-4-yl)amino)-N-methylbenzamide (Compound E13). To a solution of 2-((2-chloro-5-iodopyrimidin-4-yl)amino)-N-methylbenzamide (200 mg, 0.52 mmol), 3,4,5-trimethoxyaniline (189 mg, 1.03 mmol), and trifluoracetic acid (0.20 mL, 2.57 mmol) in n-butanol (5 mL) was heated at 80° C. for 4 hours. The reaction mixture was then concentrated in vacuo and the crude product was purified by automated normal phase chromatography to afford the title compound a tan solid (178 mg, 65%). LC-MS (ESI) calcd. for C₂₁H₂₃IN₅O₄[M+H]⁺: 536.08; found: 536.15.

### Example 26 Synthesis of Key Intermediates

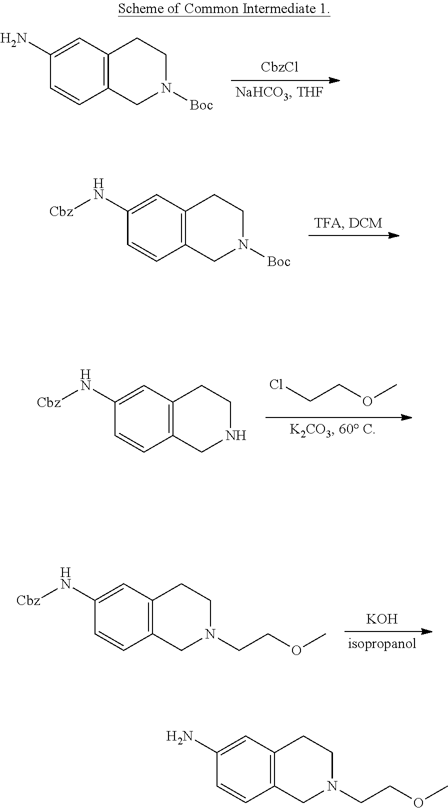

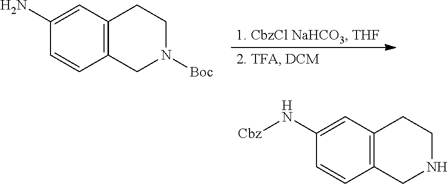 [SMILES: CC(C)(C)OC(=O)N1CCc2cc(N)ccc2C1 ; "1. CbzCl NaHCO3, THF" ; "2. TFA, DCM" ; O=C(Nc1ccc2c(c1)CCNC2)OCc1ccccc1]

Benzyl (1,2,3,4-tetrahydroisoquinolin-6-yl)carbamate. To a solution of tert-butyl 6-amino-3,4-dihydroisoquinoline-2(1H)-carboxylate (2.0 g, 8.06 mmol) in THF (50 mL) was added Cbz-Cl (4.5 mL, 32.2 mmol) and saturated sodium bicarbonate solution (16 mL, 16.1 mmol). The mixture was stirred at room temperature for 15 hrs. The reaction mixture was quenched with water (50 mL), and then extracted with EA (50 mL×3). The organic fractions were combined, then washed with water (50 mL) and brine (50 mL), dried over Na₂SO₄ and filtered. The filtrate was concentrated and purified by flash column chromatography (silica gel, eluting with 5% EA/PE) to afford tert-butyl 6-(((benzyloxy)carbonyl)amino)-3,4-dihydroisoquinoline-2(1H)-carboxylate (2.8 g, 91%) as a brown oil. HPLC/UV purity: 90%; LC-MS (ESI): 383.2 [M+H]⁺.

To a solution of tert-butyl 6-(((benzyloxy)carbonyl)amino)-3,4-dihydroisoquinoline-2(1H)-carboxylate (2.8 g, 7.3 mmol) in DCM (40 mL) was added trifluoroacetic acid (5 mL). The mixture was stirred at room temperature for 2 hrs. The reaction mixture was concentrated and diluted with water (10 mL). The pH of mixture was adjusted to pH=8 with 1 N sodium hydroxide aqueous solution and then extracted with DCM (30 mL×3). The combined organic layers were washed with water (100 mL) and brine (100 mL), dried over Na₂SO₄, and filtered. The filtrate was concentrated to afford benzyl (1,2,3,4-tetrahydroisoquinolin-6-yl)carbamate (1.7 g, 82%) as a white solid. The crude was used to the next step directly without further purification.

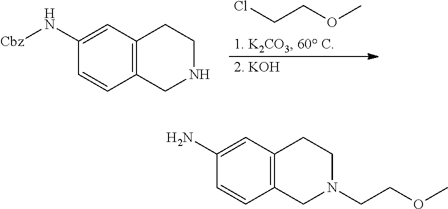

2-(2-Methoxyethyl)-1,2,3,4-tetrahydroisoquinolin-6-amine. To a solution of benzyl (1,2,3,4-tetrahydroisoquinolin-6-yl)carbamate (1.7 g, 6.0 mmol) in DMF (35 mL) were added 1-chloro-2-methoxyethane (2.2 mL, 24.1 mmol) and potassium carbonate (2.5 g, 18.1 mmol). The mixture was stirred at 60° C. for 18 hrs. The reaction mixture was quenched with water (100 mL), and then extracted with DCM (50 mL×3). The combined organic layers were washed with water (50 mL×3) and brine (150 mL), dried over Na₂SO₄ and filtered. The filtrate was concentrated and purified by flash column chromatography (silica gel, eluting with 50% EA/DCM) to afford benzyl (2-(2-methoxyethyl)-1,2,3,4-tetrahydroisoquinolin-6-yl)carbamate (1.6 g, 78%) as a brown oil. HPLC/UV purity: 90%; LC-MS (ESI): 341.2 [M+H]⁺.

To a solution of benzyl (2-(2-methoxyethyl)-1,2,3,4-tetrahydroisoquinolin-6-yl)carbamate (1.6 g, 4.7 mmol) in isopropanol (10 mL) was added potassium hydroxide aqueous solution (2 N, 9.4 mmol). The mixture was stirred at 83° C. for 16 hrs. The reaction mixture was concentrated and purified by flash column chromatography (silica gel, eluting with 5% methanol/DCM) to afford 2-(2-methoxyethyl)-1,2,3,4-tetrahydroisoquinolin-6-amine (600 mg, 62%) as an oil. HPLC/UV purity: 92%; LC-MS (ESI): 207.2 [M+H]⁺.

### Example 27 Synthesis of A117

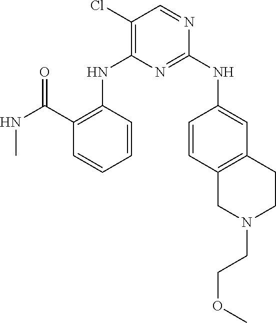 [SMILES: CNC(=O)c1ccccc1Nc1nc(Nc2ccc3c(c2)CCN(CCOC)C3)ncc1Cl]

2-((5-Chloro-2-((2-(2-methoxyethyl)-1,2,3,4-tetrahydroisoquinolin-6-yl)amino)pyrimidin-4-yl)amino)-N-methylbenzamide (A117). To a solution of 2,4,5-trichloropyrimidine (500 mg, 2.7 mmol) in DMF (5 mL) were added 2-amino-N-methylbenzamide (315 mg, 2.1 mmol) and potassium carbonate (442 mg, 3.2 mmol). The mixture was stirred at 75° C. for 16 hrs. The reaction mixture was quenched with water (30 mL), and then extracted with EA (10 mL×3). The combined organic layers were washed with water (30 mL×3) and brine (30 mL), dried over Na₂SO₄, filtered and concentrated. The residue was purified by flash column chromatography (silica gel, eluting with 20% EA/PE) to afford 2-((2,5-dichloropyrimidin-4-yl)amino)-N-methylbenzamide (400 mg, 64%) as a yellow solid. HPLC/UV purity: 92%; LC-MS (ESI): 297.0 [M+H]⁺.

To a solution of 2-((2,5-dichloropyrimidin-4-yl)amino)-N-methylbenzamide (104 mg, 0.35 mmol) in ethanol (5 mL) were added 2-(2-methoxyethyl)-1,2,3,4-tetrahydroisoquinolin-6-amine (80 mg, 0.39 mmol) and few drops of hydrochloric acid. The mixture was stirred at 60° C. for 16 hrs. The reaction mixture was concentrated and diluted with water (10 mL). The mixture was basified to pH=8 with 1 N sodium hydroxide aqueous solution and then extracted with EA (10 mL×3). The combined organic layers were washed with water (30 mL) and brine (30 mL), dried over Na₂SO₄, filtered and concentrated. The residue was purified by prep-TLC to afford 2-((5-chloro-2-((2-(2-methoxyethyl)-1,2,3,4-tetrahydroisoquinolin-6-yl)amino)pyrimidin-4-yl)amino)-N-methylbenzamide (37 mg, 20%) as a yellow solid. HPLC/UV purity: 100%; LC-MS (ESI): 467.2 [M+H]⁺. ¹H NMR (400 MHz, DMSO-d₆) δ 11.58 (s, 1H), 9.34 (s, 1H), 8.75 (s, 2H), 8.21 (s, 1H), 7.75 (d, J=7.2 Hz, 1H), 7.44-7.47 (m, 2H), 7.34 (d, J=6.8 Hz, 1H), 7.14-7.16 (m, 1H), 6.93 (d, J=7.6 Hz, 1H), 3.52-3.54 (m, 4H), 3.27 (s, 3H), 2.81 (s, 3H), 2.71-2.64 (m, 6H).

### Example 28 Synthesis of Compound A118

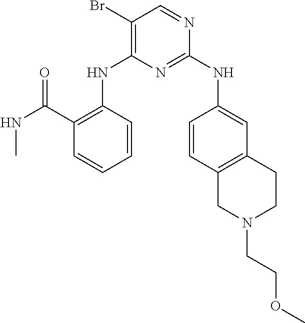 [SMILES: CNC(=O)c1ccccc1Nc1nc(Nc2ccc3c(c2)CCN(CCOC)C3)ncc1Br]

2-((5-Bromo-2-((2-(2-methoxyethyl)-1,2,3,4-tetrahydroisoquinolin-6-yl)amino)pyrimidin-4-yl)amino)-N-methylbenzamide (A118). To a solution of 5-bromo-2,4-dichloropyrimidine (200 mg, 0.88 mmol) in DMF (5 mL) were added 2-amino-N-methylbenzamide (102 mg, 0.68 mmol) and potassium carbonate (182 mg, 1.32 mmol). The mixture was stirred at 75° C. for 16 hrs. The reaction mixture was quenched with water (30 mL), and then extracted with EA (10 mL×3). The combined organic layers were washed with water (30 mL×3) and brine (30 mL), dried over Na₂SO₄, filtered and concentrated. The residue was purified by flash column chromatography (silica gel, eluting with 25% EA/PE) to afford 2-((5-bromo-2-chloropyrimidin-4-yl)amino)-N-methylbenzamide (200 mg, 86%) as a yellow solid. HPLC/UV purity: 95%; LC-MS (ESI): 341.1 [M+H]⁺.

To a solution of 2-((5-bromo-2-chloropyrimidin-4-yl)amino)-N-methylbenzamide (106 mg, 0.31 mmol) in ethanol (10 mL) were added 2-(2-methoxyethyl)-1,2,3,4-tetrahydroisoquinolin-6-amine (50 mg, 0.24 mmol) and few drops of hydrochloric acid. The mixture was stirred at 60° C. for 16 hrs. The reaction mixture was concentrated and diluted with water (10 mL). The mixture was basified to pH=8 with 1 N sodium hydroxide aqueous solution and then extracted with DCM (10 mL×3). The combined organic layers were washed with water (30 mL) and brine (30 mL), dried over Na₂SO₄, filtered and concentrated. The residue was purified by prep-TLC to afford 2-((5-bromo-2-((2-(2-methoxyethyl)-1,2,3,4-tetrahydro isoquinolin-6-yl)amino)pyrimidin-4-yl)amino)-N-methylbenzamide (37 mg, 30%) as a yellow solid. HPLC/UV purity: 98.8%, LC-MS (ESI): 511.1 [M+H]⁺. ¹H NMR (400 MHz, DMSO-d₆) δ 11.34 (s, 1H), 9.34 (s, 1H), 8.74 (d, J=4.4 Hz, 1H), 8.63 (d, J=7.2 Hz, 1H), 8.28 (s, 1H), 7.72 (d, J=7.6 Hz, 1H), 7.43-7.48 (m, 2H), 7.35 (d, J=8.0 Hz, 1H), 7.14 (t, J=7.6 Hz, 1H), 6.92 (d, J=8.4 Hz, 1H), 3.53 (t, J=5.6 Hz, 4H), 3.27 (s, 3H), 2.81 (s, 3H), 2.65-2.71 (m, 6H).

### Example 29 Synthesis of Compound A124

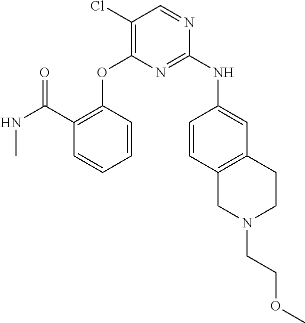 [SMILES: CNC(=O)c1ccccc1Oc1nc(Nc2ccc3c(c2)CCN(CCOC)C3)ncc1Cl]

2-((5-chloro-2-((2-(2-methoxyethyl)-1,2,3,4-tetrahydroisoquinolin-6-yl)amino)pyrimidin-4-yl)oxy)-N-methylbenzamide (A124). To a solution of 2,4,5-trichloropyrimidine (242 mg, 1.32 mmol) in n-BuOH (10 mL) were added 2-hydroxy-N-methylbenzamide (200 mg, 1.32 mmol) and N,N-diisopropylethylamine (187 mg, 1.45 mmol) at 0° C. under N₂. The mixture was stirred at room temperature for 16 hrs. The reaction mixture was concentrated and purified by flash column chromatography (silica gel, eluting with 25% EA/PE) to afford 2-((2,5-dichloropyrimidin-4-yl)oxy)-N-methylbenzamide (140 mg, 36%) as a white solid. HPLC/UV purity: 90%, LC-MS (ESI): 298.1 [M+H]⁺.

In a sealed tube: the mixture of 2-((2,5-dichloropyrimidin-4-yl)oxy)-N-methylbenzamide (140 mg, 0.47 mmol) and 2-(2-methoxyethyl)-1,2,3,4-tetrahydroisoquinolin-6-amine (194 mg, 0.94 mmol) in n-BuOH (2 mL) was stirred at 110° C. for 18 hrs. The reaction mixture was cooled to room temperature and concentrated under reduced pressure to give a crude product which was purified by prep-HPLC to afford 2-((5-chloro-2-((2-(2-methoxyethyl)-1,2,3,4-tetrahydroisoquinolin-6-yl)amino)pyrimidin-4-yl)oxy)-N-methylbenzamide (20 mg, 9%) as a yellow oil. HPLC/UV purity: 97%; LC-MS (ESI): 468.2 [M+H]⁺. ¹H NMR (400 MHz, CDCl₃) δ 8.29 (s, 1H), 8.08 (dd, J=7.6 Hz, 1.6 Hz, 1.6 Hz, 1H), 7.55 (t, J=7.6 Hz, 1H), 7.42 (t, J=7.6 Hz, 1H), 7.24-7.23 (m, 1H), 7.02-7.03 (m, 2H), 6.91 (d, J=6.4 Hz, 1H), 6.86 (d, J=4.8 Hz, 1H), 6.79 (d, J=8.4 Hz, 1H), 3.70-3.77 (m, 4H), 3.39 (s, 3H), 2.91-2.88 (m, 7H), 2.74-2.76 (m, 2H).

### Example 30 Synthesis of Compound A125

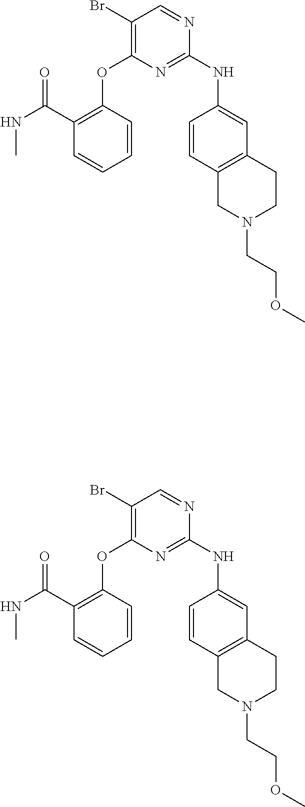 [SMILES: CNC(=O)c1ccccc1Oc1nc(Nc2ccc3c(c2)CCN(CCOC)C3)ncc1Br ; CNC(=O)c1ccccc1Oc1nc(Nc2ccc3c(c2)CCN(CCOC)C3)ncc1Br]

2-((5-Bromo-2-((2-(2-methoxyethyl)-1,2,3,4-tetrahydroisoquinolin-6-yl)amino)pyrimidin-4-yl)oxy)-N-methylbenzamide (A125). To a solution of 5-bromo-2,4-dichloropyrimidine (480 mg, 2.1 mmol) in n-BuOH (10 mL) were added 2-hydroxy-N-methylbenzamide (400 mg, 2.6 mmol) and N,N-diisopropylethylamine (323 mg, 2.5 mmol) at 0° C. under N₂. The mixture was stirred at room temperature for 16 hrs. The reaction mixture was concentrated and purified by flash column chromatography (silica gel, eluting with 25% EA/PE) to afford 2-((5-bromo-2-chloropyrimidin-4-yl)oxy)-N-methylbenzamide (280 mg, 39%) as a white solid. HPLC/UV purity: 90%, LC-MS (ESI): 341.9 [M+H]⁺.

In a sealed tube: the mixture of 2-((5-bromo-2-chloropyrimidin-4-yl)oxy)-N-methylbenzamide (180 mg, 0.52 mmol) and 2-(2-methoxyethyl)-1,2,3,4-tetrahydroisoquinolin-6-amine (215 mg, 1.04 mmol) in n-BuOH (5 mL) was stirred at 110° C. for 18 hrs. The reaction mixture was concentrated and purified by prep-HPLC to afford 2-((5-bromo-2-((2-(2-methoxyethyl)-1,2,3,4-tetrahydroisoquinolin-6-yl)amino)pyrimidin-4-yl)oxy)-N-methylbenzamide (20 mg, 7%) as a yellow oil. HPLC/UV purity: 98.6%, LC-MS (ESI): 512.1 [M+H]⁺. ¹H NMR (400 MHz, CDCl₃) δ 8.37 (s, 1H), 8.08 (d, J=8.0 Hz, 1H), 7.55 (t, J=7.6 Hz, 1H), 7.42 (t, J=7.6 Hz, 1H), 7.23 (d, J=8.4 Hz, 1H), 7.13 (s, 1H), 7.04 (s, 1H), 6.90 (s, 2H), 6.79 (d, J=8.0 Hz, 1H), 3.83-3.85 (m, 2H), 3.74-3.75 (s, 2H), 3.39 (s, 3H), 2.96-3.00 (s, 2H), 2.92-2.90 (m, 5H), 2.77-2.79 (m, 2H).

### Example 31 Synthesis of Compound A126

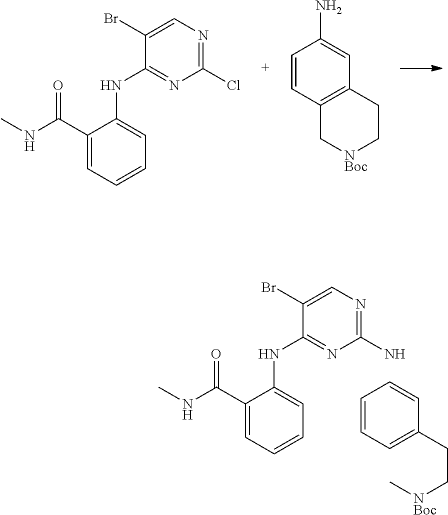

6-[5-Bromo-4-(2-methylcarbamoyl-phenylamino)-pyrimidin-2-ylamino]-3,4-dihydro-1H-isoquinoline-2-carboxylic acid tert-butyl ester. The title compound was prepared as follows. A solution of 2-(5-bromo-2-chloro-pyrimidin-4-ylamino)-N-methyl-benzamide (0.5 g, 1.47 mmol) and 6-amino-3,4-dihydro-1H-isoquinoline-2-carboxylic acid tert-butyl ester (1.5 equiv.) in DMF was heated at 120° C. for 6 hours. The reaction mixture was cooled down and solvent was removed. The mixture was washed with water and extracted into dichloromethane (3 times). The combined organic layers were washed with brine, dried over anhydrous Na₂SO₄ and concentrated. The crude product was purified by normal phase chromatography (SiO₂, MeOH/DCM gradient, 0 to 10%) to obtain the desired product as an off-white solid (0.55 g, 68%).

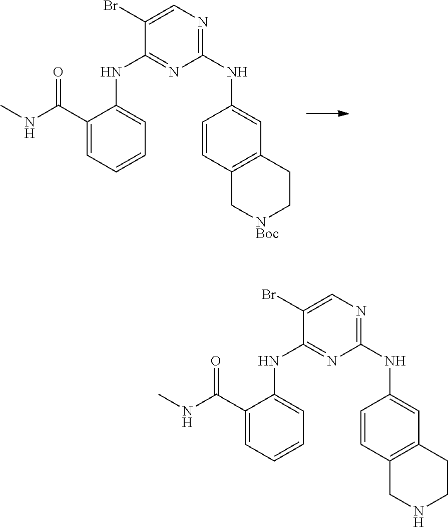 [SMILES: CNC(=O)c1ccccc1Nc1nc(Nc2ccc3c(c2)CCN(C(=O)OC(C)(C)C)C3)ncc1Br ; CNC(=O)c1ccccc1Nc1nc(Nc2ccc3c(c2)CCNC3)ncc1Br]

2-[5-Bromo-2-(1,2,3,4-tetrahydro-isoquinolin-6-ylamino)-pyrimidin-4-ylamino]-N-methyl-benzamide (A126). To 6-[5-bromo-4-(2-methylcarbamoyl-phenylamino)-pyrimidin-2-ylamino]-3,4-dihydro-1H-isoquinoline-2-carboxylic acid tert-butyl ester (0.55 g, 1 mmol) in dioxane was added 4N HCl in dioxane (4 equiv.) and stirred at room temperature for 4 hours. The reaction mixture was concentrated in vacuo and neutralized with sat. NaHCO₃. The product was extracted with DCM two times and the combined organic layers were washed with water, brine and dried over anhydrous Na₂SO₄ and concentrated to obtain the desired product (0.42 g, 93%). ¹H-NMR (400 MHz, DMSO-d₆): δ 11.36 (s, 1H), 9.44 (s, 1H), 8.75 (d, J=4.6 Hz, 1H), 8.60 (d, J=7.8 Hz, 1H), 8.24 (d, J=12.4 Hz, 1H), 7.72 (d, J=7.8 Hz, 1H), 7.45-7.49 (m, 2H), 7.41 (d, J=8.7 Hz, 1H), 7.11 (t, J=7.6 Hz, 1H), 7.02 (d, J=8.7 Hz, 1H), 4.06 (s, 2H), 3.20-3.23 (m, 2H), 2.69-2.85 (m, 6H). Observed [M+1]⁺: 453.32.

### Example 32 Synthesis of Compound A127

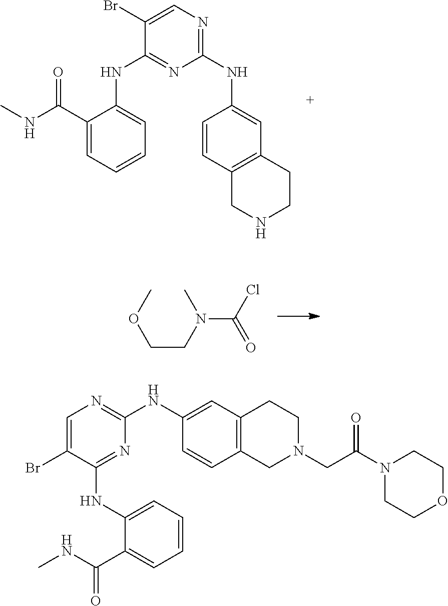

2-{5-Bromo-2-[2-(2-morpholin-4-yl-2-oxo-ethyl)-1,2,3,4-tetrahydro-isoquinolin-6-ylamino]-pyrimidin-4-ylamino}-N-methyl-benzamide (A127). To a solution of 2-[5-bromo-2-(1,2,3,4-tetrahydro-isoquinolin-6-ylamino)-pyrimidin-4-ylamino]-N-methyl-benzamide (0.05 g, 0.11 mmol) and 2-chloro-1-morpholin-4-yl-ethanone (1.2 equiv.) in DMF was added diisopropylethylamine (3.0 equiv.) and heated at 90° C. for 2 hours. The reaction mixture was cooled down and the solvent was removed in vacuo. The mixture was washed with water and extracted into dichloromethane (3 times). The combined organic layers were washed with brine, dried over anhydrous Na₂SO₄ and concentrated. The crude product was purified by normal phase chromatography (SiO₂, MeOH/DCM gradient, 0 to 10%) to obtain the desired product as an off-white solid (26 mg, 41%). ¹H-NMR (400 MHz, DMSO-D6) δ 11.31 (s, 1H), 9.32 (s, 1H), 8.71 (d, J=4.6 Hz, 1H), 8.59 (d, J=7.8 Hz, 1H), 8.24 (s, 1H), 7.69 (dd, J=8.0, 1.1 Hz, 1H), 7.42 (t, J=7.8 Hz, 2H), 7.31 (d, J=8.2 Hz, 1H), 7.10 (t, J=7.6 Hz, 1H), 6.90 (d, J=8.2 Hz, 1H), 3.42-3.53 (m), 2.77 (d, J=4.6 Hz, 3H), 2.45-2.47 (m). Observed [M+1]+: 580.8.

### Example 33 Synthesis of Compound A128

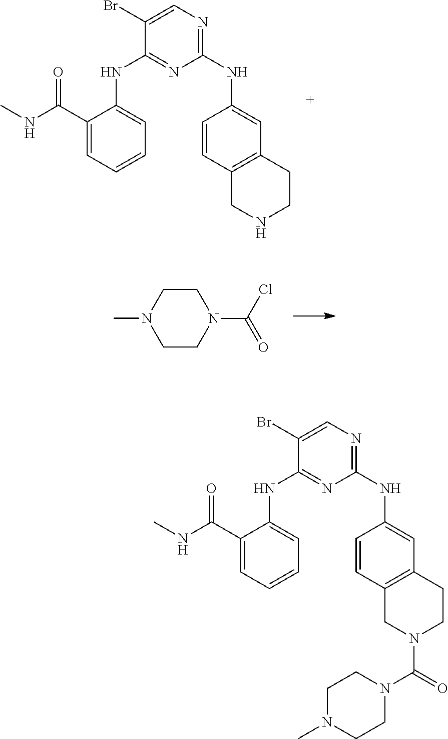

2-{5-Bromo-2-[2-(4-methyl-piperazine-1-carbonyl)-1,2,3,4-tetrahydro-isoquinolin-6-ylamino]-pyrimidin-4-ylamino}-N-methyl-benzamide (A128). To a solution of 2-[5-bromo-2-(1,2,3,4-tetrahydro-isoquinolin-6-ylamino)-pyrimidin-4-ylamino]-N-methyl-benzamide (0.05 g, 0.11 mmol) and 4-methyl-piperazine-1-carbonyl chloride (1.2 equiv.) in DMF was added diisopropylethylamine (4.0 equiv.) at 0° C. The reaction was stirred at 0° C. for 1 hour and solvent was removed. The mixture was washed with water and extracted into dichloromethane (3 times). The combined organic layers were washed with brine, dried over anhydrous NaSO4 and concentrated. The crude product was purified by normal phase chromatography (SiO₂, MeOH/DCM gradient, 0 to 10%) to obtain the desired product as an off-white solid (30 mg, 47%). ¹H NMR (400 MHz, DMSO-d6): δ 11.37 (s, 1H), 9.40 (s, 1H), 8.76 (q, J=4.5 Hz, 1H), 8.63 (d, J=8.3 Hz, 1H), 8.28 (s, 1H), 7.74 (dd, J=7.9, 1.6 Hz, 1H), 7.51-7.38 (m, 3H), 7.14 (t, J=7.6 Hz, 1H), 7.04 (d, J=8.4 Hz, 1H), 4.32 (s, 2H), 3.41 (t, J=5.7 Hz, 2H), 3.27-3.23 (m, 3H), 2.81 (d, J=4.5 Hz, 3H), 2.77-2.70 (m, 2H), 2.35 (s, 4H), 1.31-1.20 (m, 4H). Observed [M+1]+: 579.55.

### Example 34 Synthesis of Compound A129

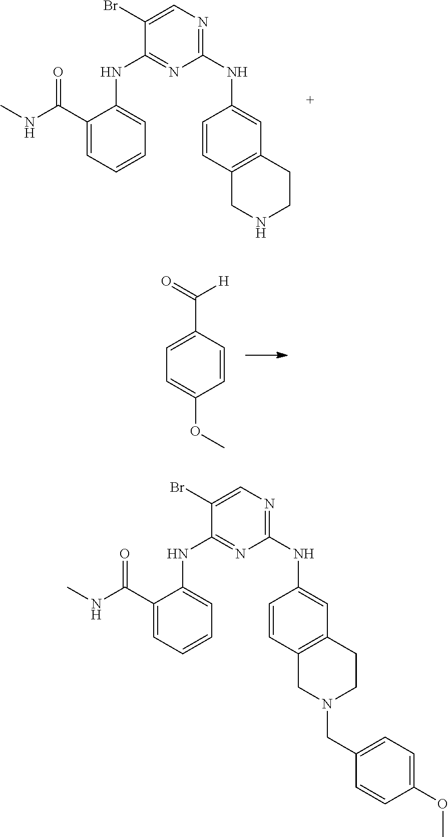

2-{5-Bromo-2-[2-(4-methoxy-benzyl)-1,2,3,4-tetrahydro-isoquinolin-6-ylamino]-pyrimidin-4-ylamino}-N-methyl-benzamide (A129). A solution of 2-[5-bromo-2-(1,2,3,4-tetrahydro-isoquinolin-6-ylamino)-pyrimidin-4-ylamino]-N-methylbenzamide (0.05 g, 0.11 mmol), 4-methoxy-benzaldehyde (4 equiv.) and sodium cyanoborohydride (2.0 equiv.) in methanol was heated at 80° C. for 2 hours. The reaction mixture was cooled down and concentrated, and the crude product was purified by normal phase chromatography (SiO₂, MeOH/DCM gradient, 0 to 10%) to obtain the desired product as an off-white solid (31 mg, 49%). ¹H NMR (400 MHz, DMSO-d6): δ 11.33 (s, 1H), 9.35 (s, 1H), 8.74 (q, J=4.6 Hz, 1H), 8.62 (d, J=8.4 Hz, 1H), 8.27 (s, 1H), 7.72 (dd, J=8.0, 1.6 Hz, 1H), 7.50-7.41 (m, 2H), 7.33 (dd, J=8.3, 2.2 Hz, 1H), 7.26 (d, J=8.5 Hz, 2H), 7.13 (t, J=8.2 Hz, 1H), 6.94-6.83 (m, 3H), 3.74 (s, 3H), 3.55 (s, 2H), 3.44 (s, 2H), 2.80 (d, J=4.5 Hz, 3H), 2.71 (t, J=5.9 Hz, 2H), 2.63 (t, J=5.6 Hz, 2H). Observed [M+1]+: 573.47.

### Example 35 Synthesis of Compound A130

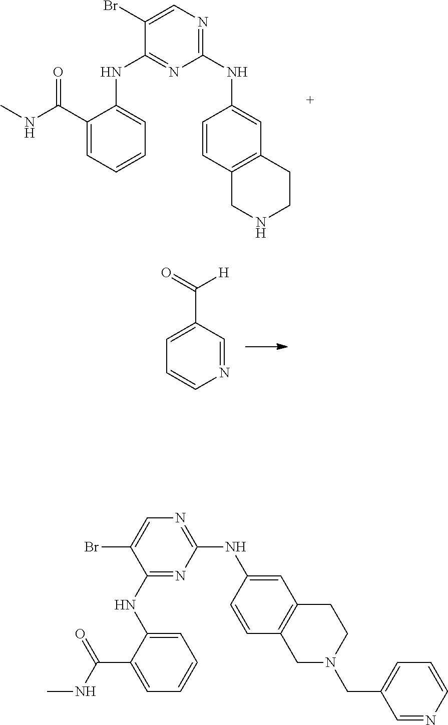 [SMILES: CNC(=O)c1ccccc1Nc1nc(Nc2ccc3c(c2)CCNC3)ncc1Br ; O=Cc1cccnc1 ; CNC(=O)c1ccccc1Nc1nc(Nc2ccc3c(c2)CCN(Cc2cccnc2)C3)ncc1Br]

2-[5-Bromo-2-(2-pyridin-3-ylmethyl-1,2,3,4-tetrahydro-isoquinolin-6-ylamino)-pyrimidin-4-ylamino]-N-methyl-benzamide (A130). The synthetic procedure is similar to the synthesis of Compound A129 except pyridine-3-carbaldehyde was used instead of 4-methoxy-benzaldehyde to obtain the desired product as an off-white solid (22 mg, 37%). ¹H NMR (400 MHz, DMSO-d₆): δ 11.33 (s, 1H), 9.36 (s, 1H), 8.74 (d, J=4.8 Hz, 1H), 8.62 (d, J=8.3 Hz, 1H), 8.56 (d, J=2.2 Hz, 1H), 8.49 (dd, J=4.7, 1.7 Hz, 1H), 8.27 (s, 1H), 7.81-7.69 (m, 2H), 7.51-7.42 (m, 2H), 7.38 (dd, J=7.8, 4.8 Hz, 1H), 7.34 (d, J=7.6 Hz, 1H), 7.13 (t, J=7.6 Hz, 1H), 6.90 (d, J=8.3 Hz, 1H), 3.67 (s, 2H), 3.50 (s, 2H), 2.80 (d, J=4.4 Hz, 3H), 2.73 (t, J=5.8 Hz, 2H), 2.66 (t, J=5.6 Hz, 2H). Observed [M+1]⁺: 544.45.

### Example 36 Synthesis of Compound A131

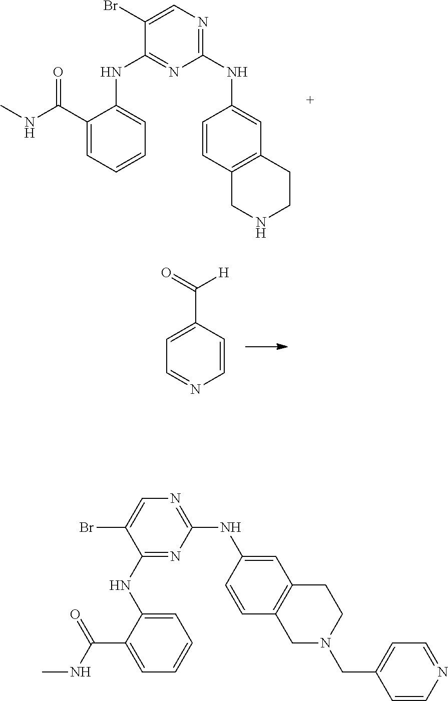 [SMILES: CNC(=O)c1ccccc1Nc1nc(Nc2ccc3c(c2)CCNC3)ncc1Br ; O=Cc1ccncc1 ; CNC(=O)c1ccccc1Nc1nc(Nc2ccc3c(c2)CCN(Cc2ccncc2)C3)ncc1Br]

2-[5-Bromo-2-(2-pyridin-4-ylmethyl-1,2,3,4-tetrahydro-isoquinolin-6-ylamino)-pyrimidin-4-ylamino]-N-methyl-benzamide (A131). The synthetic procedure is similar to the synthesis of Compound A129 except pyridine-4-carbaldehyde was used instead of 4-methoxy-benzaldehyde to obtain the desired product as an off-white solid (35 mg, 58%). ¹H NMR (400 MHz, DMSO-d₆): δ 11.34 (s, 1H), 9.36 (s, 1H), 8.74 (d, J=4.9 Hz, 1H), 8.66-8.60 (m, 1H), 8.56-8.50 (m, 2H), 8.28 (s, 1H), 7.76-7.69 (m, 1H), 7.51-7.43 (m, 2H), 7.38 (d, J=5.0 Hz, 2H), 7.37-7.31 (m, 1H), 7.14 (t, J=7.6 Hz, 1H), 6.90 (d, J=8.3 Hz, 1H), 3.68 (s, 2H), 3.51 (s, 2H), 2.80 (d, J=4.5 Hz, 3H), 2.75 (t, J=5.7 Hz, 2H), 2.67 (t, J=5.8 Hz, 2H). Observed [M+1]⁺: 544.49.

### Example 37 Synthesis of Compound A132

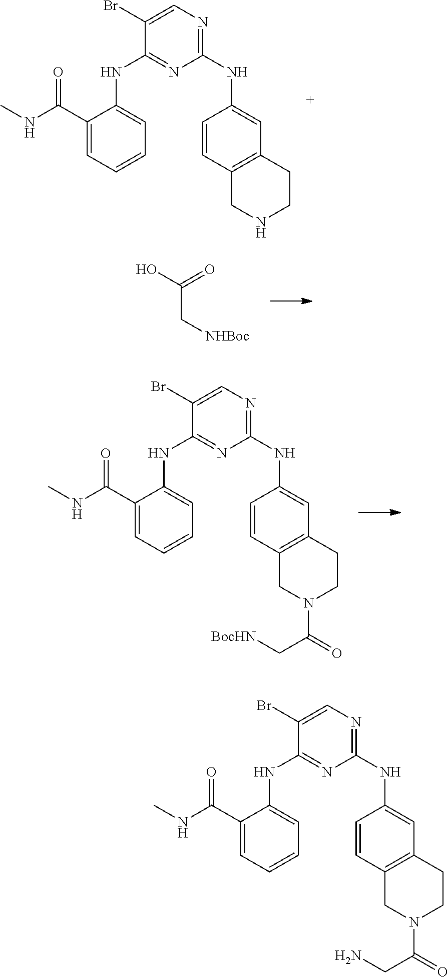 [SMILES: CNC(=O)c1ccccc1Nc1nc(Nc2ccc3c(c2)CCNC3)ncc1Br ; CC(C)(C)OC(=O)NCC(=O)O ; CNC(=O)c1ccccc1Nc1nc(Nc2ccc3c(c2)CCN(C(=O)CNC(=O)OC(C)(C)C)C3)ncc1Br ; CNC(=O)c1ccccc1Nc1nc(Nc2ccc3c(c2)CCN(C(=O)CN)C3)ncc1Br]

2-{2-[2-(2-Amino-acetyl)-1,2,3,4-tetrahydro-isoquinolin-6-ylamino]-5-bromo-pyrimidin-4-ylamino}-N-methyl-benzamide (A132). A solution of 2-[5-bromo-2-(1,2,3,4-tetrahydro-isoquinolin-6-ylamino)-pyrimidin-4-ylamino]-N-methyl-benzamide (0.1 g, 0.22 mmol), triethylamine (2.2 equiv.) and tert-butoxycarbonylamino-acetic acid (1.2 equiv.) in DMF was cooled to 0° C. and HATU (1.2 equiv.) was added. The solution was stirred at room temperature for 4 hours. The solvent was removed and residue was washed with water and extracted into dichloromethane (3 times). The combined organic layers were washed with brine, dried over anhydrous NaSO₄ and concentrated. The crude product was purified by normal phase chromatography (SiO₂, MeOH/DCM gradient, 0 to 10%) to obtain the intermediate (2-{6-[5-bromo-4-(2-methylcarbamoyl-phenylamino)-pyrimidin-2-ylamino]-3,4-dihydro-1H-isoquinolin-2-yl}-2-oxo-ethyl)-carbamic acid tert-butyl ester as an off-white solid (55 mg, 41%).

(2-{6-[5-Bromo-4-(2-methylcarbamoyl-phenylamino)-pyrimidin-2-ylamino]-3,4-dihydro-1H-isoquinolin-2-yl}-2-oxo-ethyl)-carbamic acid tert-butyl ester was dissolved in THF and treated with 4N HCl in dioxane (5 equiv.) for 4 hours. The solvent was removed and the residue was neutralized with sat. NaHCO₃ and extracted with DCM (3 times). The combined organic layers were washed with water, brine, dried over anhydrous Na₂SO₄ and concentrated to obtain the desired product as an off-white solid (29 mg, 71%). ¹H NMR (400 MHz, DMSO-d₆): δ 11.35 (s, 1H), 9.44 (d, J=6.4 Hz, 1H), 8.76 (q, J=4.4 Hz, 1H), 8.66-8.60 (m, 1H), 8.29 (s, 1H), 7.74 (d, J=7.9 Hz, 1H), 7.53 (s, 1H), 7.52-7.39 (m, 2H), 7.20-7.12 (m, 1H), 7.08 (t, J=9.0 Hz, 1H), 4.59-4.49 (m, 2H), 3.75-3.62 (m, 2H), 3.62-3.53 (m, 1H), 3.52-3.42 (m, 2H), 2.81 (d, J=4.5 Hz, 3H), 2.76 (t, J=5.8 Hz, 1H), 2.68 (t, J=6.0 Hz, 1H). Observed [M+1]⁺: 510.35.

### Example 38 Synthesis of Compound A133

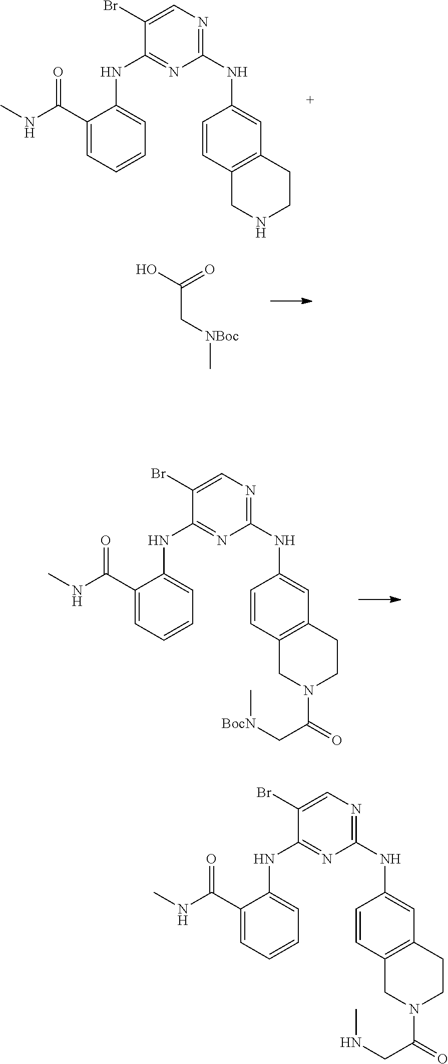 [SMILES: CNC(=O)c1ccccc1Nc1nc(Nc2ccc3c(c2)CCNC3)ncc1Br ; CN(CC(=O)O)C(=O)OC(C)(C)C ; CNC(=O)c1ccccc1Nc1nc(Nc2ccc3c(c2)CCN(C(=O)CN(C)C(=O)OC(C)(C)C)C3)ncc1Br ; CNCC(=O)N1CCc2cc(Nc3ncc(Br)c(Nc4ccccc4C(=O)NC)n3)ccc2C1]

2-{5-Bromo-2-[2-(2-methylamino-acetyl)-1,2,3,4-tetrahydro-isoquinolin-6-ylamino]-pyrimidin-4-ylamino}-N-methyl-benzamide (A133). The synthetic procedure is similar to the synthesis of Compound A132 except (tert-butoxycarbonyl-methyl-amino)-acetic acid was used instead of tert-butoxycarbonylamino-acetic acid to obtain the desired product as an off-white solid (27 mg, 46%). ¹H NMR (400 MHz, DMSO-d6): δ 11.35 (s, 1H), 9.44 (d, J=6.3 Hz, 1H), 8.78-8.72 (m, 1H), 8.64-8.60 (m, 1H), 8.29 (s, 1H), 7.74 (dd, J=7.9, 1.6 Hz, 1H), 7.56-7.38 (m, 4H), 7.22-7.12 (m, 1H), 7.08 (t, J=8.8 Hz, 1H), 4.55 (s, 2H), 3.66 (t, J=5.9 Hz, 1H), 3.61 (t, J=6.0 Hz, 1H), 3.43 (s, 2H), 2.81 (d, J=4.5 Hz, 3H), 2.76 (t, J=5.8 Hz, 1H), 2.67 (t, J=6.0 Hz, 1H), 2.30 (s, 3H). Observed [M+1]+: 524.47.

### Example 39 Synthesis of Compound A134

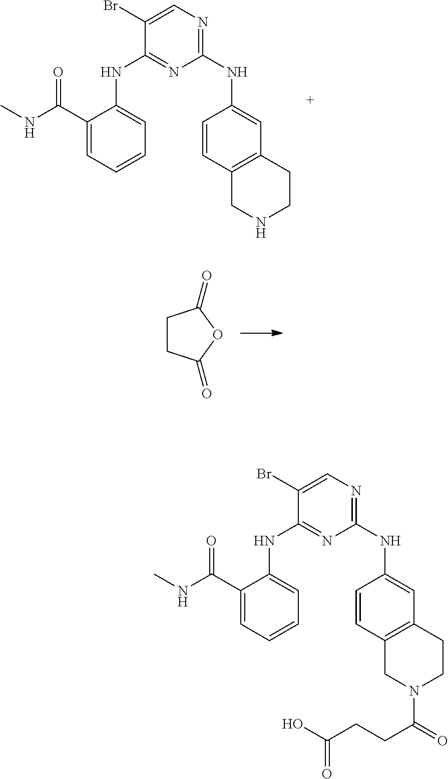 [SMILES: CNC(=O)c1ccccc1Nc1nc(Nc2ccc3c(c2)CCNC3)ncc1Br ; O=C1CCC(=O)O1 ; CNC(=O)c1ccccc1Nc1nc(Nc2ccc3c(c2)CCN(C(=O)CCC(=O)O)C3)ncc1Br]

4-{6-[5-Bromo-4-(2-methylcarbamoyl-phenylamino)-pyrimidin-2-ylamino]-3,4-dihydro-1H-isoquinolin-2-yl}-4-oxo-butyric acid (A134). The synthetic procedure is similar to the synthesis of Compound A127 except dihydro-furan-2,5-dione was used instead of 2-chloro-1-morpholin-4-yl-ethanone to obtain the desired product as an off-white solid (25 mg, 41%). ¹H NMR (400 MHz, DMSO-d6): δ 12.04 (s, 1H), 11.35 (s, 1H), 9.44 (d, J=6.0 Hz, 1H), 8.75 (q, J=4.6 Hz, 1H), 8.66-8.56 (m, 2H), 8.29 (s, 1H), 7.73 (d, J=7.8 Hz, 1H), 7.57-7.36 (m, 3H), 7.15 (t, J=7.6 Hz, 1H), 7.08 (t, J=8.6 Hz, 1H), 4.60 (s, 1H), 4.53 (s, 1H), 3.65 (q, J=6.4 Hz, 2H), 2.84-2.73 (m, 3H), 2.69-2.58 (m, 3H), 2.50-2.39 (m, 2H). Observed [M+1]+: 553.48.

### Example 40 Synthesis of Compound A135

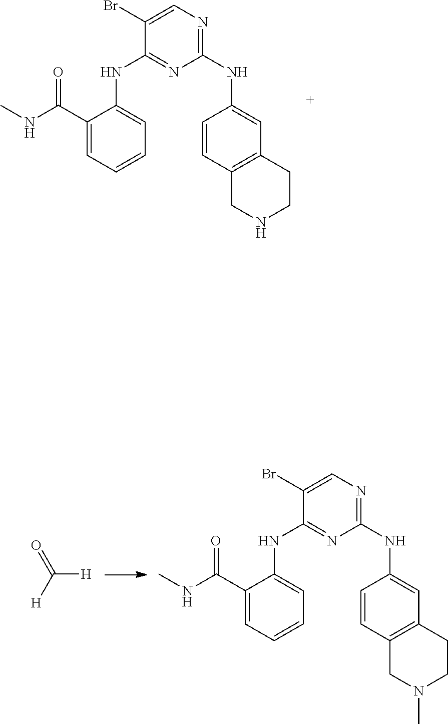 [SMILES: CNC(=O)c1ccccc1Nc1nc(Nc2ccc3c(c2)CCNC3)ncc1Br ; C=O ; CNC(=O)c1ccccc1Nc1nc(Nc2ccc3c(c2)CCN(C)C3)ncc1Br]

2-[5-Bromo-2-(2-methyl-1,2,3,4-tetrahydro-isoquinolin-6-ylamino)-pyrimidin-4-ylamino]-N-methyl-benzamide (A135). The synthetic procedure is similar to the synthesis of Compound A129 except formaldehyde was used instead of 4-methoxy-benzaldehyde to obtain the desired product as an off-white solid (22 mg, 43%). ¹H NMR (400 MHz, DMSO-d6): δ 11.34 (s, 1H), 9.34 (s, 1H), 8.74 (q, J=4.6 Hz, 1H), 8.63 (d, J=8.3 Hz, 1H), 8.27 (s, 1H), 7.73 (dd, J=7.8, 1.6 Hz, 1H), 7.50-7.41 (m, 2H), 7.33 (dd, J=8.2, 2.2 Hz, 1H), 7.14 (t, J=7.6 Hz, 1H), 6.92 (d, J=8.4 Hz, 1H), 3.42 (s, 2H), 2.81 (d, J=4.4 Hz, 3H), 2.72 (t, J=5.9 Hz, 2H), 2.56 (t, J=5.8 Hz, 2H), 2.32 (s, 3H). Observed [M+1]+: 467.44.

### Example 41 Synthesis of Compound A137

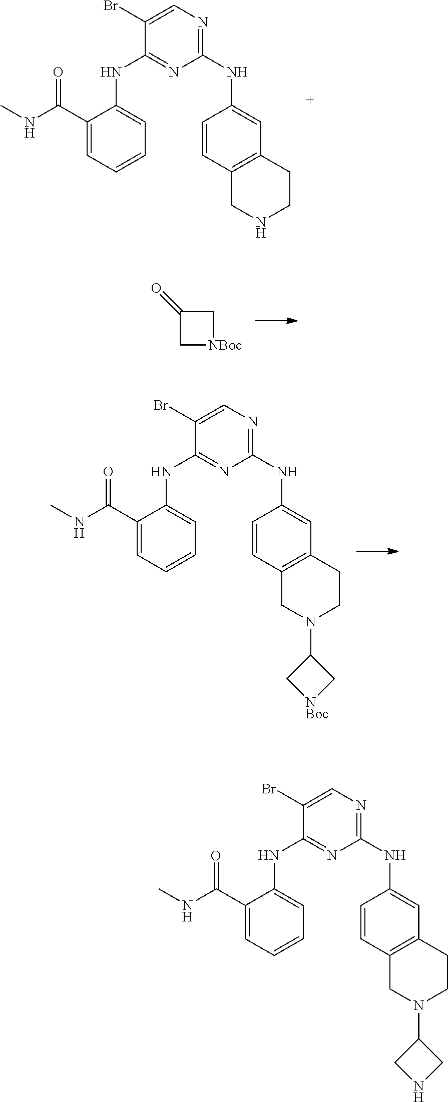 [SMILES: CNC(=O)c1ccccc1Nc1nc(Nc2ccc3c(c2)CCNC3)ncc1Br ; CC(C)(C)OC(=O)N1CC(=O)C1 ; CNC(=O)c1ccccc1Nc1nc(Nc2ccc3c(c2)CCN(C2CN(C(=O)OC(C)(C)C)C2)C3)ncc1Br ; CNC(=O)c1ccccc1Nc1nc(Nc2ccc3c(c2)CCN(C2CNC2)C3)ncc1Br]

2-{2-[2-(3-Amino-propionyl)-1,2,3,4-tetrahydro-isoquinolin-6-ylamino]-5-bromo-pyrimidin-4-ylamino}-N-methyl-benzamide (A137). The synthetic procedure is similar to the synthesis of Compound A129 except 3-oxo-azetidine-1-carboxylic acid tert-butyl ester was used instead of 4-methoxy-benzaldehyde. The Boc protected intermediate was dissolved in THF and treated with 4N HCl in dioxane (4 equiv.) for 4 hours. The solvent was removed and the residue was neutralized with sat. NaHCO₃ and extracted with DCM (3 times). The combined organic layers were washed with water, brine, dried over anhydrous Na₂SO₄ and concentrated to obtain the desired product as an off-white solid (90 mg, 27%). ¹H-NMR (400 MHz, DMSO-d6): δ 10.51 (s, 1H), 9.40 (d, J=3.2 Hz, 4H), 9.00 (s, 1H), 8.56-8.75 (m), 8.44 (s, 1H), 8.26 (d, J=7.8 Hz, 4H), 8.19 (s, OH), 7.69-7.79 (m), 7.36-7.50 (m), 7.05-7.18 (m, 8H), 6.93 (t, J=4.4 Hz, 5H), 3.86-4.10 (m, 21H), 1.79 (d, J=61.8 Hz, 5H). Observed [M+1]+: 510.51.

### Example 42 Synthesis of Compound A138

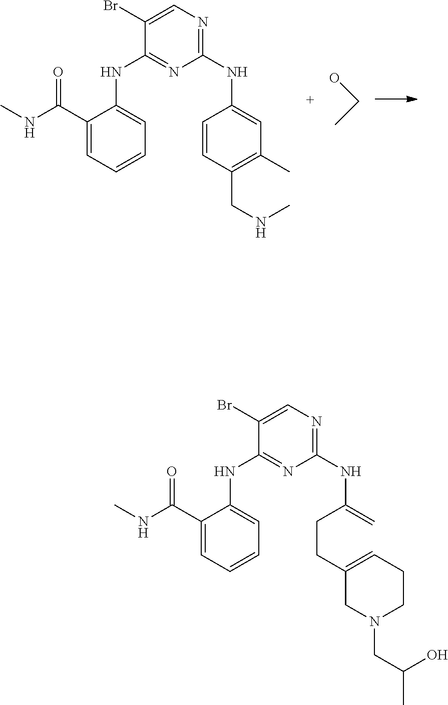

2-{5-Bromo-2-[2-(2-hydroxy-propyl)-1,2,3,4-tetrahydro-isoquinolin-6-ylamino]-pyrimidin-4-ylamino}-N-methyl-benzamide (A138). The synthetic procedure is similar to the synthesis of Compound A127 except that 2-methyl-oxirane was used instead of 2-chloro-1-morpholin-4-yl-ethanone to obtain the desired product as an off-white solid (17 mg, 30%). Observed [M+1]+: 511.52.

### Example 43 Synthesis of Compound A136

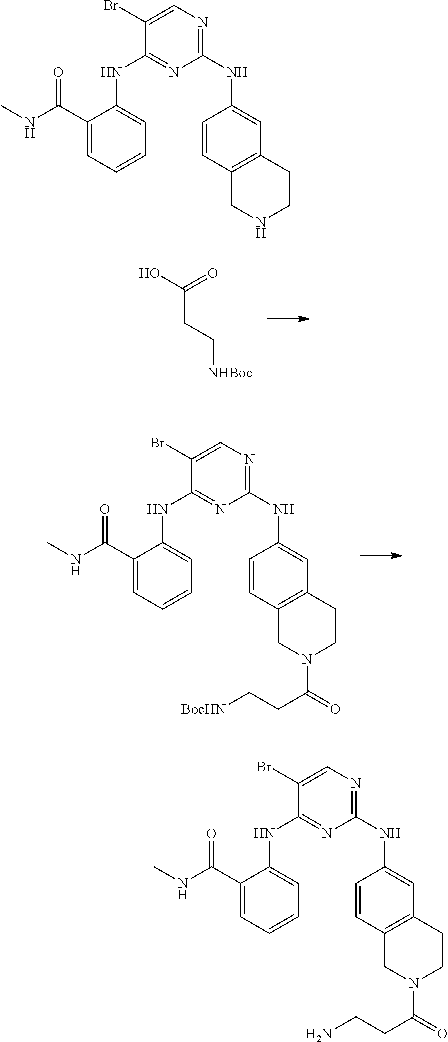 [SMILES: CNC(=O)c1ccccc1Nc1nc(Nc2ccc3c(c2)CCNC3)ncc1Br ; CC(C)(C)OC(=O)NCCC(=O)O ; CNC(=O)c1ccccc1Nc1nc(Nc2ccc3c(c2)CCN(C(=O)CCNC(=O)OC(C)(C)C)C3)ncc1Br ; CNC(=O)c1ccccc1Nc1nc(Nc2ccc3c(c2)CCN(C(=O)CCN)C3)ncc1Br]

2-{2-[2-(3-Amino-propionyl)-1,2,3,4-tetrahydro-isoquinolin-6-ylamino]-5-bromo-pyrimidin-4-ylamino}-N-methyl-benzamide (A136). The synthetic procedure is similar to the synthesis of Compound A132 except 3-tert-butoxycarbonylamino-propionic acid was used instead of tert-butoxycarbonylamino-acetic acid to obtain the desired product as an off-white solid (10 mg, 24%). Observed [M+1]+: 524.38.

### Example 44 Synthesis of Compound A139

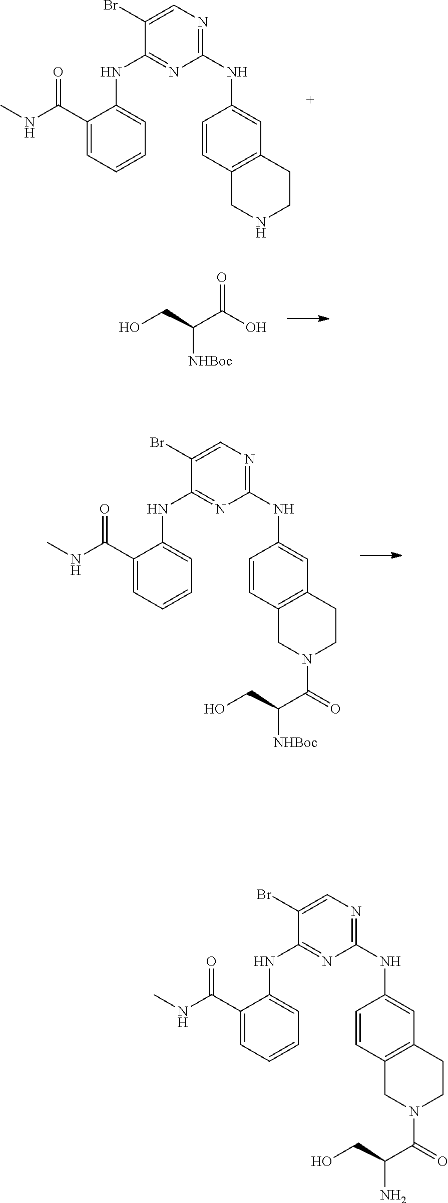 [SMILES: CNC(=O)c1ccccc1Nc1nc(Nc2ccc3c(c2)CCNC3)ncc1Br ; CC(C)(C)OC(=O)N[C@@H](CO)C(=O)O ; CNC(=O)c1ccccc1Nc1nc(Nc2ccc3c(c2)CCN(C(=O)[C@H](CO)NC(=O)OC(C)(C)C)C3)ncc1Br ; CNC(=O)c1ccccc1Nc1nc(Nc2ccc3c(c2)CCN(C(=O)[C@@H](N)CO)C3)ncc1Br]

2-{2-[2-(2-Amino-3-hydroxy-propionyl)-1,2,3,4-tetrahydro-isoquinolin-6-ylamino]-5-bromo-pyrimidin-4-ylamino}-N-methyl-benzamide (A139). The synthetic procedure is similar to the synthesis of Compound A132 except 2-tert-butoxycarbonylamino-3-hydroxy-propionic acid was used instead of tert-butoxycarbonylamino-acetic acid to obtain the desired product as an off-white solid (25 mg, 41%). ¹H-NMR (400 MHz, DMSO-d6): δ 11.32 (s, 1H), 9.40 (d, J=4.6 Hz, 1H), 8.72 (d, J=4.6 Hz, 1H), 8.59 (d, J=6.9 Hz, 1H), 8.25 (s, 1H), 7.70 (dd, J=8.2, 1.4 Hz, 1H), 7.37-7.49 (m, 2H), 7.11 (t, J=7.6 Hz, 1H), 7.04 (dd, J=8.0, 4.8 Hz, 1H), 4.49-4.72 (m, 2H), 3.92 (q, J=6.6 Hz, 1H), 3.55-3.75 (m, 1H), 2.76-2.80 (m, 3H), 2.45-2.47 (m), 1.71-1.86 (m, 1H). Observed [M+1]+: 540.51.

### Example 45 Synthesis of Compound A140

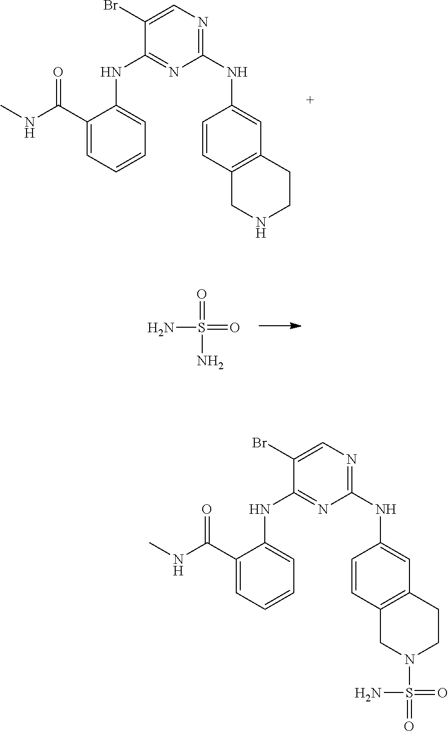 [SMILES: CNC(=O)c1ccccc1Nc1nc(Nc2ccc3c(c2)CCNC3)ncc1Br ; NS(N)(=O)=O ; CNC(=O)c1ccccc1Nc1nc(Nc2ccc3c(c2)CCN(S(N)(=O)=O)C3)ncc1Br]

2-[5-Bromo-2-(2-sulfamoyl-1,2,3,4-tetrahydro-isoquinolin-6-ylamino)-pyrimidin-4-ylamino]-N-methyl-benzamide (A140). The synthetic procedure is similar to the synthesis of Compound A127 except sulfamide was used instead of 2-chloro-1-morpholin-4-yl-ethanone to obtain the desired product as an off-white solid (31 mg, 53%). ¹H-NMR (400 MHz, DMSO-d6): δ 11.31 (s, 1H), 9.39 (s, 1H), 8.71 (d, J=4.6 Hz, 1H), 8.63-8.54 (1H), 8.25 (s, 1H), 8.21-8.17 (OH), 7.70 (d, J=8.2 Hz, 1H), 7.43-7.49 (m, 3H), 7.38 (d, J=8.7 Hz, 1H), 7.12 (t, J=7.6 Hz, 1H), 7.01 (d, J=8.2 Hz, 1H), 6.86 (s, 2H), 4.10 (s, 2H), 3.20 (t, J=5.5 Hz, 2H), 3.04 (s, 1H), 2.84 (s, OH), 2.77 (d, J=3.7 Hz, 6H). Observed [M+1]+: 532.23.

### Example 46 Synthesis of Compound A141

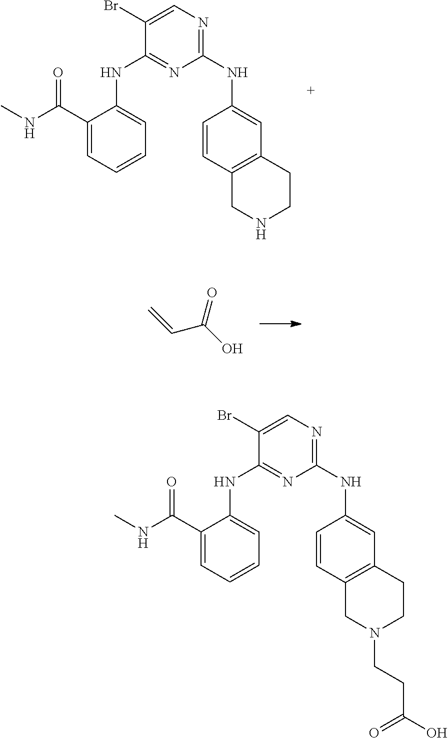

3-{6-[5-Bromo-4-(2-methylcarbamoyl-phenylamino)-pyrimidin-2-ylamino]-3,4-dihydro-1H-isoquinolin-2-yl}-propionic acid (A141). The synthetic procedure is similar to the synthesis of Compound A127 except acrylic acid was used instead of 2-chloro-1-morpholin-4-yl-ethanone to obtain the desired product as an off-white solid (21 mg, 36%). Observed [M+1]+: 525.48.

### Example 47 Synthesis of Compound A142

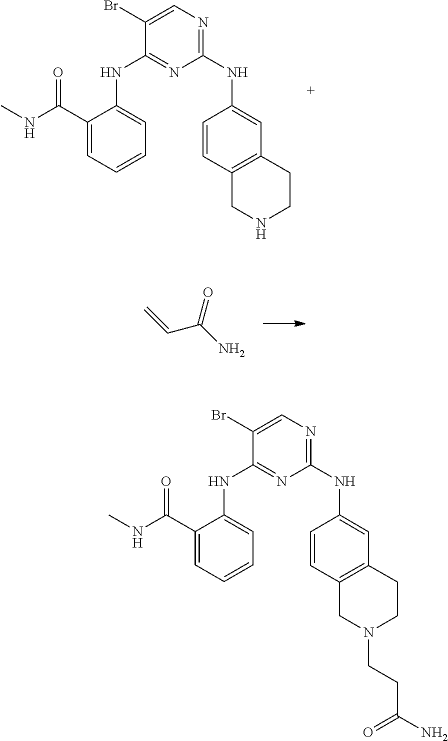

2-{5-Bromo-2-[2-(2-carbamoyl-ethyl)-1,2,3,4-tetrahydro-isoquinolin-6-ylamino]-pyrimidin-4-ylamino}-N-methyl-benzamide (A142). The synthetic procedure is similar to the synthesis of Compound A127 except acrylamide was used instead of 2-chloro-1-morpholin-4-yl-ethanone to obtain the desired product as an off-white solid (28 mg, 48%). ¹H-NMR (400 MHz, DMSO-d6): δ 11.38 (s, 1H), 9.86-9.73 (1H), 9.53 (s, 1H), 8.72 (d, J=4.6 Hz, 1H), 8.63-8.55 (1H), 8.28 (s, 1H), 7.71 (dd, J=7.8, 1.4 Hz, 1H), 7.58 (d, J=5.5 Hz, 2H), 7.47 (m, 2H), 7.09-7.13 (m), 6.947 (s, 1H), 4.41 (s, 1H), 4.28-4.15 (1H), 2.77 (d, J=4.6 Hz, 3H), 2.45-2.47 (m). Observed [M+1]+: 524.44.

### Example 48 Synthesis of Compound A143

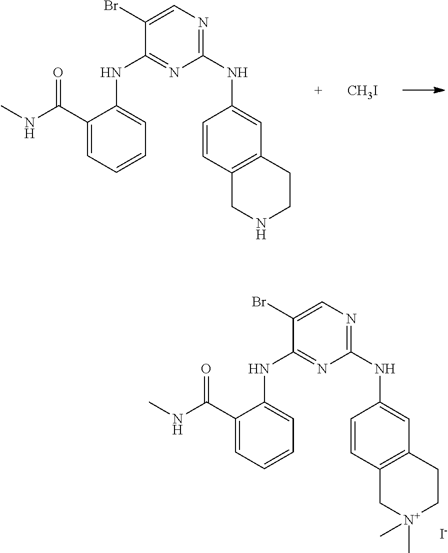 [SMILES: CNC(=O)c1ccccc1Nc1nc(Nc2ccc3c(c2)CCNC3)ncc1Br ; "CH3I" ; CNC(=O)c1ccccc1Nc1nc(Nc2ccc3c(c2)CC[N+](C)(C)C3)ncc1Br ; [I-]]

6-[5-Bromo-4-(2-methylcarbamoyl-phenylamino)-pyrimidin-2-ylamino]-2,2-dimethyl-1,2,3,4-tetrahydro-isoquinolinium; iodide (A143). The synthetic procedure is similar to the synthesis of Compound A127 except iodomethane was used instead of 2-chloro-1-morpholin-4-yl-ethanone to obtain the desired product as an off-white solid (12 mg, 15%). ¹H NMR (400 MHz, DMSO-d6): δ 11.48 (s, 1H), 9.70 (s, 1H), 8.83 (q, J=4.4 Hz, 1H), 8.62 (d, J=8.4 Hz, 1H), 8.33 (s, 1H), 7.78 (d, J=7.9 Hz, 1H), 7.67 (s, 1H), 7.58-7.48 (m, 2H), 7.16 (t, J=7.5 Hz, 1H), 7.09 (d, J=8.5 Hz, 1H), 4.54 (s, 2H), 3.68 (t, J=6.5 Hz, 2H), 3.15 (s, 6H), 3.07 (t, J=6.6 Hz, 2H), 2.81 (d, J=4.4 Hz, 3H). ¹³C NMR (101 MHz, DMSO-d6): δ 168.7, 158.0, 157.6, 156.8, 155.9, 140.0, 139.0, 131.4, 129.6, 128.1, 127.1, 122.3, 121.6, 121.4, 120.2, 118.6, 94.7, 62.2, 58.3, 50.6, 26.3, 23.7. Observed [M+1]+: 481.36.

### Example 49 Synthesis of Compound A144

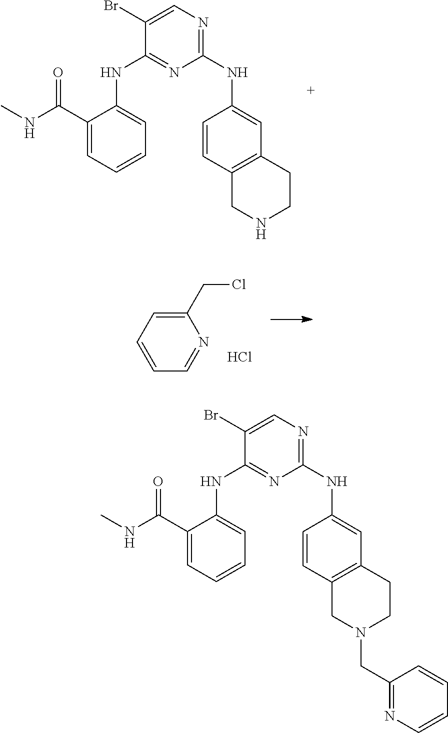 [SMILES: CNC(=O)c1ccccc1Nc1nc(Nc2ccc3c(c2)CCNC3)ncc1Br ; ClCc1ccccn1 ; Cl ; CNC(=O)c1ccccc1Nc1nc(Nc2ccc3c(c2)CCN(Cc2ccccn2)C3)ncc1Br]

2-[5-Bromo-2-(2-pyridin-2-ylmethyl-1,2,3,4-tetrahydro-isoquinolin-6-ylamino)-pyrimidin-4-ylamino]-N-methyl-benzamide (A144). The synthetic procedure is similar to the synthesis of Compound A127 except 2-chloromethylpyridine HCl salt was used instead of 2-chloro-1-morpholin-4-yl-ethanone to obtain the desired product as an off-white solid (24 mg, 40%). Observed [M+1]+: 544.38.

### Example 50 Synthesis of Compound A145

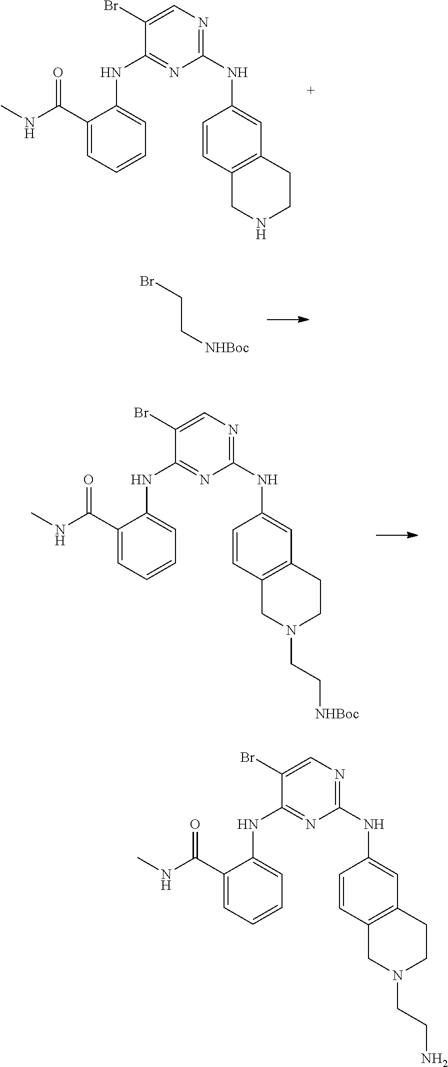

2-{2-[2-(2-Amino-ethyl)-1,2,3,4-tetrahydro-isoquinolin-6-ylamino]-5-bromo-pyrimidin-4-ylamino}-N-methyl-benzamide (A145). The synthetic procedure is similar to the synthesis of Compound A127 except that (2-bromoethyl)-carbamic acid tert-butyl ester was used instead of 2-chloro-1-morpholin-4-yl-ethanone. The Boc protected intermediate was dissolved in THF and treated with 4N HCl in dioxane (4 equiv.) for 4 hours. The solvent was removed in vacuo and the residue was neutralized with sat. NaHCO₃ and extracted with DCM (3 times). The combined organic layers were washed with water, brine and dried over anhydrous Na₂SO₄ to obtain the desired product as an off-white solid (20 mg, 44%). Observed [M+1]+: 496.32.

### Example 51 Synthesis of Compound A146

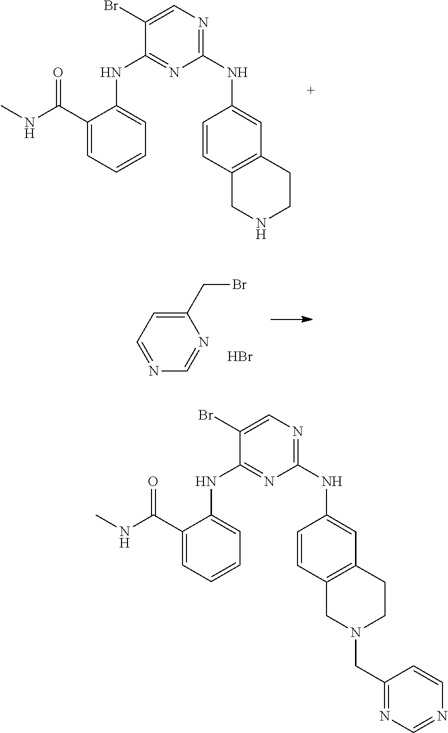 [SMILES: CNC(=O)c1ccccc1Nc1nc(Nc2ccc3c(c2)CCNC3)ncc1Br ; BrCc1ccncn1 ; Br ; CNC(=O)c1ccccc1Nc1nc(Nc2ccc3c(c2)CCN(Cc2ccncn2)C3)ncc1Br]

2-[5-Bromo-2-(2-pyrimidin-4-ylmethyl-1,2,3,4-tetrahydro-isoquinolin-6-ylamino)-pyrimidin-4-ylamino]-N-methyl-benzamide (A146). The synthetic procedure is similar to the synthesis of Compound A127 except that 4-bromomethyl-pyrimidine HBr salt was used instead of 2-chloro-1-morpholin-4-yl-ethanone to obtain the desired product as an off-white solid (28 mg, 47%). ¹H-NMR (400 MHz, DMSO-d6): δ 11.33 (s, 1H), 9.35 (s, 1H), 9.10 (d, J=1.4 Hz, 1H), 8.75 (d, J=5.0 Hz, 2H), 8.26 (s, 1H), 7.59-7.72 (m, 2H), 7.45 (s, 2H), 7.13 (d, J=7.3 Hz, 1H), 6.90 (d, J=8.2 Hz, 1H), 3.77 (s, 2H), 3.57 (s, 3H), 2.79 (d, J=4.6 Hz, 2H), 2.47-2.49 (m). Observed [M+1]+: 545.40.

### Example 52 Synthesis of Compound A147

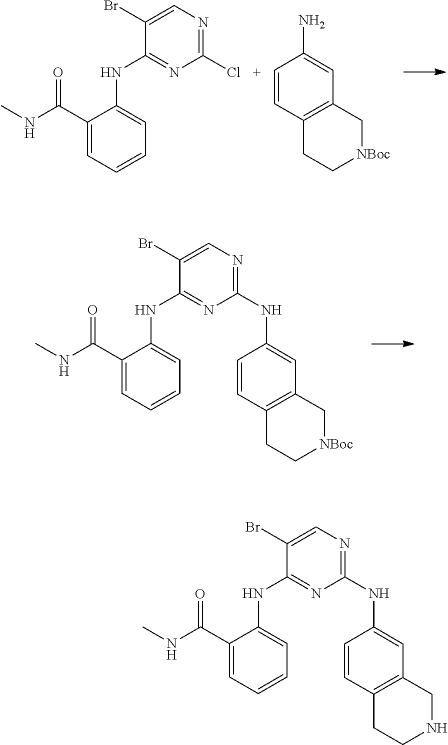

2-[5-Bromo-2-(1,2,3,4-tetrahydro-isoquinolin-7-ylamino)-pyrimidin-4-ylamino]-N-methyl-benzamide (A147). The synthetic procedure is similar to the synthesis of Compound A126 except that 7-amino-3,4-dihydro-1H-isoquinoline-2-carboxylic acid tert-butyl ester was used instead of 6-amino-3,4-dihydro-1H-isoquinoline-2-carboxylic acid tert-butyl ester to obtain the desired product as an off-white solid (0.40 g, 68%). ¹H-NMR (400 MHz, DMSO-d6): δ 11.36 (s, 1H), 9.49 (s, 1H), 8.75 (d, J=4.6 Hz, 1H), 8.59 (d, J=7.3 Hz, 1H), 8.26 (s, 1H), 7.71-7.73 (m, 1H), 7.43-7.52 (m, 3H), 7.10 (q, J=8.5 Hz, 2H), 4.11 (s, 2H), 3.03 (m, 6H), 2.77 (d, J=4.6 Hz, 3H). Observed [M+1]+: 453.45.

### Example 53 Synthesis of Compound A148

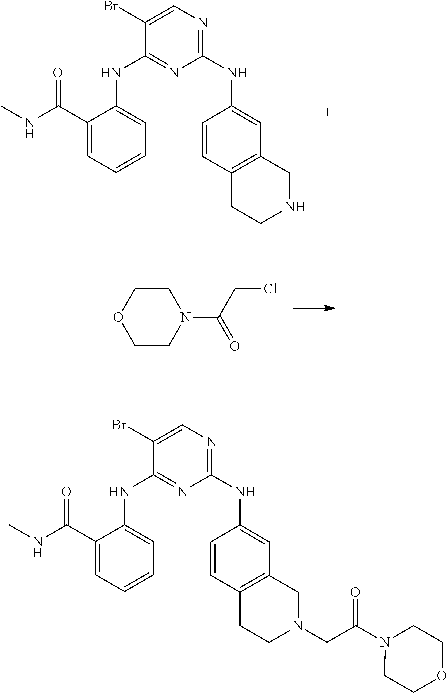 [SMILES: CNC(=O)c1ccccc1Nc1nc(Nc2ccc3c(c2)CNCC3)ncc1Br ; O=C(CCl)N1CCOCC1 ; CNC(=O)c1ccccc1Nc1nc(Nc2ccc3c(c2)CN(CC(=O)N2CCOCC2)CC3)ncc1Br]

2-{5-Bromo-2-[2-(2-morpholin-4-yl-2-oxo-ethyl)-1,2,3,4-tetrahydro-isoquinolin-7-ylamino]-pyrimidin-4-ylamino}-N-methyl-benzamide (A148). The synthetic procedure is similar to the synthesis of Compound A127 except 2-[5-bromo-2-(1,2,3,4-tetrahydro-isoquinolin-7-ylamino)-pyrimidin-4-ylamino]-N-methyl-benzamide was used instead of 2-[5-bromo-2-(1,2,3,4-tetrahydro-isoquinolin-6-ylamino)-pyrimidin-4-ylamino]-N-methyl-benzamide to obtain the desired product as an off-white solid (26 mg, 41%). ¹H-NMR (400 MHz, DMSO-d6): δ 11.27 (s, 1H), 9.32 (s, 1H), 8.70 (d, J=4.6 Hz, 1H), 8.56 (d, J=7.3 Hz, 1H), 8.23 (s, 1H), 7.69 (d, J=6.9 Hz, 1H), 7.42-7.46 (m, 1H), 7.36 (s, 1H), 7.30 (d, J=8.2 Hz, 1H), 7.11 (t, J=7.6 Hz, 1H), 6.95 (d, J=8.2 Hz, 1H), 3.37-3.53 (m), 2.77 (d, J=4.1 Hz, 3H), 2.45-2.47 (m). Observed [M+1]+: 580.51.

### Example 54 Synthesis of Compound A149

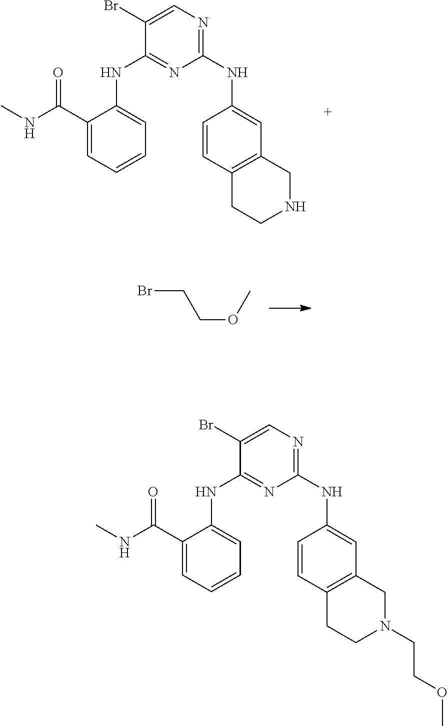

2-{5-Bromo-2-[2-(2-methoxy-ethyl)-1,2,3,4-tetrahydro-isoquinolin-7-ylamino]-pyrimidin-4-ylamino}-N-methyl-benzamide (A149). The synthetic procedure is similar to the synthesis of Compound A127 except 2-[5-bromo-2-(1,2,3,4-tetrahydro-isoquinolin-7-ylamino)-pyrimidin-4-ylamino]-N-methyl-benzamide and 1-bromo-2-methoxy-ethane were used instead of 2-[5-bromo-2-(1,2,3,4-tetrahydro-isoquinolin-6-ylamino)-pyrimidin-4-ylamino]-N-methyl-benzamide and 2-chloro-1-morpholin-4-yl-ethanone to obtain the desired product as an off-white solid (34 mg, 52%). ¹H-NMR (400 MHz, DMSO-d6): δ 11.41 (s, 1H), 9.97-9.82 (1H), 9.52 (s, 1H), 8.72 (d, J=4.1 Hz, 1H), 8.61 (d, J=8.2 Hz, 1H), 8.27 (s, 1H), 7.72 (d, J=6.9 Hz, 1H), 7.48-7.52 (m, 3H), 7.13 (t, J=9.2 Hz, 2H), 4.31 (d, J=27.0 Hz, 2H), 3.71 (t, J=4.8 Hz, 3H), 2.77 (d, J=4.6 Hz, 3H), 2.46-2.50 (m). Observed [M+1]+: 511.38.

### Example 55 Synthesis of Compound A150

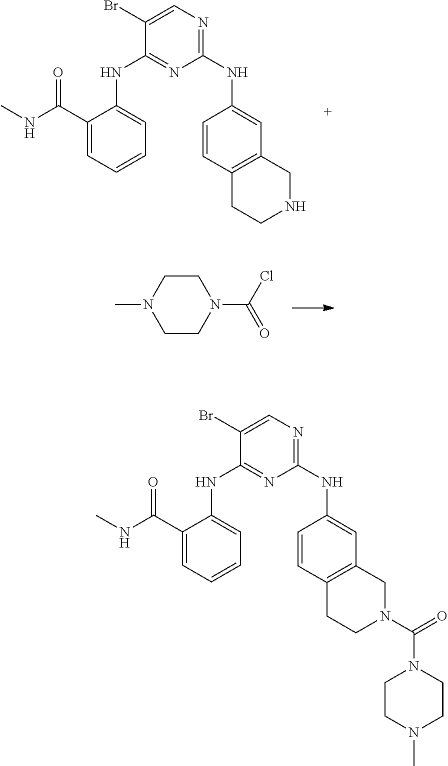

2-{5-Bromo-2-[2-(4-methyl-piperazine-1-carbonyl)-1,2,3,4-tetrahydro-isoquinolin-7-ylamino]-pyrimidin-4-ylamino}-N-methyl-benzamide (A150). The synthetic procedure is similar to the synthesis of Compound A128 except 2-[5-bromo-2-(1,2,3,4-tetrahydro-isoquinolin-7-ylamino)-pyrimidin-4-ylamino]-N-methyl-benzamide was used instead of 2-[5-bromo-2-(1,2,3,4-tetrahydro-isoquinolin-6-ylamino)-pyrimidin-4-ylamino]-N-methyl-benzamide to obtain the desired product as an off-white solid (23 mg, 36%). Observed [M+1]+: 579.54.

### Example 56 Synthesis of Compound A151

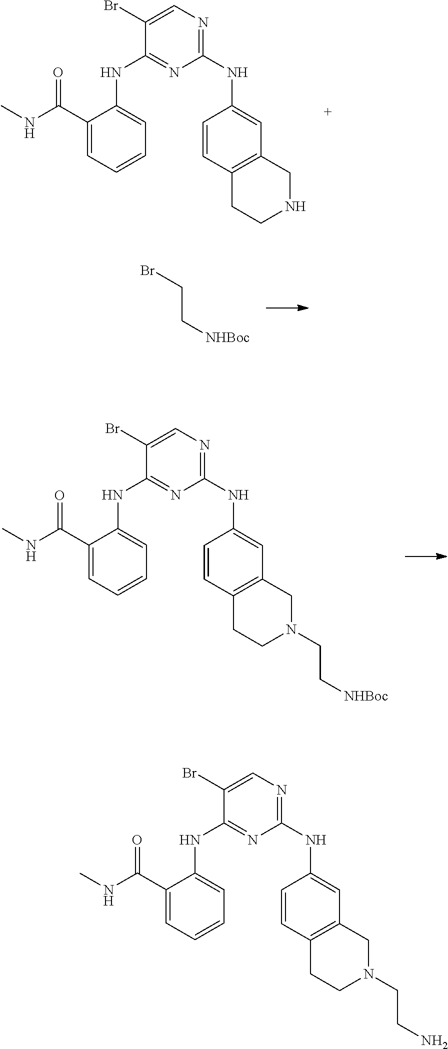 [SMILES: CNC(=O)c1ccccc1Nc1nc(Nc2ccc3c(c2)CNCC3)ncc1Br ; CC(C)(C)OC(=O)NCCBr ; CNC(=O)c1ccccc1Nc1nc(Nc2ccc3c(c2)CN(CCNC(=O)OC(C)(C)C)CC3)ncc1Br ; CNC(=O)c1ccccc1Nc1nc(Nc2ccc3c(c2)CN(CCN)CC3)ncc1Br]

2-{2-[2-(2-Amino-ethyl)-1,2,3,4-tetrahydro-isoquinolin-7-ylamino]-5-bromo-pyrimidin-4-ylamino}-N-methyl-benzamide (A151). The synthetic procedure is similar to the synthesis of Compound A145 except 2-[5-bromo-2-(1,2,3,4-tetrahydro-isoquinolin-7-ylamino)-pyrimidin-4-ylamino]-N-methyl-benzamide was used instead of 2-[5-bromo-2-(1,2,3,4-tetrahydro-isoquinolin-6-ylamino)-pyrimidin-4-ylamino]-N-methyl-benzamide to obtain the desired product as an off-white solid (21 mg, 39%). Observed [M+1]+: 496.40.

### Example 57 Synthesis of Compound A152

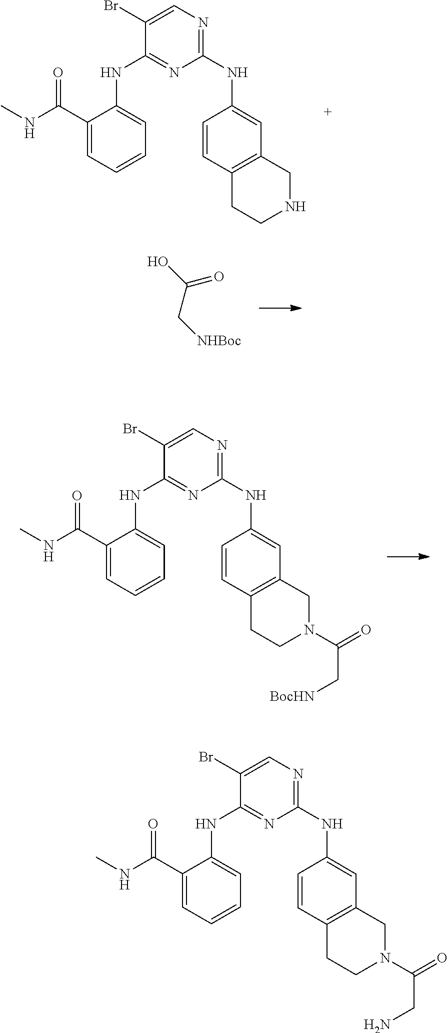 [SMILES: CNC(=O)c1ccccc1Nc1nc(Nc2ccc3c(c2)CNCC3)ncc1Br ; CC(C)(C)OC(=O)NCC(=O)O ; CNC(=O)c1ccccc1Nc1nc(Nc2ccc3c(c2)CN(C(=O)CNC(=O)OC(C)(C)C)CC3)ncc1Br ; CNC(=O)c1ccccc1Nc1nc(Nc2ccc3c(c2)CN(C(=O)CN)CC3)ncc1Br]

2-{2-[2-(2-Amino-acetyl)-1,2,3,4-tetrahydro-isoquinolin-7-ylamino]-5-bromo-pyrimidin-4-ylamino}-N-methyl-benzamide (A152). The synthetic procedure is similar to the synthesis of Compound A132 except 2-[5-bromo-2-(1,2,3,4-tetrahydro-isoquinolin-7-ylamino)-pyrimidin-4-ylamino]-N-methyl-benzamide was used instead of 2-[5-bromo-2-(1,2,3,4-tetrahydro-isoquinolin-6-ylamino)-pyrimidin-4-ylamino]-N-methyl-benzamide to obtain the desired product as an off-white solid (21 mg, 38%). Observed [M+1]+: 510.39.

### Example 58 Synthesis of Compound A153

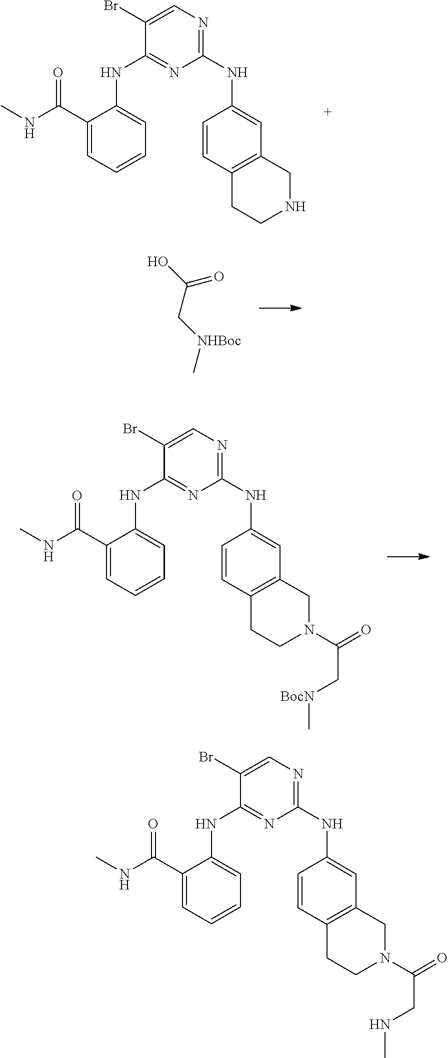

2-{5-Bromo-2-[2-(2-methylamino-acetyl)-1,2,3,4-tetrahydro-isoquinolin-7-ylamino]-pyrimidin-4-ylamino}-N-methyl-benzamide (A153). The synthetic procedure is similar to the synthesis of Compound A133 except 2-[5-bromo-2-(1,2,3,4-tetrahydro-isoquinolin-7-ylamino)-pyrimidin-4-ylamino]-N-methyl-benzamide was used instead of 2-[5-bromo-2-(1,2,3,4-tetrahydro-isoquinolin-6-ylamino)-pyrimidin-4-ylamino]-N-methyl-benzamide to obtain the desired product as an off-white solid (32 mg, 41%). ¹H-NMR (400 MHz, DMSO-d6): δ 11.29 (s, 1H), 9.38 (s, 1H), 8.72 (d, J=4.6 Hz, 1H), 8.64-8.50 (1H), 8.25 (d, J=1.8 Hz, 1H), 7.69 (d, J=7.8 Hz, 1H), 7.46-7.54 (m, 1H), 7.40-7.26 (1H), 7.11 (s, 1H), 7.00-7.04 (m, 1H), 4.46-4.64 (m, 2H), 3.55-3.71 (m, 2H), 2.77 (d, J=4.6 Hz, 3H), 2.40-2.55 (m), 2.16-2.28 (m, 2H). Observed [M+1]+: 524.31.

### Example 59 Synthesis of Compound A155

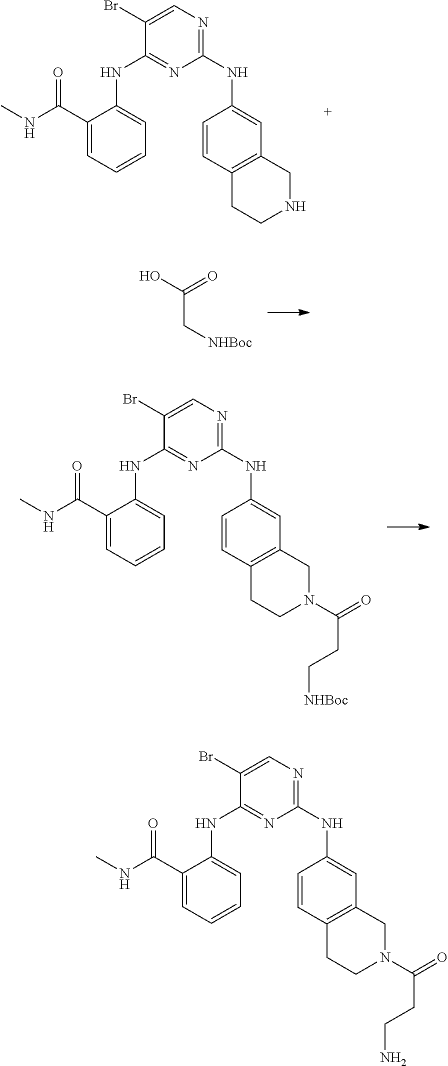 [SMILES: CNC(=O)c1ccccc1Nc1nc(Nc2ccc3c(c2)CNCC3)ncc1Br ; CC(C)(C)OC(=O)NCC(=O)O ; CNC(=O)c1ccccc1Nc1nc(Nc2ccc3c(c2)CN(C(=O)CCNC(=O)OC(C)(C)C)CC3)ncc1Br ; CNC(=O)c1ccccc1Nc1nc(Nc2ccc3c(c2)CN(C(=O)CCN)CC3)ncc1Br]

2-{2-[2-(3-Amino-propionyl)-1,2,3,4-tetrahydro-isoquinolin-7-ylamino]-5-bromo-pyrimidin-4-ylamino}-N-methyl-benzamide (A155). The synthetic procedure is similar to the synthesis of Compound A136 except 2-[5-bromo-2-(1,2,3,4-tetrahydro-isoquinolin-7-ylamino)-pyrimidin-4-ylamino]-N-methyl-benzamide was used instead of 2-[5-bromo-2-(1,2,3,4-tetrahydro-isoquinolin-6-ylamino)-pyrimidin-4-ylamino]-N-methyl-benzamide to obtain the desired product as an off-white solid (24 mg, 42%). Observed [M+1]+: 524.42.

### Example 60 Synthesis of Compound A154

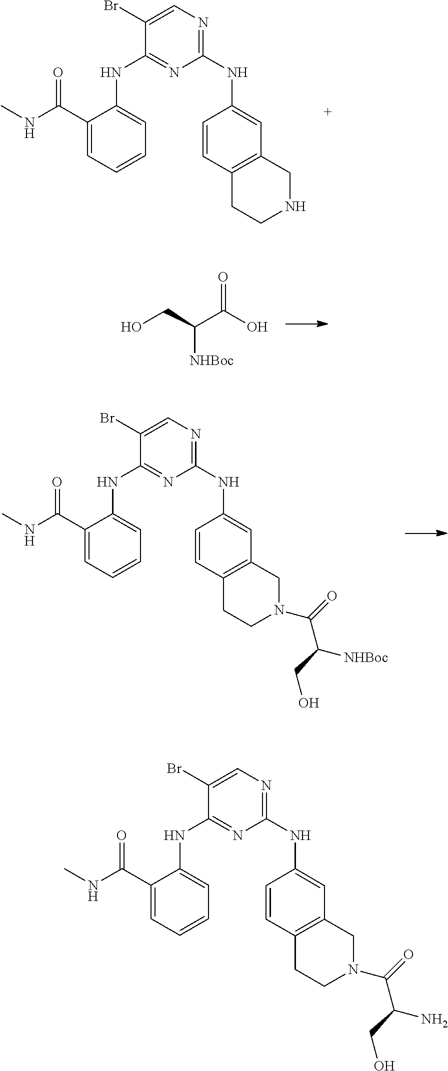 [SMILES: CNC(=O)c1ccccc1Nc1nc(Nc2ccc3c(c2)CNCC3)ncc1Br ; CC(C)(C)OC(=O)N[C@@H](CO)C(=O)O ; CNC(=O)c1ccccc1Nc1nc(Nc2ccc3c(c2)CN(C(=O)[C@H](CO)NC(=O)OC(C)(C)C)CC3)ncc1Br ; CNC(=O)c1ccccc1Nc1nc(Nc2ccc3c(c2)CN(C(=O)[C@@H](N)CO)CC3)ncc1Br]

2-{2-[2-(2-Amino-3-hydroxy-propionyl)-1,2,3,4-tetrahydro-isoquinolin-7-ylamino]-5-bromo-pyrimidin-4-ylamino}-N-methyl-benzamide (A154). The synthetic procedure is similar to the synthesis of Compound A139 except 2-[5-bromo-2-(1,2,3,4-tetrahydro-isoquinolin-7-ylamino)-pyrimidin-4-ylamino]-N-methyl-benzamide was used instead of 2-[5-bromo-2-(1,2,3,4-tetrahydro-isoquinolin-6-ylamino)-pyrimidin-4-ylamino]-N-methyl-benzamide to obtain the desired product as an off-white solid (29 mg, 49%). ¹H-NMR (400 MHz, DMSO-d6): δ 11.30 (s, 1H), 9.41 (d, J=8.7 Hz, 1H), 8.73 (s, 1H), 8.56 (s, 1H), 8.25 (s, 1H), 7.70 (d, J=7.3 Hz, 1H), 7.51 (d, J=15.6 Hz, 1H), 7.33 (d, J=7.8 Hz, 1H), 7.11 (s, 1H), 7.01 (d, J=7.8 Hz, 1H), 4.46-4.67 (m, 2H), 3.59-3.74 (m, 2H), 3.37-3.47 (m, 1H), 2.77 (d, J=4.1 Hz, 3H), 2.45-2.47 (m, 3H). Observed [M+1]+: 540.57.

### Example 61 Synthesis of Compound A156

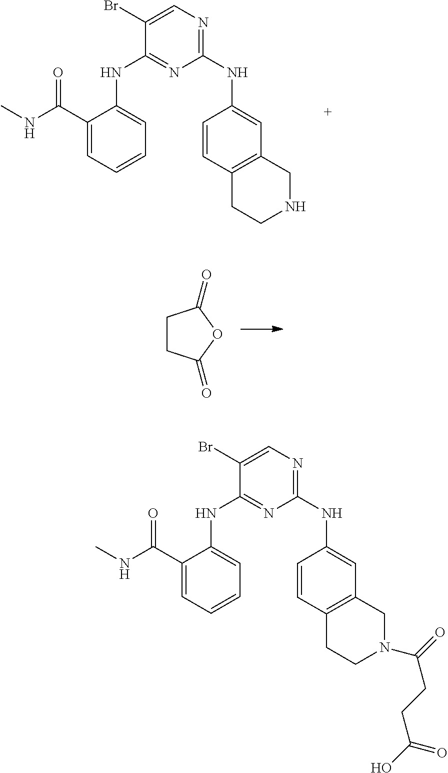 [SMILES: CNC(=O)c1ccccc1Nc1nc(Nc2ccc3c(c2)CNCC3)ncc1Br ; O=C1CCC(=O)O1 ; CNC(=O)c1ccccc1Nc1nc(Nc2ccc3c(c2)CN(C(=O)CCC(=O)O)CC3)ncc1Br]

4-{7-[5-Bromo-4-(2-methylcarbamoyl-phenylamino)-pyrimidin-2-ylamino]-3,4-dihydro-1H-isoquinolin-2-yl}-4-oxo-butyric acid (A156). The synthetic procedure is similar to the synthesis of Compound A134 except 2-[5-bromo-2-(1,2,3,4-tetrahydro-isoquinolin-7-ylamino)-pyrimidin-4-ylamino]-N-methyl-benzamide was used instead of 2-[5-bromo-2-(1,2,3,4-tetrahydro-isoquinolin-6-ylamino)-pyrimidin-4-ylamino]-N-methyl-benzamide to obtain the desired product as an off-white solid (26 mg, 43%). ¹H-NMR (400 MHz, DMSO-d6): δ 12.10 (s, 1H), 11.30 (s, 1H), 9.44 (d, J=13.3 Hz, 1H), 8.72 (s, 1H), 8.55 (d, J=6.0 Hz, 1H), 8.26 (s, 1H), 7.70 (d, J=7.8 Hz, 1H), 7.58 (s, 1H), 7.49 (q, J=8.7 Hz, 1H), 7.33 (d, J=7.3 Hz, 1H), 7.10-7.16 (m, 1H), 7.03 (d, J=8.2 Hz, 1H), 5.72 (d, J=1.4 Hz, 1H), 4.48 (d, J=16.5 Hz, 1H), 3.63 (q, J=6.4 Hz, 1H), 2.77 (d, J=3.7 Hz, 3H), 2.65 (m), 2.38-2.50 (m). Observed [M+1]+: 553.41.

### Example 62 Synthesis of Compound A157

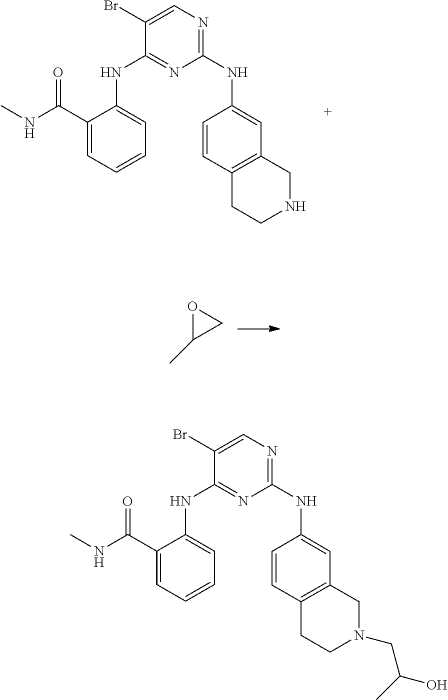

2-{5-Bromo-2-[2-(2-hydroxy-propyl)-1,2,3,4-tetrahydro-isoquinolin-7-ylamino]-pyrimidin-4-ylamino}-N-methyl-benzamide (A157). The synthetic procedure is similar to the synthesis of Compound A138 except 2-[5-bromo-2-(1,2,3,4-tetrahydro-isoquinolin-7-ylamino)-pyrimidin-4-ylamino]-N-methyl-benzamide was used instead of 2-[5-bromo-2-(1,2,3,4-tetrahydro-isoquinolin-6-ylamino)-pyrimidin-4-ylamino]-N-methyl-benzamide to obtain the desired product as an off-white solid (24 mg, 43%). Observed [M+1]+: 511.41.

### Example 63 Synthesis of Compound A158

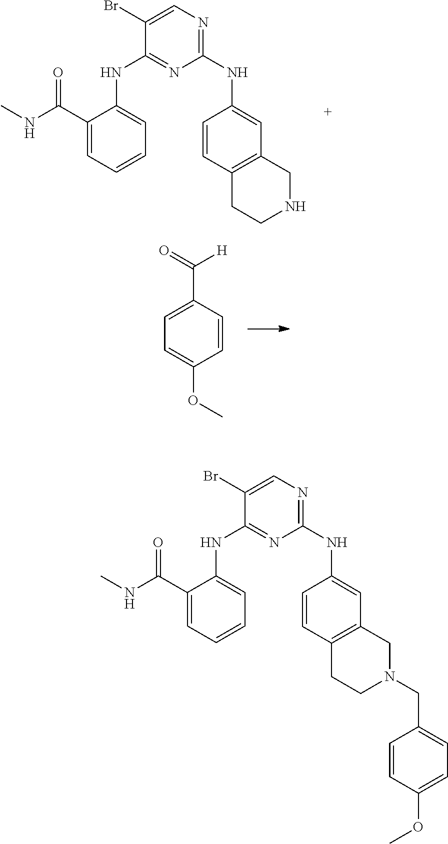

2-{5-Bromo-2-[2-(4-methoxy-benzyl)-1,2,3,4-tetrahydro-isoquinolin-7-ylamino]-pyrimidin-4-ylamino}-N-methyl-benzamide (A158). The synthetic procedure is similar to the synthesis of Compound A129 except 2-[5-bromo-2-(1,2,3,4-tetrahydro-isoquinolin-7-ylamino)-pyrimidin-4-ylamino]-N-methyl-benzamide was used instead of 2-[5-bromo-2-(1,2,3,4-tetrahydro-isoquinolin-6-ylamino)-pyrimidin-4-ylamino]-N-methyl-benzamide to obtain the desired product as an off-white solid (23 mg, 36%). Observed [M+1]+: 573.34.

### Example 64 Synthesis of Compound A159

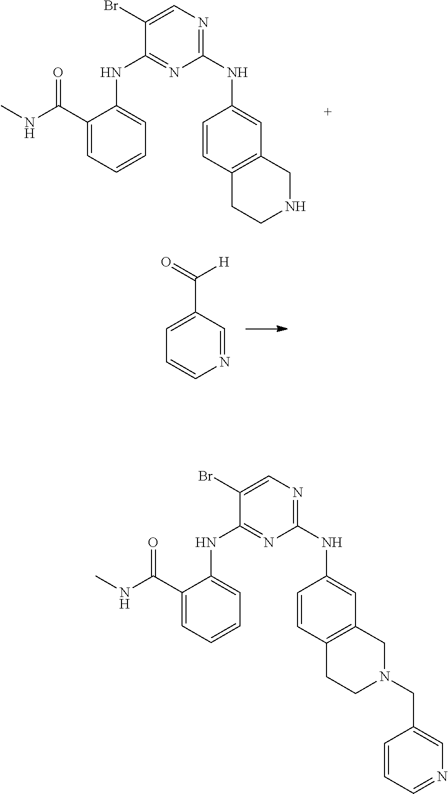 [SMILES: CNC(=O)c1ccccc1Nc1nc(Nc2ccc3c(c2)CNCC3)ncc1Br ; O=Cc1cccnc1 ; CNC(=O)c1ccccc1Nc1nc(Nc2ccc3c(c2)CN(Cc2cccnc2)CC3)ncc1Br]

2-[5-Bromo-2-(2-pyridin-3-ylmethyl-1,2,3,4-tetrahydro-isoquinolin-7-ylamino)-pyrimidin-4-ylamino]-N-methyl-benzamide (A159). The synthetic procedure is similar to the synthesis of Compound A130 except 2-[5-bromo-2-(1,2,3,4-tetrahydro-isoquinolin-7-ylamino)-pyrimidin-4-ylamino]-N-methyl-benzamide was used instead of 2-[5-bromo-2-(1,2,3,4-tetrahydro-isoquinolin-6-ylamino)-pyrimidin-4-ylamino]-N-methyl-benzamide to obtain the desired product as an off-white solid (24 mg, 40%). Observed [M+1]+: 544.30.

### Example 65 Synthesis of Compound A160

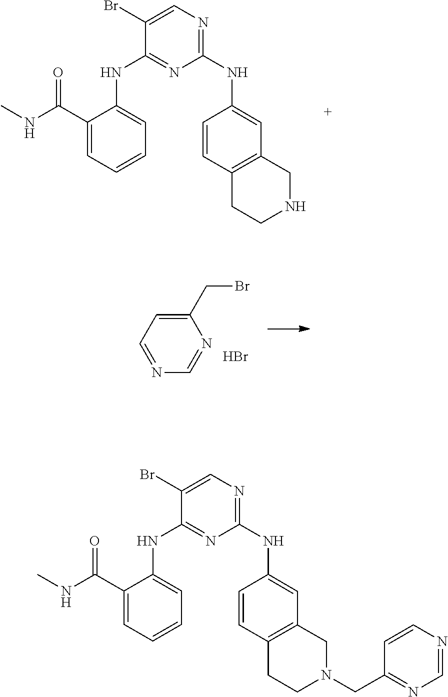 [SMILES: CNC(=O)c1ccccc1Nc1nc(Nc2ccc3c(c2)CNCC3)ncc1Br ; BrCc1ccncn1 ; Br ; CNC(=O)c1ccccc1Nc1nc(Nc2ccc3c(c2)CN(Cc2ccncn2)CC3)ncc1Br]

2-[5-Bromo-2-(2-pyrimidin-4-ylmethyl-1,2,3,4-tetrahydro-isoquinolin-7-ylamino)-pyrimidin-4-ylamino]-N-methyl-benzamide (A160). The synthetic procedure is similar to the synthesis of Compound A146 except 2-[5-bromo-2-(1,2,3,4-tetrahydro-isoquinolin-7-ylamino)-pyrimidin-4-ylamino]-N-methyl-benzamide was used instead of 2-[5-bromo-2-(1,2,3,4-tetrahydro-isoquinolin-6-ylamino)-pyrimidin-4-ylamino]-N-methyl-benzamide to obtain the desired product as an off-white solid (42 mg, 58%). ¹H-NMR (400 MHz, DMSO-D6) δ 11.27 (s, 1H), 9.33 (s, 1H), 9.11 (d, J=0.9 Hz, 1H), 8.75 (d, J=5.5 Hz, 1H), 8.23-8.27 (m), 7.66 (d, J=6.4 Hz, 1H), 7.60 (d, J=4.1 Hz, 1H), 7.28-7.37 (m), 6.97-7.00 (m), 3.77 (s, 2H), 3.52-3.57 (m), 2.75 (q, J=4.9 Hz, 3H), 2.47 (q, J=1.8 Hz, 4H), 1.15-1.23 (m, 4H). Observed [M+1]+: 545.54.

### Example 66 Synthesis of Compound A161

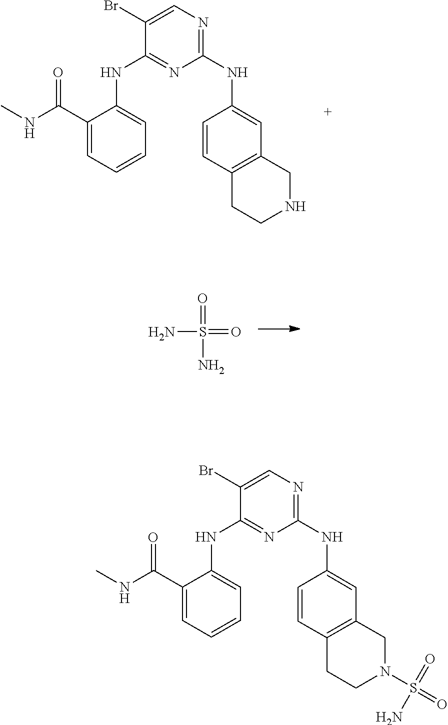 [SMILES: CNC(=O)c1ccccc1Nc1nc(Nc2ccc3c(c2)CNCC3)ncc1Br ; NS(N)(=O)=O ; CNC(=O)c1ccccc1Nc1nc(Nc2ccc3c(c2)CN(S(N)(=O)=O)CC3)ncc1Br]

2-[5-Bromo-2-(2-sulfamoyl-1,2,3,4-tetrahydro-isoquinolin-7-ylamino)-pyrimidin-4-ylamino]-N-methyl-benzamide (A161). The synthetic procedure is similar to the synthesis of Compound A140 except 2-[5-bromo-2-(1,2,3,4-tetrahydro-isoquinolin-7-ylamino)-pyrimidin-4-ylamino]-N-methyl-benzamide was used instead of 2-[5-bromo-2-(1,2,3,4-tetrahydro-isoquinolin-6-ylamino)-pyrimidin-4-ylamino]-N-methyl-benzamide to obtain the desired product as an off-white solid (23 mg, 39%). Observed [M+1]+: 532.20.

### Example 67 Synthesis of Compound A162

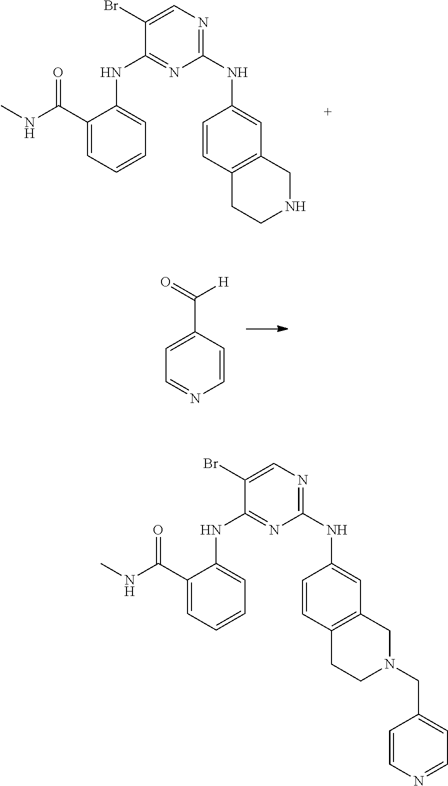 [SMILES: CNC(=O)c1ccccc1Nc1nc(Nc2ccc3c(c2)CNCC3)ncc1Br ; O=Cc1ccncc1 ; CNC(=O)c1ccccc1Nc1nc(Nc2ccc3c(c2)CN(Cc2ccncc2)CC3)ncc1Br]

2-[5-Bromo-2-(2-pyridin-4-ylmethyl-1,2,3,4-tetrahydro-isoquinolin-7-ylamino)-pyrimidin-4-ylamino]-N-methyl-benzamide (A162). The synthetic procedure is similar to the synthesis of Compound A131 except 2-[5-bromo-2-(1,2,3,4-tetrahydro-isoquinolin-7-ylamino)-pyrimidin-4-ylamino]-N-methyl-benzamide was used instead of 2-[5-bromo-2-(1,2,3,4-tetrahydro-isoquinolin-6-ylamino)-pyrimidin-4-ylamino]-N-methyl-benzamide to obtain the desired product as an off-white solid (29 mg, 48%). ¹H-NMR (400 MHz, DMSO-d6): δ 11.31 (s, 1H), 9.33 (s, 1H), 8.78 (d, J=4.6 Hz, 1H), 8.44-8.51 (m, 2H), 8.22 (s, 1H), 7.69-7.71 (m, 1H), 7.36 (d, J=6.0 Hz, 2H), 7.27 (d, J=8.2 Hz, 1H), 6.97 (t, J=8.0 Hz, 1H), 3.64 (s, 1H), 3.42 (s, 1H), 2.73-2.76 (m, 3H), 2.46 (t, J=1.8 Hz, 2H). Observed [M+1]+: 544.42.

### Example 68 Synthesis of Compound A163

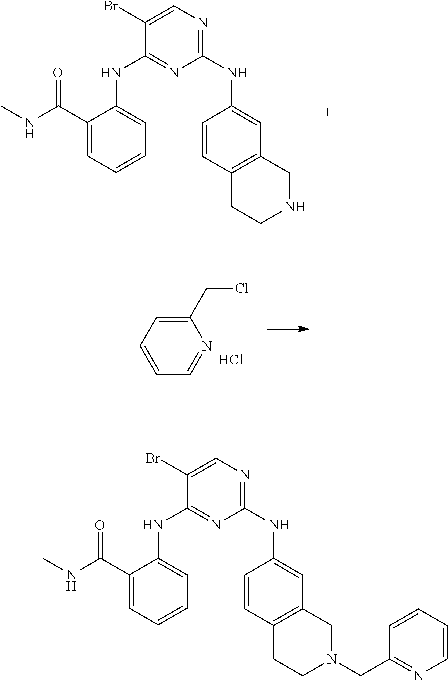 [SMILES: CNC(=O)c1ccccc1Nc1nc(Nc2ccc3c(c2)CNCC3)ncc1Br ; ClCc1ccccn1 ; Cl ; CNC(=O)c1ccccc1Nc1nc(Nc2ccc3c(c2)CN(Cc2ccccn2)CC3)ncc1Br]

2-[5-Bromo-2-(2-pyridin-2-ylmethyl-1,2,3,4-tetrahydro-isoquinolin-7-ylamino)-pyrimidin-4-ylamino]-N-methyl-benzamide (A163). The synthetic procedure is similar to the synthesis of Compound A144 except 2-[5-bromo-2-(1,2,3,4-tetrahydro-isoquinolin-7-ylamino)-pyrimidin-4-ylamino]-N-methyl-benzamide was used instead of 2-[5-bromo-2-(1,2,3,4-tetrahydro-isoquinolin-6-ylamino)-pyrimidin-4-ylamino]-N-methyl-benzamide to obtain the desired product as an off-white solid (25 mg, 42%). ¹H-NMR (400 MHz, DMSO-d6): δ 11.26 (s, 1H), 9.31 (s, 1H), 8.69 (d, J=4.6 Hz, 1H), 8.49-8.53 (m, 2H), 8.22 (s, 1H), 7.75 (td, J=7.7, 1.7 Hz, 1H), 7.66 (dd, J=7.8, 1.4 Hz, 1H), 7.47 (d, J=7.8 Hz, 1H), 7.24-7.35 (m, 5H), 6.95-7.02 (m, 3H), 3.73 (s, 3H), 3.47 (s, 2H), 2.69-2.77 (m, 9H), 2.45-2.47 (m, 4H). Observed [M+1]+: 544.44.

### Example 69 Synthesis of Compound A164

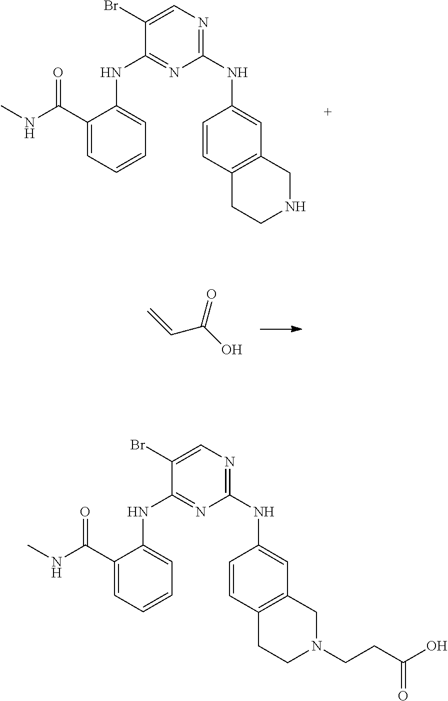

3-{7-[5-Bromo-4-(2-methylcarbamoyl-phenylamino)-pyrimidin-2-ylamino]-3,4-dihydro-1H-isoquinolin-2-yl}-propionic acid (A164). The synthetic procedure is similar to the synthesis of Compound A141 except 2-[5-bromo-2-(1,2,3,4-tetrahydro-isoquinolin-7-ylamino)-pyrimidin-4-ylamino]-N-methyl-benzamide was used instead of 2-[5-bromo-2-(1,2,3,4-tetrahydro-isoquinolin-6-ylamino)-pyrimidin-4-ylamino]-N-methyl-benzamide to obtain the desired product as an off-white solid (27 mg, 47%). ¹H-NMR (400 MHz, DMSO-d6): δ 11.34 (s, 1H), 9.45 (s, 1H), 8.74 (d, J=4.6 Hz, 1H), 8.58 (d, J=7.8 Hz, 1H), 8.26 (d, J=6.4 Hz, 1H), 7.72 (d, J=7.8 Hz, 1H), 7.40-7.52 (m, 4H), 7.03-7.14 (m, 3H), 3.95 (s, 2H), 3.09-3.16 (m, 2H), 2.87 (d, J=16.0 Hz), 2.68-2.77 (m), 2.45-2.47 (m). Observed [M+1]+: 525.35.

### Example 70 Synthesis of Example A165

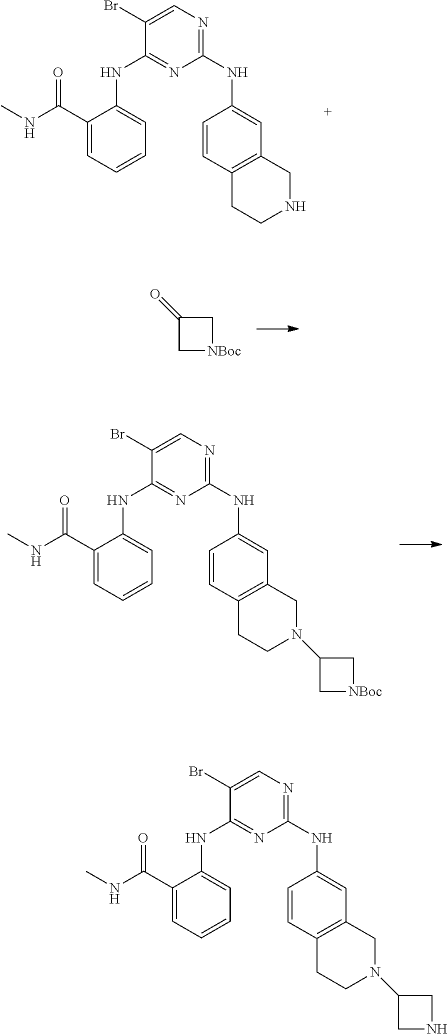 [SMILES: CNC(=O)c1ccccc1Nc1nc(Nc2ccc3c(c2)CNCC3)ncc1Br ; CC(C)(C)OC(=O)N1CC(=O)C1 ; CNC(=O)c1ccccc1Nc1nc(Nc2ccc3c(c2)CN(C2CN(C(=O)OC(C)(C)C)C2)CC3)ncc1Br ; CNC(=O)c1ccccc1Nc1nc(Nc2ccc3c(c2)CN(C2CNC2)CC3)ncc1Br]

3-{7-[5-Bromo-4-(2-methylcarbamoyl-phenylamino)-pyrimidin-2-ylamino]-3,4-dihydro-1H-isoquinolin-2-yl}-propionic acid (A165). The synthetic procedure is similar to the synthesis of Compound A137 except 2-[5-bromo-2-(1,2,3,4-tetrahydro-isoquinolin-7-ylamino)-pyrimidin-4-ylamino]-N-methyl-benzamide was used instead of 2-[5-bromo-2-(1,2,3,4-tetrahydro-isoquinolin-6-ylamino)-pyrimidin-4-ylamino]-N-methyl-benzamide to obtain the desired product as an off-white solid (24 mg, 42%). Observed [M+1]+: 508.33.

### Example 71 Synthesis of Compound A166

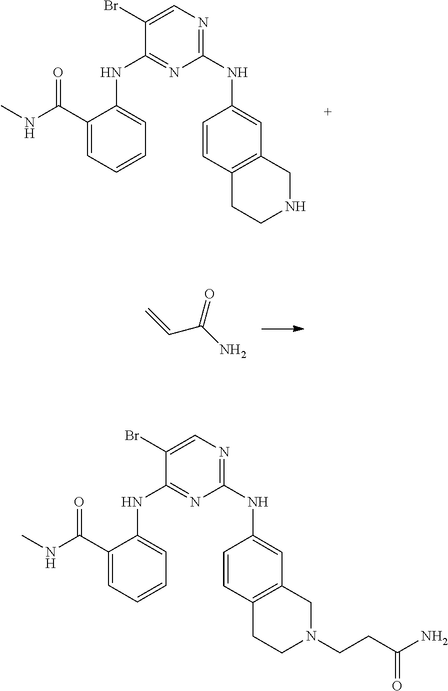

2-{5-Bromo-2-[2-(2-carbamoyl-ethyl)-1,2,3,4-tetrahydro-isoquinolin-7-ylamino]-pyrimidin-4-ylamino}-N-methyl-benzamide (A166). The synthetic procedure is similar to the synthesis of Compound A142 except 2-[5-bromo-2-(1,2,3,4-tetrahydro-isoquinolin-7-ylamino)-pyrimidin-4-ylamino]-N-methyl-benzamide was used instead of 2-[5-bromo-2-(1,2,3,4-tetrahydro-isoquinolin-6-ylamino)-pyrimidin-4-ylamino]-N-methyl-benzamide to obtain the desired product as an off-white solid (28 mg, 48%). ¹H-NMR (400 MHz, DMSO-d6): δ 11.36 (s, 1H), 10.50 (s, 1H), 9.51 (s, 1H), 8.75 (d, J=4.6 Hz, 1H), 8.59 (d, J=8.2 Hz, 1H), 8.26 (d, J=4.6 Hz, 1H), 7.73 (dd, J=7.8, 1.4 Hz, 1H), 7.60 (s, 1H), 7.47-7.54 (m, 2H), 7.08-7.15 (m, 3H), 2.74-2.78 (d, 3H), 2.45-2.47 (m). Observed [M+1]+: 524.26.

### Example 72 Synthesis of Compound A167

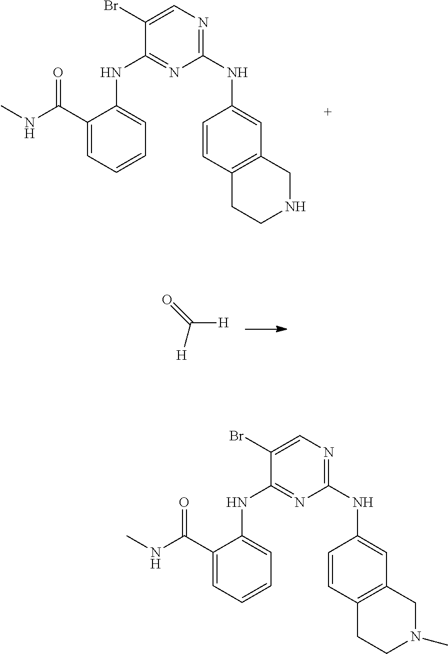

2-[5-Bromo-2-(2-methyl-1,2,3,4-tetrahydro-isoquinolin-7-ylamino)-pyrimidin-4-ylamino]-N-methyl-benzamide (A167). The synthetic procedure is similar to the synthesis of Compound A135 except 2-[5-bromo-2-(1,2,3,4-tetrahydro-isoquinolin-7-ylamino)-pyrimidin-4-ylamino]-N-methyl-benzamide was used instead of 2-[5-bromo-2-(1,2,3,4-tetrahydro-isoquinolin-6-ylamino)-pyrimidin-4-ylamino]-N-methyl-benzamide to obtain the desired product as an off-white solid (24 mg, 47%). Observed [M+1]+: 467.25.

### Example 73 Synthesis of Compound A168

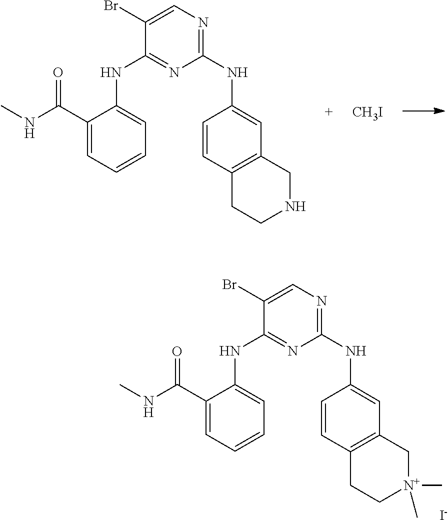

7-[5-Bromo-4-(2-methylcarbamoyl-phenylamino)-pyrimidin-2-ylamino]-2,2-dimethyl-1,2,3,4-tetrahydro-isoquinolinium; iodide (A168). The synthetic procedure is similar to the synthesis of Compound A143 except 2-[5-bromo-2-(1,2,3,4-tetrahydro-isoquinolin-7-ylamino)-pyrimidin-4-ylamino]-N-methyl-benzamide was used instead of 2-[5-bromo-2-(1,2,3,4-tetrahydro-isoquinolin-6-ylamino)-pyrimidin-4-ylamino]-N-methyl-benzamide to obtain the desired product as an off-white solid (14 mg, 27%). Observed [M+1]+: 481.29.

### Example 74 Synthesis of Compound C28

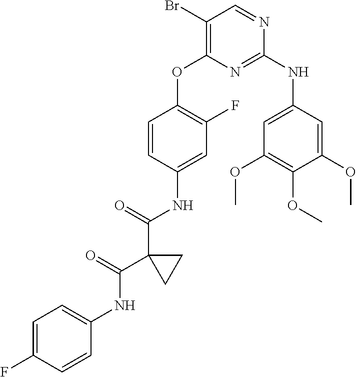 [SMILES: COc1cc(Nc2ncc(Br)c(Oc3ccc(NC(=O)C4(C(=O)Nc5ccc(F)cc5)CC4)cc3F)n2)cc(OC)c1OC]

N-(4-((5-Bromo-2-((3,4,5-trimethoxyphenyl)amino)pyrimidin-4-yl)oxy)-3-fluorophenyl)-N-(4-fluorophenyl)cyclopropane-1,1-dicarboxamide (C28). N-(4-((5-Bromo-2-chloropyrimidin-4-yl)oxy)-3-fluorophenyl)-N-(4-fluorophenyl)cyclopropane-1,1-dicarboxamide (0.220 g, 0.420 mmol) and 3,4,5-trimethoxyaniline (115 mg, 0.630 mmol) were dissolved in n-butanol (4 mL) and heated at 120° C. until completion by LCMS. The reaction mixture was concentrated in vacuo and purified by column chromatography. Solid (125 mg, 44%). ¹H-NMR (400 MHz, DMSO-d6) δ 10.31 (s, 1H), 9.96 (s, 1H), 9.58 (s, 1H), 8.52 (d, J=3.7 Hz, 1H), 7.77 (dd, J=12.8, 2.3 Hz, 1H), 7.59-7.63 (m, 2H), 7.34-7.42 (m, 2H), 7.08-7.15 (m, 2H), 6.81 (s, 2H), 3.47-3.55 (m, 9H), 1.39-1.46 (m, 4H). Observed [M+1]+: 670.10

### Example 75 Synthesis of Compound E28

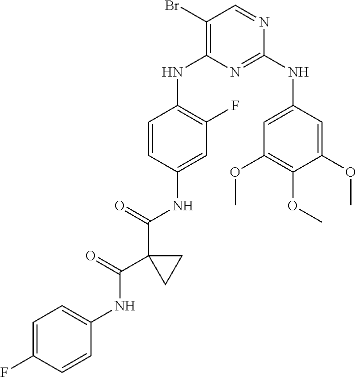 [SMILES: COc1cc(Nc2ncc(Br)c(Nc3ccc(NC(=O)C4(C(=O)Nc5ccc(F)cc5)CC4)cc3F)n2)cc(OC)c1OC]

N-(4-((5-Bromo-2-((3,4,5-trimethoxyphenyl)amino)pyrimidin-4-yl)amino)-3-fluorophenyl)-N-(4-fluorophenyl)cyclopropane-1,1-dicarboxamide (E28). N-(4-((5-Bromo-2-chloropyrimidin-4-yl)amino)-3-fluorophenyl)-N-(4-fluorophenyl)cyclopropane-1,1-dicarboxamide (97.1 mg, 0.186 mmol) and 3,4,5-trimethoxyaniline (68.1 mg, 0.372 mmol) were dissolved in n-butanol (4 mL) and heated at reflux until completion by LCMS. The reaction mixture was concentrated in vacuo and purified by column chromatography. Solid (89 mg, 72%). ¹H-NMR (400 MHz, DMSO-d6): δ 10.25 (s, 1H), 9.97 (s, 1H), 9.04 (s, 1H), 8.41 (s, 1H), 8.15 (s, 1H), 7.59-7.67 (m, 2H), 7.32-7.43 (m, 2H), 7.11 (m, 2H), 6.86 (s, 2H), 5.82 (s, 1H), 3.45-3.71 (m, 9H), 1.44 (m, 4H). Observed [M+1]⁺: 669.12.

### Example 76 Synthesis of Compound C29

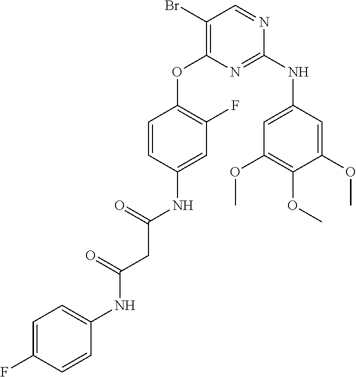 [SMILES: COc1cc(Nc2ncc(Br)c(Oc3ccc(NC(=O)CC(=O)Nc4ccc(F)cc4)cc3F)n2)cc(OC)c1OC]

N¹-(4-((5-Bromo-2-((3,4,5-trimethoxyphenyl)amino)pyrimidin-4-yl)oxy)-3-fluorophenyl)-N³-(4-fluorophenyl)malonamide (C29). N1-(4-((5-Bromo-2-chloropyrimidin-4-yl)oxy)-3-fluorophenyl)-N3-(4-fluorophenyl)malonamide (135 mg, 0.271 mmol) and 3,4,5-trimethoxyaniline (99.4 mg, 0.543 mmol) were dissolved in n-butanol (4 mL) and heated at 120° C. until completion by LCMS. The reaction mixture was concentrated in vacuo and purified by column chromatography. Solid (68 mg, 39%). ¹H NMR (400 MHz, DMSO-d₆) δ 10.49 (s, 1H), 10.26 (s, 1H), 8.52 (s, 1H), 7.73-7.77 (m, 1H), 7.59 (m, 2H), 7.33-7.41 (m, 2H), 7.13 (m, 2H), 6.80 (s, 2H), 3.47-3.55 (m, 12H). Observed [M+1]⁺: 644.11.

### Example 77 Synthesis of Compound E14

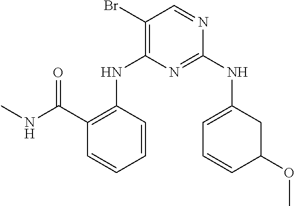

2-((5-Bromo-2-((3-methoxyphenyl)amino)pyrimidin-4-yl)amino)-N-methylbenzamide (E14). A solution of 2-((5-bromo-2-chloropyrimidin-4-yl)amino)-N-methylbenzamide (256 mg, 0.749 mmol) and 3-methoxyaniline (143 mg, 1.16 mmol) in 1:1 butanol/acetic acid (2 mL) was microwaved at 120° C. for 10 min similar to Method 5a. The reaction mixture was concentrated in vacuo and the crude product was purified by automated liquid chromatography to afford the title compound as a solid (38 mg, 11%). ¹H NMR (400 MHz, DMSO-d6) δ 11.36 (s, 1H), 9.40 (s, 1H), 8.64-8.72 (m, 2H), 8.27 (s, 1H), 7.70 (dd, J=8.2, 1.4 Hz, 1H), 7.41-7.46 (m, 1H), 7.28 (d, J=2.3 Hz, 1H), 7.22 (d, J=7.8 Hz, 1H), 7.09-7.14 (m, 2H), 6.51 (m, 1H), 3.65 (s, 3H), 2.77 (d, J=4.6 Hz, 3H). Observed [M+1]⁺: 428.06.

### Example 78 Synthesis of Compound E15

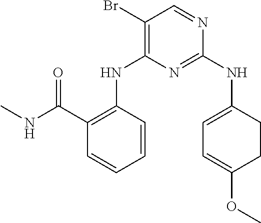

2-((5-Bromo-2-((4-methoxyphenyl)amino)pyrimidin-4-yl)amino)-N-methylbenzamide (E15). A solution of 2-((5-bromo-2-chloropyrimidin-4-yl)amino)-N-methylbenzamide (250 mg, 0.732 mmol), and 4-methoxyaniline (135 mg, 1.10 mmol) in n-butanol:AcOH (1:1, 6 mL) was heated at 120° C. for 10 min, similar to Method 5a. The reaction mixture was concentrated in vacuo and the crude product was purified by automated liquid chromatography to afford the title compound as a solid (54 mg, 17%). ¹H NMR (400 MHz, DMSO-d₆): δ 11.30 (s, 1H), 9.23 (s, 1H), 8.62-8.70 (m, 2H), 8.20 (s, 1H), 7.68 (dd, J=7.8, 1.4 Hz, 1H), 7.40-7.49 (m, 3H), 7.07-7.11 (m, 1H), 6.82 (td, J=6.2, 3.7 Hz, 2H), 3.69 (s, 3H), 2.77 (d, J=4.6 Hz, 3H). Observed [M+1]⁺: 428.06.

### Example 79 Synthesis of Compound E16

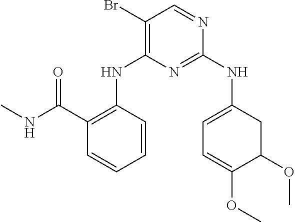

2-((5-Bromo-2-((3,4-dimethoxyphenyl)amino)pyrimidin-4-yl)amino)-N-methylbenzamide (E16). A solution of 2-((5-bromo-2-chloropyrimidin-4-yl)amino)-N-methylbenzamide (300 mg, 0.878 mmol) and 3,4-dimethoxyaniline (202 mg, 1.32 mmol) in n-butanol:AcOH (1:1, 6 mL) was heated at 120° C. for 2 h. The reaction mixture was then concentrated in vacuo and the crude product was purified by automated liquid chromatography to afford the title compound as a tan solid (53 mg, 13%). ¹H NMR (400 MHz, DMSO-d6): δ 11.36 (s, 1H), 9.21 (s, 1H), 8.64-8.71 (m, 2H), 8.22 (s, 1H), 7.69 (dd, J=7.8, 1.4 Hz, 1H), 7.39 (t, J=7.8 Hz, 1H), 7.07-7.19 (m, 3H), 6.82 (d, J=8.7 Hz, 1H), 3.66-3.71 (m, 3H), 3.60 (s, 3H), 2.77 (d, J=4.6 Hz, 3H). Observed [M+1]+: 458.07.

### Example 80 Synthesis of Compound E17

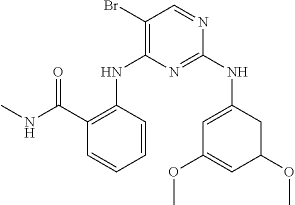

2-((5-Bromo-2-((3,5-dimethoxyphenyl)amino)pyrimidin-4-yl)amino)-N-methylbenzamide (E17). A solution of 2-((5-bromo-2-chloropyrimidin-4-yl)amino)-N-methylbenzamide (300 mg, 0.878 mmol), and 3,5-dimethoxyaniline (202 mg, 1.32 mmol) in n-butanol/AcOH (1:1, 6 mL) was heated at 120° C. for 2 h, similar to Method 5a. The reaction mixture was then concentrated in vacuo and the crude product was purified by automated liquid chromatography to afford the title compound as a tan solid (53 mg, 13%). ¹H NMR (400 MHz, DMSO-d6): δ 11.40 (s, 1H), 9.36 (s, 1H), 8.67-8.72 (m, 2H), 8.27 (s, 1H), 7.69 (dd, J=7.8, 1.4 Hz, 1H), 7.40-7.45 (m, 1H), 7.08-7.12 (m, 1H), 6.90 (d, J=2.3 Hz, 2H), 6.10 (t, J=2.3 Hz, 1H), 3.63-3.68 (m, 6H), 2.77 (d, J=4.6 Hz, 3H). Observed [M+1]+: 458.07.

### Example 81 Synthesis of Compound E18

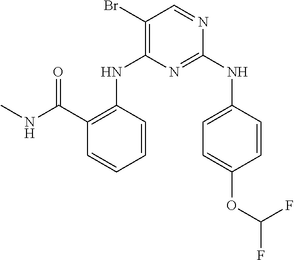 [SMILES: CNC(=O)c1ccccc1Nc1nc(Nc2ccc(OC(F)F)cc2)ncc1Br]

2-((5-Bromo-2-((4-(difluoromethoxy)phenyl)amino)pyrimidin-4-yl)amino)-N-methylbenzamide (E18). A solution of 2-((5-bromo-2-chloropyrimidin-4-yl)amino)-N-methylbenzamide (300 mg, 0.878 mmol), and 4-(difluoromethoxy)aniline (0.16 mL, 1.32 mmol) in n-butanol/AcOH (1:1, 6 mL) was heated at 120° C. for 2 h. The reaction mixture was then concentrated in vacuo and the crude product was purified by automated liquid chromatography to afford the title compound as a solid (82 mg, 20%). ¹H NMR (400 MHz, DMSO-d6): δ 11.51 (s, 1H), 9.70 (s, 1H), 8.75 (d, J=4.6 Hz, 1H), 8.53 (d, J=7.3 Hz, 1H), 8.29 (s, 1H), 7.71 (d, J=7.8 Hz, 1H), 7.61 (d, J=9.2 Hz, 2H), 7.45 (t, J=8.0 Hz, 1H), 7.31 (s, 1H), 7.07-7.16 (m, 3H), 2.77 (d, J=4.6 Hz, 3H). Observed [M+1]+: 464.05.

### Example 82 Synthesis of Compound E19

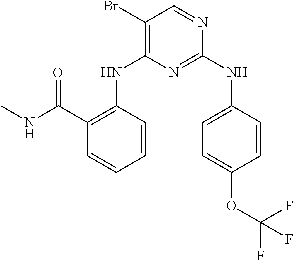 [SMILES: CNC(=O)c1ccccc1Nc1nc(Nc2ccc(OC(F)(F)F)cc2)ncc1Br]

2-((5-Bromo-2-((4-(trifluoromethoxy)phenyl)amino)pyrimidin-4-yl)amino)-N-methylbenzamide (E19). A solution of 2-((5-bromo-2-chloropyrimidin-4-yl)amino)-N-methylbenzamide (300 mg, 0.878 mmol), and 4-(trifluoromethoxy)aniline (0.18 mL, 1.34 mmol) in n-butanol/AcOH (1:1, 6 mL) was heated at 120° C. for 2 h. The reaction mixture was then concentrated in vacuo and the crude product was purified by automated liquid chromatography to afford the title compound as a solid (82 mg, 19%). ¹H NMR (400 MHz, DMSO-d6) δ 11.34 (s, 1H), 9.62 (s, 1H), 8.71 (d, J=4.6 Hz, 1H), 8.58 (d, J=7.8 Hz, 1H), 8.28 (s, 1H), 7.69-7.73 (m, 3H), 7.44-7.53 (m, 1H), 7.22 (d, J=8.2 Hz, 1H), 7.10-7.14 (m, 1H), 2.77 (d, J=4.6 Hz, 3H). Observed [M+1]+: 482.04.

### Example 83 Synthesis of Compound E20

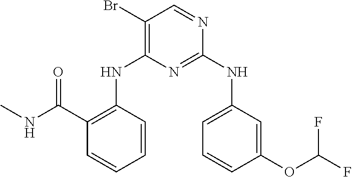 [SMILES: CNC(=O)c1ccccc1Nc1nc(Nc2cccc(OC(F)F)c2)ncc1Br]

2-((5-Bromo-2-((3-(difluoromethoxy)phenyl)amino)pyrimidin-4-yl)amino)-N-methylbenzamide (E20). A solution of 2-((5-bromo-2-chloropyrimidin-4-yl)amino)-N-methylbenzamide (300 mg, 0.878 mmol), and 3-(difluoromethoxy)aniline (210 mg, 1.32 mmol) in n-butanol/AcOH (1:1, 6 mL) was heated at 120° C. for 2 h. The reaction mixture was then concentrated in vacuo and the crude product was purified by automated liquid chromatography to afford the title compound as a white solid (217 mg, 53%). ¹H-NMR (400 MHz, DMSO-d6): δ 11.36 (s, 1H), 9.62 (s, 1H), 8.72 (d, J=4.6 Hz, 1H), 8.60 (d, J=8.2 Hz, 1H), 8.30 (s, 1H), 7.70 (dd, J=7.8, 1.4 Hz, 1H), 7.59 (d, J=1.8 Hz, 1H), 7.45-7.49 (m, 2H), 7.25 (t, J=8.2 Hz, 1H), 7.10-7.14 (m, 1H), 6.71 (dd, J=7.8, 1.8 Hz, 1H), 2.77 (d, J=4.6 Hz, 3H). Observed [M+1]+: 464.05.

### Example 84 Synthesis of Compound E21

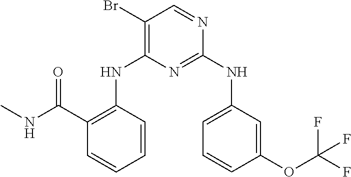 [SMILES: CNC(=O)c1ccccc1Nc1nc(Nc2cccc(OC(F)(F)F)c2)ncc1Br]

2-((5-Bromo-2-((3-(trifluoromethoxy)phenyl)amino)pyrimidin-4-yl)amino)-N-methylbenzamide (E21). A solution of 2-((5-bromo-2-chloropyrimidin-4-yl)amino)-N-methylbenzamide (300 mg, 0.878 mmol), and 3-(trifluoromethoxy)aniline (233 mg, 1.32 mmol) in n-butanol/AcOH (1:1, 6 mL) was heated at 120° C. for 2 h. The reaction mixture was then concentrated in vacuo and the crude product was purified by automated liquid chromatography to afford the title compound as a white solid (154 mg, 36%). ¹H-NMR (400 MHz, DMSO-d6): δ 11.34 (s, 1H), 9.73 (s, 1H), 8.73 (d, J=4.6 Hz, 1H), 8.52-8.57 (m, 1H), 8.32 (s, 1H), 7.81 (s, 1H), 7.71 (dd, J=8.2, 1.4 Hz, 1H), 7.61 (dd, J=8.2, 1.4 Hz, 1H), 7.45-7.49 (m, 1H), 7.32 (t, J=8.2 Hz, 1H), 7.11-7.15 (m, 1H), 6.86 (d, J=7.8 Hz, 1H), 2.77 (d, J=4.6 Hz, 3H). Observed [M+1]+: 482.04.

### Example 85 Synthesis of Compound E22

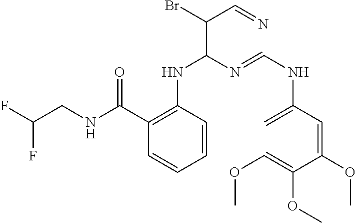 [SMILES: C=C(/C=C(OC)\C(=C\OC)OC)N/C=N/C(Nc1ccccc1C(=O)NCC(F)F)C(Br)C=N]

2-((5-Bromo-2-((3,4,5-trimethoxyphenyl)amino)pyrimidin-4-yl)amino)-N-(2,2-difluoroethyl)benzamide (E22). To a solution of 2-((5-bromo-2-chloropyrimidin-4-yl)amino)-N-(2,2-difluoroethyl)benzamide (260 mg, 0.664 mmol) and 3,4,5-trimethoxyaniline (243 mg, 1.33 mmol) in n-butanol/AcOH (1:1, 6 mL) was heated at 120° C. for 2 h. The reaction mixture was then concentrated in vacuo and the crude product was purified by automated liquid chromatography to afford the title compound as a brown solid (183 mg, 51%). ¹H-NMR (400 MHz, DMSO-d6): δ 11.09 (s, 1H), 9.30 (s, 1H), 9.11 (t, J=5.7 Hz, 1H), 8.67 (d, J=7.3 Hz, 1H), 8.27 (s, 1H), 7.74 (d, J=6.9 Hz, 1H), 7.42 (t, J=7.6 Hz, 1H), 7.13 (t, J=7.6 Hz, 1H), 6.97 (s, 2H), 3.53-3.72 (m, 12H). Observed [M+1]+: 538.08.

### Example 86 Synthesis of Compound E23

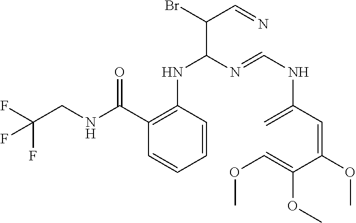 [SMILES: C=C(/C=C(OC)\C(=C\OC)OC)N/C=N/C(Nc1ccccc1C(=O)NCC(F)(F)F)C(Br)C=N]

2-((5-Bromo-2-((3,4,5-trimethoxyphenyl)amino)pyrimidin-4-yl)amino)-N-(2,2,2-trifluoroethyl)benzamide (E23). To a solution of 2-((5-bromo-2-chloropyrimidin-4-yl)amino)-N-(2,2,2-trifluoroethyl)benzamide (325 mg, 0.793 mmol) and 3,4,5-trimethoxyaniline (291 mg, 1.59 mmol) in n-butanol/AcOH (1:1, 6 mL) was heated at 120° C. for 2 h. The reaction mixture was then concentrated in vacuo and the crude product was purified by automated chromatography (152 mg, 34%). ¹H-NMR (400 MHz, DMSO-d6): δ 10.92 (s, 1H), 9.37 (t, J=6.2 Hz, 1H), 9.31 (s, 1H), 8.67 (d, J=7.3 Hz, 1H), 8.27 (s, 1H), 7.74 (dd, J=7.8, 1.4 Hz, 1H), 7.44 (t, J=7.8 Hz, 1H), 7.12-7.16 (m, 1H), 6.97 (s, 2H), 4.04-4.13 (m, 2H), 3.53-3.67 (m, 9H). Observed [M+1]+: 556.07.

### Example 87 Synthesis of Compound E24

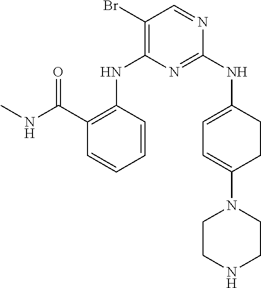 [SMILES: CNC(=O)c1ccccc1Nc1nc(NC2=CC=C(N3CCNCC3)CC2)ncc1Br]

2-((5-Bromo-2-((4-(piperazin-1-yl)phenyl)amino)pyrimidin-4-yl)amino)-N-methylbenzamide (E24). 2-((5-Bromo-2-chloropyrimidin-4-yl)amino)-N-methylbenzamide (0.700 g, 2.05 mmol) and tert-butyl 4-(4-aminophenyl)piperazine-1-carboxylate (710 mg, 2.56 mmol) were dissolved in n-butanol/acetic acid (3:1, 8 mL). The reaction mixture was stirred at 85° C. until completion shown by LCMS. The reaction mixture was cooled to room temperature and purified by normal phase chromatography to afford the Boc protected intermediate as a tan solid (547 mg, 46%). The Boc protected intermediate was treated with 4 N HCl in dioxane to afford the title compound. (54 mg, 46%). ¹H-NMR (400 MHz, DMSO-d₆): δ 11.31 (s, 1H), 9.16 (s, 1H), 8.67 (d, J=21.5 Hz, 2H), 8.18 (s, 1H), 7.69 (dd, J=7.8, 1.4 Hz, 1H), 7.42 (d, J=8.7 Hz, 3H), 7.09 (t, J=7.3 Hz, 1H), 6.81 (d, J=9.2 Hz, 2H), 3.34 (d, J=49.9 Hz, 3H), 2.92-3.00 (m), 2.76-2.79 (m, 4H). Observed [M+1]⁺: 482.18.

### Example 88 Synthesis of Compound E25

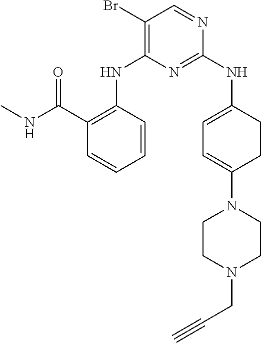 [SMILES: C#CCN1CCN(C2=CC=C(Nc3ncc(Br)c(Nc4ccccc4C(=O)NC)n3)CC2)CC1]

2-((5-Bromo-2-((4-(4-(prop-2-yn-1-yl)piperazin-1-yl)phenyl)amino)pyrimidin-4-yl)amino)-N-methylbenzamide (E25). The synthetic procedure is similar to the synthesis of Compound A127 except that 2-((5-bromo-2-((4-(piperazin-1-yl)phenyl)amino)pyrimidin-4-yl)amino)-N-methylbenzamide (315 mg, 607 μmol, 1 equiv.) was used instead of 2-[5-bromo-2-(1,2,3,4-tetrahydro-isoquinolin-6-ylamino)-pyrimidin-4-ylamino]-N-methyl-benzamide, 3-bromoprop-1-yne (107 mg, 718 μmol, 1.18 equiv.) was used instead of 2-chloro-1-morpholin-4-yl-ethanone, and potassium carbonate (252 mg, 1.82 mmol, 3 equiv.) was used instead of diisopropylethylamine. The reaction mixture was heated at 80° C. in DMF (5 mL) to provide the product following work-up and purification (152 mg, 48%). ¹H-NMR (400 MHz, DMSO-d6) δ 11.31 (s, 1H), 9.17 (s, 1H), 8.65-8.70 (m, 2H), 8.19 (s, 1H), 7.68 (dd, J=7.8, 1.4 Hz, 1H), 7.40-7.44 (m, 3H), 7.07-7.11 (m, 1H), 6.84 (d, J=8.7 Hz, 2H), 3.15 (m), 3.05 (t, J=4.6 Hz, 4H), 2.77 (d, J=4.1 Hz, 3H), 2.56 (t, J=4.8 Hz, 4H). Observed [M+1]+: 521.14.

### Example 89 Synthesis of Compound E26

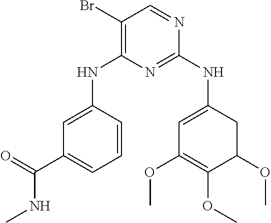 [SMILES: CNC(=O)c1cccc(Nc2nc(NC3=CC(OC)=C(OC)C(OC)C3)ncc2Br)c1]

3-((5-Bromo-2-((3,4,5-trimethoxyphenyl)amino)pyrimidin-4-yl)amino)-N-methylbenzamide (E26). 3-((5-Bromo-2-chloropyrimidin-4-yl)amino)-N-methylbenzamide was prepared according to Method 4a by reaction of 5-bromo-2,4-dichloropyrimidine (1.14 g, 4.99 mmol), 3-amino-N-methylbenzamide (600 mg, 4.0 mmol), and potassium carbonate (718 mg, 5.19 mmol) in DMF (16 mL) at 80° C. for 4 h to afford the intermediate compound as a yellow solid (1.265 g, 93%). A solution of 3-((5-bromo-2-chloropyrimidin-4-yl)amino)-N-methylbenzamide (300 mg, 0.878 mmol) and 3,4,5-trimethoxyaniline (241 mg, 1.32 mmol) in n-butanol/AcOH (3:1, 4 mL) was heated at 120° C. for 2 h. The reaction mixture was then concentrated in vacuo and the crude product was purified by automated liquid chromatography to afford the title compound as a purple solid (203 mg, 47%). ¹H-NMR (400 MHz, DMSO-d6): δ 9.23 (s, 1H), 8.67 (s, 1H), 8.30 (d, J=4.6 Hz, 1H), 8.24 (s, 1H), 7.73 (dd, J=15.1, 8.7 Hz, 4H), 6.95 (s, 2H), 3.51-3.59 (m, 9H), 2.72-2.75 (m, 3H). Observed [M+1]+: 488.09.

### Example 90 Synthesis of Compound E27

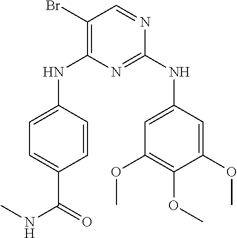

4-((5-Bromo-2-((3,4,5-trimethoxyphenyl)amino)pyrimidin-4-yl)amino)-N-methylbenzamide (E27). 4-((5-Bromo-2-chloropyrimidin-4-yl)amino)-N-methylbenzamide was prepared according to Method 4a by reaction of 5-bromo-2,4-dichloropyrimidine (1.14 g, 4.99 mmol), 4-amino-N-methylbenzamide (600 mg, 4.0 mmol), and potassium carbonate (718 mg, 5.19 mmol) in DMF (16 mL) at 80° C. for 4 h to afford the intermediate compound as a brown solid (1.239 g, 91%).

To a solution of 4-((5-bromo-2-chloropyrimidin-4-yl)amino)-N-methylbenzamide (300 mg, 878 μmol) and 3,4,5-trimethoxyaniline (241 mg, 1.32 mmol) in n-butanol/AcOH (3:1, 4 mL) was heated at 120° C. for 2 h. The reaction mixture was then concentrated in vacuo and the crude product was purified by automated liquid chromatography to afford the title compound as a purple solid (285 mg, 67%). ¹H NMR (400 MHz, DMSO-d6): δ 9.15 (s, 1H), 8.69 (s, 1H), 8.30 (d, J=4.6 Hz, 1H), 8.21 (s, 1H), 7.88-7.91 (m, 1H), 7.79 (d, J=8.2 Hz, 1H), 7.53 (d, J=7.8 Hz, 1H), 7.34 (t, J=8.0 Hz, 1H), 6.91 (s, 2H), 3.53 (s, 3H), 3.46 (s, 6H), 2.72 (d, J=4.6 Hz, 3H). Observed [M+1]+: 488.09.

Compounds for which synthesis protocols were not supplied were prepared using analogous methods to those provided above.

While preferred embodiments of the present invention have been shown and described herein, it will be obvious to those skilled in the art that such embodiments are provided by way of example only. Numerous variations, changes, and substitutions will now occur to those skilled in the art without departing from the invention. It should be understood that various alternatives to the embodiments of the invention described herein may be employed in practicing the invention. It is intended that the following claims define the scope of the invention and that methods and structures within the scope of these claims and their equivalents be covered thereby

What is claimed is:

1. A compound, having a structure of Formula (IA): 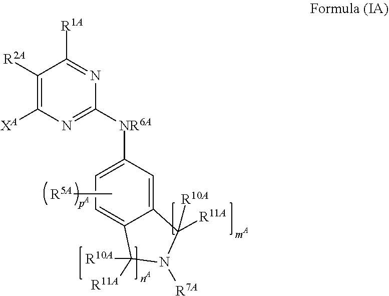
wherein;
R^{1A} is H, halogen, or substituted or unsubstituted alkyl;
R^{2A} is H, haloalkyl, —C(═O)R^{A}, NH₂, or halogen;
X^{A} is —NR^{3A}R^{4A} or —OR^{4A};
R^{3A} is H, substituted or unsubstituted alkyl, or a bond with a substituent on an R^{4A} to form a heterocycle;
R^{4A} is aryl, heteroaryl, cycloalkyl, or heterocycloalkyl;
wherein the aryl or heteroaryl of R^{4A} is optionally substituted with one or more halogen, —CN, —OR^{A}, —SR^{A}, —NO₂, —NR^{A}R^{A}, —NR^{A}S(═O)₂R^{A}, —C(═O)R^{A}, —OC(═O)R^{A}, —C(═O)C(═O)R^{A}, —C(═O)OR^{A}, —C(═O)NR^{A}OR^{A}, —OC(═O)OR^{A}, —C(═O)NR^{A}R^{A}, —OC(═O)NR^{A}R^{A}, —NR^{A}C(═O)NR^{A}R^{A}, —NR^{A}S(═O)₂NR^{A}R^{A}, —NR^{A}C(═O)R^{A}, —NR^{A}C(═O)OR^{A}, substituted or unsubstituted alkyl, substituted or unsubstituted cycloalkyl, substituted or unsubstituted heterocycloalkyl, substituted or unsubstituted aryl, or substituted or unsubstituted heteroaryl;
wherein the cycloalkyl or heterocycloalkyl of R^{4A} is optionally substituted with one or more halogen, —CN, —OR^{A}, —SR^{A}, —S(═O)R^{A}, —S(═O)₂R^{A}, —NO₂, —NR^{A}R^{A}, —NR^{A}S(═O)₂R^{A}, —S(═O)₂NR^{A}R^{A}, —C(═O)R^{A}, —OC(═O)R^{A}, —C(═O)C(═O)R^{A}, —C(═O)OR^{A}, —C(═O)NR^{A}OR^{A}, —OC(═O)OR^{A}, —OC(═O)NR^{A}R^{A}, —NR^{A}C(═O)NR^{A}R^{A}, —NR^{A}S(═O)₂NR^{A}R^{A}, —NR^{A}C(═O)R^{A}, —NR^{A}C(═O)OR^{A}, substituted or unsubstituted alkyl, substituted or unsubstituted cycloalkyl, substituted or unsubstituted heterocycloalkyl, substituted or unsubstituted aryl, or substituted or unsubstituted heteroaryl;
each R^{5A} is independently halogen, —CN, —OR^{A}, —SR^{A}, —S(═O)R^{A}, —S(═O)₂R^{A}, —NO₂, —NR^{A}R^{A}, —NR^{A}S(═O)₂R^{A}, —S(═O)₂NR^{A}R^{A}, —C(═O)R^{A}, —OC(═O)R^{A}, —C(═O)C(═O)R^{A}, —C(═O)OR^{A}, —C(═O)NR^{A}OR^{A}, —OC(═O)OR^{A}, —C(═O)NR^{A}R^{A}, —OC(═O)NR^{A}R^{A}, —NR^{A}C(═O)NR^{A}R^{A}, —NR^{A}S(═O)₂NR^{A}R^{A}, —NR^{A}C(═O)R^{A}, —NR^{A}C(═O)OR^{A}, substituted or unsubstituted alkyl, substituted or unsubstituted cycloalkyl, substituted or unsubstituted heterocycloalkyl, substituted or unsubstituted aryl, or substituted or unsubstituted heteroaryl;
R^{6A} is H or substituted or unsubstituted alkyl;
R^{7A} is —S(═O)R^{A}, —S(═O)₂R^{A}, —S(═O)₂NR^{A}R^{A}, —C(═O)NR^{A}R^{A}, —C(═O)R^{71A}, —C(═O)C(═O)R^{A}, —C(═O)OR^{72A}, —C(═O)NR^{A}OR^{A}, substituted or unsubstituted alkyl, substituted or unsubstituted cycloalkyl, or substituted or unsubstituted heterocycloalkyl;
each R^{10A} and R^{11A} is independently H, substituted or unsubstituted alkyl, substituted or unsubstituted alkoxy, substituted or unsubstituted cycloalkyl, substituted or unsubstituted heterocycloalkyl, hydroxyl, halogen, or R^{10A} and R^{11A} on the same atom join to form a cycloalkyl or heterocycloalkyl, or R^{10A} and R^{11A} on the same atom are taken together to form an oxo;
R^{71A} is H, —CN, substituted or unsubstituted methyl, substituted or unsubstituted ethyl, substituted or unsubstituted C₃-C₁₀ alkyl, substituted or unsubstituted C₄-C₁₀ cycloalkyl, substituted or unsubstituted heterocycloalkyl, substituted or unsubstituted aryl, or substituted or unsubstituted;
R^{72A} is H, —CN, substituted or unsubstituted methyl, substituted or unsubstituted ethyl, linear C₃-C₅ alkyl, substituted or unsubstituted C₃-C₁₀ cycloalkyl, substituted or unsubstituted heterocycloalkyl, substituted or unsubstituted aryl, or substituted or unsubstituted heteroaryl;
each R^{A} is independently H, substituted or unsubstituted alkyl, substituted or unsubstituted heteroalkyl, substituted or unsubstituted aryl, or substituted or unsubstituted heteroaryl;
n^{A} is 1 or 2;
m^{A} is 1 or 2; wherein the sum of n and m is 2 or 3;
p^{A} is an integer from 0-3; and
the nitrogen in the fused ring system is optionally quaternized with C₁-C₆ alkyl,
or pharmaceutically acceptable salt thereof.

2. The compounds of claim 1, wherein R^{1A} is H, halogen, or C₁-C₆ alkyl.

3. The compounds of claim 1 or 2, wherein R^{1A} is H or fluorine.

4. The compound of any one of the preceding claims, wherein R^{1A} is H.

5. The compound of any one of the preceding claims, wherein R^{2A} is H, C₁-C₆ haloalkyl, or halogen.

6. The compound of any one of the preceding claims, wherein R^{2A} is —CF₃, or halogen.

7. The compound of any one of the preceding claims, wherein R^{2A} is —CF₃, —Cl, or —Br.

8. The compound of any one of the preceding claims, wherein R^{2A} is —CF₃.

9. The compound of any one of claims 1-7, wherein R^{2A} is Br.

10. The compound of any one of the preceding claims, wherein X^{A} is —NR^{3A}R^{4A}.

11. The compound of any one of the preceding claims, wherein R^{3A} is H or C₁-C₆ alkyl.

12. The compound of any one of the preceding claims, wherein R^{3A} is H, or —CH₃.

13. The compound of any one of the preceding claims, wherein R^{3A} is H.

14. The compound of any one of the preceding claims, wherein R^{4A} is aryl or heteroaryl;
wherein the aryl or heteroaryl of R^{4A} is optionally substituted with one or more halogen, —CN, —OR^{A}, —SR^{A}, —NO₂, —NR^{A}R^{A}, —NR^{A}S(═O)₂R^{A}, —C(═O)R^{A}, —OC(═O)R^{A}, —C(═O)C(═O)R^{A}, —C(═O)OR^{A}, —C(═O)NR^{A}OR^{A}, —OC(═O)OR^{A}, —C(═O)NR^{A}R^{A}, —OC(═O)NR^{A}R^{A}, —NR^{A}C(═O)NR^{A}R^{A}, —NR^{A}S(═O)₂NR^{A}R^{A}, —NR^{A}C(═O)R^{A}, —NR^{A}C(═O)OR^{A}, substituted or unsubstituted alkyl, or substituted or unsubstituted cycloalkyl.

15. The compound of any one of the preceding claims, wherein R^{4A} is aryl or heteroaryl;
wherein the aryl or heteroaryl of R^{4A} is optionally substituted with one or more halogen, —CN, —OR^{A}, —NR^{A}R^{A}, —C(═O)R^{A}, —OC(═O)R^{A}, —C(═O)C(═O)R^{A}, —C(═O)OR^{A}, —C(═O)NR^{A}OR^{A}, —OC(═O)OR^{A}, —C(═O)NR^{A}R^{A}, —OC(═O)NR^{A}R^{A}, —NR^{A}C(═O)NR^{A}R^{A}, —NR^{A}C(═O)R^{A}, —NR^{A}C(═O)OR^{A}, substituted or unsubstituted alkyl, or substituted or unsubstituted cycloalkyl.

16. The compound of any one of the preceding claims, wherein R^{4A} is aryl or heteroaryl;
wherein the aryl or heteroaryl of R^{4A} is optionally substituted with one or more halogen, —CN, —OR^{A}, —NR^{A}R^{A}, —C(═O)R^{A}, —OC(═O)R^{A}, —C(═O)OR^{A}, —C(═O)NR^{A}OR^{A}, —C(═O)NR^{A}R^{A}, —NR^{A}C(═O)R^{A}, substituted or unsubstituted alkyl, or substituted or unsubstituted cycloalkyl.

17. The compound of any one of the preceding claims, wherein R^{4A} is aryl or heteroaryl;
wherein the aryl or heteroaryl of R^{4A} is optionally substituted with one or more halogen, —OR^{A}, —C(═O)R^{A}, —C(═O)NR^{A}R^{A}, —NR^{A}C(═O)R^{A}, or substituted or unsubstituted alkyl.

18. The compound of any one of the preceding claims, wherein R^{4A} is aryl or heteroaryl;
wherein the aryl or heteroaryl of R^{4A} is optionally substituted with one or more halogen, —OR^{A}, —C(═O)NR^{A}R^{A}, or substituted or unsubstituted alkyl.

19. The compound of any one of the preceding claims, wherein R^{4A} is 6-membered aryl or heteroaryl.

20. The compound of any one of the preceding claims, wherein R^{4A} is phenyl, pyridyl, or pyrimidinyl.

21. The compound of any one of the preceding claims, wherein R^{4A} is phenyl.

22. The compound of any one of the preceding claims, wherein R^{4A} is phenyl substituted with

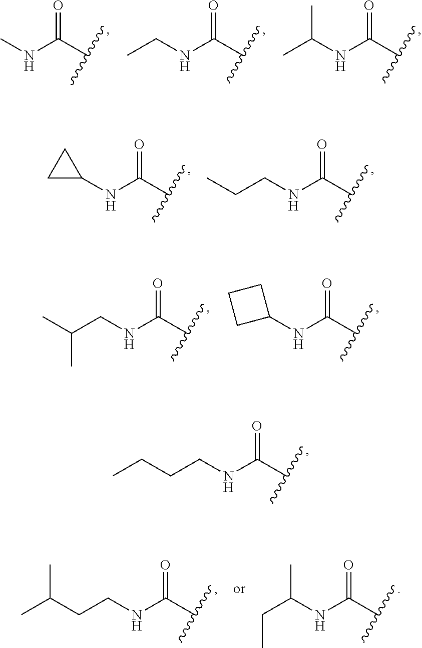

23. The compound of any one of the preceding claims, wherein R^{4A} is

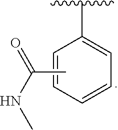

24. The compound of any one of the preceding claims, wherein R^{4A} is

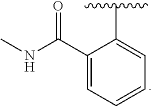 [SMILES: *c1ccccc1C(=O)NC]

25. The compound of any one of claims 1-13, wherein R^{4A} is cycloalkyl, or heterocycloalkyl, wherein the cycloalkyl or heterocycloalkyl of R^{4A} is optionally substituted with one or more halogen, —CN, —OR^{A}, —NR^{A}R^{A}, —C(═O)R^{A}, —OC(═O)R^{A}, —C(═O)C(═O)R^{A}, —C(═O)OR^{A}, —C(═O)NR^{A}OR^{A}, —OC(═O)OR^{A}, —C(═O)NR^{A}R^{A}, —OC(═O)NR^{A}R^{A}, —NR^{A}C(═O)NR^{A}R^{A}, —NR^{A}C(═O)R^{A}, —NR^{A}C(═O)OR^{A}, substituted or unsubstituted alkyl, or substituted or unsubstituted cycloalkyl.

26. The compound of any one of claims 1-13 or 25, wherein R^{4A} is cycloalkyl optionally substituted with one or more halogen, —CN, —OR^{A}, —NR^{A}R^{A}, —C(═O)R^{A}, —OC(═O)R^{A}, —C(═O)C(═O)R^{A}, —C(═O)OR^{A}, —C(═O)NR^{A}OR^{A}, —OC(═O)OR^{A}, —C(═O)NR^{A}R^{A}, —OC(═O)NR^{A}R^{A}, —NR^{A}C(═O)NR^{A}R^{A}, —NR^{A}C(═O)R^{A}, —NR^{A}C(═O)OR^{A}, substituted or unsubstituted alkyl, or substituted or unsubstituted cycloalkyl.

27. The compound of any one of claims 1-13 or 25-26, wherein R^{4A} is cyclopropyl, cyclobutyl, cyclopentyl, or cyclohexyl optionally substituted with one or more halogen, —CN, —OR^{A}, —NR^{A}R^{A}, —C(═O)R^{A}, —OC(═O)R^{A}, —C(═O)C(═O)R^{A}, —C(═O)OR^{A}, —C(═O)NR^{A}OR^{A}, —OC(═O)OR^{A}, —C(═O)NR^{A}R^{A}, —OC(═O)NR^{A}R^{A}, —NR^{A}C(═O)NR^{A}R^{A}, —NR^{A}C(═O)R^{A}, —NR^{A}C(═O)OR^{A}, substituted or unsubstituted alkyl, or substituted or unsubstituted cycloalkyl.

28. The compound of any one of claims 1-13 or 25-27, wherein R^{4A} is cyclopropyl optionally substituted with one or more halogen, —CN, —OR^{A}, —NR^{A}R^{A}, —C(═O)R^{A}, —OC(═O)R^{A}, —C(═O)C(═O)R^{A}, —C(═O)OR^{A}, —C(═O)NR^{A}OR^{A}, —OC(═O)OR^{A}, —C(═O)NR^{A}R^{A}, —OC(═O)NR^{A}R^{A}, —NR^{A}C(═O)NR^{A}R^{A}, —NR^{A}C(═O)R^{A}, —NR^{A}C(═O)OR^{A}, substituted or unsubstituted alkyl, or substituted or unsubstituted cycloalkyl.

29. The compound of any one of claims 1-13 or 25-28, wherein R^{4A} is cyclopropyl optionally substituted with one or more halogen, —OR^{A}, —C(═O)R^{A}, —C(═O)OR^{A}, —C(═O)NR^{A}R^{A}, or substituted or unsubstituted alkyl.

30. The compound of any one of claims 1-13 or 25-29, wherein R^{4A} is cyclopropyl optionally substituted with one or more-OR^{A} or substituted or unsubstituted alkyl.

31. The compound of any one of claims 1-13 or 25-30, wherein R^{4A} is cyclopropyl optionally substituted with OH or C₁-C₆ alkyl.

32. The compound of any one of claims 1-13 or 25-31, wherein each R^{4A} is unsubstituted cyclopropyl.

33. The compound of any one of the preceding claims, wherein each R^{5A} is independently halogen, —CN, —OR^{A}, —NR^{A}R^{A}, —C(═O)R^{A}, —OC(═O)R^{A}, —C(═O)OR^{A}, —C(═O)NR^{A}OR^{A}, —OC(═O)OR^{A}, —C(═O)NR^{A}R^{A}, —OC(═O)NR^{A}R^{A}, —NR^{A}C(═O)NR^{A}R^{A}, —NR^{A}S(═O)₂NR^{A}R^{A}, —NR^{A}C(═O)R^{A}, —NR^{A}C(═O)OR^{A}, substituted or unsubstituted alkyl, substituted or unsubstituted cycloalkyl, substituted or unsubstituted heterocycloalkyl, substituted or unsubstituted aryl, or substituted or unsubstituted heteroaryl.

34. The compound of any one of the preceding claims, wherein each R^{5A} is independently halogen, —CN, —OR^{A}, —NR^{A}R^{A}, —C(═O)R^{A}, —OC(═O)R^{A}, —C(═O)OR^{A}, substituted or unsubstituted alkyl, or substituted or unsubstituted cycloalkyl.

35. The compound of any one of the preceding claims, wherein each R^{5A} is independently halogen, —OR^{A}, —NR^{A}R^{A}, or unsubstituted C₁-C₆ alkyl.

36. The compound of any one of the preceding claims, wherein p is 0 or 1.

37. The compound of any one of the preceding claims, wherein p is 0.

38. The compound of any one of the preceding claims, wherein R^{6A} is H or —CH₃.

39. The compound of any one of the preceding claims, wherein R^{6A} is H.

40. The compound of any one of the preceding claims, wherein R^{7A} is —S(═O)₂R^{A}, —S(═O)₂NR^{A}R^{A}, —C(═O)NR^{A}R^{A}, —C(═O)R^{71A}, —C(═O)OR^{72A}, —C(═O)NR^{A}OR^{A}, substituted or unsubstituted C₁-C₆ alkyl, substituted or unsubstituted C₃-C₈ cycloalkyl, or substituted or unsubstituted C₂-C₈ heterocycloalkyl.

41. The compounds of any one of the preceding claims, wherein R^{7A} is —S(═O)₂R^{A}, —S(═O)₂NR^{A}R^{A}, —C(═O)R^{71A}, substituted or unsubstituted C₁-C₆ alkyl, or substituted or unsubstituted C₂-C₈ heterocycloalkyl.

42. The compounds of any one of the preceding claims, wherein R^{7A} is —C(═O)R^{71A} or substituted or unsubstituted C₁-C₆ alkyl.

43. The compound of any one of claims 1-40, wherein R^{7A} is

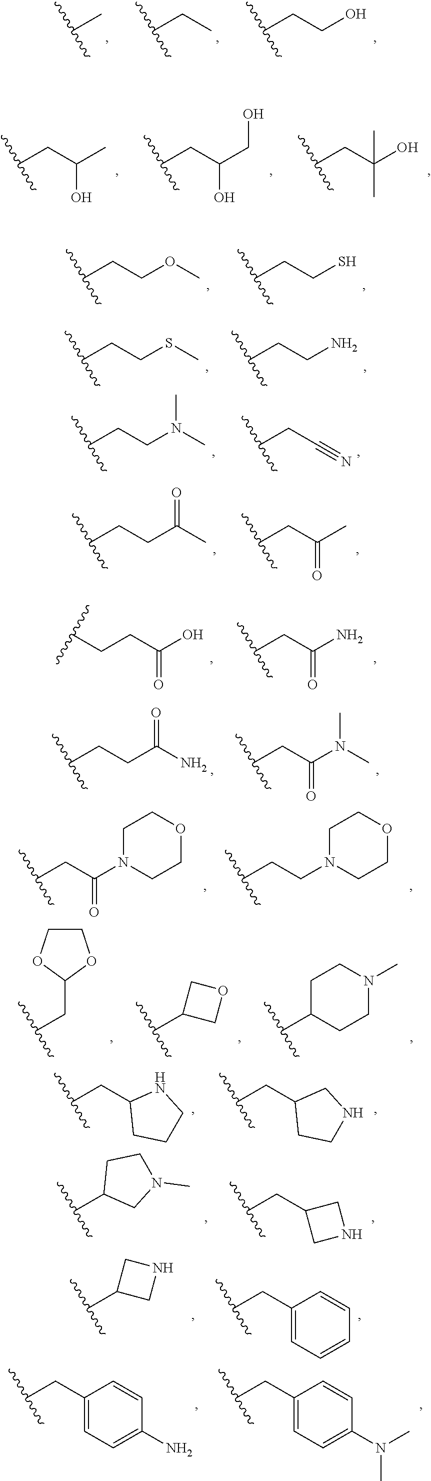

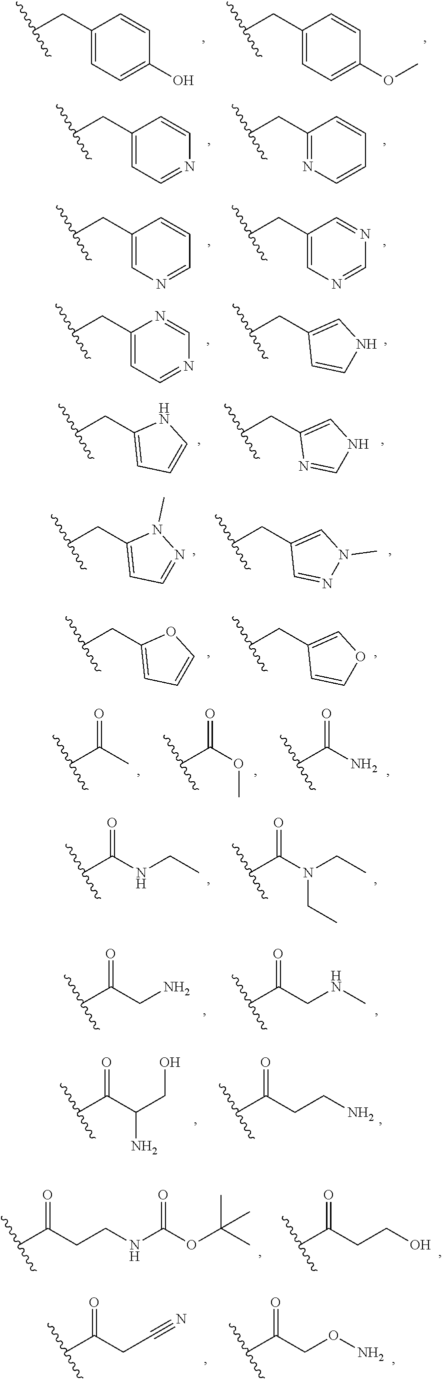

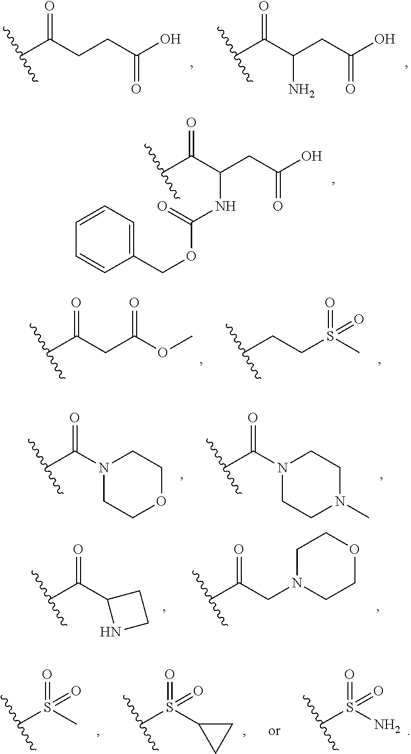

44. The compound of any one of the preceding claims, wherein each R^{10A} and R^{11A} is independently H or substituted or unsubstituted alkyl, or R^{10A} and R^{11A} on the same atom join to form a cycloalkyl, or R^{10A} and R^{11A} on the same atom are taken together to form an oxo.

45. The compound of any one of the preceding claims, wherein each R^{10A} and R^{11A} is independently H or substituted or unsubstituted alkyl, or R^{10A} and R^{11A} on the same atom are taken together to form an oxo.

46. The compound of any one of the preceding claims, wherein each R^{10A} and R^{11A} is independently H.

47. The compound of any one of the preceding claims, wherein n^{A} is 1 and m^{A} is 1.

48. The compound of any one of claims 1-46, wherein n^{A} is 1 and m^{A} is 2.

49. The compound of any one of claims 1-46, wherein n^{A} is 2 and m^{A} is 1.

50. A compound having a structure of 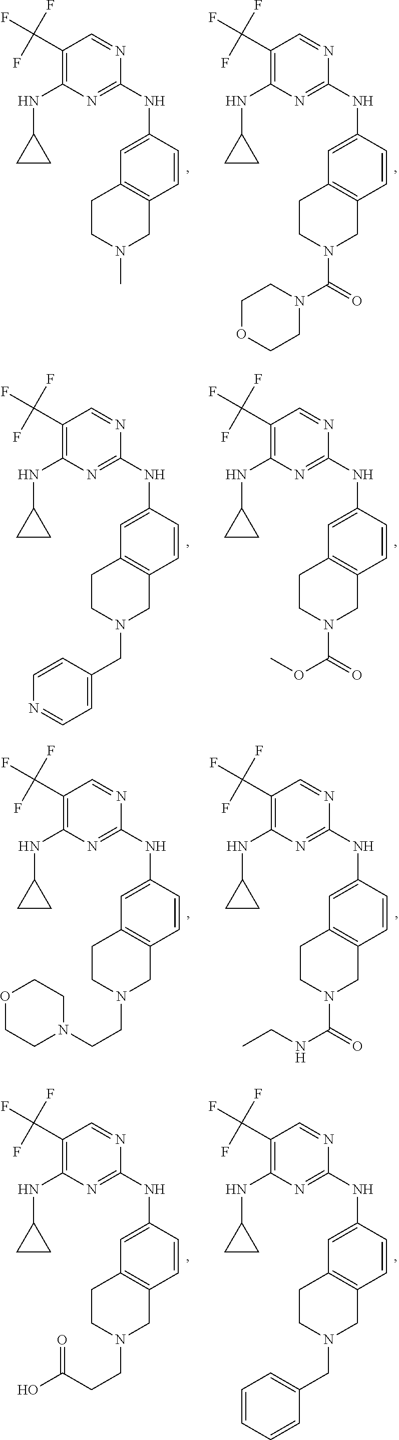 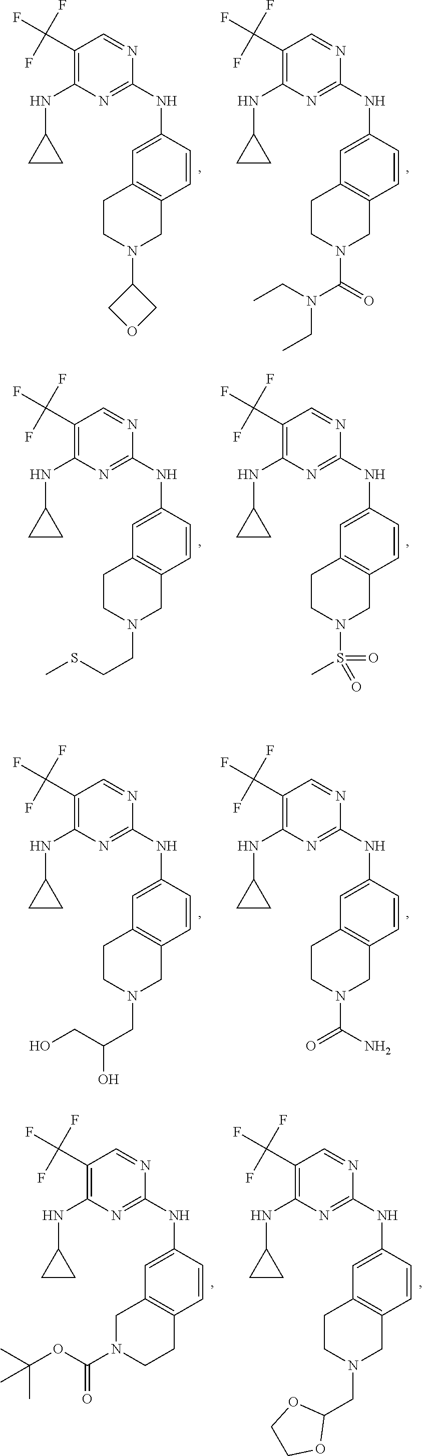 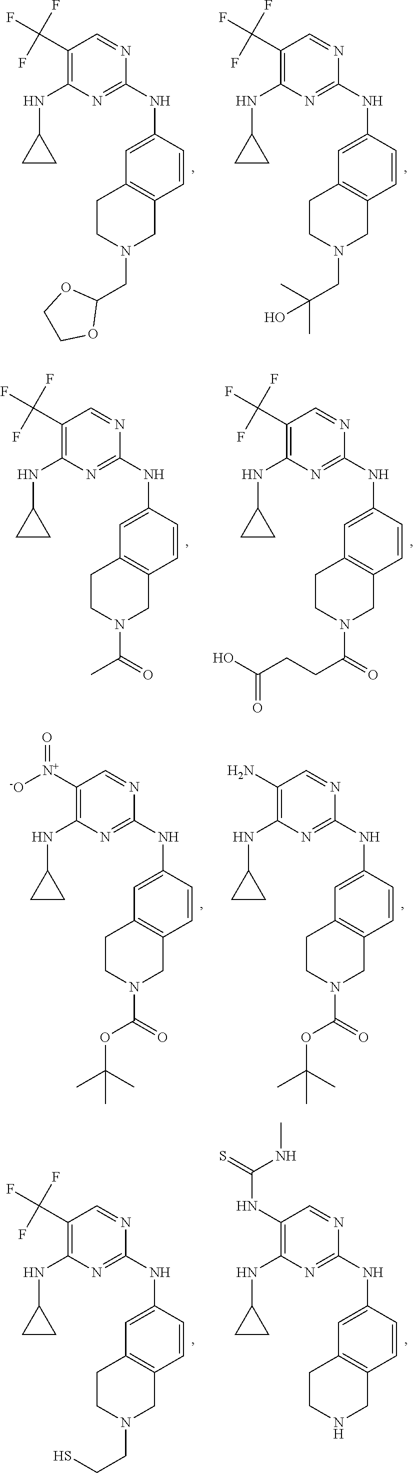 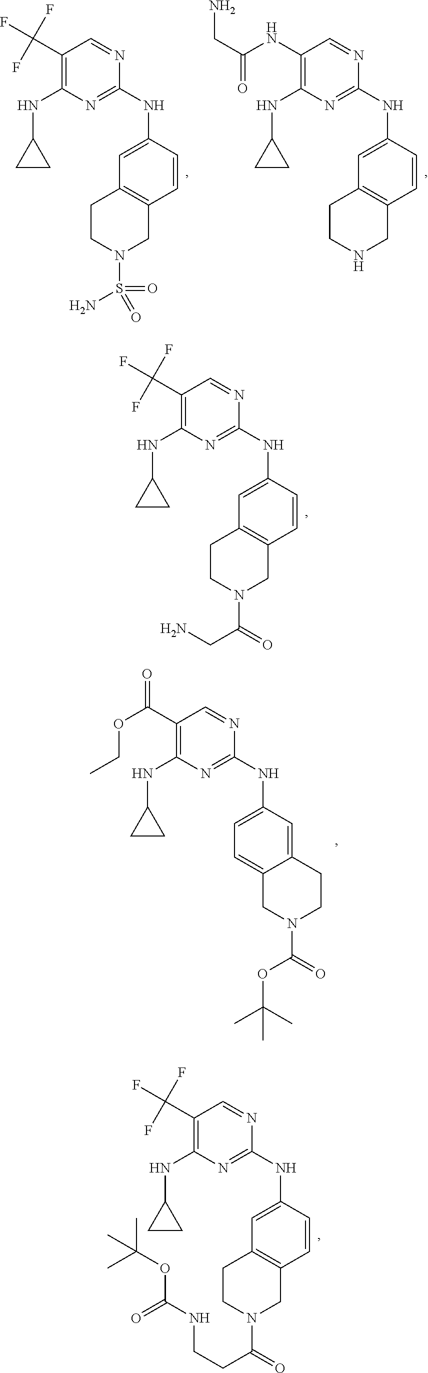 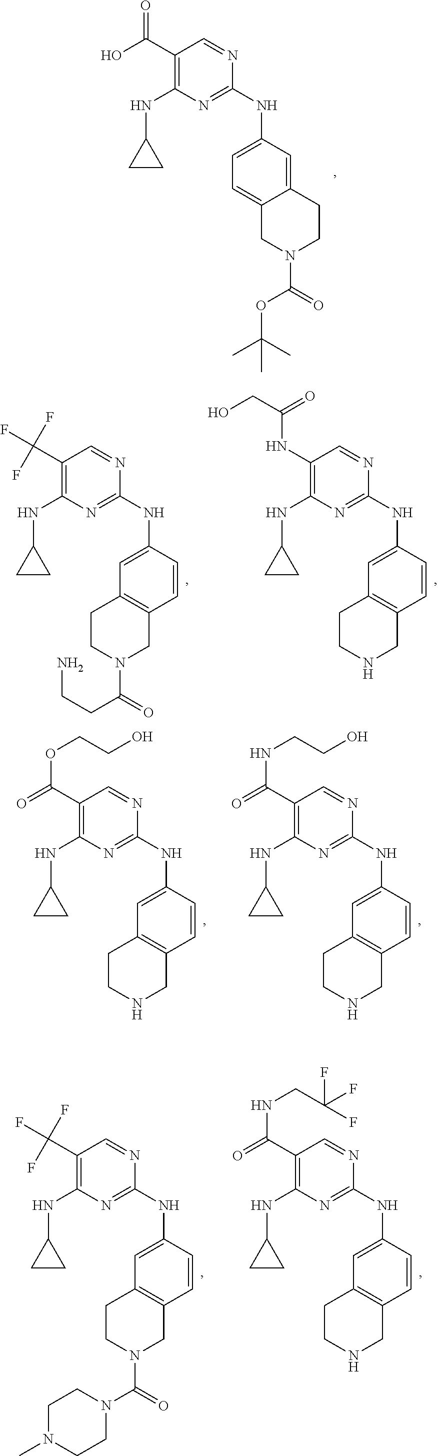 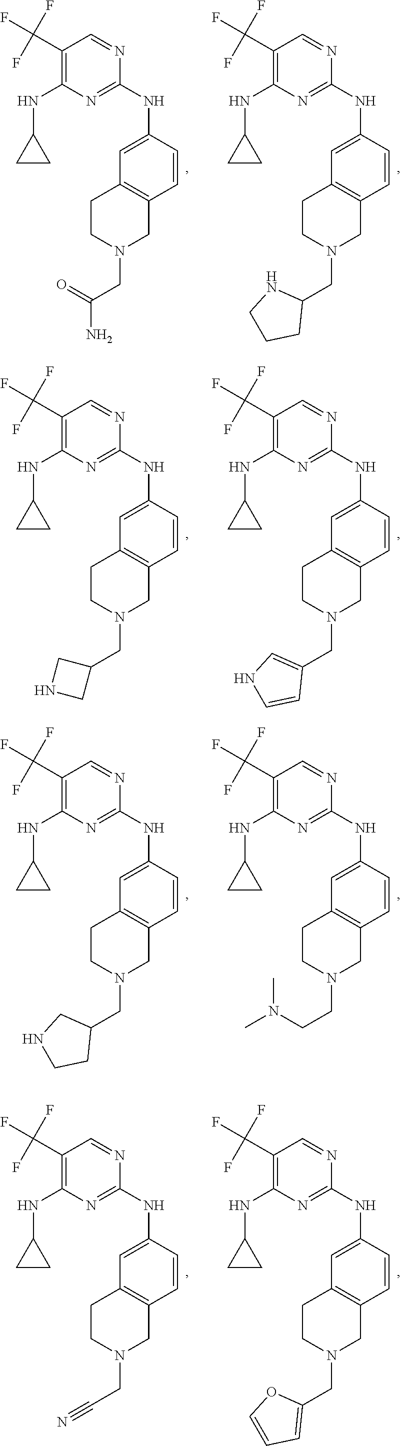 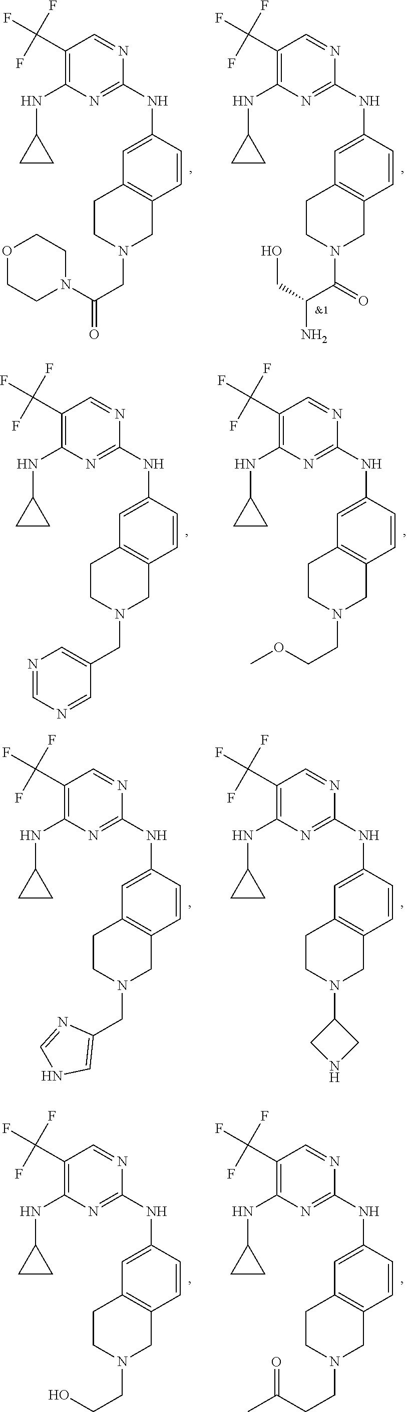 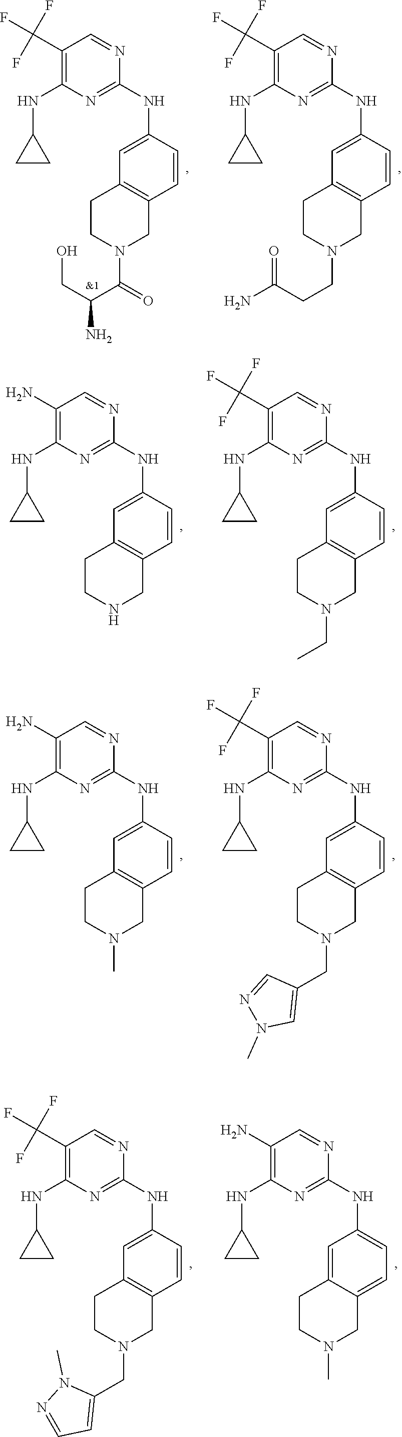 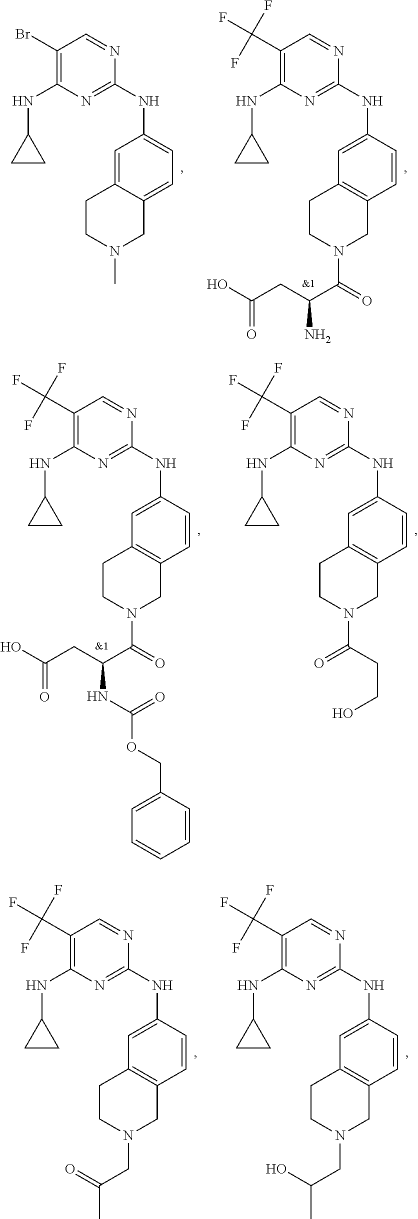 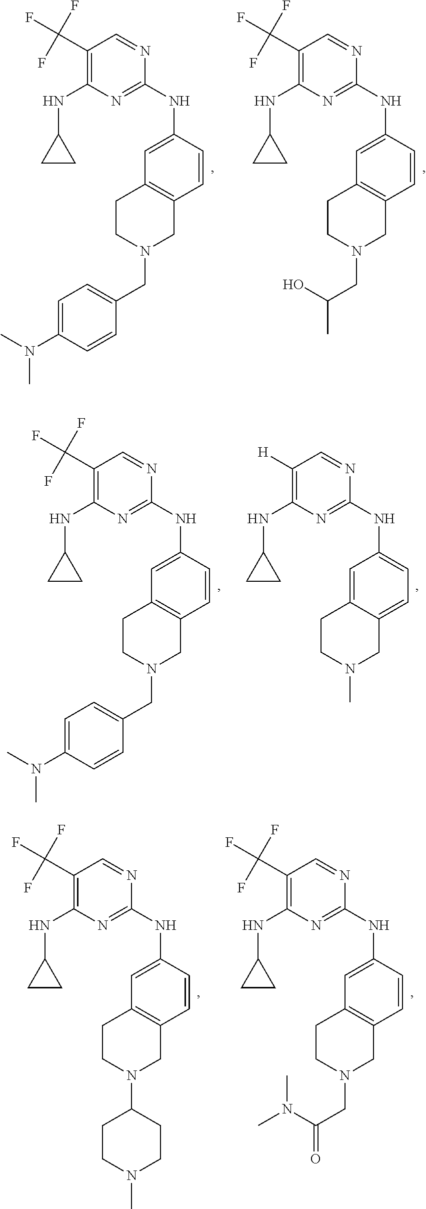 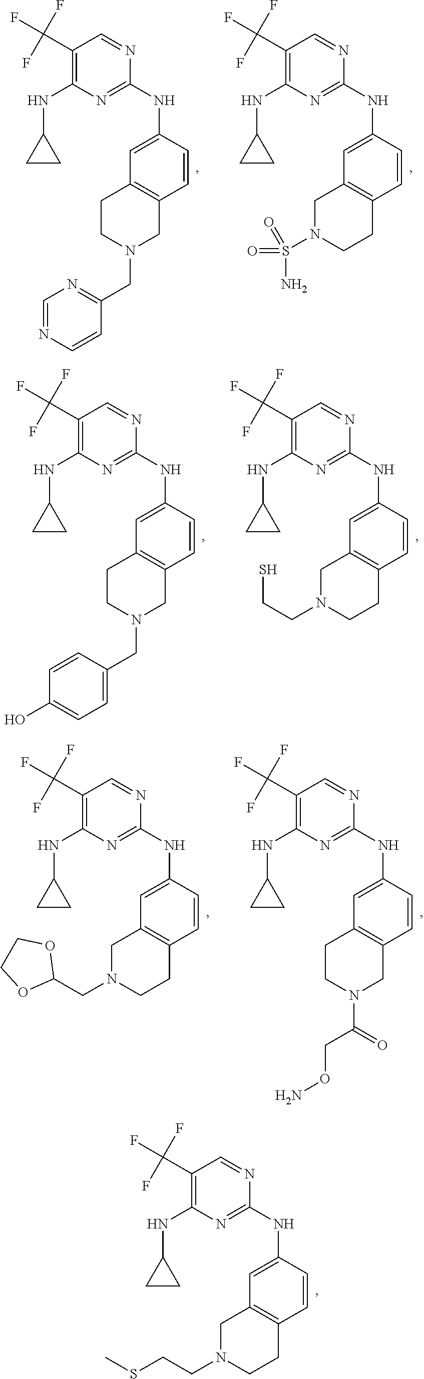 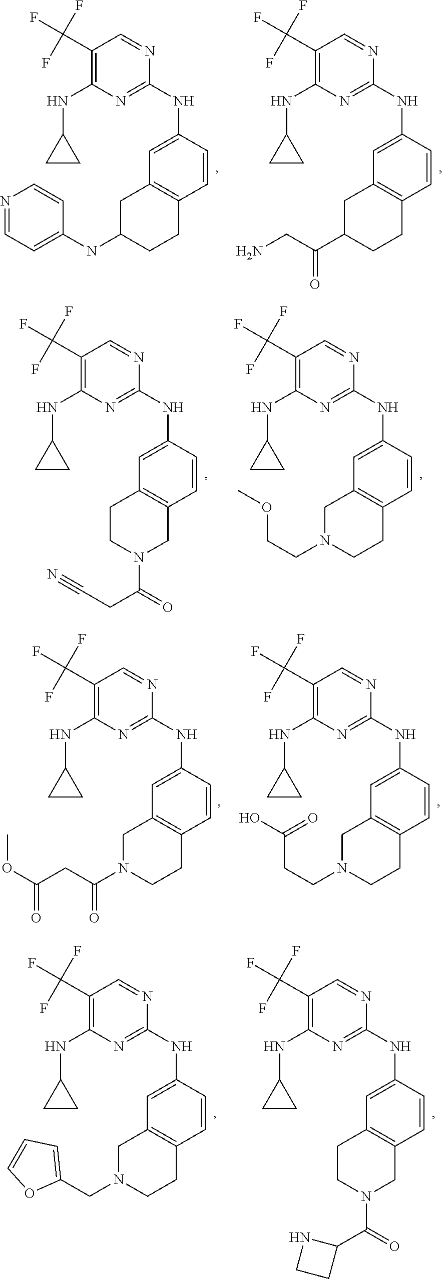 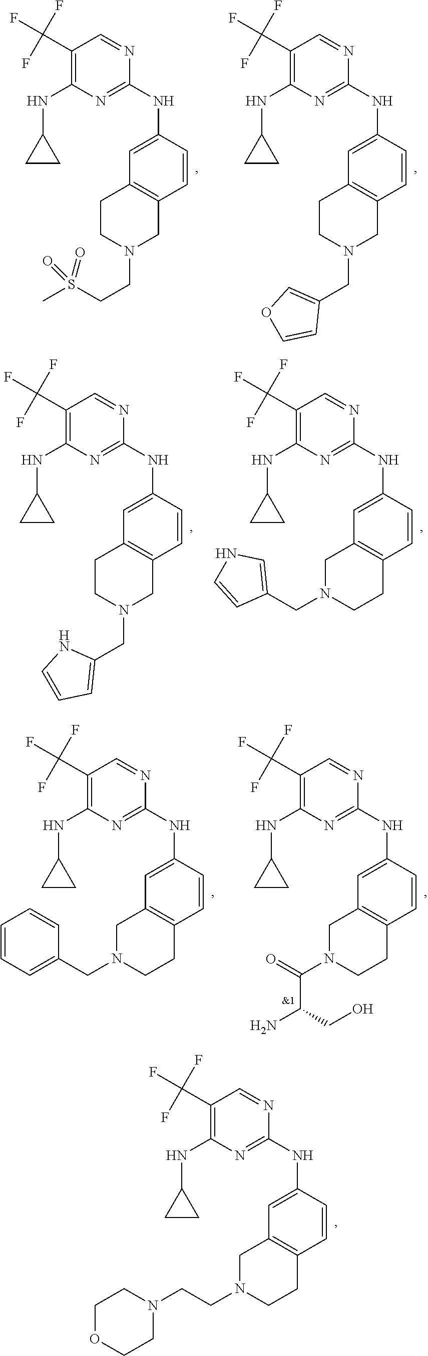 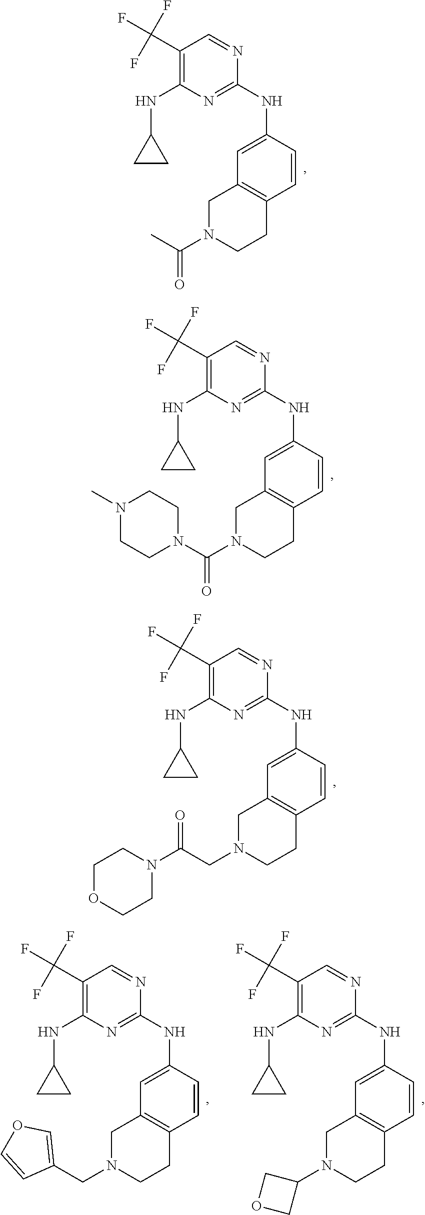 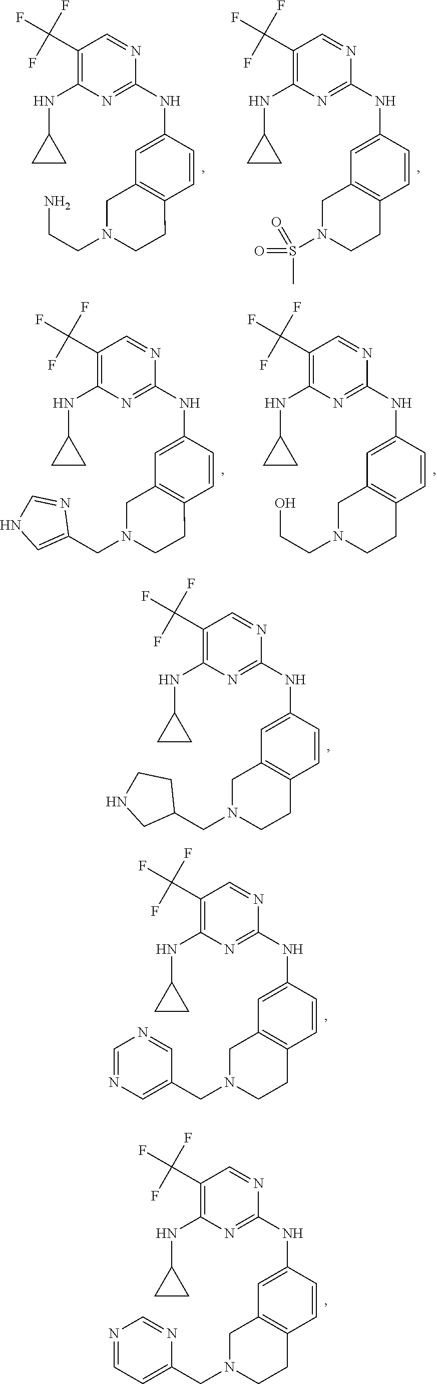 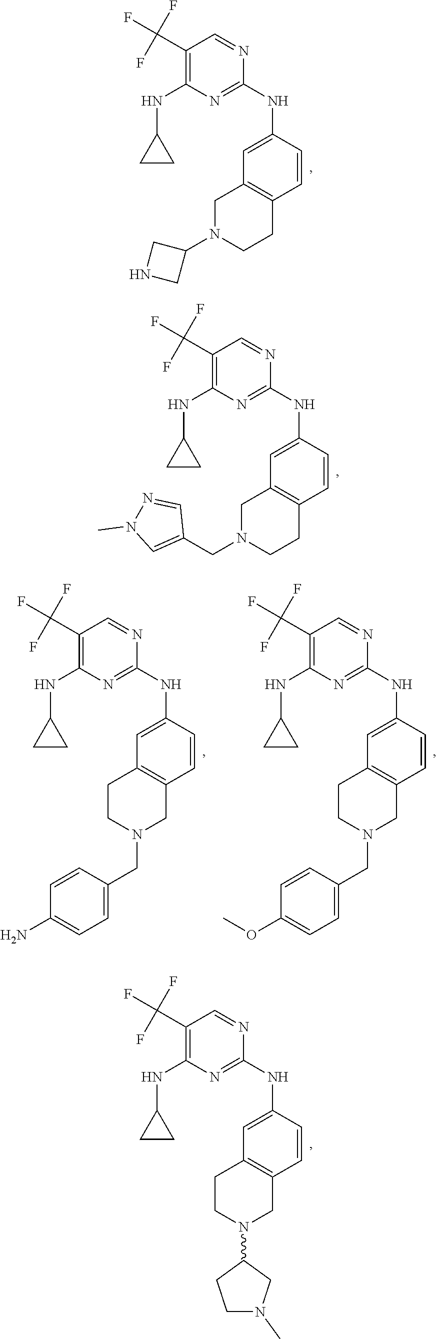 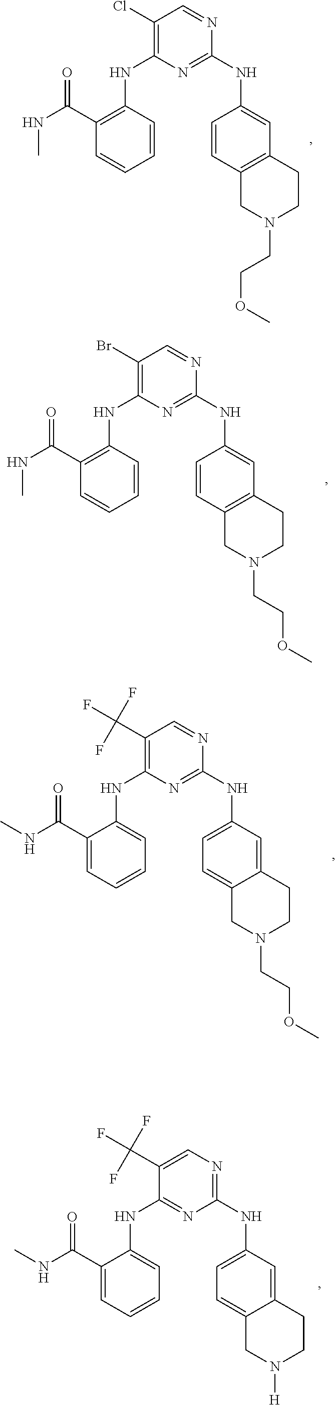 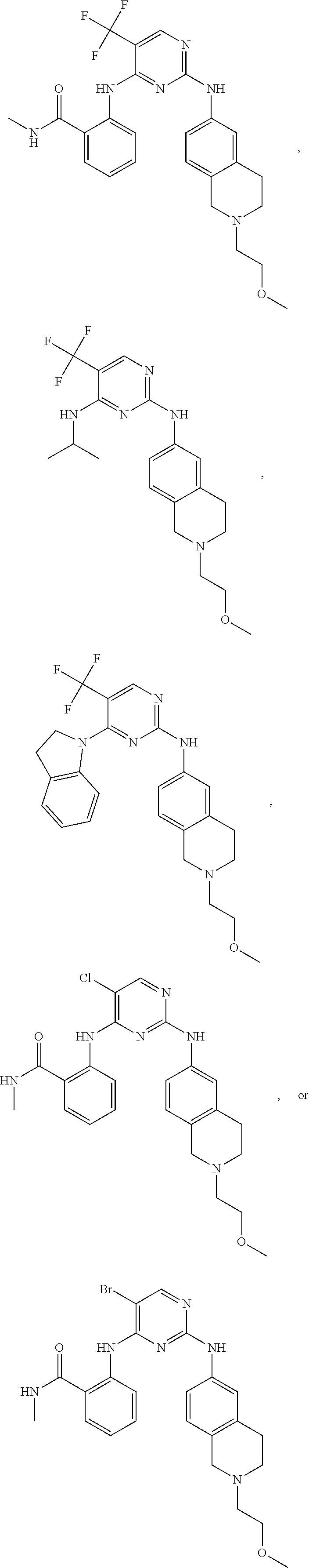
or a pharmaceutically acceptable salt thereof.

51. A compound having a structure of Formula (IIB): 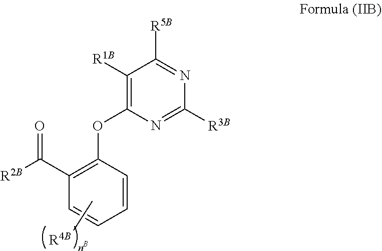
wherein:
R^{1B} is H, halogen, substituted or unsubstituted alkyl, or substituted or unsubstituted haloalkyl;
R^{2B} is substituted C₂ alkyl, substituted or unsubstituted C₃-C₁₀ alkyl, —NR^{21B}R^{22B}, or —OR²³B;
R^{3B} is —OR^{31B}, —SR^{31B}, or —NR^{32B}R^{33B};
each R^{4B} is independently halogen, —CN, —OR^{B}, —SR^{B}, —S(═O)R^{B}, —S(═O)₂R^{B}, —NO₂, —NR^{B}R^{B}, —NR^{B}S(═O)₂R^{B}, —S(═O)₂NR^{B}R^{B}, —C(═O)R^{B}, —OC(═O)R^{B}, —C(═O)C(═O)R^{B}, —C(═O)OR^{B}, —C(═O)NR^{B}OR^{B}, —OC(═O)OR^{B}, —C(═O)NR^{B}R^{B}, —OC(═O)NR^{B}R^{B}, —NR^{B}C(═O)NR^{B}R^{B}, —NR^{B}S(═O)₂NR^{B}R^{B}, —NR^{B}C(═O)R^{B}, —NR^{B}C(═O)OR^{B}, substituted or unsubstituted alkyl, substituted or unsubstituted cycloalkyl, substituted or unsubstituted heterocycloalkyl, substituted or unsubstituted aryl, or substituted or unsubstituted heteroaryl;
R^{5B} is H or halogen;
R^{21B} is —OR^{26B}, NR^{27B}R^{28B}, substituted methyl, or substituted or unsubstituted C₂-C₁₀ alkyl;
R^{22B} is H or substituted or unsubstituted alkyl; or
R^{21B} and R^{22B} are taken together with the nitrogen atom to which they are attached to form a substituted or unsubstituted heterocycloalkyl containing at least one additional heteroatom selected from the group consisting of O, N, and S;
R^{23B} is H or substituted or unsubstituted alkyl;
R^{26B} is H or substituted or unsubstituted alkyl;
R^{27B} and R^{28B} are each independently H or substituted or unsubstituted alkyl; or
R^{27B} and R^{28B} are taken together with the nitrogen atom to which they are attached to form a substituted or unsubstituted heterocycloalkyl;
R^{31B} is substituted or unsubstituted aryl or substituted or unsubstituted heteroaryl;
R^{32B} is substituted or unsubstituted aryl or substituted or unsubstituted heteroaryl;
R^{33B} is H or substituted or unsubstituted alkyl; or
R^{32B} and R^{33B} are taken together with the nitrogen atom to which they are attached to form a substituted or unsubstituted heterocycloalkyl;
each R^{B} is independently H, substituted or unsubstituted alkyl, substituted or unsubstituted heteroalkyl, substituted or unsubstituted aryl, or substituted or unsubstituted heteroaryl; and
n^{B} is an integer from 0-4;
or a pharmaceutically acceptable salt thereof.

52. The compound of claim 51, wherein R^{1B} is halogen or C₁-C₆ haloalkyl.

53. The compound of claim 51 or 52, wherein R^{1B} is Cl, Br, or —CF₃.

54. The compound of any one of claims 51-53, wherein R^{1B} is —CF₃.

55. The compound of any one of claims 51-53, wherein R^{1B} is Br.

56. The compound of any one of claims 51-55, wherein R^{2B} is —NR^{21B}R^{22B} or —OR^{23B}.

57. The compound of any one of claims 51-56, wherein R^{2B} is —NR^{21B}R^{22B}.

58. The compound of any one of claims 51-57, wherein R^{21B} is substituted methyl or substituted or unsubstituted C₂-C₆ alkyl.

59. The compound of any one of claims 51-58, wherein R^{21B} is substituted methyl or substituted C₂-C₄ alkyl.

60. The compound of any one of claims 51-59, wherein R^{21B} is

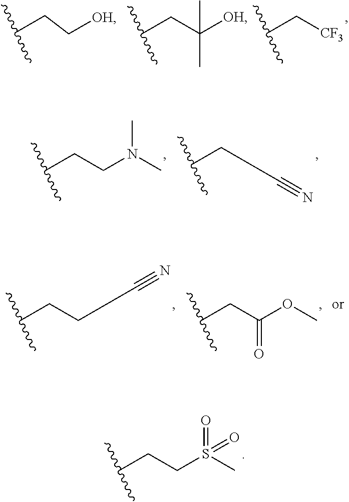

61. The compound of any one of claims 51-60, wherein R^{22B} is H or —CH₃.

62. The compound of any one of claims 51-61, wherein R^{22B} is —CH₃.

63. The compound of any one of claims 51-61, wherein R^{22B} is H.

64. The compound of any one of claims 51-56, wherein R^{2B} is —OR^{23B}.

65. The compound of any one of claims 51-56 or 64, wherein R^{23B} is H or —CH₃.

66. The compound any one of claims 51-56 or 64-65, wherein R^{23B} is —CH₃.

67. The compound of any one of claims 51-66, wherein R^{A}B is —NR^{32B}R^{33B}.

68. The compound of any one of claims 51-67, wherein R^{32B} is substituted or unsubstituted aryl or substituted or unsubstituted heteroaryl, wherein the aryl or heteroaryl is

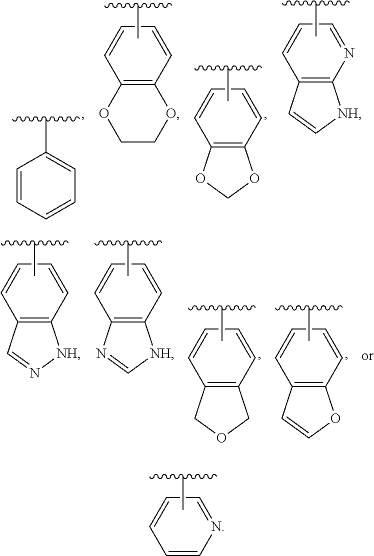

69. The compound of any one of claims 51-68, wherein R^{32B} is substituted or unsubstituted aryl or substituted or unsubstituted heteroaryl, wherein the aryl or heteroaryl is

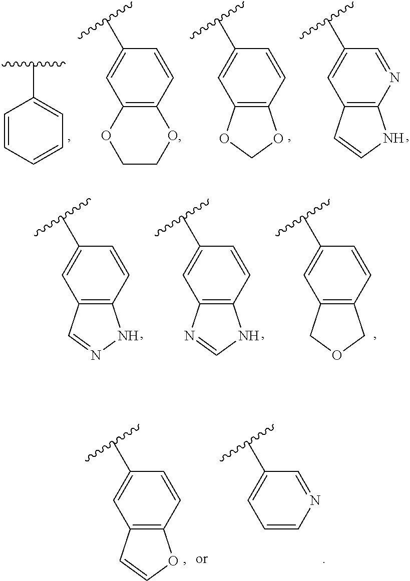

70. The compound of any one of claims 51-67, wherein R^{32B} is aryl optionally substituted with one or more halogen, —CN, —OR^{B}, —SR^{B}, —S(═O)R^{B}, —S(═O)₂R^{B}, —NO₂, —NR^{B}R^{B}, —NR^{B}S(═O)₂R^{B}, —S(═O)₂NR^{B}R^{B}, —C(═O)R^{B}, —OC(═O)R^{B}, —C(═O)C(═O)R^{B}, —C(═O)OR^{B}, —C(═O)NR^{B}OR^{B}, —OC(═O)OR^{B}, —C(═O)NR^{B}R^{B}, —OC(═O)NR^{B}R^{B}, —NR^{B}C(═O)NR^{B}R^{B}, —NR^{B}S(═O)₂NR^{B}R^{B}, —NR^{B}C(═O)R^{B}, —NR^{B}C(═O)OR^{B}, substituted or unsubstituted alkyl, substituted or unsubstituted cycloalkyl, or substituted or unsubstituted heterocycloalkyl.

71. The compound of any one of claims 51-70, wherein R^{32B} is phenyl optionally substituted with one or more halogen, —CN, —OR^{B}, —SR^{B}, —S(═O)R^{B}, —S(═O)₂R^{B}, —NO₂, —NR^{B}R^{B}, —NR^{B}S(═O)₂R^{B}, —S(═O)₂NR^{B}R^{B}, —C(═O)R^{B}, —OC(═O)R^{B}, C(═O)C(═O)R^{B}, —C(═O)OR^{B}, —C(═O)NR^{B}OR^{B}, —OC(═O)OR^{B}, —C(═O)NR^{B}R^{B}, —OC(═O)NR^{B}R^{B}, —NR^{B}C(═O)NR^{B}R^{B}, —NR^{B}S(═O)₂NR^{B}R^{B}, —NR^{B}C(═O)R^{B}, —NR^{B}C(═O)OR^{B}, substituted or unsubstituted alkyl, substituted or unsubstituted cycloalkyl, or substituted or unsubstituted heterocycloalkyl.

72. The compound of any one of claims 51-71, wherein R^{32B} is

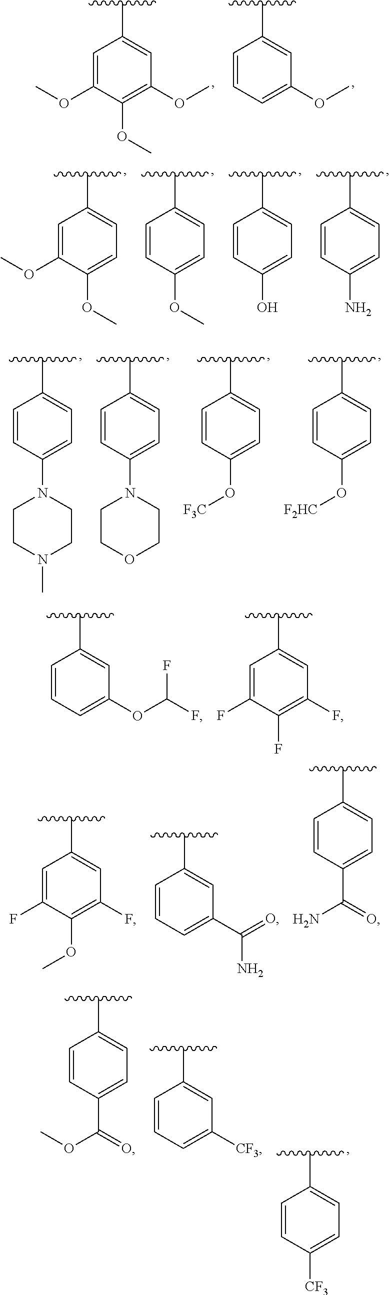

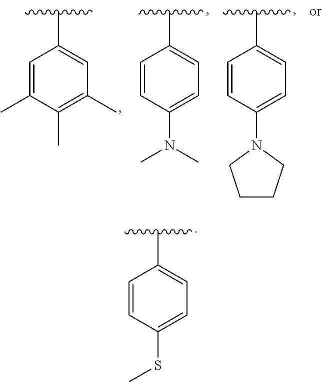

73. The compound of any one of claims 51-72, wherein R^{33B} H or C₁-C₆ alkyl.

74. The compound of any one of claims 51-73, wherein R^{33B} is H or —CH₃.

75. The compound of any one of claims 51-74, wherein R^{33B} is H.

76. The compound of any one of claims 51-75, wherein each R^{4B} is independently halogen, —CN, —OR^{B}, —C(═O)R^{B}, —OC(═O)R^{B}, —OC(═O)OR^{B}, —C(═O)NR^{B}R^{B}, OC(═O)NR^{B}R^{B}, C₁-C₆ alkyl, or C₁-C₆ haloalkyl.

77. The compound of any one of claims 51-76, wherein each R^{4B} is independently halogen, —OR^{B}, or C₁-C₆ alkyl.

78. The compound of any one of claims 51-77, wherein each R^{4B} is independently —OR^{B}.

79. The compound of any one of claims 51-78, wherein n^{B} is 0, 1, or 2.

80. The compound of any one of claims 51-79, wherein n^{B} is 0 or 1.

81. The compound of any one of claims 51-80, wherein n^{B} is 0.

82. The compound of any one of claims 51-81, wherein R^{5B} is H or F.

83. The compound of any one of claims 51-82, wherein R^{5B} is H.

84. A compound having a structure of 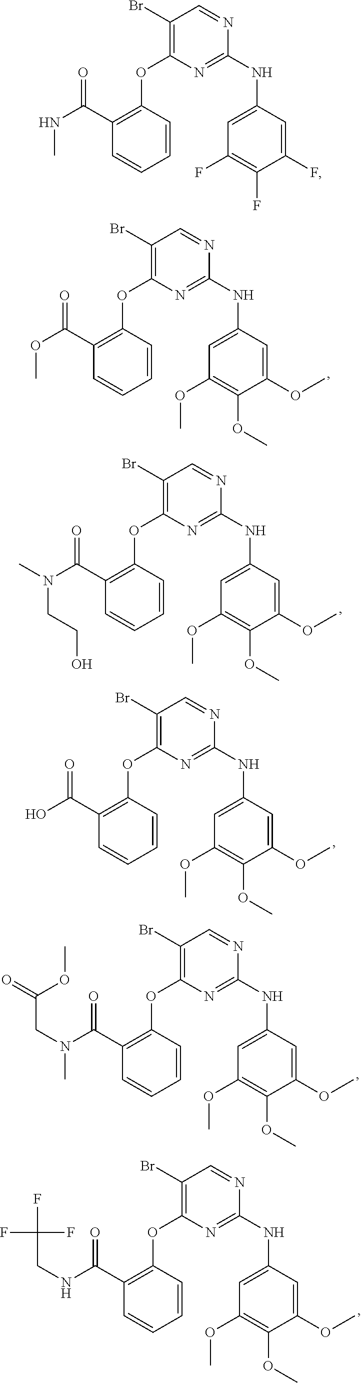 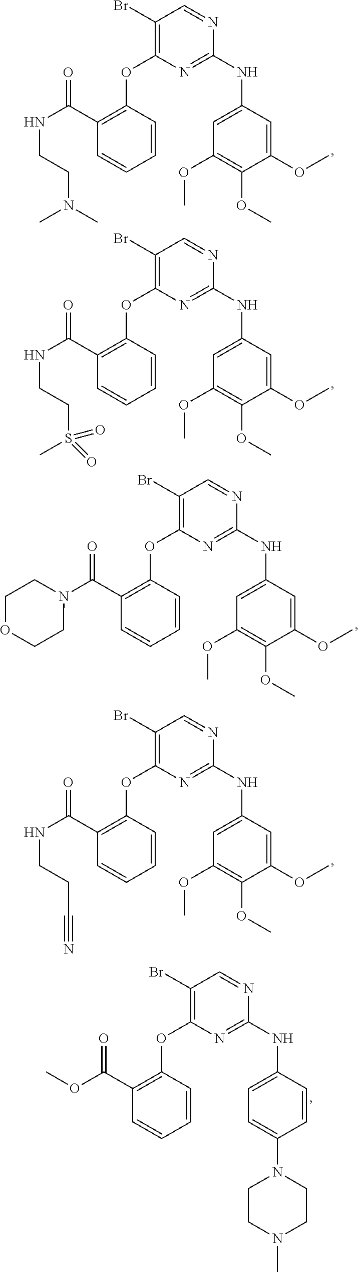 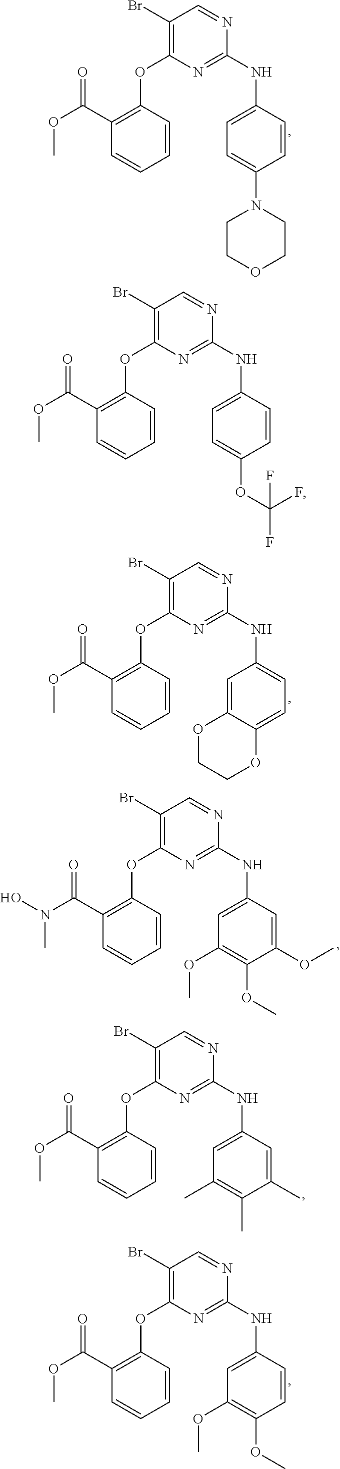 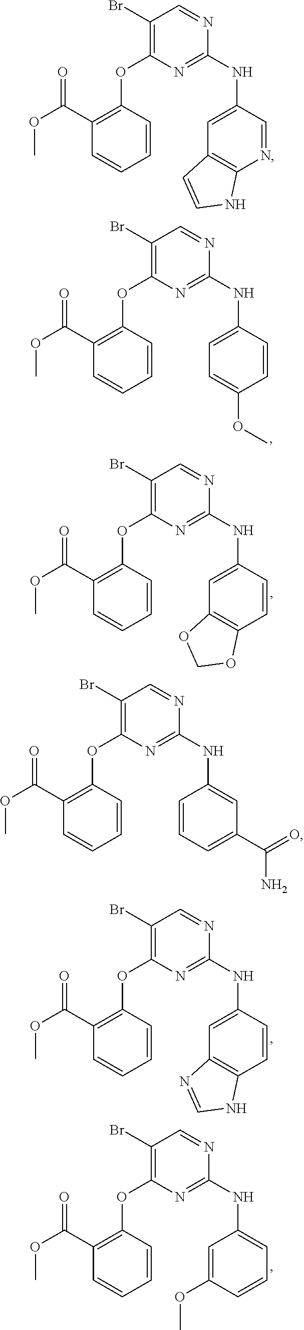 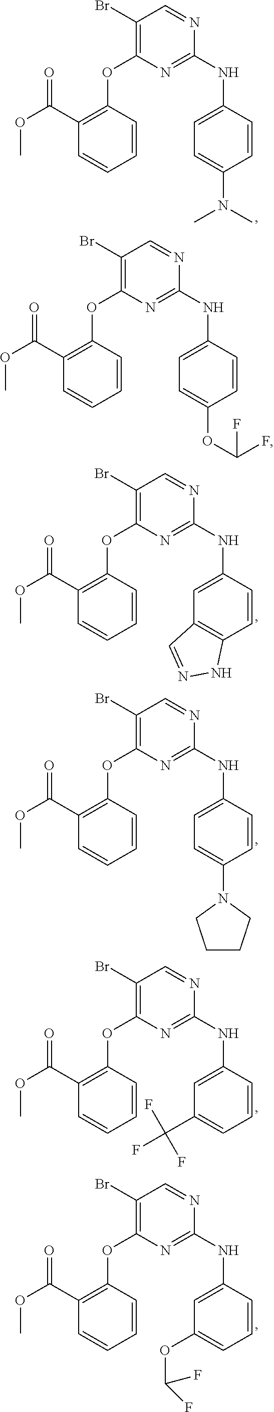 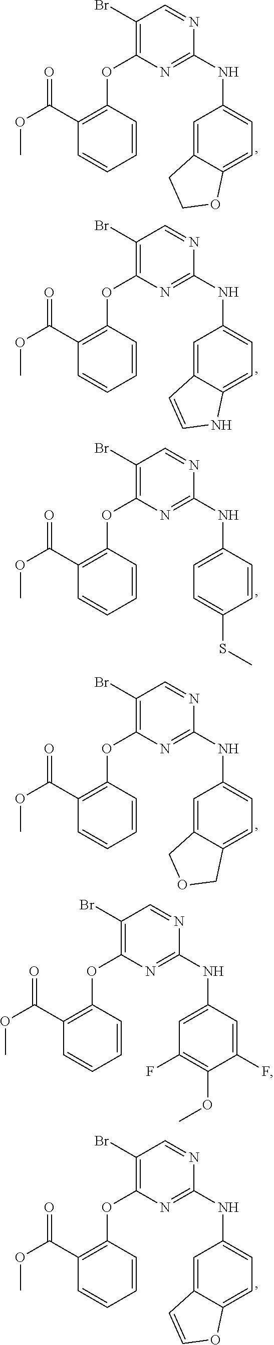 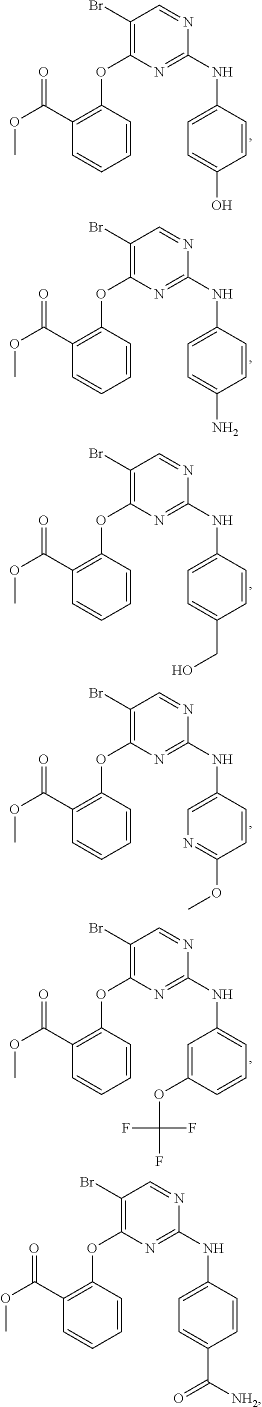 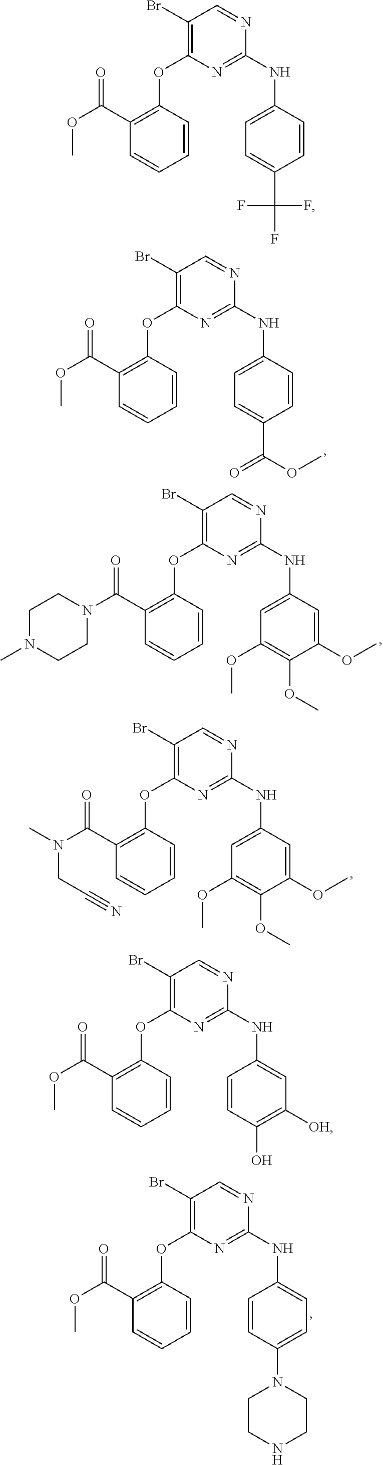 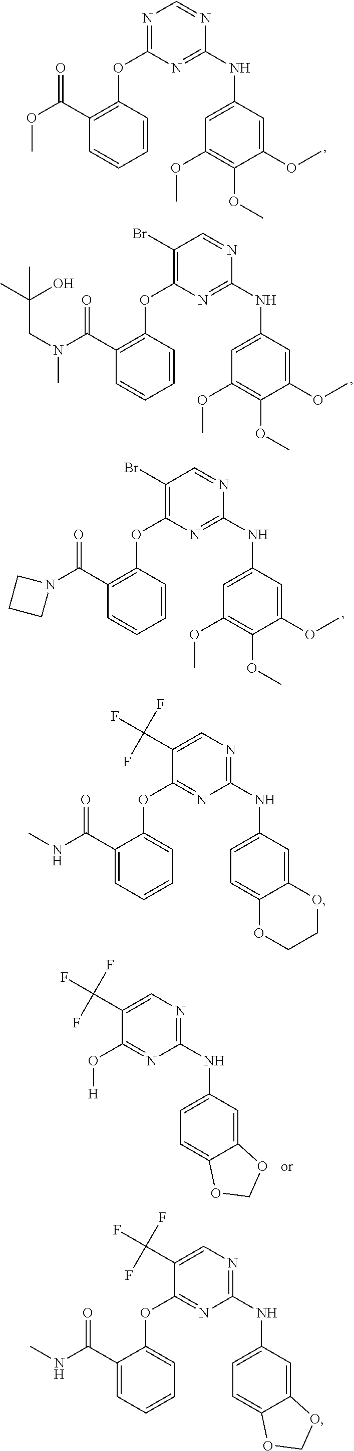
or a pharmaceutically acceptable salt thereof.

85. A compound, having a structure of Formula (IIIC): 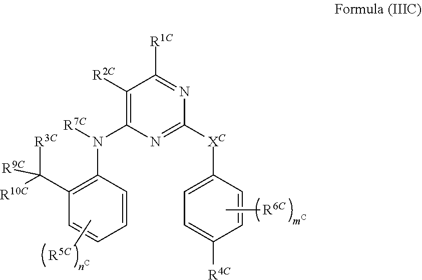
wherein:
R^{1C} is H, substituted or unsubstituted alkyl, or halogen;
R^{2C} is H, halogen, substituted or unsubstituted alkyl, or substituted or unsubstituted haloalkyl;
R^{3C} is —NR^{C}R^{C}, —OR^{C}, —O(C═O)R^{C}, —O(C═O)NR^{C}R^{C}, —NR^{C}(C═O)NR^{C}R^{C}, —NR^{C}(C═O)R^{C}, or —SR^{C};
R^{4C} is —NR^{41C}R^{42C}, —OR^{43C}, —C(═O)OR^{44C}, —C(═O)NR^{C}R^{C}, or —NR^{C}C(═O)R^{C};
each R^{5C} and R^{6C} is independently halogen, —CN, —OR^{C}, —SR^{C}, —S(═O)R^{C}, —S(═O)₂R^{C}, —NO₂, —NR^{C}R^{C}, —NR^{C}S(═O)₂R^{C}, —S(═O)₂NR^{C}R^{C}, —C(═O)R^{C}, —OC(═O)R^{C}, —C(═O)C(═O)R^{C}, —C(═O)OR^{C}, —C(═O)NR^{C}OR^{C}, —OC(═O)OR^{C}, —C(═O)NR^{C}R^{C}, —OC(═O)NR^{C}R^{C}, —NR^{C}C(═O)NR^{C}R^{C}, —NR^{C}S(═O)₂NR^{C}R^{C}, —NR^{C}C(═O)R^{C}, —NR^{C}C(═O)OR^{C}, substituted or unsubstituted alkyl, substituted or unsubstituted cycloalkyl, substituted or unsubstituted heterocycloalkyl, substituted or unsubstituted aryl, or substituted or unsubstituted heteroaryl;
R^{7C} is H or substituted or unsubstituted alkyl;
X^{C} is —O— or —NR^{8C}—;
R^{8C} is H or substituted or unsubstituted alkyl;
R^{9C} and R^{10C} are each independently H, substituted or unsubstituted alkyl, substituted or unsubstituted cycloalkyl, substituted or unsubstituted heterocycloalkyl, substituted or unsubstituted aryl, or substituted or unsubstituted heteroaryl;
or R^{9C} and R^{10C} are taken together with the carbon atom to which they are attached to form a substituted or unsubstituted cycloalkyl or substituted or unsubstituted heterocycloalkyl;
R^{41C} and R^{42C} are each independently hydrogen, alkyl, cycloalkyl, heterocycloalkyl, aryl, or heteroaryl; wherein the alkyl, cycloalkyl, heterocycloalkyl, aryl, and heteroaryl are independently optionally substituted with one or more R^{45C};
or R^{41C} and R^{42C} are taken together with the nitrogen atom to which they are attached to form a substituted or unsubstituted heterocycloalkyl;
R^{43C} is hydrogen, —CN, alkyl, cycloalkyl, heterocycloalkyl, aryl, or heteroaryl; wherein the alkyl, cycloalkyl, heterocycloalkyl, aryl, and heteroaryl are independently optionally substituted with one or more R^{45C};
R^{44C} is substituted or unsubstituted alkyl, substituted or unsubstituted cycloalkyl, substituted or unsubstituted heterocycloalkyl, substituted or unsubstituted aryl, or substituted or unsubstituted heteroaryl;
each R^{45C} is independently oxo, halogen, —CN, —OR^{C}, —S(═O)₂R^{C}, —S(═O)₂NR^{C}R^{C}, —C(═O)R^{C}, —OC(═O)R^{C}, —C(═O)OR^{C}, —OC(═O)OR^{C}, —C(═O)NR^{C}R^{C}, —OC(═O)NR^{C}R^{C}, —NR^{C}C(═O)NR^{C}R^{C}, —NR^{C}C(═O)R^{C}, alkyl, haloalkyl, or hydroxyalkyl;
each R^{C} is independently H, substituted or unsubstituted alkyl, substituted or unsubstituted heteroalkyl, substituted or unsubstituted aryl, or substituted or unsubstituted heteroaryl;
n^{C} is an integer from 0-4;
m^{C} is an integer from 0-4; and
with the proviso that when R^{4C} is —OMe and R^{2C} is halogen, then R^{3C} is not OH,
or pharmaceutically acceptable salt thereof.

86. The compound of claim 85, wherein R^{1C} is H or halogen.

87. The compound of claim 85 or 86, wherein R^{1C} is H or F.

88. The compound of any of claims 85-87, wherein R^{1C} is H.

89. The compound of any one of claims 85-88, wherein R^{2C} is halogen or C₁-C₆ haloalkyl.

90. The compound of any one claims 85-89, wherein R^{2C} is Br, Cl, or —CF₃.

91. The compound of any one of claims 85-90, wherein R^{2C} is Br.

92. The compound of any one of claims 85-91, wherein R^{3C} is —NR^{C}R^{C}, —OR^{C}, —O(C═O)R^{C}, or —O(C═O)NR^{C}R^{C}.

93. The compound of any one of claims 85-92, wherein R^{3C} is —NR^{C}R^{C} or —O(C═O)NR^{C}R^{C}.

94. The compound of any one of claims 85-93, wherein R^{3C} is —NR^{C}R^{C} and each R^{C} is independently hydrogen, substituted or unsubstituted C₁-C₆ alkyl, or substituted or unsubstituted cycloalkyl.
or both R^{C}s are taken together with the nitrogen atom to which they are attached to form a substituted or unsubstituted heterocycloalkyl.

95. The compound of any one of claims 85-94 wherein R^{3C} is —NR^{C}R^{C} each R^{C} is independently selected from H,

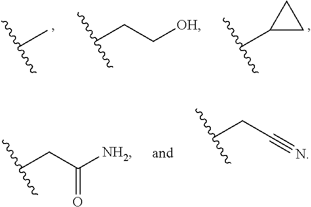

96. The compound of any one of claims 85-94, wherein R^{3C} is —NR^{C}R^{C} and both R^{C}s are taken together to form a heterocycloalkyl selected from

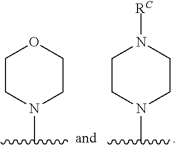

97. The compound of any one of claims 85-94 or 96, wherein R^{3C} is selected from

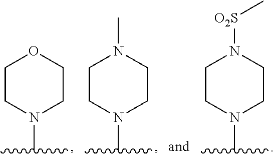

98. The compound of any one of claims 85-92, wherein R^{3C} is —OR^{C} or —O(C═O)R^{C}.

99. The compound of any one of claims 85-92 or 98, wherein R^{3C} is OR^{C} and the R^{C} of R^{3C} is hydrogen, substituted or unsubstituted C₁-C₆ alkyl, substituted or unsubstituted cycloalkyl, substituted or unsubstituted heterocycloalkyl, substituted or unsubstituted aryl, or substituted or unsubstituted heteroaryl.

100. The compound of any one of claims 85-92 or 98-99, wherein R^{3C} is —OR^{C} or —O(C═O)R^{C} and the R^{C} of R^{3C} is

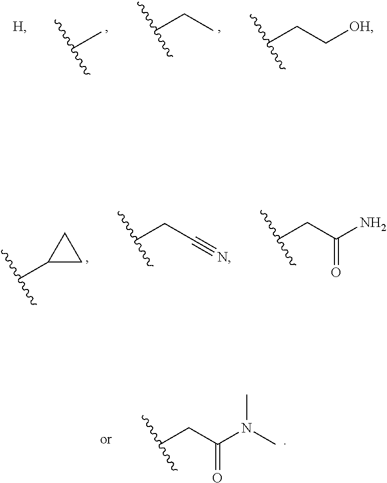

101. The compound of any one of claims 85-92 or 98-100, wherein R^{3C} is —OR^{C} or —O(C═O)R^{C} and the R^{C} of R^{3C} is

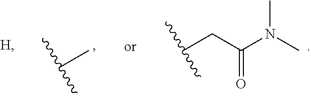

102. The compound of any one of claims 85-92, wherein R^{3C} is

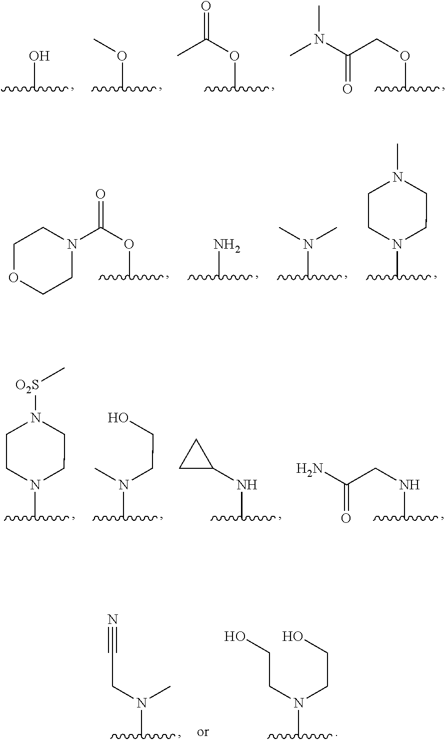

103. The compound of any one of claims 85-102, wherein R^{4C} is —NR^{41C}R^{42C}, —OR^{43C}, —C(═O)NR^{C}R^{C}, or —NR^{C}C(═O)R^{C}.

104. The compound of any one of claims 85-103, wherein R^{4C} is —NR^{41C}R^{42C}.

105. The compound of any one of claims 85-104, wherein R^{41C} and R^{42C} are each independently hydrogen, alkyl, or cycloalkyl, wherein the alkyl or cycloalkyl is optionally substituted with one or more R^{45C};
or R^{41C} and R^{42C} are taken together with the nitrogen atom to which they are attached to form a substituted or unsubstituted heterocycloalkyl.

106. The compound of any one claims 85-105, wherein each R^{41C} and R^{42C} is independently

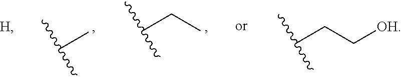

107. The compound of any one of claims 85-105, wherein R^{41C} and R^{42C} are taken together with the nitrogen atom to which they are attached to form a heterocycloalkyl, wherein the heterocycloalkyl is

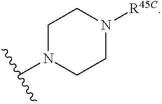

108. The compound of any one of claims 85-105 or 107, wherein R^{41C} and R^{42C} are taken together with the nitrogen atom to which they are attached to form

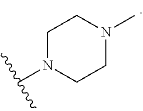 [SMILES: *N1CCN(C)CC1]

109. The compound of any one of claims 85-103, wherein R^{4C} is —OR^{43C}.

110. The compound of any one of claims 85-103 or 109, wherein R^{43C} is hydrogen or C₁-C₆ alkyl optionally substituted with one or more R^{45C}.

111. The compound of any one of claims 85-103 or 109-110, wherein R^{43C} is H, —CH₃, —CH₂CH₃, CH₂F, —CHF₂, or CF₃.

112. The compound of any one of claims 85-103, wherein R^{4C} is —C(═O)NR^{41C}R^{42C}.

113. The compound of any one of claims 85-103 or 112, wherein R^{41C} and R^{42C} are each independently hydrogen, substituted or unsubstituted C₁-C₆ alkyl, or cycloalkyl; wherein each alkyl or cycloalkyl is independently optionally substituted with one or more R^{45C}.

114. The compound of any one of claims 85-103 or 112-113, wherein R^{41C} and R^{42C} are each independently H, —CH₃, or —CH₂CH₃.

115. The compound of any one of claims 85-103, wherein R^{4C} is —NR^{C}C(═O)R^{C}.

116. The compound of any one of claims 85-103 or 115, wherein R^{4C} is —NR^{C}C(═O)R^{C} and one R^{C} of R^{4C} is H or —CH₃; and the other R^{C} of R^{4C} is substituted or unsubstituted alkyl or substituted or unsubstituted cycloalkyl.

117. The compound of any one of claims 85-103 or 115-116, wherein R^{4C} is

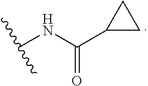 [SMILES: *NC(=O)C1CC1]

118. The compound of any one of claims 85-103, wherein R^{4C} is

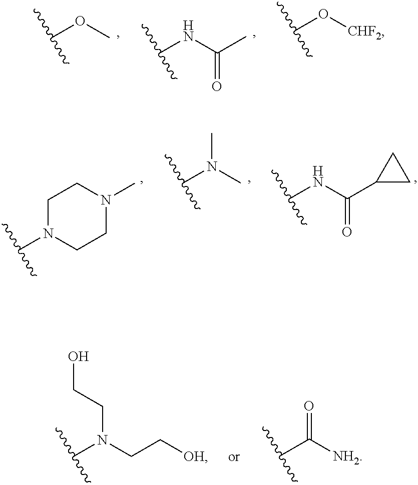

119. The compound of any one of claims 85-118, wherein each R^{5C} is independently halogen, —CN, —OR^{C}, —NR^{C}R^{C}, —NR^{C}S(═O)₂R^{C}, —S(═O)₂NR^{C}R^{C}, —OC(═O)OR^{C}, —C(═O)NR^{C}R^{C}, —OC(═O)NR^{C}R^{C}, —NR^{C}C(═O)R^{C}, or substituted or unsubstituted alkyl.

120. The compound of any one of claims 85-119, wherein each R^{5C} is independently halogen, —CN, —OR^{C}, or substituted or unsubstituted alkyl.

121. The compound of any one of claims 85-120, wherein each R^{5C} is independently halogen or —OR^{C}.

122. The compound of any one of claims 85-121, wherein each R^{5C} is independently —O(C₁-C₆ alkyl).

123. The compound of any one of claims 85-122, wherein each R^{5C} is independently —OCH₃.

124. The compound of any one of claims 85-123, wherein n^{C} is 0, 1, or 2.

125. The compound of any one of claims 85-124, wherein n^{C} is 0 or 1.

126. The compound of any one of claims 85-125, wherein n^{C} is 0.

127. The compound of any one of claims 85-126, wherein each R^{6C} is independently halogen, —CN, —OR^{C}, —C(═O)R^{C}, —OC(═O)R^{C}—C(═O)OR^{C}, —OC(═O)OR^{C}, —OC(═O)NR^{C}R^{C}, or substituted or unsubstituted alkyl.

128. The compound of any one of claims 85-127, wherein each R^{6C} is independently halogen or —OR^{C}.

129. The compound of any one of claims 85-128, wherein each R^{6C} is —O(C₁-C₆ alkyl).

130. The compound of any one of claims 85-129, wherein m^{C} is 0, 1, or 2.

131. The compound of any one of claims 85-130, wherein m^{C} is 2.

132. The compound of any one of claims 85-131, wherein m^{C} is 2 and R^{6C} is —OCH₃.

133. The compound of any one of claims 85-130, wherein m^{C} is 0 or 1.

134. The compound of any one of claims 85-130 or 133, wherein m^{C} is 0.

135. The compound of any one of claims 85-134, wherein R^{7C} is H or —CH₃.

136. The compound of any one of claims 85-135, wherein R^{7C} is H.

137. The compound of any one of claims 85-136, wherein X^{C} is —NR⁸—.

138. The compound of any one of claims 85-137, wherein R^{8C} is H or —CH₃.

139. The compound of any one of claims 85-138, wherein R^{8C} is H.

140. The compound of any one of claims 85-139, wherein R^{9C} and R^{10C} are each independently H or substituted or unsubstituted alkyl.

141. The compound of any one of claims 85-140, wherein R^{9C} and R^{10C} are each independently H or C₁-C₆ alkyl.

142. The compound of any one of claims 85-141, wherein R^{9C} is —CH₃ and R^{10C} is H.

143. The compound of any one of claims 85-141, wherein R^{9C} and R^{10C} are each H.

144. A compound having a structure of 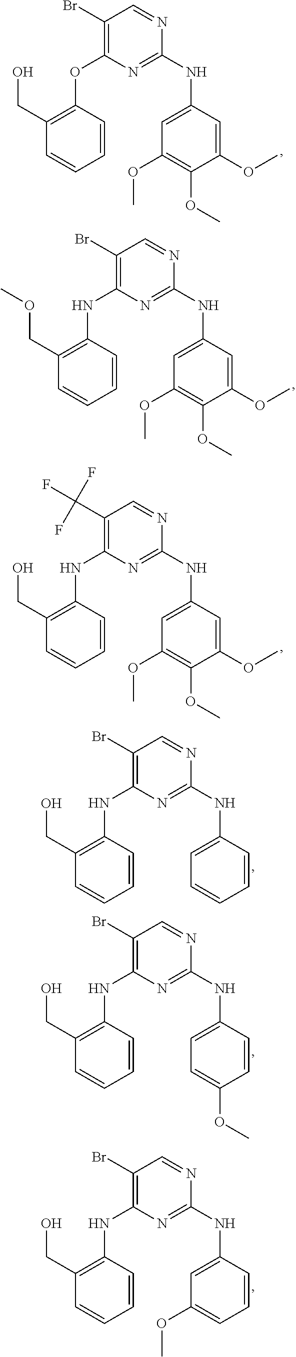 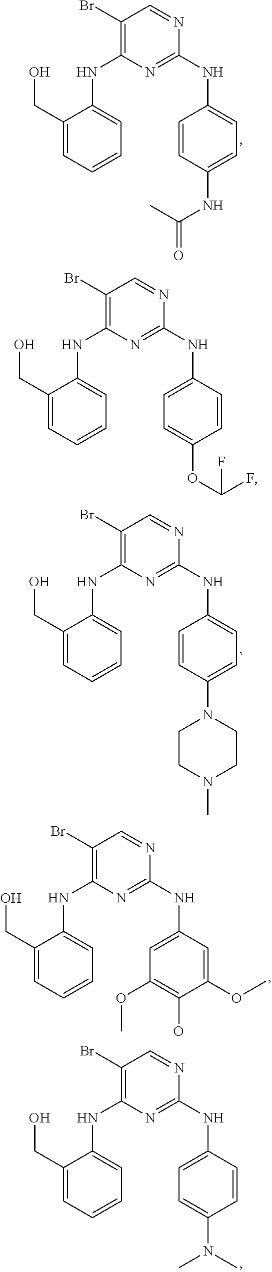 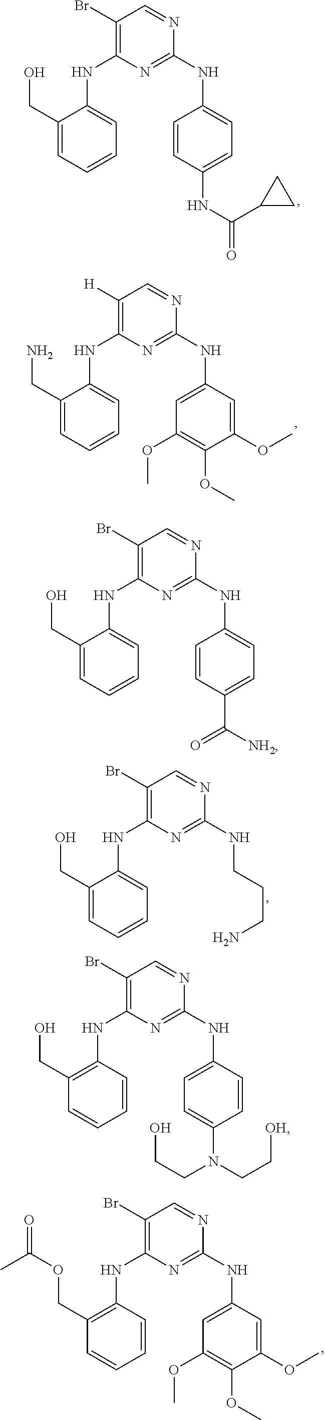 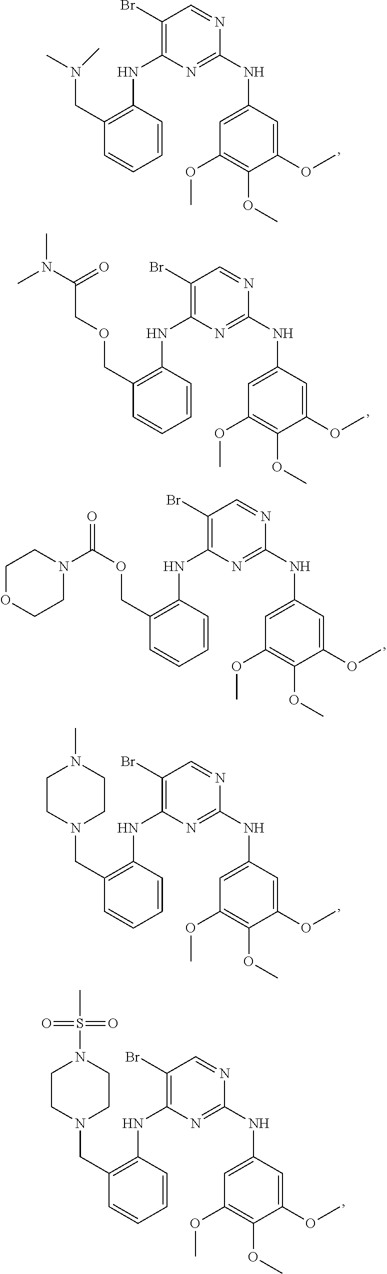 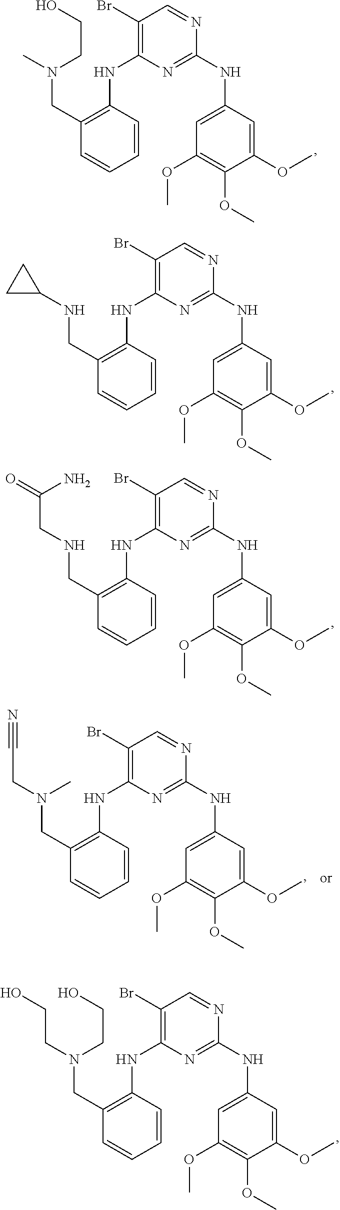
or a pharmaceutically acceptable salt thereof.

145. A compound having a structure of Formula (IVD): 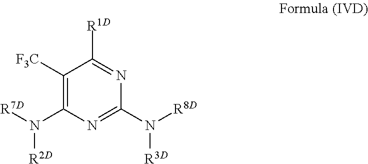
wherein:
R^{1D} is H or halogen;
R^{2D} is 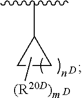
m^{D} is an integer from 1 to 3;
n^{D} is an integer from 1 to 6;
R^{3D} is 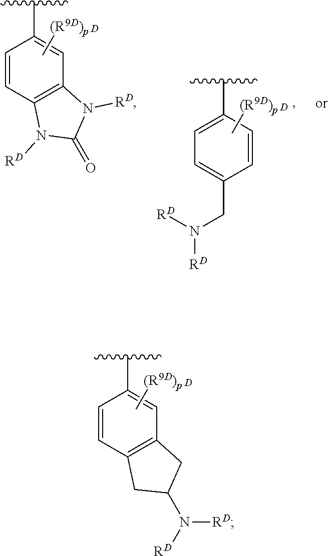
R^{7D} and R^{8D} are each independently H or substituted or unsubstituted alkyl;
each R^{9D} is independently halogen, —CN, —OR^{D}, S(═O)₂R^{D}, —NR^{D}R^{D}, —S(═O)₂NR^{D}R^{D}, —C(═O)R^{D}, —OC(═O)R^{D}, —C(═O)OR^{D}, —OC(═O)OR^{D}, —C(═O)NR^{D}R^{D}, OC(═O)NR^{D}R^{D}, —NR^{D}C(═O)NR^{D}R^{D}, —NR^{D}C(═O)R^{D}, alkyl, haloalkyl, or hydroxyalkyl;
p^{D} is an integer from 0 to 2;
each R^{20D} is independently halogen, —CN, —OR^{D}, S(═O)₂R^{D}, —S(═O)₂NR^{D}R^{D}, —C(═O)R^{D}, —OC(═O)R^{D}, —C(═O)OR^{D}, —OC(═O)OR^{D}, —OC(═O)NR^{D}R^{D}, —NR^{D}C(═O)NR^{D}R^{D}, —NR^{D}C(═O)R^{D}, alkyl, haloalkyl, or hydroxyalkyl;
each R^{D} is independently H, substituted or unsubstituted alkyl, substituted or unsubstituted heteroalkyl, substituted or unsubstituted aryl, or substituted or unsubstituted heteroaryl
or pharmaceutically acceptable salt thereof.

146. The compound of claim 145, wherein R^{1D} is H or fluorine.

147. The compound of any one of claims 145 or 146, wherein R^{1D} is H.

148. The compound of any one of claims 145-147, wherein R^{2D} is

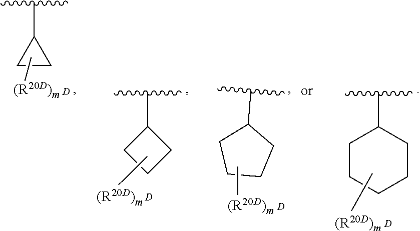

149. The compound of any one of claims 145-148, wherein R² is

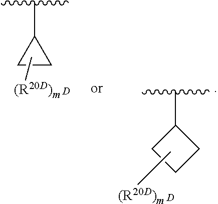

150. The compound of any one of claims 145-149, wherein R² is

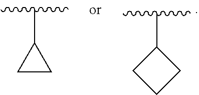

151. The compound of any one of claims 145-149, wherein each R^{20D} is independently halogen, —CN, —OR^{D}, —C(═O)R^{D}, —OC(═O)R^{D}, —OC(═O)OR^{D}, —OC(═O)NR^{D}R^{D}, —NR^{D}C(═O)R^{D}, or C₁-C₆ alkyl.

152. The compound of any one of claims 145-149 or 151, wherein each R^{20D} is independently halogen, —CN, —OR^{D}, or C₁-C₆ alkyl.

153. The compound of any one of claims 145-149 or 151-152, wherein m^{D} is 0 or 1.

154. The compound of any one of claims 145-153, wherein m^{D} is 0.

155. The compound of any one of claims 145-154, wherein R^{3D} is

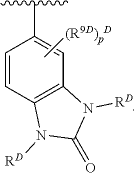

156. The compound of any one of claims 145-155, wherein each R^{D} is independently H or —CH₃.

157. The compound of any one of claims 145-156, wherein each R^{D} is independently H.

158. The compound of any one of claims 145-154, wherein R^{3D} is

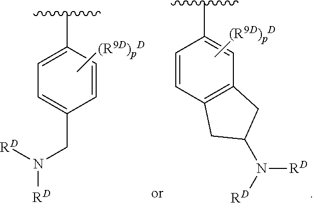

159. The compound of any one of claims 145-154 or 158, wherein each R^{D} is independently hydrogen, —C(═O)C₁-C₆ alkyl, —C(═O)OC₁-C₆ alkyl, or C₁-C₆ alkyl, wherein each alkyl of each R^{D} is substituted or unsubstituted.

160. The compound of any one of claims 145-154 or 158-159, wherein each R^{D} and R^{D} is independently H or —CH₃.

161. The compound of any one of claims 145-154 or 158-159, wherein one R^{D} is H and one

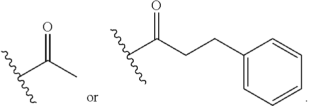

162. The compound of any one of claims 145-161, wherein R^{7D} and R^{8D} are each independently H or —CH₃.

163. The compound of any one of claims 145-162, wherein R^{7D} and R^{8D} are each independently H.

164. The compound of any one of claims 145-163, wherein each R^{9D} is independently halogen, —CN, —OR^{D}, —NR^{D}R^{D}, —C(═O)R^{D}, —OC(═O)R^{D}, —OC(═O)OR^{D}, —C(═O)NR^{D}R^{D}, —NR^{D}C(═O)R^{D}, or C₁-C₆ alkyl.

165. The compound of any one claims 145-164, wherein each R^{9D} is independently halogen, —CN, —OR^{D}, or C₁-C₆ alkyl.

166. The compound of any one of claims 145-165, wherein p^{D} is 0 or 1.

167. The compound of any one of claims 145-166, wherein p^{D} is 0.

168. A compound having a structure of 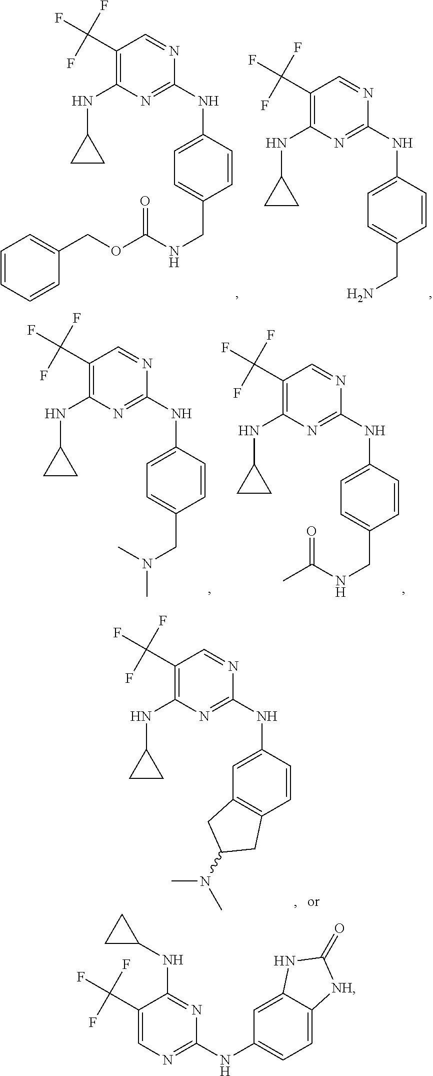
or a pharmaceutically acceptable salt thereof.

169. A compound having a structure of Formula (VE): 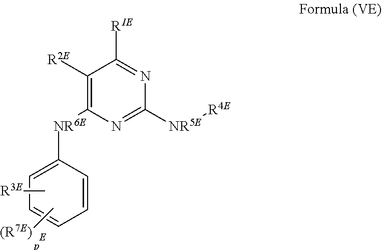
wherein:
R^{1E} is H, nitrile, or halogen;
R^{2E} is halogen, nitrile, methyl, cyclopropyl, or —CF₃;
R^{3E} is halogen, 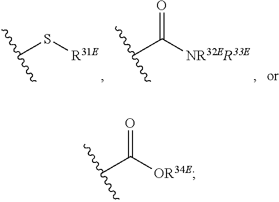
R^{4E} is is aryl substituted with one or more —OR^{35E}, substituted or unsubstituted cycloalkyl, or substituted or unsubstituted heterocycloalkyl, 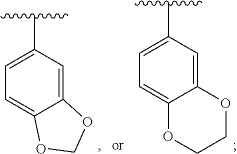 [SMILES: *c1ccc2c(c1)OCO2 ; *c1ccc2c(c1)OCCO2]
R^{5E} and R^{6E} are each independently H or C₁-C₆ alkyl;
each R^{7E} is independently halogen, —CN, —OR^{E}, —S(═O)₂R^{E}, —NR^{E}R^{E}, —S(═O)₂NR^{E}R^{E}, —C(═O)R^{E}, —OC(═O)R^{E}, —C(═O)OR^{E}, —OC(═O)OR^{E}, —C(═O)NR^{E}R^{E}, OC(═O)NR^{E}R^{E}, —NR^{E}C(═O)NR^{E}R^{E}, —NR^{E}C(═O)R^{E}, C₁-C₆ alkyl, C₁-C₆ haloalkyl, or C₁-C₆ hydroxyalkyl;
p^{E} is an integer from 0 to 3;
R^{31E} is H, C₁-C₆ alkyl, or cycloalkyl;
R^{32E} and R^{33E} are each independently H, substituted or unsubstituted C₁-C₆ alkyl, or cycloalkyl;
R^{34E} is H, C₁-C₆ alkyl, or cycloalkyl;
each R^{35E} is independently substituted or unsubstituted alkyl, or substituted or unsubstituted heteroalkyl;
each R^{E} is independently hydrogen, C₁-C₆ alkyl, cycloalkyl, heterocycloalkyl, aryl, or heteroaryl; wherein the alkyl, cycloalkyl, heterocycloalkyl, aryl, and heteroaryl are independently optionally substituted with one or more halogen, —OH, —NH₂, substituted amino, cycloalkyl, oxo, or C₁-C₆ alkyl;
wherein when R^{3E} is 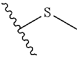 [SMILES: *SC]
then R^{2E} is not Br; and
wherein when R^{3E} is 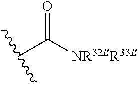
then R^{2E} is not Cl and R^{4E} is not 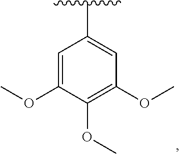
or pharmaceutically acceptable salt thereof.

170. The compound of claim 169, wherein R^{1E} is H or F.

171. The compound of any one of claims 169 or 170, wherein R^{1E} is H.

172. The compound of any one of claims 169-171, wherein R^{2E} is Cl, Br, or —CF₃.

173. The compound of any one of claims 169-172, wherein R^{2E} is Br or —CF₃.

174. The compound of any one of claims 169-173, wherein R^{3E} is —SR^{31E}.

175. The compound of any one of claims 169-174, wherein R^{3E} is —SH, —SCH₃, or —SCH₂CH₃.

176. The compound of any one of claims 169-175, wherein R^{3E} is —SCH₃.

177. The compound of any one of claims 169-173, wherein R^{3E} is

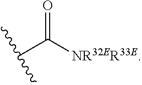

178. The compound of any one of claims 169-173 or 177, wherein R^{3E} is

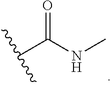 [SMILES: *C(=O)NC]

179. The compound of any one of claims 169-173, wherein R^{3E} is

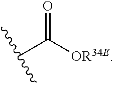

180. The compound of any one of claims 169-173 or 179, wherein R^{3E} is

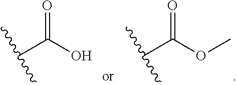

181. The compound of any one of claims 169-180, wherein R^{4E} is

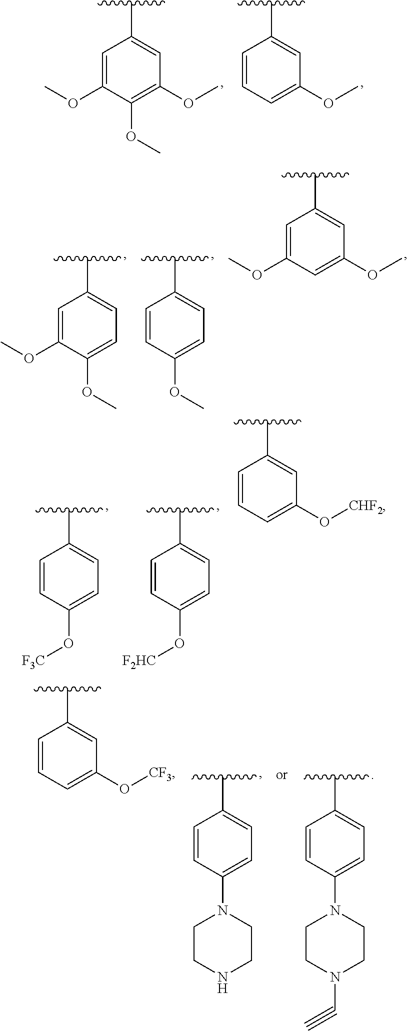

182. The compound of any one of claims 169-180, wherein R^{4E} is

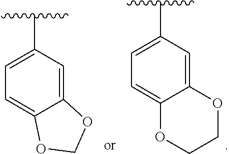 [SMILES: *c1ccc2c(c1)OCO2 ; *c1ccc2c(c1)OCCO2]

183. The compound of any one of claims 169-182, wherein R^{5E} and R^{6E} are each independently H or —CH₃.

184. The compound of any one of claims 169-183, wherein R^{5E} and R^{6E} are each independently H.

185. The compound of any one of claims 169-184, wherein each R^{7E} is independently halogen, —CN, —OR^{E}, —NR^{E}R^{E}, —C(═O)R^{E}, —OC(═O)R^{E}, —C(═O)OR^{E}, —C(═O)NR^{E}R^{E}, or C₁-C₆ alkyl.

186. The compound of any one of claims 169-185, wherein each R^{7E} is independently halogen, —OR^{E}, —OC(═O)R^{E}, or C₁-C₆ alkyl.

187. The compound of any one of claims 169-186, wherein each R^{7E} is independently halogen or —OCH₃.

188. The compound of any one of claims 169-187, wherein p^{E} is 0 or 1.

189. The compound of any one of claims 169-188, wherein p^{E} is 0.

190. A compound having a structure of 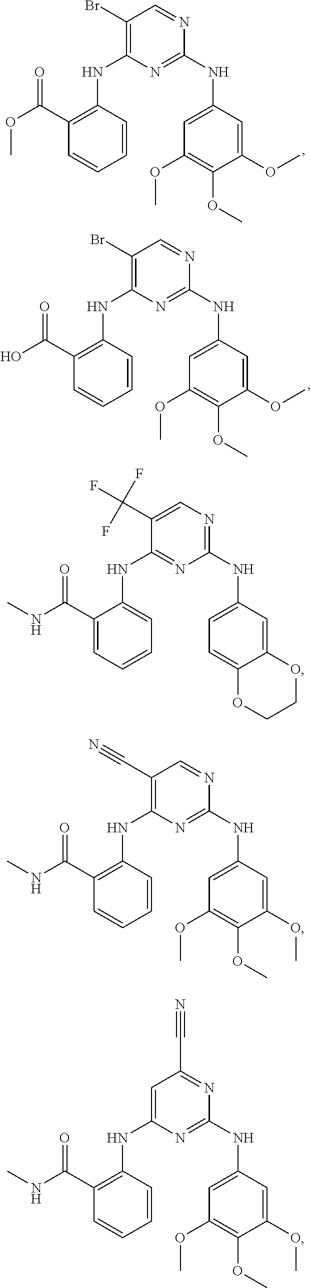 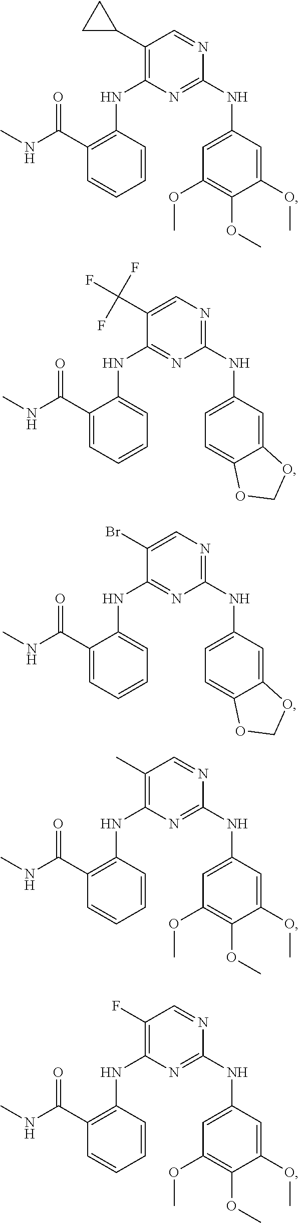 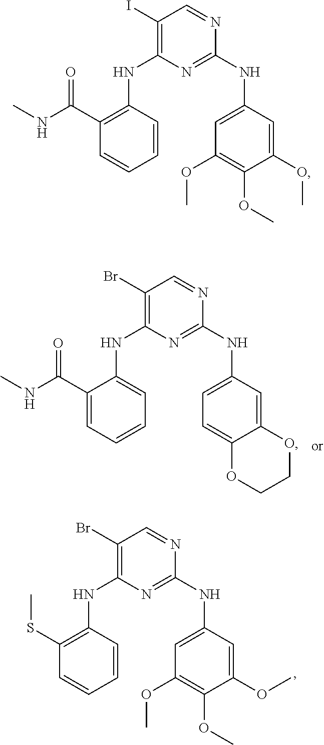
or a pharmaceutically acceptable salt thereof.

191. A pharmaceutical composition comprising the compound or pharmaceutically acceptable salt thereof of any one of the preceding claims and a pharmaceutically acceptable carrier.

192. A method of treating a ULK1 mediated disease in a subject in need thereof, the method comprising administering to the subject a compound of any one of claims 1-190 or pharmaceutical composition of claim 191.

193. The method of claim 192, wherein the ULK1 mediated disease is characterized by abnormal autophagy.

194. The method of claim 193, wherein the abnormal autophagy has been therapeutically induced.

195. The method of any one of claims 192-194, wherein the disease is cancer.

196. The method of claim 195, wherein the cancer is lung cancer, breast cancer, or pancreatic cancer.

197. The method of claim 196, wherein the lung cancer is non-small cell lung cancer, the breast cancer is triple negative breast cancer, or the pancreatic cancer is pancreatic ductal adenocarcinoma.

198. The method of any one of claims 192-194, wherein the disease is Tuberous Sclerosis Complex (TSC) or lymphangioleiomyomatosis (LAM).

199. The method of any one of claims 192-198, wherein the compound is co-administered with an additional therapeutic agent.

200. The method of claim 199, wherein the standard of care therapy is an mTOR inhibitor, carboplatin, an MEK inhibitor, or a PARP inhibitor.

201. The method of any of claims 199-200, wherein the additional therapeutic agent is a standard of care therapy.

202. The method of any one of claims 192-201, wherein administering the compound or pharmaceutical composition degrades ATG13 in the subject.

203. Use of a compound of any one of claims 1-190 in the preparation of a medicament for the treatment of a ULK1 mediated disease.

204. A compound of any one of claims 1-190, for use in the treatment of a ULK1 mediated disease.
